(19) United States
(12) Patent Application Publication
(10) Pub. No.: US 2009/0087436 A1
(43) Pub. Date: Apr. 2, 2009

(54) COMPOSITIONS AND METHODS FOR TREATING DISEASES

(75) Inventors: Jean-Marc Roch, Salt Lake City, UT (US); Karen Heichman, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/862,978

(22) Filed: Sep. 27, 2007

Related U.S. Application Data
(63) Continuation-in-part of application No. 11/035,152, filed on Jan. 12, 2005, now abandoned.
Application No. 11/035,152 is a continuation-in-part of application No. 10/194,964, filed on Jul. 15, 2002, now abandoned.
Application No. 11/035,152 is a continuation-in-part of application No. 10/194,966, filed on Jul. 15, 2002, now abandoned.
Application No. 11/035,152 is a continuation-in-part of application No. 10/194,967, filed on Jul. 15, 2002, now abandoned.
Application No. 11/035,152 is a continuation-in-part of application No. 10/194,998, filed on Jul. 15, 2002, now abandoned.
Application No. 11/035,152 is a continuation-in-part of application No. 10/285,655, filed on Oct. 30, 2002, now abandoned.
Application No. 11/035,152 is a continuation-in-part of application No. 10/285,932, filed on Oct. 31, 2002, now abandoned.
(60) Provisional application No. 60/304,737, filed on Jul. 13, 2001.
(60) Provisional application No. 60/304,767, filed on Jul. 13, 2001.
(60) Provisional application No. 60/304,775, filed on Jul. 13, 2001.
(60) Provisional application No. 60/304,795, filed on Jul. 13, 2001.
(60) Provisional application No. 60/330,758, filed on Oct. 30, 2001.
(60) Provisional application No. 60/330,828, filed on Oct. 31, 2001.

(51) Int. Cl. A61K 39/395; C07K 14/00; G01N 33/68; C12Q 1/02; G01N 33/48; A61P 25/00; A61P 9/00; A61P 3/04; A61P 3/10; C12Q 1/68; A61K 31/7052; C12N 15/87; C40B 40/10

(52) U.S. Cl. 424/139.1; 530/402; 436/86; 435/29; 435/440; 506/18; 514/44; 436/94; 435/6

(57) ABSTRACT

Protein complexes are provided comprising at least one interacting pair of proteins. The protein complexes are useful in screening assays for identifying compounds effective in modulating the protein complexes, and in treating and/or preventing diseases and disorders associated with the protein complexes and/or their constituent interacting members.

### RELATED U.S. APPLICATIONS

This application is a continuation-in-part of U.S. patent application Ser. No. 11/035,152 filed on 12 Jan. 2005; which was a continuation-in-part of U.S. patent application Ser. No. 10/194,964, filed on 15 Jul. 2002, which is related to U.S. provisional patent application Ser. No. 60/304,737, filed on 13 Jul. 2001; and was a continuation-in-part of U.S. patent application Ser. No. 10/194,966, filed on 15 Jul. 2002, which is related to U.S. provisional patent application Ser. No. 60/304,767, filed on 13 Jul. 2001; and was a continuation-in-part of U.S. patent application Ser. No. 10/194,967, filed on 15 Jul. 2002, which is related to U.S. provisional patent application Ser. No. 60/304,775, filed on 13 Jul. 2001; and was a continuation-in-part of U.S. patent application Ser. No. 10/194,998 filed on 15 Jul. 2002, which is related to U.S. provisional patent application Ser. No. 60/304,795, filed on 13 Jul. 2001; and was a continuation-in-part of U.S. patent application Ser. No. 10/285,655, filed on 30 Oct. 2002, which is related to U.S. provisional patent application Ser. No. 60/330,758 filed on Oct. 30, 2001; and was a continuation-in-part of U.S. patent application Ser. No. 10/285,932, filed on 31 Oct. 2002, which is related to U.S. provisional patent application Ser. No. 60/330,828 filed on Oct. 31, 2001; all of which are incorporated by reference herein in their entirety.

### SEQUENCE LISTING

This application is being filed with a formal Sequence Listing submitted electronically as a text file. This text file, which is named “1837-01-1X-2007-09-27-SEQ-LIST-ST25.txt”, was created on Sep. 27, 2007 and is 102,400 bytes in size. Its contents are incorporated by reference herein in their entirety.

### FIELD OF THE INVENTION

The present invention generally relates to methods and compositions for treating diseases, particularly to methods of using and modulating specific proteins and protein-protein interactions for purposes of drug screening and treatment of diseases.

### BACKGROUND OF THE INVENTION

Most drug discovery efforts today employ approaches to empirically identify small molecules that bind particular biological targets in vitro. These approaches generally involve “primary” high throughput screens designed to search vast combinatorial libraries of small molecules for “lead compounds” that often show a relatively weak affinity for the chosen target. However, once such lead compounds are identified in a “primary” high throughput screen, they can be subjected to further iterative rounds of chemical modification and testing by the process known to medicinal chemists as Structure Activity Relationship, or SAR. Generally, after several rounds of SAR-guided modification and in vitro screening, a set of optimized and related drug candidate compounds are subjected to the next phase of testing. This next phase generally involves the in vivo screening of the drug candidates in cell-based assays specifically designed to test the efficacy, toxicity and bioavailablity of the candidates. If the desired effects are obtained with reasonable dosages in these cell-based assays, animal studies are then initiated to determine whether the drug candidates have the desired activity in vivo. Only after careful study in well-defined animal models will a drug candidate be administered to humans in carefully regulated clinical trials.

The success or failure of a drug discovery program is heavily dependent on the identification and selection of druggable targets. In addition, once an appropriate drug target has been identified an efficient, preferably high throughput, screening assay needs to be established for drug screening against that particular drug target, which can be often be difficult to pragmatically achieve. The present invention provides novel drug targets for diseases such as Alzheimer's disease (including mild cognitive impairment), Huntington's disease, Parkinson's disease, schizophrenia, depression, dementia and other neurodegenerative diseases, as well as diabetes, obesity and coronary artery disease and discloses screening assays for identifying potential drugs that may be effective against the diseases through modulating the drug targets.

### SUMMARY OF THE INVENTION

The present invention is based on the discovery of novel interactions between pairs of proteins described in the tables below. The specific interactions lead to the identification of desirable novel drug targets. Specifically, the interactions implicate several newly discovered interactors in the Alzheimer's disease (including mild cognitive impairment), Huntington's disease, Parkinson's disease, schizophrenia, depression, dementia and other neurodegenerative diseases, as well as diabetes, obesity and coronary artery disease and other disease pathways, and suggest that modulation of such interactors may lead to alleviation or treatment of the diseases. In addition, the interactions can lead to the formation of protein complexes both in vitro and in vivo. This enables novel approaches for drug screening to select not only drug candidates that modulate the well-known drug targets used as baits in the interaction discovery, but also modulators of the newly discovered interactors and protein-protein interactions. For example, screening assays can be established based on the interaction between a protein known to be involved in a disease pathway and one of its newly discovered protein interactors. Compounds that modulate or interact with the known target protein can be selected based on their ability either to compete with a newly discovered interactor for interaction with the target protein, or to promote the interaction between the target protein and the interactor.

Thus, in accordance with a first aspect of the present invention, isolated protein complexes are provided which are formed by the protein-protein interactions provided in the tables. In addition, homologues, derivatives, and fragments of the interacting proteins may also be used in forming protein complexes. In a specific embodiment, fragments of an interacting pair of proteins described in the tables containing regions responsible for the protein-protein interaction are used in forming a protein complex of the present invention. In another embodiment, at least one interacting protein member in a protein complex of the present invention is a fusion protein containing a protein in the tables or a homologue, derivative, or fragment thereof. In yet another embodiment, a protein complex is provided from a hybrid protein, which comprises, covalently linked together, directly or through a linker, a pair of interacting proteins described in the tables, or homologues, derivatives, or fragments thereof. In addition, nucleic acids encoding the hybrid protein are also provided.

In yet another aspect, the present invention also provides a method for making the protein complexes. The method includes the steps of providing the first protein and the second protein in the protein complexes of the present invention and contacting said first protein with said second protein. In addition, the protein complexes can be prepared by isolation or purification from tissues and cells or produced by recombinant expression of their protein members. The protein complexes can be incorporated into a protein microchip or microarray, which are useful in large-scale high throughput screening assays involving the protein complexes.

In accordance with a second aspect of the invention, antibodies are provided that are immunoreactive with a protein complex of the present invention. In one embodiment, an antibody is selectively immunoreactive with a protein complex of the present invention. In another embodiment, a bifunctional antibody is provided that has two different antigen binding sites, each being specific to a different interacting protein member in a protein complex of the present invention. The antibodies of the present invention can take various forms including polyclonal antibodies, monoclonal antibodies, chimeric antibodies, antibody fragments such as Fv fragments, single-chain Fv fragments (scFv), Fab′ fragments, and F(ab′)₂ fragments. Preferably, the antibodies are partially or fully humanized antibodies. The antibodies of the present invention can be readily prepared using procedures generally known in the art. For example, recombinant libraries such as phage display libraries and ribosome display libraries may be used to screen for antibodies with desirable specificities. In addition, various mutagenesis techniques such as site-directed mutagenesis and PCR diversification may be used in combination with the screening assays.

The present invention also provides detection methods for determining whether there is any aberration in a patient with respect to a protein complex formed by one or more interactions provided in accordance with this invention. In one embodiment, the method comprises detecting an aberrant concentration of the protein complexes of the present invention. Alternatively, the concentrations of one or more interacting protein members (at the protein or cDNA or mRNA level) of a protein complex of the present invention are measured. In addition, the cellular localization, or tissue or organ distribution of a protein complex of the present invention is determined to detect any aberrant localization or distribution of the protein complex. In another embodiment, mutations in one or more interacting protein members of a protein complex of the present invention can be detected. In particular, it is desirable to determine whether the interacting protein members have any mutations that will lead to, or are associated with, changes in the functional activity of the proteins or changes in their binding affinity to other interacting protein members in forming a protein complex of the present invention. In yet another embodiment, the binding constant of the interacting protein members of one or more protein complexes is determined. A kit may be used for conducting the detection methods of the present invention. Typically, the kit contains reagents useful in any of the above-described embodiments of the detection methods, including, e.g., antibodies specific to a protein complex of the present invention or interacting members thereof, and oligonucleotides selectively hybridizable to the cDNAs or mRNAs encoding one or more interacting protein members of a protein complex. The detection methods may be useful in diagnosing a disease or disorder such as Alzheimer's disease (including mild cognitive impairment), Huntington's disease, Parkinson's disease, schizophrenia, depression, dementia and other neurodegenerative diseases, as well as diabetes, obesity and coronary artery disease, staging the disease or disorder, or identifying a predisposition to the disease or disorder.

The present invention also provides screening methods for selecting modulators of a protein complex provided according to the present invention. Screening methods are also provided for selecting modulators of the individual interacting proteins. The compounds identified in the screening methods of the present invention can be useful in modulating the functions or activities of the individual interacting proteins, or the protein complexes of the present invention. They may also be effective in modulating the cellular processes involving the proteins and protein complexes, and in preventing or ameliorating diseases or disorders such as Alzheimer's disease (including mild cognitive impairment), Huntington's disease, Parkinson's disease, schizophrenia, depression, dementia and other neurodegenerative diseases, as well as diabetes, obesity and coronary artery disease.

Thus, test compounds may be screened in in vitro binding assays to identify compounds capable of binding a protein complex of the present invention, or its individual interacting protein members. The assays may include the steps of contacting the protein complex with a test compound and detecting the interaction between the interacting partners. In addition, in vitro dissociation assays may also be employed to select compounds capable of dissociating or destabilizing the protein complexes identified in accordance with the present invention. For example, the assays may entail (1) contacting the interacting members of a protein complex with each other in the presence of a test compound; and (2) detecting the interaction between the interacting members. An in vitro screening assay may also be used to identify compounds that trigger or initiate the formation of, or stabilize, a protein complex of the present invention.

In preferred embodiments, in vivo assays such as yeast two-hybrid assays and various derivatives thereof, preferably reverse two-hybrid assays, are utilized in identifying compounds that interfere with or disrupt the protein-protein interactions discovered according to the present invention. In addition, systems such as yeast two-hybrid assays are also useful in selecting compounds capable of triggering or initiating, enhancing or stabilizing the protein-protein interactions provided in the tables. In a specific embodiment, the screening method includes: (a) providing in a host cell a first fusion protein having a first protein of an interacting protein pair, or a homologue, derivative or fragment thereof, and a second fusion protein having the second protein of the pair, or a homologue, derivative or fragment thereof, wherein a DNA binding domain is fused to one of the first and second proteins while a transcription-activating domain is fused to the other of said first and second proteins; (b) providing in the host cell a reporter gene, wherein the transcription of the reporter gene is determined by the interaction between the first protein and the second protein; (c) allowing the first and second fusion proteins to interact with each other within the host cell in the presence of a test compound; and (d) determining the presence or absence of expression of the reporter gene.

In addition, the present invention also provides a method for selecting a compound capable of modulating a protein-protein interaction in accordance with the present invention, which comprises the steps of (1) contacting a test compound with an interacting protein disclosed in the tables, or a homologue, derivative or fragment thereof, and (2) determining whether said test compound is capable of binding said protein. In a preferred embodiment, the method further includes testing a selected test compound capable of binding said interacting protein for its ability to interfere with a protein-protein interaction according to the present invention involving said interacting protein, and optionally further testing the selected test compound for its ability to modulate cellular activities associated with said interacting protein and/or said protein-protein interaction.

The present invention also relates to a virtual screen method for providing a compound capable of modulating the interaction between the interacting members in a protein complex of the present invention. In one embodiment, the method comprises the steps of providing atomic coordinates defining a three-dimensional structure of a protein complex of the present invention, and designing or selecting, based on said atomic coordinates, compounds capable of interfering with the interaction between the interacting protein members of the protein complex. In another embodiment, the method comprises the steps of providing atomic coordinates defining a three-dimensional structure of an interacting protein described in the tables, and designing or selecting compounds capable of binding the interacting protein based on said atomic coordinates. In preferred embodiments, the method further includes testing a selected test compound for its ability to interfere with a protein-protein interaction provided in accordance with the present invention involving said interacting protein, and optionally further testing the selected test compound for its ability to modulate cellular activities associated with the interacting protein.

The present invention further provides a composition having two expression vectors. One vector contains a nucleic acid encoding a protein of an interacting protein pair according to the present invention, or a homologue, derivative or fragment thereof. Another vector contains the other protein of the interacting pair, or a homologue, derivative or fragment thereof. In addition, an expression vector is also provided containing (1) a first nucleic acid encoding one protein of an interacting protein pair of the present invention, or a homologue, derivative or fragment thereof, and (2) a second nucleic acid encoding the other protein of the interacting pair, or a homologue, derivative or fragment thereof.

Host cells are also provided containing the first and second nucleic acids or comprising the expression vector(s). In addition, the present invention also provides a host cell having two expression cassettes. One expression cassette includes a promoter operably linked to a nucleic acid encoding one protein of an interacting pair of the present invention, or a homologue, derivative or fragment thereof. Another expression cassette includes a promoter operably linked to a nucleic acid encoding the other protein of the interacting pair, or a homologue, derivative or fragment thereof. Preferably, the expression cassettes are chimeric expression cassettes with heterologous promoters included.

In specific embodiments of the host cells or expression vectors, one of the two nucleic acids is linked to a nucleic acid encoding a DNA binding domain, and the other is linked to a nucleic acid encoding a transcription-activation domain, whereby two fusion proteins can be encoded.

In accordance with yet another aspect of the present invention, methods are provided for modulating the functions and activities of a protein complex of the present invention, or interacting protein members thereof. The methods may be used in treating or preventing diseases and disorders such as Alzheimer's disease (including mild cognitive impairment), Huntington's disease, Parkinson's disease, schizophrenia, depression, dementia and other neurodegenerative diseases, as well as diabetes, obesity and coronary artery disease.

In one embodiment, the method comprises reducing a protein complex concentration and/or inhibiting the functional activities of the protein complex. Alternatively, the concentration and/or activity of one or more interacting members of a protein complex may be reduced or inhibited. Thus, the methods may include administering to a patient an antibody specific to a protein complex or an interacting protein member thereof, or an siRNA or antisense oligo or ribozyme selectively hybridizable to a gene or mRNA encoding an interacting member of the protein complex. Also useful is a compound identified in a screening assay of the present invention capable of disrupting the interaction between two interacting members of a protein complex, or inhibiting the activities of an interacting member of the protein complex. In addition, gene therapy methods may also be used in reducing the expression of the gene(s) encoding one or more interacting protein members of a protein complex.

In another embodiment, the methods for modulating the functions and activities of a protein complex of the present invention or interacting protein members thereof comprise increasing the protein complex concentration and/or activating the functional activities of the protein complex. Alternatively, the concentration and/or activity of one or more interacting members of a protein complex of the present invention may be increased. Thus, one or more interacting protein members of a protein complex of the present invention may be administered directly to a patient. Or, exogenous genes encoding one or more protein members of a protein complex of the present invention may be introduced into a patient by gene therapy techniques. In addition, a patient needing treatment or prevention may also be administered with compounds identified in a screening assay of the present invention capable of triggering or initiating, enhancing or stabilizing a protein-protein interaction of the present invention.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE FIGURES

FIGS. 1-42 depict siRNA molecules directed towards the identified target.

### DETAILED DESCRIPTION OF THE INVENTION

### 1. Definitions

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

The term “isolated polypeptide” as used herein is defined as a polypeptide molecule that is present in a form other than that found in nature. Thus, an isolated polypeptide can be a non-naturally occurring polypeptide. For example, an “isolated polypeptide” can be a “hybrid polypeptide.” An “isolated polypeptide” can also be a polypeptide derived from a naturally occurring polypeptide by additions or deletions or substitutions of amino acids. An isolated polypeptide can also be a “purified polypeptide” which is used herein to mean a specified polypeptide in a substantially homogeneous preparation substantially free of other cellular components, other polypeptides, viral materials, or culture medium, or when the polypeptide is chemically synthesized, chemical precursors or by-products associated with the chemical synthesis. A “purified polypeptide” can be obtained from natural or recombinant host cells by standard purification techniques, or by chemically synthesis, as will be apparent to skilled artisans.

The terms “hybrid protein,” “hybrid polypeptide,” “hybrid peptide,” “fusion protein,” “fusion polypeptide,” and “fusion peptide” are used herein interchangeably to mean a non-naturally occurring polypeptide or isolated polypeptide having a specified polypeptide molecule covalently linked to one or more other polypeptide molecules that do not link to the specified polypeptide in nature. Thus, a “hybrid protein” may be two naturally occurring proteins or fragments thereof linked together by a covalent linkage. A “hybrid protein” may also be a protein formed by covalently linking two artificial polypeptides together. Typically but not necessarily, the two or more polypeptide molecules are linked or “fused” together by a peptide bond forming a single non-branched polypeptide chain.

As used herein, the term “interacting” or “interaction” means that two protein domains, fragments or complete proteins exhibit sufficient physical affinity to each other so as to bring the two “interacting” protein domains, fragments or proteins physically close to each other. An extreme case of interaction is the formation of a chemical bond that results in continual and stable proximity of the two entities. Interactions that are based solely on physical affinities, although usually more dynamic than chemically bonded interactions, can be equally effective in co-localizing two proteins. Examples of physical affinities and chemical bonds include but are not limited to, forces caused by electrical charge differences, hydrophobicity, hydrogen bonds, van der Waals force, ionic force, covalent linkages, and combinations thereof. The state of proximity between the interaction domains, fragments, proteins or entities may be transient or permanent, reversible or irreversible. In any event, it is in contrast to and distinguishable from contact caused by natural random movement of two entities. Typically, although not necessarily, an “interaction” is exhibited by the binding between the interaction domains, fragments, proteins, or entities. Examples of interactions include specific interactions between antigen and antibody, ligand and receptor, enzyme and substrate, and the like.

An “interaction” between two protein domains, fragments or complete proteins can be determined by a number of methods. For example, an interaction is detectable by any commonly accepted approaches, including functional assays such as the two-hybrid systems. Protein-protein interactions can also be determined by various biophysical and biochemical approaches based on the affinity binding between the two interacting partners. Such biochemical methods generally known in the art include, but are not limited to, protein affinity chromatography, affinity blotting, immunoprecipitation, and the like. The binding constant for two interacting proteins, which reflects the strength or quality of the interaction, can also be determined using methods known in the art. See Phizicky and Fields, Microbiol. Rev., 59:94-123 (1995).

As used herein, the term “protein complex” means a composite unit that is a combination of two or more proteins formed by interaction between the proteins. Typically but not necessarily, a “protein complex” is formed by the binding of two or more proteins together through specific non-covalent binding affinities. However, covalent bonds may also be present between the interacting partners. For instance, the two interacting partners can be covalently crosslinked so that the protein complex becomes more stable.

The term “isolated protein complex” means a naturally occurring protein complex present in a composition or environment that is different from that found in its native or original cellular or biological environment in nature. An “isolated protein complex” may also be a protein complex that is not found in nature.

The term “protein fragment” as used herein means a polypeptide that represents a portion of a protein. When a protein fragment exhibits interactions with another protein or protein fragment, the two entities are said to interact through interaction domains that are contained within the entities.

As used herein, the term “domain” means a functional portion, segment or region of a protein, or polypeptide. “Interaction domain” refers specifically to a portion, segment or region of a protein, polypeptide or protein fragment that is responsible for the physical affinity of that protein, protein fragment or isolated domain for another protein, protein fragment or isolated domain.

The term “isolated” when used in reference to nucleic acids (which include gene sequences) of this invention is intended to mean that a nucleic acid molecule is present in a form other than that found in nature.

Thus, an isolated nucleic acid can be a non-naturally occurring nucleic acid. For example, the term “isolated nucleic acid” encompasses “recombinant nucleic acid” which is used herein to mean a hybrid nucleic acid produced by recombinant DNA technology having the specified nucleic acid molecule covalently linked to one or more nucleic acid molecules that are not the nucleic acids naturally flanking the specified nucleic acid in the naturally existing chromosome. One example of recombinant nucleic acid is a hybrid nucleic acid encoding a fusion protein. Another example is an expression vector having the specified nucleic acid inserted in a vector.

The term “isolated nucleic acid” also encompasses nucleic acid molecules that are present in a form other than that found in its original environment in nature with respect to its association with other molecules. In this respect, an “isolated nucleic acid” as used herein means a nucleic acid molecule having only a portion of the nucleic acid sequence in the chromosome but not one or more other portions present on the same chromosome. Thus, an isolated nucleic acid present in a form other than that found in its original environment in nature with respect to its association with other molecules typically includes no more than 10 kb of the naturally occurring nucleic acid sequences that immediately flank the gene in the naturally existing chromosome or genomic DNA. Thus, the term “isolated nucleic acid” encompasses the term “purified nucleic acid,” which means an isolated nucleic acid in a substantially homogeneous preparation substantially free of other cellular components, other nucleic acids, viral materials, or culture medium, or chemical precursors or by-products associated with chemical reactions for chemical synthesis of nucleic acids. Typically, a “purified nucleic acid” can be obtained by standard nucleic acid purification methods, as will be apparent to skilled artisans.

An isolated nucleic acid can be in a vector. However, it is noted that an “isolated nucleic acid” as used herein is distinct from a clone in a conventional library such as a genomic DNA library or a cDNA library in that the clones in a library are still in admixture with almost all the other nucleic acids from a chromosome or a cell.

The term “high stringency hybridization conditions,” when used in connection with nucleic acid hybridization, means hybridization conducted overnight at 42 degrees C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 0.1×SSC at about 65° C. The term “moderate stringent hybridization conditions,” when used in connection with nucleic acid hybridization, means hybridization conducted overnight at 37 degrees C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

As used herein, the term “homologue,” when used in connection with a first native protein or fragment thereof that is discovered, according to the present invention, to interact with a second native protein or fragment thereof, means a polypeptide that exhibits a sufficient amino acid sequence homology (greater than 20%) and structural resemblance to the first native interacting protein, or to one of the interacting domains of the first native protein such that it is capable of interacting with the second native protein. Typically, a protein homologue of a native protein may have an amino acid sequence that is at least about 50%, 55%, 60%, 65% or 70%, preferably at least about 75%, more preferably at least about 80%, 85%, 86%, 87%, 88% or 89%, even more preferably at least 90%, 91%, 92%, 93% or 94%, and most preferably about 95%, 96%, 97%, 98% or 99% identical to the native protein. Examples of homologues may be the ortholog proteins of other species including animals, plants, yeast, bacteria, and the like. Homologues may also be selected by, e.g., mutagenesis in a native protein. For example, homologues may be identified by site-specific mutagenesis in combination with assays for detecting protein-protein interactions, e.g., the yeast two-hybrid system described below, as will be apparent to skilled artisans apprised of the present invention. Other techniques for detecting protein-protein interactions include, e.g., protein affinity chromatography, affinity blotting, in vitro binding assays, and the like.

For the purpose of comparing two different nucleic acid or polypeptide sequences, one sequence (test sequence) may be described to be a specific “percent identical to” another sequence (reference sequence) in the present disclosure. In this respect, the percentage identity is determined by the algorithm of Karlin and Altschul, Proc. Natl. Acad. Sci. USA, 90:5873-5877 (1993), which is incorporated into various BLAST programs. Specifically, the percentage identity is determined by the “BLAST 2 Sequences” tool, which is available at the National Center for Biotechnology Information's website. See Tatusova and Madden, FEMS Microbiol. Lett., 174(2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter). Percent identity of two sequences is calculated by aligning a test sequence with a reference sequence using BLAST 2.1.2., determining the number of amino acids or nucleotides in the aligned test sequence that are identical to amino acids or nucleotides in the same position of the reference sequence, and dividing the number of identical amino acids or nucleotides by the number of amino acids or nucleotides in the reference sequence. When BLAST 2.1.2 is used to compare two sequences, it aligns the sequences and yields the percent identity over defined, aligned regions. If the two sequences are aligned across their entire length, the percent identity yielded by the BLAST 2.1.1 is the percent identity of the two sequences. If BLAST 2.1.2 does not align the two sequences over their entire length, then the number of identical amino acids or nucleotides in the unaligned regions of the test sequence and reference sequence is considered to be zero and the percent identity is calculated by adding the number of identical amino acids or nucleotides in the aligned regions and dividing that number by the length of the reference sequence.

The term “derivative,” when used in connection with a first native protein (or fragment thereof) that is discovered, according to the present invention, to interact with a second native protein (or fragment thereof), means a modified form of the first native protein prepared by modifying the side chain groups of the first native protein without changing the amino acid sequence of the first native protein. The modified form, i.e., the derivative should be capable of interacting with the second native protein. Examples of modified forms include glycosylated forms, phosphorylated forms, myristylated forms, ribosylated forms, ubiquitinated forms, and the like. Derivatives also include hybrid or fusion proteins containing a native protein or a fragment thereof. Methods for preparing such derivative forms should be apparent to skilled artisans. The prepared derivatives can be easily tested for their ability to interact with the native interacting partner using techniques known in the art, e.g., protein affinity chromatography, affinity blotting, in vitro binding assays, yeast two-hybrid assays, and the like.

The term “antibody” as used herein encompasses both monoclonal and polyclonal antibodies that fall within any antibody classes, e.g., IgG, IgM, IgA, IgE, or derivatives thereof. The term “antibody” also includes antibody fragments including, but not limited to, Fab, F(ab′)₂, and conjugates of such fragments, and single-chain antibodies comprising an antigen recognition epitope. In addition, the term “antibody” also means humanized antibodies, including partially or fully humanized antibodies. An antibody may be obtained from an animal, or from a hybridoma cell line producing a monoclonal antibody, or obtained from cells or libraries recombinantly expressing a gene encoding a particular antibody.

The term “selectively immunoreactive” as used herein means that an antibody is reactive thus binds to a specific protein or protein complex, but not other similar proteins or fragments or components thereof.

The term “activity” when used in connection with proteins or protein complexes means any physiological or biochemical activities displayed by or associated with a particular protein or protein complex including but not limited to activities exhibited in biological processes and cellular functions, ability to interact with or bind another molecule or a moiety thereof, binding affinity or specificity to certain molecules, in vitro or in vivo stability (e.g., protein degradation rate, or in the case of protein complexes, the ability to maintain the form of a protein complex), antigenicity and immunogenicity, enzymatic activities, etc. Such activities may be detected or assayed by any of a variety of suitable methods as will be apparent to skilled artisans.

The term “compound” as used herein encompasses all types of organic or inorganic molecules, including but not limited proteins, peptides, polysaccharides, lipids, nucleic acids, small organic molecules, inorganic compounds, and derivatives thereof.

As used herein, the term “interaction antagonist” means a compound that interferes with, blocks, disrupts or destabilizes a protein-protein interaction; blocks or interferes with the formation of a protein complex; or destabilizes, disrupts or dissociates an existing protein complex.

The term “interaction agonist” as used herein means a compound that triggers, initiates, propagates, nucleates, or otherwise enhances the formation of a protein-protein interaction; triggers, initiates, propagates, nucleates, or otherwise enhances the formation of a protein complex; or stabilizes an existing protein complex.

Unless otherwise specified, the names of interacting proteins, as used herein, and when referring to the protein complexes of the present invention, are meant to refer to full-length proteins, as well as any fragments thereof that are capable of interacting with a specified interacting partner protein as disclosed in the tables below. Additionally, while the names of interacting proteins, as used herein, generally refer to the human form of the named protein, they may also refer to homologous proteins from other species that interact in a manner analogous to the human protein. Preferably, such homologous proteins are at least about 80%, 85%, 90%, 92%, 93%, 94%, 95%, 96%, 97%, 98%, 99% or more, identical to the human form of the protein. Also, preferably, such homologous proteins interact with the human form of the corresponding interacting partner protein disclosed in the tables. Finally, the names of interacting proteins, as used herein, are also meant to refer to fragments of such human proteins, or their homologues, that interact to form complexes with the interacting partner proteins, as disclosed in the tables. For example, the term “BAT3” refers not only to the full length human HLA-B Associated Transcript 3 (BAT3) protein, but also refers to fragments of human BAT3, homologues of human BAT3, and fragments of these homologues of human BAT3, that retain the ability to interact with a BAT3 partner or prey protein specified in the tables.

### 2. Protein Complexes

Novel protein-protein interactions have been discovered. The protein-protein interactions are provided in the tables below. Specific fragments capable of conferring interacting properties on the interacting proteins have also been identified. The GenBank reference numbers for the cDNA sequences encoding the interacting proteins are also noted in the tables.

### Tables

TABLE 1

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| HLA-B | 235 | 430 | CGI-59 | NM_016007 | 34 | 242 |
| Associated | | | | | | |
| Transcript 3 | | | | | | |
| (BAT3) | | | | | | |
| (GenBank | | | | | | |
| Accession No. | | | | | | |
| M33519) | | | | | | |

TABLE 2

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| HLA-B | 740 | 1040 | calnexin | L10284 | 1 | 145 |
| Associated | | | | | | |
| Transcript 3 | | | | | | |
| (BAT3) | | | | | | |
| (GenBank | | | | | | |
| Accession No. | | | | | | |
| M33519) | | | | | | |

TABLE 3

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| HLA-B | 740 | 1040 | endoplasmic | X94910 | 1 | 262 |
| Associated | | | reticulum | | | |
| Transcript 3 | | | protein | | | |
| (BAT3) | | | ERP31 | | | |
| (GenBank | | | (ERP31 | | | |
| Accession No. | | | precursor) | | | |
| M33519) | | | | | | |

TABLE 4

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| CGI-59 | 1 | 392 | protein | D78360 | 1 | 35 |
| (GenBank | | | phosphatase | | | |
| Accession No. | | | 2A 74 kDa | | | |
| NM_016007) | | | regulatory | | | |
| | | | subunit | | | |
| | | | (PP2A) | | | |

TABLE 5

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| CGI-59 | 1 | 392 | KIAA0864 | AB020671 | 898 | 1214 |
| (GenBank | | | | | | |
| Accession No. | | | | | | |
| NM_016007) | | | | | | |

TABLE 6

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| Chimaerin A2 | 1 | 265 | β-catenin | Z19054 | 118 | 299 |
| (GenBank | | | | | | |
| Accession No. | | | | | | |
| X51408) | | | | | | |

TABLE 7

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Chimaerin A2 | 1 | 265 | trinucleotide | D83783 | 51 | 465 |
| (GenBank | | | repeat | | | |
| Accession No. | | | containing | | | |
| X51408) | | | 11 (THR- | | | |
| | | | associated | | | |
| | | | protein, | | | |
| | | | 230 kDa | | | |
| | | | subunit) | | | |
| | | | (TRAP230) | | | |

TABLE 8

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Chimaerin A2 | 1 | 265 | hypothetical | NM_014887 | 176 | 349 |
| (GenBank | | | protein from | | | |
| Accession No. | | | BCRA2 | | | |
| X51408) | | | region | | | |
| | | | (CG005) | | | |

TABLE 9

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| δ-catenin | 516 | 833 | TRIP1 | D44467 | 19 | 98 |
| (GenBank | | | | | | |
| Accession No. | | | | | | |
| U96136) | | | | | | |

TABLE 10

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| CIB (GenBank | 1 | 137 | cisplatin | AB034205 | 62 | 246 |
| Accession No. | | | resistance- | | | |
| U82226) | | | associated | | | |
| | | | overexpressed | | | |
| | | | protein | | | |
| | | | (CROP) | | | |

TABLE 11

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| CIB (GenBank | 1 | 137 | F-box only | AB020682 | 107 | 395 |
| Accession No. | | | protein 21 | | | |
| U82226) | | | isoform | | | |
| | | | (FBX21) | | | |

TABLE 12

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| CIB (GenBank | 1 | 917 | KIAA0127 | D50917 | 43 | 314 |
| Accession No. | | | | | | |
| U82226) | | | | | | |

TABLE 13

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| CIB (GenBank | 1 | 191 | KIAA0281 | D87457 | 122 | 192 |
| Accession No. | | | | | | |
| U82226) | | | | | | |

TABLE 14

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| GABARAPL1 | 1 | 117 | ANK2 | NM_001148 | 1511 | 1661 |
| (GenBank | | | | | | |
| Accession No. | | | | | | |
| NM_031412) | | | | | | |

TABLE 15

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| GABARAPL1 | 1 | 117 | Calreticulin | M84739 | 164 | 362 |
| (GenBank | | | | | | |
| Accession No. | | | | | | |
| NM_031412) | | | | | | |

TABLE 16

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| GABARAPL1 | 1 | 117 | KIAA0443 | AB007903 | 167 | 448 |
| (GenBank | | | | | | |
| Accession No. | | | | | | |
| NM_031412) | | | | | | |

TABLE 17

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| GABARAPL1 | 1 | 117 | MAP1A | U38291 | 1656 | 1845 |
| (GenBank | | | | | | |
| Accession No. | | | | | | |
| NM_031412) | | | | | | |

TABLE 18

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| GABARAPL1 | 1 | 117 | KIAA1855 | AB058758 | 467 | 679 |
| (GenBank | | | | | | |
| Accession No. | | | | | | |
| NM_031412) | | | | | | |

TABLE 19

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| GABARAP | 1 | 117 | Calreticulin | M84739 | 80 | 360 |
| (GenBank | | | | | | |
| Accession | | | | | | |
| No. | | | | | | |
| NM_007278) | | | | | | |

TABLE 20

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| GABARAP | 1 | 117 | KIAA0443 | AB007903 | 167 | 448 |
| (GenBank | | | | | | |
| Accession | | | | | | |
| No. | | | | | | |
| NM_007278) | | | | | | |

TABLE 21

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| GABARAP | 1 | 117 | MAP1A | U38291 | 1656 | 1812 |
| (GenBank | | | | | | |
| Accession | | | | | | |
| No. | | | | | | |
| NM_007278) | | | | | | |

TABLE 22

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| GABARAP | 1 | 117 | AKAP11 | NM_016248 | 1378 | 1901 |
| (GenBank | | | | | | |
| Accession | | | | | | |
| No. | | | | | | |
| NM_007278) | | | | | | |

TABLE 23

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| GABARAP | 1 | 117 | ANK2 | NM_001148 | 1511 | 1661 |
| (GenBank | | | | | | |
| Accession | | | | | | |
| No. | | | | | | |
| NM_007278) | | | | | | |

TABLE 24

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| GEF-2 | 1 | 117 | Calreticulin | M84739 | 147 | 362 |
| (GenBank | | | | | | |
| Accession | | | | | | |
| No. | | | | | | |
| AJ010569) | | | | | | |

TABLE 25

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| GEF-2 | 1 | 117 | ANK2 | NM_001148 | 1511 | 1661 |
| (GenBank | | | | | | |
| Accession | | | | | | |
| No. | | | | | | |
| AJ010569) | | | | | | |

TABLE 26

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| PSD95 | 149 | 255 | PN7740 | NM_152542 | 27 | 321 |
| (GenBank | | | | | | |
| Accession No. | | | | | | |
| NM_001365) | | | | | | |

TABLE 27

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| PS1 | 252 | 393 | DLG2 | U32376 | 350 | 601 |
| (GenBank | | | | | | |
| Accession | | | | | | |
| No. L42110) | | | | | | |

TABLE 28

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| DLG2 | 411 | 616 | DAGK6 | U51477 | 828 | 928 |
| (GenBank | | | | | | |
| Accession No. | | | | | | |
| U32376) | | | | | | |

TABLE 29

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| NE-dlg | 216 | 578 | DAGK6 | U51477 | 778 | 928 |
| (GenBank | | | | | | |
| Accession No. | | | | | | |
| U49089) | | | | | | |

TABLE 30

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| PSD95 | 302 | 542 | DAGK6 | U51477 | 892 | 928 |
| (GenBank | | | | | | |
| Accession No. | | | | | | |
| NM_001365) | | | | | | |

TABLE 31

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| LRP1 | 4435 | 4544 | IB1 | NM_005456 | 451 | 589 |
| (GenBank | | | | | | |
| Accession | | | | | | |
| No. | | | | | | |
| NM_002332) | | | | | | |

TABLE 32

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| S-SCAM | 370 | 519 | NET1 | NM_005863 | 330 | 460 |
| (GenBank | | | | | | |
| Accession | | | | | | |
| No. | | | | | | |
| AF038563) | | | | | | |

TABLE 33

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| NE-dlg | 216 | 578 | NET1 | NM_005863 | 330 | 460 |
| (GenBank | | | | | | |
| Accession | | | | | | |
| No. U49089) | | | | | | |

TABLE 34

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| DLG2 | 411 | 870 | NET1 | NM_005863 | 330 | 460 |
| (GenBank | | | | | | |
| Accession | | | | | | |
| No. | | | | | | |
| U32376) | | | | | | |

TABLE 35

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| DLG2 | 411 | 616 | p38 | Y10487 | 225 | 367 |
| (GenBank | | | gamma | | | |
| Accession | | | | | | |
| No. | | | | | | |
| U32376) | | | | | | |

TABLE 36

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| NE-dlg | 216 | 578 | PN18543 | AB037843 | 1039 | 1237 |
| (GenBank | | | | | | |
| Accession | | | | | | |
| No. | | | | | | |
| U49089) | | | | | | |

TABLE 37

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| GIPC | 119 | 233 | SK3 | NM_002249 | 606 | 736 |
| (GenBank | | | | | | |
| Accession | | | | | | |
| No. | | | | | | |
| AF089816) | | | | | | |

TABLE 38

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| NE-dlg | 216 | 578 | REC8 | AF132734 | 794 | 974 |
| (GenBank | | | | | | |
| Accession | | | | | | |
| No. | | | | | | |
| U49089) | | | | | | |

TABLE 39

| Bait Protein | | | Prey Proteins | | | |
|---|---|---|---|---|---|---|
| Name and | Amino Acid | | | | Amino Acid | |
| GenBank | Coordinates | | | GenBank | Coordinates | |
| Accession No. | Start | Stop | Names | Accession Nos. | Start | Stop |
| S-SCAM | 370 | 519 | REC8 | AF132734 | 794 | 974 |
| (GenBank | | | | | | |
| Accession | | | | | | |
| No. | | | | | | |
| AF038563) | | | | | | |

TABLE 40

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| ADAM10 | 20 | 200 | ZFN198 | NM_003453 | 439 | 785 |
| (GenBank | | | | | | |
| Accession | | | | | | |
| No. | | | | | | |
| NM_001110) | | | | | | |

TABLE 41

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| ADAM10 | 20 | 200 | PAPSS1 | NM_005443 | 12 | 284 |
| (GenBank | | | | | | |
| Accession | | | | | | |
| No. | | | | | | |
| NM_001110) | | | | | | |

TABLE 42

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| ADAM10 | 20 | 200 | KIAA0356 | AB002354 | 851 | 1058 |
| (GenBank | | | | | | |
| Accession | | | | | | |
| No. | | | | | | |
| NM_001110) | | | | | | |

TABLE 43

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| GIPC | 119 | 233 | ADAM17 | NM_003183 | 697 | 824 |
| (GenBank | | | | | | |
| Accession | | | | | | |
| No. | | | | | | |
| AF089816) | | | | | | |

TABLE 44

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| GIPC | 119 | 233 | GLP2R | NM_004246 | 512 | 533 |
| (GenBank | | | | | | |
| Accession | | | | | | |
| No. | | | | | | |
| AF089816) | | | | | | |

TABLE 45

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| KIAA0443 | 901 | 1200 | PPP2R2B | M64930 | 195 | 440 |
| (GenBank | | | | | | |
| Accession No. | | | | | | |
| AB007903) | | | | | | |

TABLE 46

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| KIAA0443 | 901 | 1200 | MAP1B | L06237 | 2243 | 2468 |
| (GenBank | | | | | | |
| Accession | | | | | | |
| No. | | | | | | |
| AB007903) | | | | | | |

TABLE 47

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| KIAA0443 | 901 | 1200 | NET-7 | NM_012339 | 1 | 219 |
| (GenBank | | | | | | |
| Accession No. | | | | | | |
| AB007903) | | | | | | |

TABLE 48

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| CASK | 1 | 325 | TIAM1 | U16296 | 1472 | 1541 |
| (GenBank | | | | | | |
| Accession No. | | | | | | |
| NM_003688) | | | | | | |

TABLE 49

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| DLG2 | 411 | 616 | HAPIP | NM_003947 | 1593 | 1663 |
| (GenBank | | | | | | |
| Accession No. | | | | | | |
| U32376) | | | | | | |

TABLE 50

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| CIB | 1 | 191 | ZFN127 | U19107 | 313 | 507 |
| (GenBank | | | | | | |
| Accession | | | | | | |
| No. U82226) | | | | | | |

TABLE 51

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| BAT3 | 740 | 1040 | DKK-3 | AB033421 | 1 | 129 |
| (GenBank | | | | | | |
| Accession | | | | | | |
| No. M33519) | | | | | | |

TABLE 52

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| BAT3 | 740 | 1040 | PN9113 | NM_181784 | 306 | 415 |
| (GenBank | | | | | | |
| Accession | | | | | | |
| No. | | | | | | |
| M33519) | | | | | | |

TABLE 53

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| Axin | 451 | 750 | LRP1 | NM_002332 | 2524 | 2714 |
| (GenBank | | | | | | |
| Accession | | | | | | |
| No. | | | | | | |
| AF009674) | | | | | | |

TABLE 54

| Bait Protein | | | | | | |
|---|---|---|---|---|---|---|
| Name and | | | Prey Proteins | | | |
| GenBank | Amino Acid | | | GenBank | Amino Acid | |
| Accession | Coordinates | | | Accession | Coordinates | |
| No. | Start | Stop | Names | Nos. | Start | Stop |
| Axin | 451 | 750 | LRP2 | U04441 | 938 | 1196 |
| (GenBank | | | | | | |
| Accession | | | | | | |
| No. | | | | | | |
| AF009674) | | | | | | |

TABLE 55

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Axin | 451 | 750 | LRPDIT | NM_018557 | 2475 | 2711 |
| (GenBank | | | | | | |
| Accession | | | | | | |
| No. | | | | | | |
| AF009674) | | | | | | |

TABLE 56

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Axin | 451 | 750 | PN9113 | NM_181784 | 306 | 415 |
| (GenBank | | | | | | |
| Accession | | | | | | |
| No. | | | | | | |
| AF009674) | | | | | | |

TABLE 57

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| ERRγ | 160 | 436 | RanBP2 | D42063 | 813 | 1155 |
| (GenBank | | | | | | |
| Accession | | | | | | |
| No. | | | | | | |
| AF058291) | | | | | | |

TABLE 58

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| ERRγ | 160 | 436 | G19P1 | J03075 | 1 | 225 |
| (GenBank | | | | | | |
| Accession No. | | | | | | |
| AF058291) | | | | | | |

TABLE 59

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| ERRγ | 160 | 436 | AIP2 | U37547 | 316 | 565 |
| (GenBank | | | | | | |
| Accession No. | | | | | | |
| AF058291) | | | | | | |

TABLE 60

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| ERRγ | 160 | 436 | Prox1 | U44060 | 61 | 302 |
| (GenBank | | | | | | |
| Accession | | | | | | |
| No. | | | | | | |
| AF058291) | | | | | | |

TABLE 61

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Caspase-3 | 1 | 185 | neurocalcin | NM_032041 | 130 | 193 |
| (GenBank | | | | | | |
| Accession | | | | | | |
| No. | | | | | | |
| U13737) | | | | | | |

TABLE 62

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Caspase-3 | 1 | 185 | calcineurin B | M30773 | 25 | 170 |
| (GenBank | | | B | | | |
| Accession No. | | | | | | |
| U13737) | | | | | | |

TABLE 63

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| FAK2 | 673 | 866 | PQBP1 | AJ242829 | 1 | 170 |
| (GenBank | | | | | | |
| Accession | | | | | | |
| No. L49207) | | | | | | |

TABLE 64

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| BAT3 | 740 | 1040 | pleiotrophin | M57399 | 1 | 168 |
| (GenBank | | | | | | |
| Accession | | | | | | |
| No. | | | | | | |
| M33519) | | | | | | |

TABLE 65

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| PN7018 | 1 | 82 | phogrin | U66702 | 138 | 517 |
| (GenBank | | | | | | |
| Accession No. | | | | | | |
| AK025522) | | | | | | |

TABLE 66

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| LSF | 393 | 502 | neurabin | NM_032595 | 363 | 410 |
| (GenBank | | | II | | | |
| Accession | | | | | | |
| No. | | | | | | |
| U03494) | | | | | | |

TABLE 67

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 1 | 252 | KIAA0410 | AB007870 | 41 | 389 |
| Accession | | | | | | |
| No.M14752) | | | | | | |

TABLE 68

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl | 1 | 252 | nucleoporin | S59346 | 439 | 505 |
| (GenBank | | | p62 | | | |
| Accession | | | | | | |
| No.M14752) | | | | | | |

TABLE 69

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 1 | 252 | SOCS-2 | AF037989 | 1 | 65 |
| Accession | | | | | | |
| No.M14752) | | | | | | |

TABLE 70

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 1 | 252 | AF6 | U02478 | 225 | 904 |
| Accession | | | | | | |
| No. M14752) | | | | | | |

TABLE 71

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 1 | 252 | calcineurin B | M30773 | 31 | 170 |
| Accession | | | | | | |
| No. M14752) | | | | | | |

TABLE 72

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 1 | 252 | WAVE1 | D87459 | 178 | 490 |
| Accession | | | | | | |
| No. M14752) | | | | | | |

TABLE 73

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 1 | 252 | KIAA0779 | AB018322 | 1 | 195 |
| Accession | | | | | | |
| No. M14752) | | | | | | |

TABLE 74

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 1 | 252 | KIAA0820 | AB020627 | 518 | 694 |
| Accession | | | | | | |
| No. M14752) | | | | | | |

TABLE 75

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 1 | 252 | KIAA0846 | AB020653 | 536 | 669 |
| Accession | | | | | | |
| No. M14752) | | | | | | |

TABLE 76

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 1 | 252 | Sox-2 | L07335 | 149 | 317 |
| Accession | | | | | | |
| No. M14752) | | | | | | |

TABLE 77

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 1 | 252 | CAP2 | U02390 | 1 | 271 |
| Accession | 1 | 252 | | | 1984 | 2708 |
| No. M14752) | | | | | | |

TABLE 78

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 484 | 792 | kendrin | U52962 | 1707 | 2133 |
| Accession | | | | | | |
| No. M14752) | | | | | | |

TABLE 79

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 1 | 252 | KIAA0886 | AB020693 | 1032 | 1168 |
| Accession | | | | | | |
| No. M14752) | | | | | | |

TABLE 80

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| BAX (GenBank | 50 | 107 | KIAA0886 | AB020693 | 556 | 990 |
| Accession | | | | | | |
| No. L22474) | | | | | | |

TABLE 81

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| FAK2 (GenBank | 673 | 866 | KIAA0886 | AB020693 | 556 | 990 |
| Accession | | | | | | |
| No. L49207) | | | | | | |

TABLE 82

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| Abl (GenBank | 1 | 252 | ZFM1 | D26120 | 272 | 531 |
| Accession | | | | | | |
| No. M14752) | | | | | | |

TABLE 83

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| LSF (GenBank | 393 | 502 | ZFM1 | D26120 | 80 | 276 |
| Accession | | | | | | |
| No. U03494) | | | | | | |

TABLE 84

| Bait Protein | | | Prey Proteins | | | |
|---|---|---|---|---|---|---|
| Name and | Amino Acid | | | GenBank | Amino Acid | |
| GenBank | Coordinates | | | Accession | Coordinates | |
| Accession No. | Start | Stop | Names | Nos. | Start | Stop |
| BAX (GenBank | 50 | 107 | bleomycin | X92106 | 1 | 292 |
| Accession | | | hydrolase | | | |
| No. L22474 | | | | | | |

### 2.1. Biological Significance

Also called HLA-B associated transcript 3, BAT3 is a protein of unknown function that contains a ubiquitin-like domain in the N-terminal region (aa 17 to 77) and two proline-rich domains (aa 202 to 207 and 657 to 670) (Banerji et al., 1990; Wang and Liew, 1994; Spies et al., 1989b; Spies et al., 1989a). Because of its interactions with various proteins in the Alzheimer's Disease protein-protein interaction network, BAT3 might be involved in APP recycling or intracellular trafficking, which is a crucial event that modulates Aβ production. In addition, BAT3 could also be involved in the brain-specific (neurotrophic, synaptotrophic) functions of APP. A recent study showed that the domain of BAT3 from aa 246 to 360 bind to CAP1, an adenylate cyclase associated protein (Hubberstey et al., 1996). CAP1 is a 475 amino acid protein with two functionally different domains separated by a proline-rich region. The activation of adenylate cyclase by CAP1 might result in elevation of intracellular cAMP levels, a phenomenon that has been linked to long-term potentiation (LTP) (Sah and Bekkers, 1996; Kimura et al., 1998; Storm et al., 1998; Villacres et al., 1998), considered as the cellular and biochemical substrate for memory (Matzel et al., 1998; Davis and Laroche, 1998).

We report an interaction between BAT3 and the protein CGI-59. The cDNA for CGI-59 was first cloned in a random cloning project using a C. elegans proteome template (Lai et al., 2000). Two clones from the NEDO project were subsequently found to encode the same protein. These clones, FLJ10614 (GenBank entry AK001476) and FLJ10657 (GenBank entry AK001519) add more sequence in the 5′ and 3′ direction. In all three entries, the protein encoded by the CGI-59/FLJ10614/FLJ10657 cDNA contains 392 amino acids. The putative ATG initiation codon is in a fair Kozak environment and is preceded by an upstream STOP codon. No domain was found in this predicted protein sequence. The C-terminal 100 residues of CGI-59 are extremely basic, and the protein has an overall isoelectric point of 10.4.

We report the interactions of BAT3 with calnexin and with the Endoplasmic Reticulum Protein 31 (ERP31). Calnexin is a 90 kDa transmembrane protein that binds calcium with high affinity and is found exclusively in the endoplasmic reticulum (ER) (Schrag et al., 2001). The function of this protein is unknown. ERP31 resides in the lumen of the ER and maybe involved in the processing of proteins destined for secretion (Hubbard and McHugh, 2000). These two interactions suggest that some BAT3 molecules are found in the ER, which is the site of Aβ42 production.

We report an interaction between the protein CGI-59 and the delta isoform of the regulatory subunit B of the protein phosphatase 2A (PP2A). PP2A is an enzyme playing an important regulatory function in the wingless signaling pathway. Another major component of the pathway is GSK-3β, which phosphorylates β-catenin, targeting it for degradation in the cytosol (Dierick and Bejsovec, 1999). GSK-3β also phosphorylates other proteins, including neurofilament proteins, synapsin I, various transcription factors, and the product of the APC (adenomatous polyposis coli) gene, and most importantly for Alzheimer's disease, tau (Mandelkow et al., 1992; Ishiguro et al., 1993; Sperber et al. 1995; Baum et al. 1996). The phosphorylation state of these proteins depends on the coordinated activities of GSK-3β and PP2A. The CGI-59 bait used in the search was from amino acids 1 to 392 (full-length). We found one clone in the hippocampus library encoding amino acids 1 to 35 of PP2A.

We report an interaction between the proteins CGI-59 and KIAA0864. The report from the Kazusa DNA Research Institute (KDRI) shows a sequence of 4319 bp with an open reading frame (ORF) of 3597 bp coding for 1199 aa. The putative ATG initiation codon is in a fair Kozak environment and is not preceded by an upstream in-frame STOP codon, opening the possibility for a larger ORF. The mRNA expression profile performed at the KDRI by rt-PCR shows medium to high levels of KIAA0864 message in all tissues examined, except for pancreas and spleen (medium levels). Very high levels are observed in all brain regions examined, except for medium to high levels in the corpus callosum. The domain analysis performed at the KDRI reveals the presence of two bZIP transcription factor domains (aa 859 to 895 and aa 1091 to 1109), a K box region (from aa 829 to 935), and a domain signature of the ezrin/radixin/moesin family (from aa 889 to 1128), which is not encoded by our interacting clone (aa 800 to 895). Interestingly, KIAA0864 is 92% identical to a rat protein named RB109 and described in GenBank as a brain-specific protein (GenBank entry D26154). Beside the species difference, RB109 appears to be a truncated version of KIAA0864. The protein alignment shows that RB109 is almost identical to KIAA0864, except for the C-terminal 9 amino acids. Likewise, DNA analysis shows that the KIAA0864 and RB109 cDNAs have almost identical coding regions but diverge significantly in the 5′ and 3′ UTRs. Although the KIAA0864 mRNA is found in all tissues examined, the data suggest that a splice variant of KIAA0864, the human homolog of rat RB109, might be brain-specific. KIAA0864 is also 86% identical to the mouse protein p116Rip (GenBank entry U73200). This brain-specific protein was shown to interact with both the GDP- and GTP-bound forms of RhoA and to stimulate cell flattening and neurite outgrowth (Gebbink et al., 1997). The protein analysis shows that the two proteins share their highest similarity in the C-terminal region, but are quite divergent in the N-terminal and middle regions.

The alpha 1 (A1) and alpha 2 (A2) forms of n-chimaerin arise by alternative splicing of a primary transcript expressed specifically in brain and testis (Hall et al, 1990; Hall et al., 1993; Ahmed et al., 1990). Chimaerin A1 is found only in brain, at highest levels in the Purkinje cells of the cerebellum. It contains 334 amino acids and is 50% identical to the protein kinase C (PKC) in its N-terminal half and 40% identical to break point cluster (bcr) in its C-terminal part (Hall et al., 1990; Ahmed et al., 1990). Like PKC, the N-terminal region of chimaerin A1 contains a domain that binds phorbol esters and diacylglycerol (a known PKC activator). Chimaerin A2 is found in the cortical and hippocampal regions of the brain and in testis (Hall et al., 1993). It contains 459 amino acids with an SH2 domain from amino acids 49 to 117, in addition to the phorbol ester/diacylglycerol binding domain from amino acids 206 to 255. Because of its similarity with bcr, which contains a GTPase activating protein (GAP) domain (Pendergast et al., 1991; Braselmann and McCormick, 1995; Diekmann et al., 1995), n-chimaerin was found to be a GTPase activating protein for the p21Rac GTPase (Ahmed et al., 1993). Moreover, chimaerin A2 was shown to be involved in the stimulation of neurite outgrowth (Hall et al., 2001).

We report an interaction between chimaerin alpha 2 and β-catenin. β-catenin is a member of the wingless pathway that is the target of glycogen synthase kinase-3β (GSK-3β) and the protein phosphatase 2A (PP2A) (Nelson and Gumbiner, 1998; Nakamura et al., 1998). The phosphorylation balance of β-catenin by those two enzymes determines its stability or degradation in the cytosol. When β-catenin is phosphorylated, it is targeted for degradation by the proteasome. When it is not phosphorylated, it is stable and can migrate into the nucleus where it acts as a transcription factor stimulating the expression of specific genes such as siamois and LEF (Nelson and Gumbiner, 1998; Dierick and Bejsovec, 1999; Shtutman et al., 1999). β-catenin also interacts with PS1 and mutations in PS1 associated with FAD are known to affect the stability of the complex that contains β-catenin and PS1 (Kang et al., 1999; Nishimura et al., 1999; Soriano et al., 2001).

We report an interaction between chimaerin alpha 2 and the thyroid receptor associated protein 230 (TRAP230). As its name indicates, TRAP230 was identified as a component of the thyroid hormone receptor complex (Ito et al., 1999). In addition to its involvement in thyroid hormone receptor signalling, a study in C. elegans showed that TRAP2 may also play a role in the wingless pathway (Zhang and Emmons, 2000). Another study in fly showed that TRAP230 plays a crucial role in eye development (Treisman, 2001). The TARP2 gene is on the X chromosome and the protein contains long poly-glutamine repeats in the C-terminal region. In fact, TRAP230 (also called HOPA) is a member of the family of genes containing large trinucleotide repeats and involved in a wide range of human disorders including neurodegeneration and mental retardation (Philibert et al., 1998; Margolis et al., 1997). Because of is association with the thyroid hormone receptor, the TRAP230-chimaerin A2 complex could modulate the effects of thyroid hormone, including the regulation of the transcriptional activity of the APP gene (Belandia et al., 1998), and the splicing of the APP primary transcript, and the secretion of APP metabolites (Latasa et al., 1998).

We report an interaction between chimaerin alpha 2 and the hypothetical protein CG005. The gene for CG005 was cloned form a region of chromosome 13 that also contains BRCA2 (Couch et al., 1996). This hypothetical protein contains 583 amino acids, with an ATP/GTP binding site between aa 409 and 416. Because chimaerin A2 contains a GTPase activating protein domain, CG005 might be an accessory protein that bring GTP in the proximity of chimaerin A2 and its GTPases targets.

δ-catenin is a member of the Armadillo protein family and was identified as a brain-specific interactor with PS1 (Zhou et al., 1997b; Zhou et al., 1997a). The protein contains 1225 amino acids with ten Armadillo domains, and it interacts with the hydrophilic loop of PS1, but not with that of PS2 (Tanahashi and Tabira, 1999). It is believed that δ-catenin interactors might alter APP metabolism and Aβ production. Beside its potential role in Alzheimer, δ-catenin was also shown to be involved in the mental retardation associated with the cri-du-chat syndrome (Medina et al., 2000).

We report an interaction between δ-catenin and the thyroid hormone interacting protein 1 (TRIP1). TRIP1 was identified in a search aiming at the identification of candidate human transcriptional mediator that interacts with the thyroid-hormone receptor in a ligand-dependent fashion (Lee et al., 1995). TRIP1 is very similar (but not identical) to the p45 protein of the 26S proteasome subunit, and it was proposed that both proteins could function equivalently in protein degradation pathways as well as transcription activation (Akiyama et al., 1995). The relevance of this interaction to Alzheimer is two-fold. On one hand, it is well documented that protein degradation through the proteasome system plays a major role in apoptosis (Orlowski, 1999; Grimm and Osborne, 1999). Moreover, the C-terminal fragments (CTF) of both PS1 and PS2 are degraded by the 20S proteasome (da Costa et al., 1999; Van Gassen et al., 1999), while full-length PS1 is degraded by the 26S proteasome (Fraser et al., 1998). In this respect, δ-catenin might be considered as an adaptor molecule targeting the presenilins to the proteasome. It is conceivable that FAD mutations in the presenilins could activate their proteasome-mediated degradation, and down the road, apoptosis. On the other hand, there is also evidence that thyroid hormone reduces the transcriptional activity of the APP gene (Belandia et al., 1998), and controls the splicing of the APP primary transcript, and the secretion of APP metabolites (Latasa et al., 1998). In this respect, TRIP1 could be an intermediate molecule bridging the PS1-δ-catenin complex to the thyroid receptor signaling, with obvious effects on APP expression and secretion.

As its name indicates, CIB was initially identified for its calcium and integrin binding properties (Naik et al., 1997). CIB contains two EF-hands and is 58% similar to calcineurin B (regulatory subunit) and 56% similar to calmodulin. The authors suggested that CIB might be the regulatory subunit of a novel (yet to be discovered) calcium-dependent phosphatase. Also known as calmyrin, CIB was reported as an interactor with both presenilins, although the interaction with PS2 is stronger than with PS1 (Stabler et al., 1999). Because mutations in both PS1 (Chan et al., 2000) and PS2 (Leissring et al., 1999) disrupt calcium homeostasis, CIB might be involved in the neuronal apoptotic cascade elicited by the presenilin mutations.

We report an interaction between the calcium and integrin binding protein (CIB) and cisplatin resistance-associated overexpressed protein (CROP), also called Luc7A. CROP is a protein of 432 amino acids. Its cDNA was cloned from cisplatin-resistant cells by differential display (Nishii et al., 2000). The N-terminal half of CROP contains cysteine/histidine motifs and leucine zipper-like repeats. The C-terminal half consists mostly of charged and polar amino acids: arginine (58 residues or 25%), glutamate (36 residues or 16%), serine (35 residues or 15%), lysine (30 residues, 13%), and aspartate (20 residues or 9%). The C-terminal half is thus extremely hydrophilic. The arginine/serine-rich domain is dominated by a series of 8 amino acid imperfect repetitive motif, which has been found in RNA splicing factors. CROP is the human homologue of yeast Luc7p, which is supposed to be involved in 5′-splice site recognition and is essential for vegetative growth.

We report an interaction between the calcium and integrin binding protein (CIB) and the F-box protein 21 (Fbx21). The cDNA encoding the Fbx21 protein was cloned as a member of a family of 26 proteins (Cenciarelli et al., 1999). F-box proteins are an expanding family of eukaryotic proteins characterized by an approximately 40 amino acid motif, the F box (so named because cyclin F was one of the first proteins in which this motif was identified). Some F-box proteins have been shown to be critical for the controlled degradation of cellular regulatory proteins. In fact, F-box proteins are one of the four subunits of ubiquitin protein ligases called SCFs. One of these SCFs, the F box protein beta-Trcp, associates with the Cull/Skp1 complex and regulates the stability of β-catenin (Latres et al., 1999). The present interaction suggests that Fbx1 might control the stability of CIB, or protein complexes that contain CIB. The sequence of Fbx21 is identical to that of the KIAA0875 protein. The KDRI report for KIAA0875 shows a sequence of 621 amino acids, with an ATP synthase domain between aa 212 and 224. The mRNA for KIAA0875 is found at highest levels in brain, heart, and spleen, followed by testis, ovary, kidney, liver, pancreas, skeletal muscle, and finally lung.

We report an interaction between the calcium and integrin binding protein (CIB) and KIAA0127. The KDRI report for KIAA0127 shows a full-length sequence of 5544 base pairs that contains an ORF coding for 315 amino acids. The Northern blot data indicate that the KIAA0127 mRNA is at highest levels in thymus, peripheral leukocytes, and skeletal muscle, and at lowest levels in brain.

We report an interaction between the calcium and integrin binding protein (CIB) and KIAA0281. The KDRI report for KIAA0281 shows a full length sequence for a protein of 271 amino acids, in which a pleckstrin homology domain and a leucine zipper domain were identified.

The proteins disclosed in the present invention were found to interact with APP or other proteins in the AD pathway. Because of the involvement of these proteins in AD, the proteins disclosed herein also participate in the pathogenesis of AD. Modulation (preferably disruption) of the protein-protein interactions can lead to the modulation of APP production and APP metabolism, Aβ production and Aβ metabolism, regulation of the wingless pathway, and modulation of thyroid receptor signaling in cells, and treatment of diseases such as Alzheimer's disease, Huntington's disease, Parkinson's disease, dementia and other neurodegenerative diseases, diabetes, obesity and coronary artery disease.

Type-A receptors for the neurotransmitter GABA are ligand-gated chloride channels that mediate inhibitory neurotransmission. Each subunit of the pentameric receptor protein has ligand-binding sites in the amino-terminal extracellular domain, four membrane-spanning regions (one of which forms a wall of the ion channel), and a large intracellular loop that may be involved in subcellular targeting and membrane clustering of the receptor. Using this intracellular loop as a bait, the cDNA encoding a novel intracellular protein of 117 amino acids was identified in a human brain library and the protein was named GABARAP. Wang et al., Nature, 397:69-72 (1999). Sequence analysis predicted that GABARAP contains a basic N-terminus and an acidic C-terminus, with an overall pI of 9.6. Binding analysis showed that the N-terminal 21 amino acids of GABARAP form an alpha helix that interacts with tubulin, thus potentially linking the GABA receptor to the cytoskeleton. The same authors suggested that GABARAP is involved in the targeting and clustering of the GABA receptor. The GABARAP-like protein 1 (GABARAPL1) also contains 117 aa and is 87% identical (94% similar) to GABARAP. This protein was identified recently as a paralogue of GABARAP. Xin et al., Genomics, 74:408-413 (2001). Expression studies showed that the GABARAPL1 mRNA is ubiquitous, at very high levels in brain, heart, peripheral blood leukocytes, liver, kidney, placenta, and skeletal muscle, lowest levels in thymus and small intestine. Xin et al., Genomics, 74:408-413 (2001). The ganglioside expression factor 2 (GEF-2) also contains 117 aa and is 57% identical to GABARAP and 61% identical to GABARAPL1. GEF-2 is also known as GABARAP-like protein 2 (GABARAPL2) and was identified by the same group who found GABARAPL1. Xin et al., Genomics, 74:408-413 (2001). Expression studies showed that the GABARAPL2 mRNA is ubiquitous, at highest levels in heart, brain, testis, prostate, ovary, spleen, and skeletal muscle, at lowest levels in lung, thymus, and small intestine. In spite of high sequence similarity with GABARAP, it is not known if GABARAPL1 and GABARAPL2 are indeed GABA receptor associated proteins. These two proteins could potentially be associated with other neurotransmitter systems.

We report interactions of each of the three proteins GABARAP, GABARAPL1, and GEF-2 with ankyrin 2 (ANK2). Also called brain ankyrin and non-erythroid ankyrin, ANK2 is a large protein (3957 amino acids) found at the inner surface of the plasma membrane of neurons and glial cells throughout the brain. Otto et al., J. Cell Biol., 114:241-253 (1991). ANK2 links integral membrane proteins (such as receptors) with components of the cytoskeleton (such as tubulin, fodrin, and others). The N-terminal half of the protein contains 23 ankyrin repeats (from amino acids 63 to 822) and 15 repeats of a new type (12 amino acids each, from amino acids 1773 to 1950). The C-terminal region contains one death domain (amino acids 3536 to 3620). These three interactions suggest that the clustering function of GABARAP on the GABA receptor is mediated by its interaction with ANK2. Likewise, the putative clustering functions of GABARAPL1 and GEF-2 on the GABA receptor or other type of receptor are mediated by ANK2.

We report interactions of each of the three proteins GABARAP, GABARAPL1, and GEF-2 with calreticulin. Calreticulin is a multifunctional protein that acts as a major calcium-binding (storage) protein in the lumen of the ER (endoplasmic reticulum). It is also found in the nucleus, suggesting that it may have a role in transcription regulation. Calreticulin binds to the synthetic peptide KLGFFKR, which is almost identical to an amino acid sequence in the DNA-binding domain of the superfamily of nuclear receptors. Dedhar et al., Nature, 367:480-483 (1994). These authors showed that calreticulin can inhibit the binding of androgen receptor to its hormone-responsive DNA element and can inhibit androgen receptor and retinoic acid receptor transcriptional activities in vivo, as well as retinoic acid-induced neuronal differentiation. Furthermore, another report showed that the amino terminus of calreticulin interacts with the DNA-binding domain of the glucocorticoid receptor and prevents the receptor from binding to its specific glucocorticoid response element. Burns et al., Nature, 367:476-480 (1994). A recent study reported that in brain neurons, CALR staining is mostly cytoplasmic and nucleolar, while in glial cells, CALR staining is mainly perinuclear (a few glial processes are also positive). Taguchi et al., Acta. Neuropathol. (Berl), 100: 153-160 (2000). By comparison with normal brain, the levels of CALR mRNA and protein are reduced in Alzheimer's brain. This observation was made by measuring both the steady-state levels of the mRNA (by rtPCR) and the number of positive cells (by in situ hybridization). In brief, CALR is found in the ER, in the cytoplasm, in the nucleus, in the nucleoli, in the perinuclear region, and in some cell processes. CALR contains 417 amino acids, including a putative signal-peptide at the N-terminus (amino acids 1 to 17), three calreticulin family repeat motifs (twelve amino acids each, from amino acids 208 to 254), and an ER targeting motif at the C-terminus (amino acids 414 to 417).

We report interactions of each of the two proteins GABARAP and GABARAPL1 with KIAA0443. The KDRI report for KIAA0443 shows a cDNA sequence of 5190 base pairs (bp) with an open reading frame of 4212 bp coding for 1404 amino acids. KIAA0443 is also known as G protein-coupled receptor-associated sorting protein (GASP). The protein contains four lipocalin domains scattered between amino acids 409 and 1017, suggesting that this KIAA protein might be involved in the transport of small lipophilic molecules. An ATP binding site is located between amino acids 1385 to 1392. The message for KIAA0443 is at highest levels in brain, kidney, prostate, and ovary. Medium levels are found in thymus and small intestine. Low levels are found in placenta, lung, pancreas, heart, and testis. In liver, skeletal muscle, and spleen, the message is barely detectable. The KIAA0443 protein was first reported as a δ-catenin interactor (see U.S. patent application Ser. No. 09/466,139 filed 21 Dec. 1999 and published PCT application WO 00/37483). The interactions reported here suggest that the 6-catenin and KIAA0443 complex could modulate GABAergic transmission (in case the complex contains GABARAP) or transmission mediated by other neurotransmitters (in case the complex contains GABARAPL1). Disruption or interfering with the KIAA0443-GABARAP interaction or KIAA0443-GABARAPL1 interaction could lead to modulated neurotransmission and decreased Aβ42 production or secretion from neuronal cells, thus leading to the treatment or prevention of neurodegenerative disorders such as Alzheimer's disease (including mild cognitive impairment), Parkinson's disease, Huntington's disease, schizophrenia and depression.

We report interactions of each of the two proteins GABARAP and GABARAPL1 with the microtubule-associated protein 1A (MAP1A). The assembly of microtubules is an essential step in neurogenesis and is modulated by a family of proteins called microtubule-associated proteins (MAPs). These proteins have been divided by size into two main groups: high molecular weight MAPs, which include MAP 1A, MAP1B, and MAP2, and another group of intermediate-sized proteins, which include the abundant tau MAPs. MAP1B, also named MAP5, is a component of long cross-bridges between microtubules and is a filamentous molecule with a small spherical segment at one end. Cloning of the MAP1A cDNA revealed some striking structural similarity with MAP1B, a protein associated with neurite outgrowth and process plasticity. Langkopf et al., J. Biol. Chem., 267:16561-16566 (1992). The two MAPs exhibit regional amino acid sequence similarities spanning their potential microtubule binding domains. In addition, the authors also showed that the mRNA for MAP1A also codes for its associated light chains (LC) called LC2. MAP1A and LC2 are translated as one unique pro-protein chain that is proteolytically cleaved to generate the mature MAP1A and LC2 proteins, in a way identical to the formation of MAP1B and LC1. Hammarback et al., Neuron, 7:129-39 (1991).

The 10 kb MAP1A message is expressed specifically in brain and the gene for MAP1A is on chromosome 15. Fukuyama & Rapoport, J. Neurosci. Res., 40:820-825 (1995). MAP1A and MAP1B are phosphorylated by kinases that are insensitive to cAMP, cGMP, and calcium. From amino acids 415 to 541, MAP1A contains nine repeats of three amino acids composed of two lysine residues and either one aspartic acid or glutamic acid residue.

We report an interaction between GABARAP and the protein kinase A (PKA) anchor protein 11 (AKAP11). A-kinase anchoring proteins (AKAPs) function to compartmentalize type II cyclic AMP-dependent kinase (PKA) by associating with the PKA regulatory subunit (RII). AKAP11 was first identified in the rat by screening a rat pituitary and olfactory bulb cDNA libraries with a protein kinase A regulatory (RII) probe. Lester et al., J. Biol. Chem., 271:9460-9465 (1996). The protein (which the authors call AKAP220 due to its 220 kDa size as determined by Western blot analysis) contains a PKA binding region and a peroxisome targeting motif. Northern blot analysis revealed two AKAP11 mRNA transcripts (9.7 kb and 7.3 kb) in several rat tissues with highest expression in brain and testis. In agreement with these findings, immunoprecipitation confirmed that AKAP11 associates with the type II PKA holoenzyme. Furthermore, AKAP11 and some of the cellular RII regulatory subunit were found to co-localize in microbodies thought to be a subset of peroxisomes. Human AKAP11 was identified by the KDRI as KIAA0629. The human AKAP11 is a 1901 amino acid protein with an ATP/GTP-binding site motif A (P-loop) (amino acids 1802 to 1809), four cAMP/cGMP-dependent kinase phosphorylation consensus sites (amino acids 413 to 416, 1118 to 1121, 1286 to 1289, and 1424 to 1427), and three tyrosine kinase phosphorylation consensus sites (amino acids 418 to 424, 898 to 906, 1568 to 1575). The KDRI reports high expression in brain, ovary, and kidney, moderate expression in heart, lung, liver, testis, and spinal cord, and lower expression in skeletal muscle, pancreas, and spleen. AKAP11 is differentially expressed throughout the brain with higher expression in the amygdala, thalamus, caudate nucleus, and corpus callosum as compared to the hippocampus, substantia nigra, and cerebellum. The interaction of GABARAP with AKAP11 supports the finding that cAMP dependent phosphorylation of type A GABA receptors might control GABAergic transmission. Moss et al., Science, 257:661-665 (1992b); McDonald et al, Nat. Neurosci., 1:23-28 (1998); Nusser et al., J. Physiol., 521 Pt 2:421-435 (1999). As GABA receptors are abundant in the hippocampus, GABA and cAMP could modulate hippocampal long term potentiation (LTP) and long term depression (LTD). In fact, type A GABA receptors can be modulated by both PKA and PKC in the hippocampus. Poisbeau et al., J. Neurosci., 19:674-683 (1999). Furthermore, a recent work showed that type A GABA receptors can modulate cAMP-mediated LTP and LTD at monosynaptic junctions between the CA3 and CA1 regions. Yu et al., Proc Natl Acad Sci USA, 98:5264-5269 (2001).

We report an interaction between GABARAPL1 and KIAA1855. The KDRI database report for KIAA1855 shows a cDNA sequence of 6997 base pairs (bp) containing an open reading frame (ORF) of 3810 bp coding for 1270 amino acids. The putative ATG initiation codon starts at the second nucleotide of the cDNA, thus leaving the frame open in the 5′ direction. An EST from human brain (AI197837) allows one to extend the sequence by 109 bp in the 5′ direction. However no in-frame upstream STOP codon is found in this additional sequence. The putative ATG initiation codon is in a reasonable Kozak environment, suggesting that it might indeed be the authentic initiation codon. Amino acid sequence analysis by Pfam identified a protein kinase domain from amino acids 5 to 217. The specificity of this kinase domain cannot be predicted (could be either a Ser/Thr kinase domain or a Tyr kinase domain). The mRNA for this KIAA protein, measured by rt-PCR-ELISA at the KDRI, is found at high levels in brain, medium to low levels in testis and lung, and very low levels in all other tissues examined (heart, liver, skeletal muscle, kidney, pancreas, spleen, and ovary). Within the brain, the KIAA1855 mRNA is at medium to high levels in all regions examined (including the hippocampus, amygdala, cerebellum, and caudate nucleus). Thus, we suggest that GABARAPL1 interacts with a brain-enriched KIAA protein that is a putative kinase.

APP metabolism is a critical event in the pathogenesis of Alzheimer's, because it leads to the release of either toxic (Aβ) or trophic (sAPP) metabolites (Cummings et al., 1998; Roch and Puttfarcken, 1996). In this respect, it is very important to identify proteins involved in the intracellular trafficking of APP. Proteins that interact with the cytosolic C-terminal region of APP play a major role in this process. The interaction of APP with Fe65, with Fe65 L, with Mint1, and with Mint2 have been well documented (Russo et al., 1998; Sastre et al., 1998). In turn, Fe65 was shown to interact with Mena, the mammalian homolog of the Drosophila enabled protein (Ermekova et al., 1997), and with the transcription factor LSF (Zambrano et al., 1998). The functional significance of those interactions have been discussed (Russo et al., 1998). Basically, this interaction network centered around the cytosolic tail of APP is proposed to be involved in 1) endocytosis and intracellular trafficking of APP, and 2) intracellular signalling events mediated by APP. Another work has shown that Fe65 and the mammalian homolog of the Drosophila disabled protein (DAB) bind to the cytosolic tail of several proteins involved in AD, like the LDL receptor, the LDL receptor related protein (LRP), and APP (Trommsdorff et al., 1998). The authors proposed that Fe65 and DAB can serve as molecular scaffold that bring together components of intracellular signalling complexes, including non-receptor tyrosine kinases such as the Abl protein. Thus, searching for interactors for LRP, Abl, and other proteins involved in this network may yield novel drug targets and therapeutic opportunities.

Abl is a cytoplasmic and nuclear protein tyrosine kinase that has been implicated in processes of cell differentiation, cell division, cell adhesion, and stress response (Mauro and Druker, 2001; Fernandez-Luna, 2000). The Abl protein contains 1130 amino acids (Fainstein et al., 1989) with an SH3 domain from amino acids 61 to 121, and SH2 domain from amino acids 127 to 217, and a protein kinase domain from amino acids 242 to 493. This kinase domain contains an ATP binding site from amino acids 248 to 271 and a tyrosine kinase active site from amino acids 359 to 371. The SH3 domain negatively regulates the tyrosine kinase activity, while deletion of the SH3 domain from Abl results in a protein with constitutive kinase activity. Furthermore, a very recent report (Zambrano et al., 2001) showed that, in cells expressing a constitutively active form of Abl, the cyto-tail of the amyloid protein precursor (APP) is tyrosine phosphorylated. Thus, Abl activity could modulate APP interaction with proteins such as Fe65, Fe65L, Mint1, and Mint2, which have been implicated in APP metabolism and Aβ production. There is also ample evidence that apoptosis is a major factor responsible for the neuronal loss observed in Aβ and other neurodegenerative conditions (Cotman et al., 1994; Smale et al., 1995; Shimohama, 2000; Behl, 2000; Offen et al., 2000). In particular, the role of the Bax protein in amyloid-induced neurotoxicity has been established (Selznick et al., 2000). Likewise, caspase-3 was shown to be activated in apoptotic neurons in response to exposure to the Aβ peptide (Harada and Sugimoto, 1999; Robertson et al., 2000). In addition, APP and the presenilins are targets of caspase-3 (Tesco et al., 1998; Weidemann et al., 1999; Gervais et al., 1999; Walter et al., 1998). Thus, Bax and caspase-3 interactors are also worth searching for. The protein CASK was identified as an interactor with the X11 (Mint1) protein (Borg et al., 1998; Borg et al., 1999). CASK is a member of the membrane-associated guanylate kinase (MAGUK) family. Proteins from this family typically contain one to three PDZ domains, an SH3 domain, and a guanylate kinase (GK) domain. In addition, CASK contains a serine/threonine protein kinase domain in the N-terminal region. MAGUK proteins are mainly localized at the synapses and function as synaptic scaffolding and clustering molecules for signalling proteins such as NMDA receptors and potassium channels (Kornau et al., 1995; Nagano et al., 1998; Muller et al., 1996; Nehring et al., 2000). Because of the effect of the Mint1-CASK complex on APP trafficking and metabolism (Tomita et al., 1999; Mueller et al., 2000), searching for CASK interactors may also yield drug target opportunities that could modulate amyloid production. Likewise, searching for novel MAGUK interactors is also a strategy to find new signalling proteins involved in synaptic function and that represent therapeutic opportunities against neurodegeneration. Members of the MAGUK family that we used in searches include the post-synaptic density protein 95 (PSD95), the discs-large protein 1 (DLG1), the discs-large protein 2 (DLG2), the discs-large protein 3 (DLG3), and the neuroendocrine discs-large protein (NE-dlg).

We report interactions between PSD95 and the novel protein PN7740. PSD95 is a member of the MAGUK family (membrane associated guanylate kinase) and contains three PDZ domains, one SH3 domain, and one guanylate kinase (GK) domain (Wheal et al., 1998; Dimitratos et al., 1999). Also called DLG4 and SAP-90 (Kistner et al., 1993; Stathakis et al., 1997), PSD95 is found at the post-synaptic density where it interacts with other synaptic scaffolding proteins and with synaptic signalling proteins such as neurotransmitter receptors and channels (e.g. NMDA receptors, potassium channels) (Kornau et al., 1995; Nagano et al., 1998; Lau et al., 1996; Nehring et al., 2000). PN7740 is a novel protein that we previously reported as an Fe65 interactor (see U.S. patent application Ser. No. 60/240,790 filed 17 Oct. 2000). The full-length cDNA for PN7740 contains a open reading frame (ORF) coding for 372 amino acids. The putative ATG initiation codon is preceded by a purine (G) residue in position −3, and by several upstream STOP codons, suggesting that it represents the authentic initiation codon. At the end of the 3′ UTR (untranslated region), we found a canonical polyadenylation signal (AATAAA) shortly before the poly A itself. A phosphatase 2C domain was found from amino acids 104 to 339. Thus, we identified a novel phosphatase that binds to the first PTB domain of Fe65.

Confirming a report from the literature (Kawachi et al., 1999), we also found an interaction between the protein tyrosine phosphatase zeta (PTPZ) and PSD95. PTPZ is a large type I transmembrane protein of 2314 amino acids, expressed specifically in the central nervous system (Krueger and Saito, 1992; Shintani et al., 1998). It has the typical structure of a cell surface receptor, with a signal peptide from amino acids 1 to 24 and a single transmembrane domain from amino acids 1636 to 1661. Amino acids 25 to 1635 are extracellular, while amino acids 1662 to 2314 are cytoplasmic. Two tyrosine phosphatase domains are from amino acids 1744 to 1997 and from amino acids 1998 to 2314. Interestingly, PTPZ expression is increased in response to injury (Li et al., 1998). It is also expressed at high levels by neurons and astrocytes during brain development. PTPZ belongs to a large family of phosphatases that play important roles in neuronal functions. We previously reported an interaction between an extracellular region of APP that carries the neurotrophic domain and an extracellular domain of PTPZ (see U.S. patent application Ser. No. 09/466,139 filed 21 Dec. 1999 and published PCT application WO 00/37483) and we suggested that PTPZ is a receptor that transduces the neurotrophic effects elicited by sAPP. The PTPZ bait used in the search is a domain from amino acids 1998 to 2314 (the C-terminus). This bait ends with amino acids -LESLV-COOH, which is a good PDZ domain binding motif. Using this bait, we found a region of PSD95 from amino acids 66 to 443, containing all three PDZ domains of the protein. Thus, we and others (Kawachi et al., 1999) propose that PSD95 participates in the clustering of PTPZ, a receptor for sAPP. Combined together, our data suggest that Fe65, APP, PN7740, PTPZ, and PSD95 might be part of a large protein complex. The activity of PN7740 and PTPZ could control APP metabolism, as it is well documented that the balance of α-secretion vs β-secretion of APP is regulated by phosphorylation (Farber et al., 1995; Caporaso et al., 1992; Buxbaum et al., 1990; Buxbaum et al., 1993; Sabo et al., 1999). Alternatively, PN7740 might also be involved in the biochemical events elicited by sAPP binding to PTPZ, a phenomenon that is expected to elicit a neurotrophic response.

We report an interaction between presenilin 1 (PS1) and the discs-large protein 2 (DLG2). Mutations in PS1 have been shown to cause early onset familial Alzheimer's disease (FAD) (Hardy, 1997; Lippa, 1999) and PS1 has been proposed to be the g-secretase enzyme that cleaves APP at the C-terminus of the Ab peptide (Wolfe et al., 1999b; Selkoe and Wolfe, 2000; Li et al., 2000a; Li et al., 2000b). DLG2 is a member of the MAGUK family (membrane associated guanylate kinase) and contains three PDZ domains, one SH3 domain, and one guanylate kinase (GK) domain (Wheal et al., 1998; Dimitratos et al., 1999). As most MAGUK proteins, DLG2 is found at the post-synaptic density where it interacts with other synaptic scaffolding proteins and with synaptic signalling proteins such as neurotransmitter receptors and channels (e.g. NMDA receptors, potassium channels) (Nagano et al., 1998; Makino et al., 1997; Muller et al., 1996). The PS1 baits that we used spanned amino acids 253 to 293, and were either wild-type or carried the FAD mutation E280A. The carboxy terminus of these baits have a six amino acid motif which is a PDZ-binding peptide. Although the major PS1 cleavage site is at aa Met298 in the loop number 6, the cleavage is heterogeneous and occurs between Thr291 and Ala299 (Podlisny et al., 1997). If the cleavage occurs at V293, it would leave behind an N-terminal fragment of PS1 with a PDZ binding C-terminus, capable of interacting with DLG2. Interestingly, the E280A FAD mutation does not affect this interaction. in any event, this interaction suggests that DLG2 (and other MAGUKs) interactors could be involved in PS1 function and therefore in AD pathogenesis.

We now report that the three MAGUK proteins PSD95, DLG2, and neuroendocrine-dlg (NE-dlg) interact with the enzyme diacylglycerol (DAG) kinase 6 (DAGK6). Also known as DAG kinase zeta or DAGKZ, DAGK6 is the enzyme that converts 1,2-diacylglycerol into 1,2-diacylglycerol 3-phosphate (phosphatidic acid), with the concomitant hydrolysis of ATP into ADP. The cDNA for DAGK6 was cloned from human endothelial cells (Bunting et al., 1996). These authors report that DAGK6 contains two zinc fingers, an ATP binding site, and four ankyrin repeats near the carboxyl terminus. A unique feature, as compared with other DAG kinases, is the presence of a sequence homologous to the MARCKS (myristoylated, alanine-rich C-kinase substrate) phosphorylation site (MARCKS is a major substrate for protein kinase C (PKC), which is activated by DAG). Northern blot analysis revealed highest levels of DAGK6 in brain, although skeletal muscle, heart, and pancreas also have substantial levels. In the brain, DAGK6 is found in the cerebellar and cerebral cortices (Goto and Kondo, 1999). DAGK6 is both nuclear and cytoplasmic. A nuclear localization signal (NLS) is found in the MARCKS homology domain. Phosphorylation of this MARCKS domain by PCK reduces the nuclear levels of DAGK6 (Topham et al., 1998). Because DAG is a PKC activator, the authors suggest the existence of a regulatory loop in which DAG activates PKC, which then regulates the metabolism of DAG by alternating the intracellular location of DAGK6.

The PSD95, DLG2, and NE-dlg baits used in the searches all contain the third PDZ domain. We found clones encoding amino acids 806 to 928 (C-terminus) of DAGK6 in a brain library, and clones encoding amino acids 827 to 928 (C-terminus) of DAGK6 in a hippocampus library. The C-terminus of DAGK6 is TAV-COOH, which is a good PDZ domain binding motif. Thus, we suggest that the third PDZ domain of PSD95, DLG2, and NE-dlg bind to the C-terminus of DAGK6. Diacylglycerol (DAG) is a complex lipid with important structural and signalling functions in the brain. DAG is the precursor to glycerolipids (phosphatidyl-choline, -serine, -ethanolamine), is an allosteric regulator of protein kinase C (PKC), and the cellular levels of DAG may influence a variety of processes including growth, differentiation, and synaptic vesicle fusion with presynaptic membranes. DAG can be phosphorylated by a number of kinases (DAGKs) among which DAGK6 (or DAGK zeta) shows a marked selectivity for 1,2-DAG. Phosphorylation occurs in position three, resulting in the formation of phosphatidic acid (PtdOH), which is itself a precursor to other signalling molecules, the phosphoinositol phosphates. Remarkably, severe dysfunctions in the phosphoinositide signalling pathway have been reported in the brains of Alzheimer's Disease (AD) patients (Fowler, 1997). Thus, DAGK6 activity is important in controlling the balance between DAG and PtdOH levels, leading to the regulation of PKC activity and levels of intracellular calcium.

This interaction of DAGK6 with three MAGUK proteins makes sense in the light of the DAG and PtdOH involvement in synaptic vesicle fusion (Betz et al., 1997; Siddhanta and Shields, 1998), and the importance of PKC and calcium in synaptic function. The regulation of APP alpha secretion by PKC is well documented (Gandy and Greengard, 1994; Jolly-Tometta and Wolf, 2000). In a very recent study, the epsilon isoform of PKC was shown to stimulate APP alpha secretion (Yeon et al., 2001). Because the alpha cleavage occurs within the amyloid domain of APP, the alpha secretion precludes Aβ formation. Thus, in addition to stimulating the secretion of αsAPP, a protein with neurotrophic and neuroprotective properties (Roch et al., 1993; Saitoh and Roch, 1995; Roch and Puttfarcken, 1996; Saitoh et al., 1995; Mattson et al., 1999; Mattson and Duan, 1999), PKC activation is expected to reduce the production of the Aβ protein, a hypothesis that was confirmed (Hung et al., 1993). The activity of DAGK6 (phosphorylation of DAG into PtdOH) is thus expected to result in reduced PKC activity, and thus in elevated Ab levels and lower αsAPP levels. It is also interesting that PtdOH stimulates the fibrillization of the Aβ protein, while DAG does not (Chauhan et al., 2000). It is thus tempting to speculate that pharmacological modulation (inhibition) of DAGK6 could result in elevated DAG levels, leading to PKC activation, reduced Aβ production and increased (sAPP secretion.

We also reported that PSD95 interacts with the novel protein PN7740 (see above), which was previously found as an Fe65 interactor (see U.S. patent application Ser. No. 60/240,790 filed 17 Oct. 2000). Analysis of the amino acid sequence of PN7740 suggested that it might be a novel phosphatase. The MARCKS (myristoylated, alanine-rich C-kinase substrate) domain of DAGK6 can be phosphorylated by PKC, which reduces the nuclear localization of DAGK6. As PKC itself is activated by DAG, it was suggested that a regulatory loop in which DAG activates PKC, might control the metabolism of DAG by alternating the phosphorylation state (and thus the intracellular localization) of DAGK6 (Topham et al., 1998). In this respect, it is tempting to speculate that PN7740 might also participate in the regulation of the phosphorylation state of DAGK6, by virtue of their common interactor, PSD95 (see above). Beside its potential involvement in neuronal degeneration or survival, DAGK6 activity is also related to obesity. A very recent work (Liu et al., 2001) showed that DAGK6 interacts with the cytoplasmic domain of the leptin receptor in the hypothalamus. The authors suggested that reduced DAGK6 activity might be associated with obesity. In brief, pharmacological modulation of DAGK6 activity could result in either demented thin patients, or non-demented obese patients.

We report an interaction between the cytosolic domain of the low-density lipoprotein receptor related protein 1 (LRP1) and the islet-brain protein (IB1), so named because it is expressed in pancreatic beta cells and in brain. IB1 was originally isolated from pancreatic beta-cell by its ability to bind to GTII, a cis-regulatory element of the glucose transporter 2 (GLUT2) gene promoter (Bonny et al., 1998). IB1 was found to be very similar to JIP-1, a cytoplasmic inhibitor of the c-Jun amino-terminal kinase activated pathway, that was cloned from mouse brain. IB1 is 97% identical to JIP-1 and contains an insertion of 47 aa in the C-terminal region. Localized in both the cytoplasm and the nucleus, IB1 transactivates the GLUT2 gene. It also contains a phosphotyrosine interaction domain (PID) and an SH3 domain. A recent work showed that in mouse brain, IB1 is found in the synaptic regions of the olfactory bulb, retina, cerebral and cerebellar cortex and hippocampus (Pellet et al., 2000). IB1 was also detected in a restricted number of axons, as in the mossy fibers from dentate gyrus in the hippocampus, and was found in soma, dendrites and axons of cerebellar Purkinje cells. A human IB1 gene was recently identified on chromosome 11, containing 12 exons (Mooser et al., 1999). IB1 (JIP-1) was also found to interact with rhoGEF, a neuron-specific guanine nucleotide exchange factor for the GTPase rhoA (Meyer et al., 1999). In this respect, it is interesting that LRP was found to interact with a GTPase (Gas), further supporting the notion that LRP is a receptor that mediates signal transduction (Goretzki and Mueller, 1998). Along those lines, it should be noted that Disabled (DAB) binds to the cytosolic tail of LRP1, the LDL receptor, and APP (Trommsdorff et al., 1998). As DAB can recruit nonreceptor tyrosine kinases (e.g. src, abl) to the cytoplasmic tails of the receptors to which it binds, LRP might possibly also signal through a tyrosine phosphorylation cascade. The interaction between LRP and IB1 suggests that LRP might also signal through transcriptional activation of the GLUT2 gene or through activation of the rhoA GTPase.

We also report interactions between the product of the neuroepithelioma transforming gene 1 (NET1) and three proteins of the MAGUK family, S-SCAM, the neuroendocrine discs large protein (NE-dlg) the and the discs-large protein 2 (DLG2). NE-dlg and DLG2 are members of the membrane-associated guanylate kinase family (MAGUK) and contains three PDZ domains, one SH3 domain, and one guanylate kinase (GK) domain. S-SCAM is a particular member of the MAGUK family (membrane associated guanylate kinase) that contains 1455 amino acids. Instead of the usual three PDZ domains found in MAGUK proteins, S-SCAM has five or six PDZ domains (depending on the tool used for sequence analysis) scattered between amino acids 17 and 1229. The guanylate kinase (GK) domain, normally found in the C-terminal region of MAGUK proteins is in the N-terminal region of S-SCAM (amino acids 109 185). Finally, the SH3 domain is replaced by two WW domains between amino acids 302 and 381. S—SCAM was first identified as an interactor with atrophin-1 (Wood et al., 1998), a protein that contains a polyglutamine repeat, the expansion of which is responsible for dentatorubral and pallidoluysian atrophy (DRPLA). S-SCAM was subsequently found to also interact with δ-catenin (Ide et al., 1999), a protein that also interacts with PS1 (Zhou et al., 1997b) and which is involved in the mental retardation observed in the cri-du-chat syndrome (Medina et al., 2000). MAGUKs are found at the post-synaptic density where it interacts with other synaptic scaffolding proteins and with synaptic signalling proteins such as neurotransmitter receptors and channels (e.g. NMDA receptors, potassium channels) (Nagano et al., 1998; Makino et al., 1997; Muller et al., 1996). The cDNA encoding NET1 was isolated by expression cloning from a human neuroepithelioma library (Chan et al., 1996). NET1 is a Rho-family guanine-nucleotide exchange factor (GEF), which contains a Dbl homology (DH) domain and the typical neighboring pleckstrin homology (PH) domain (Alberts and Treisman, 1998). The DH domain is required for GEF activity, and the PH domain apparently mediates subcellular localization. The NE-dlg bait used in the search was from amino acids 216 to 578, a region of the protein that contains the second and third PDZ domains and the SH3 domain. The DLG2 bait used in the search was from amino acids 411 to 870, a region of the protein that contains the third PDZ domain, the SH3 domain, and the GK domain. The S-SCAM bait used in the search was from amino acids 370 to 519, a region of the protein that contains the second PDZ domain.

Using these three baits, we identified clones in a hippocampus library coding for amino acids 330 to 460 of NET1 (C-terminus). This fragment overlaps the PH domain of NET1 (amino acids 245-361). The amino acids at the C-terminus of NET1 are RLWCREGSVC-COOH and do not contain a canonical PDZ-binding motif (-S/TXV/L-COOH). However, PDZ domains are known to interact not only with such a C-terminal motif, but also with other PDZ domains or kinase domains (Ponting et al. 1997). A recent study showed that NET1 can directly activate the stress-activated protein kinase/c-Jun N-terminal kinase (SAPK/JNK) pathway (Alberts and Treisman, 1998). By virtue of its interactions with the MAGUKs DLG2, S-SCAM, and NE-dlg, NET1 might be found in synaptic compartments. Thus, degenerating neurons, as those found in the brains of Alzheimer's patients, might contain activated SAPK/JNK, in response to oxidative stress elicited by amyloid. This hypothesis was recently confirmed (Zhu et al., 2001). In addition, the interaction of NET1 with S-SCAM generates a connection with δ-catenin and PS1, thus strengthening the potential involvement of NET1 in Alzheimer's disease.

We also report an interaction between the discs large 2 protein (DLG2) and the protein kinase p38 gamma (p38). Also known as mitogen-activated protein kinase (MAPK) 12, extracellular signal-regulated kinase (ERK)₆, ERK5, stress-activated protein kinase (SAPK)3, and MAPK p38-gamma, the p38 protein was identified as a new member of the p38 group of MAP kinases (Li et al., 1996). It is 60% identical to other members of the p38 group, and its expression was said to be restricted to skeletal muscle. However, a more recent study (Lee et al, 1999) found that p38 gamma (SAPK3) is also expressed at high levels in brain. The p38 gamma expression generally overlaps that of p38 beta, although there are some exceptions such as in hippocampus, where p38 gamma is restricted to CA3 and CA4 regions while p38 beta is evenly expressed. Also, p38 beta was identified in nucleus as well as in cytoplasm of neurons, while p38 gamma was detected mainly in cytoplasm and dendrites.

The DLG2 bait used in the search was from amino acids 411 to 870, a region of the protein that contains the third PDZ domain, the SH3 domain, and the GK domain. We identified one clone in a hippocampus library coding for amino acids 224 to 367 (C-terminus) of p38 gamma. The last five amino acids of p38 gamma are -KETPL-COOH, which is a good PDZ binding motif. Thus, we suggest that the second or third PDZ domain of DLG2 interacts with the C-terminus of p38 gamma. This interaction suggests that p38 gamma might be a synaptic protein, which is compatible with the dendritic localization of p38 gamma reported (Lee et al., 1999). Thus, we found that the discs large 2 protein (DLG2) interacts with both the product of the neuroepithelioma transforming gene 1 (NET1) and the protein kinase p38 gamma (p38), also known as stress-activated kinase (SAPK)₃. As NET1 was shown to activate the stress-activated protein kinase/c-Jun N-terminal kinase (SAPK/JNK) pathway (Alberts and Treisman, 1998), we suggest that DLG2 might act as an adaptor molecule that brings together two component of a signalling complex, NET1 and p38 gamma, and that NET1 could activate p38 gamma in response to stress. In this respect, it is interesting that degenerating neurons in the brains of Alzheimer's patients contain high levels of activated SAPK/JNK (Zhu et al., 2001). It is also worth noting that the SAPK/JNK pathway and the p38 kinase are activated in response to Aβ, as a part of the microglia activation phenomenon (Pyo et al., 1998). The activity of JNK is inhibited by its binding partner, JIP, and recent study showed that this inhibition of JNK by overexpression of the JNK binding domain of JIP-1 prevents apoptosis in sympathetic neurons (Harding et al. 2001). JIP is also known as the islet-brain protein 1 (IB1), a protein that we report as an interactor with the low density lipoprotein (LDL) receptor related protein (LRP, see above), a protein that is known to interact with APP (Hyman et al., 2000). Thus, we propose that LRP, S—SCAM, and DLG2 bring together the components of a large complex that contains APP, NET1, PS1, δ-catenin, the p38 kinase (SAPK3/JNK), and JIP (IB1). The activity of the p38 kinase in the complex could be modulated by NET1 (activation) or by JIP (inhibition). It is also of great interest that p38 and JNK are strong candidates as tau kinases that may be involved in the pathogenic hyperphosphorylation of tau in Alzheimer's disease (Reynolds et al., 2000). Thus, we propose that activity of the p38 and JNK kinases, modulated by NET1 and JIPs proteins, could be involved in tau phosphorylation and APP metabolism, by virtue of the interactions reported here (DLG2, NE-dlg and S-SCAM with NET1, DLG2 with p38, LRP with JIP).

We report a interaction between the MAGUK protein NE-dlg and the novel protein PN18543 which is to be an alternative splice variant of the KIAA1422 protein (GenBank AB037843) and the human homolog of the rat Slack potassium channel (94% identical, GB AF089730). The sequence of PN18543 is in the appendix. Slack is a potassium channel that was initially cloned from rat brain (Joiner et al., 1998). It contains 1237 aa, with six transmembrane domains in the N-terminal region (first 370 residues) and a very large cytosolic C-terminal fragment. Slack channels rectify outwardly with a unitary conductance of about 25-65 pS and are inhibited by intracellular calcium. In rat, the Slack mRNA is expressed mostly in brain and kidney. In human, expression data from the KDRI indicate that KIAA1422 mRNA (Slack splice variant) is expressed at highest levels in liver, closely followed by brain and kidney, ovary, and testis. Very high levels are observed in almost all brain regions examined (including hippocampus and amygdala), with slightly lower levels in substantia nigra and corpus callosum. Our own analysis of mRNA expression by northern blot shows that human Slack is expressed in brain and skeletal muscle. The NE-dlg bait used in the search was from aa 216 to 578, a region of the protein that contains the second and third PDZ domains and the SH3 domain. We found one clone in a hippocampus library encoding the 200 C-terminal residues of PN18543 (human Slack). The C-terminus of PN18543 is TQL-COOH, a good PDZ-binding motif. Potassium channels (K channels) are very diverse in structure and function.

We previously reported an interaction between BAX and the alpha (pore-forming) subunit of the Slo (Ca2+ activated) potassium channel (see U.S. patent application Ser. No. 60/240,790 filed 17 Oct. 2000). The Slo channel (its name comes from the fly slowpoke K channel) is a member of the subfamily of large-conductance (>200 pS) calcium activated potassium channels (also called Maxi K or BK or KCa) which belong to the voltage gated K channel (Kv) family. The BK family contains many splice variants, all of which have the typical structure of Kv channels: the alpha subunit is a homotetrameric complex formed by four polypeptides, each of which contains six transmembrane (TM) domains and often large cytosolic N-terminal and C-terminal domains. The channel (pore) region is between TM5 and TM6, while TM4 acts as a voltage sensor, and calcium binding sites are found in the C-terminal cytosolic domain. Tetraethylammonium (TEA) blocks the activity of these channels. The structure and function of these potassium channels have been reviewed (Jan and Jan, 1997; Christie, 1995). Slack is also a member of the Kv family, with the typical six transmembrane domains, but contrary to the Maxi K channels (like Slo), Slack is inhibited by calcium and shows a conductance in the 25 to 65 pS range (Joiner et al. 1998). However, when Slack is co-expressed with Slo, hybrid channels with pharmacological properties and single-channel conductances that do not match either Slack or Slo are formed. The Slack/Slo channels have intermediate conductances of about 60-180 pS (medium to large) and are activated by cytoplasmic calcium. Some authors speculated that intermediate-conductance channels in the nervous system may result from an interaction between Slack and Slo channel subunits (Joiner et al., 1998). A defect in a TEA-sensitive channel with a conductance of 113 pS (compatible with a Slo-Slack hybrid) was identified in fibroblasts from AD patients (Etcheberrigaray et al., 1994). Recently, a large conductance (240 pS) TAE-sensitive channel (compatible with Slo) was found to be activated in response to sAPP (Furukawa et al., 1996a), resulting in shut down of neuronal activity and protection against a variety of insults including Aβ toxicity (Goodman and Mattson, 1996; Chi et al. 1999). It is interesting that BAX, a mediator of apoptosis, interacts with the Slo channel, which can form calcium activated channels of intermediate to large conductance with Slack, another channel potentially located at the post synaptic density (by virtue of its interaction with NE-dlg). Thus, it is tempting to speculate that the Slo channel is activated in response to sAPP, while the activity of the Slo-Slack hybrid channel is impaired in AD fibroblasts.

We report an interaction between GIPC and the small-conductance calcium-activated potassium channel KCNN3, also called SK3. GIPC is a protein of 333 amino acids that contains a PDZ domain from amino acids 133 to 213. It was first identified as an interactor with the C-terminus of RGS-GAIP, a GTPase activating protein for Gai heterotrimeric G-proteins (De Vries et al., 1998b). The same authors previously showed that GAIP is located on clathrin-coated vesicles (De Vries et al., 1998a). They also showed that GIPC is present in two pools of molecules, one soluble or cytosolic pool (70%) and one membrane-associated pool (30%). Through its GAIP interaction GIPC was suggested to be involved in G protein signalling and intracellular vesicular trafficking. Interestingly, a mouse homolog of GIPC was identified as a semaphorin F interactor (Wang et al., 1999b), hence its name Semcap. Thus, GIPC is also thought be involved in axonal outgrowth and guidance. The control of vesicle trafficking and membrane fusion is clearly important in such phenomenon. SK channels are potassium selective and are activated by an increase in the level of intracellular calcium, such as occurs during an action potential. Their activation causes membrane hyperpolarization, which inhibits cell firing and limits the firing frequency of repetitive action potentials (Bond et al., 1999). The intracellular calcium increase evoked by action potential firing decays slowly, allowing SK channel activation to generate a long-lasting hyperpolarization termed the slow afterhyperpolarization (sAHP). This spike-frequency adaptation protects the cell from the deleterious effects of continuous tetanic activity and is essential for normal neurotransmission (Bond et al. 1999). SK3 contains two polyglutamine repeats in the amino terminal region (12 and 19 residues in length) and was first cloned as a gene for spinocerebellar ataxia 2 (SCA2) (Imbert et al., 1996) and for bipolar disorder or schizophrenia (Chandy et al., 1998; Wittekindt et al., 1998). However, there are conflicting reports suggesting that SK3 may or may not be a candidate gene for psychotic disorders or SCA2. Associations between a CAG repeat expansion in SK3 and psychosis have been reported (Gargus et al., 1998; Dror et al., 1999; Bowen et al., 1998). However, these results have been challenged by other authors (Antonarakis et al., 1999; Guy et al., 1999; Bonnet-Brilhault et al., 1999; Tsai et al., 1999). The involvement of CAG repeats in genes (including SK3) in major psychosis has recently been reviewed (Vincent et al., 2000). The amino acid sequence of SK3 is very similar to that of two other channels of the SK family, SK1 (67% identical) and SK2 (73% identical). SK channels are expressed at high levels in brain. While SK1 and SK2 are found mainly in the cortex and the CA1 and CA3 regions of the hippocampus, SK3 is more broadly distributed (highest levels in the supraoptic nucleus and the inferior olivary nucleus) (Stocker and Pedarzani, 2000).

The GIPC bait used in the search was from amino acids 119 to 233, a region of the protein that contains the unique PDZ domain. We found one clone in a hippocampus library coding for amino acids 606 to 736 (C-terminus) of SK3. Interestingly, the C-terminal sequences of SK channels are not PDZ-binding motif (e.g. the C-terminus of SK3 is -TSSSSC-COOH). Thus we suggest that SK3 interacts with the PDZ domain of GIPC through an internal PDZ-binding domain. Furthermore, the GIPC interacting domain of SK3 is highly similar to the corresponding domains of SK1 and SK2. Stretches of ten to twenty residues are perfectly identical between SK3 and the two other channels. Thus, we suggest that GIPC may also interact with SK1 and SK2. Small-conductance, calcium-activated potassium channels (SK channels) are voltage-insensitive channels that have been identified molecularly within the last few years. As SK channels play a fundamental role in most excitable cells and participate in afterhyperpolarization (AHP) and spike-frequency adaptation, pharmacological modulation of SK channels may be of significant clinical importance. Specifically, the presence of SK1 and SK2 in the cortex and hippocampus, suggests that some of the alterations observed in Alzheimer's (such as disruption of calcium homeostasis) could result in dysregulation of SK channels. The effects of mutations in PS1 and PS2 on calcium homeostasis have been well documented (Mattson et al., 2001). A recent work (Yoo et al., 2000) showed that the presenilins are involved in the regulation of capacitative calcium entry (CCE, a refilling mechanism for depleted intracellular calcium stores). Abrogation of functional PS1 (by knocking out PS1) markedly potentiated CCE. In contrast, familial Alzheimer's disease (FAD) mutant PS1 or PS2 significantly attenuated CCE and store depletion-activated currents. While inhibition of CCE selectively increased the amyloidogenic amyloid beta peptide (Aβ42), increased accumulation of the peptide had no effect on CCE. The authors suggested that reduced CCE is an early cellular event leading to increased Aβ42 generation associated with FAD mutant presenilins. If changes in calcium homeostasis is an early cellular event in AD pathogenesis, then one would expect SK channels (that are calcium-activated) to be dysregulated. Therefore, drugs targeted to SK channels might reduce the effects of the neuronal cellular events leading to Alzheimer's disease. Likewise, pharmacological modulation of the GIPC-KCNN3 interaction opens novel therapeutic avenues against the neuronal and synaptic loss observed in AD.

We report the interaction of each of the two MAGUK proteins S-SCAM and NE-dlg with the REC8 protein. The neuroendocrine discs large protein (NE-dlg) and the atrophin 1 interacting protein (S-SCAM) are members of the membrane-associated guanylate kinase family (MAGUK) and contains three PDZ domains, one SH3 domain, and one guanylate kinase (GK) domain. S-SCAM is a particular member of the MAGUK family (membrane associated guanylate kinase) that contains 1455 amino acids. Instead of the usual three PDZ domains found in MAGUK proteins, S-SCAM has five or six PDZ domains (depending on the tool used for sequence analysis) scattered between amino acids 17 and 1229. The guanylate kinase (GK) domain, normally found in the C-terminal region of MAGUK proteins is in the N-terminal region of S-SCAM (amino acids 109 185). Finally, the SH3 domain is replaced by two WW domains between amino acids 302 and 381. S-SCAM was first identified as an interactor with atrophin-1 (Wood et al., 1998), a protein that contains a polyglutamine repeat, the expansion of which is responsible for dentatorubral and pallidoluysian atrophy (DRPLA). S-SCAM was subsequently found to also interact with δ-catenin (Ide et al., 1999), a protein that also interacts with PS1 (Zhou et al., 1997b) and which is involved in the mental retardation observed in the cri-du-chat syndrome (Medina et al., 2000). MAGUKs are found at the post-synaptic density where it interacts with other synaptic scaffolding proteins and with synaptic signalling proteins such as neurotransmitter receptors and channels (e.g. NMDA receptors, potassium channels) (Nagano et al., 1998; Makino et al., 1997; Muller et al., 1996).

APP metabolism is a critical event in the pathogenesis of Alzheimer's, because it leads to the release of either toxic (Aβ) or trophic (sAPP) metabolites (Cummings et al., 1998; Roch and Puttfarcken, 1996). In this respect, it is very important to identify proteins involved in the intracellular trafficking of APP. Proteins that interact with the cytosolic C-terminal region of APP play a major role in this process. The interaction of APP with Fe65, with Fe65 L, with Mint1, and with Mint2 have been well documented (Russo et al., 1998; Sastre et al., 1998). In turn, Fe65 was shown to interact with Mena, the mammalian homolog of the Drosophila enabled protein (Ermekova et al., 1997), and with the transcription factor LSF (Zambrano et al., 1998). The functional significance of those interactions have been discussed (Russo et al., 1998). Basically, this interaction network centered around the cytosolic tail of APP is proposed to be involved in 1) endocytosis and intracellular trafficking of APP, and 2) intracellular signalling events mediated by APP. Another work has shown that Fe65 and the mammalian homolog of the Drosophila disabled protein (DAB) bind to the cytosolic tail of several proteins involved in AD, like the LDL receptor, the LDL receptor related protein (LRP), and APP (Trommsdorff et al., 1998). The authors proposed that Fe65 and DAB can serve as molecular scaffold that bring together components of intracellular signalling complexes, including non-receptor tyrosine kinases such as the Abl protein. Thus, searching for interactors for LRP, Abl, and other proteins involved in this network may yield novel drug targets and therapeutic opportunities.

Abl is a cytoplasmic and nuclear protein tyrosine kinase that has been implicated in processes of cell differentiation, cell division, cell adhesion, and stress response (Mauro and Druker, 2001; Fernandez-Luna, 2000). The Abl protein contains 1130 amino acids (Fainstein et al., 1989) with an SH3 domain from amino acids 61 to 121, and SH2 domain from amino acids 127 to 217, and a protein kinase domain from amino acids 242 to 493. This kinase domain contains an ATP binding site from amino acids 248 to 271 and a tyrosine kinase active site from amino acids 359 to 371. The SH3 domain negatively regulates the tyrosine kinase activity, while deletion of the SH3 domain from Abl results in a protein with constitutive kinase activity. Furthermore, a very recent report (Zambrano et al., 2001) showed that, in cells expressing a constitutively active form of Abl, the cyto-tail of the amyloid protein precursor (APP) is tyrosine phosphorylated. Thus, Abl activity could modulate APP interaction with proteins such as Fe65, Fe65L, Mint1, and Mint2, which have been implicated in APP metabolism and Aβ production. There is also ample evidence that apoptosis is a major factor responsible for the neuronal loss observed in AD and other neurodegenerative conditions (Cotman et al., 1994; Smale et al., 1995; Shimohama, 2000; Behl, 2000; Offen et al., 2000). In particular, the role of the Bax protein in amyloid-induced neurotoxicity has been established (Selznick et al., 2000). Likewise, caspase-3 was shown to be activated in apoptotic neurons in response to exposure to the Aβ peptide (Harada and Sugimoto, 1999; Robertson et al., 2000). In addition, APP and the presenilins are targets of caspase-3 (Tesco et al., 1998; Weidemann et al., 1999; Gervais et al., 1999; Walter et al., 1998). Thus, Bax and caspase-3 interactors are also worth searching for. The protein CASK was identified as an interactor with the X11 (Mint1) protein (Borg et al., 1998; Borg et al., 1999). CASK is a member of the membrane-associated guanylate kinase (MAGUK) family. Proteins from this family typically contain one to three PDZ domains, an SH3 domain, and a guanylate kinase (GK) domain. In addition, CASK contains a serine/threonine protein kinase domain in the N-terminal region. MAGUK proteins are mainly localized at the synapses and function as synaptic scaffolding and clustering molecules for signalling proteins such as NMDA receptors and potassium channels (Kornau et al., 1995; Nagano et al., 1998; Muller et al., 1996; Nehring et al., 2000). Because of the effect of the Mint 1-CASK complex on APP trafficking and metabolism (Tomita et al., 1999; Mueller et al., 2000), searching for CASK interactors may also yield drug target opportunities that could modulate amyloid production. Likewise, searching for novel MAGUK interactors is also a strategy to find new signalling proteins involved in synaptic function and that represent therapeutic opportunities against neurodegeneration. Members of the MAGUK family that we used in searches include the post-synaptic density protein 95 (PSD95), the discs-large protein 1 (DLG1), the discs-large protein 2 (DLG2), the discs-large protein 3 (DLG3), and the neuroendocrine discs-large protein (NE-dlg).

ADAM10 is a protein that was initially identified as a metalloprotease that cleaves pro-TNF-a to release soluble TNF-a (Rosendahl et al., 1997), and was subsequently proposed to be an APP α-secretase (Lammich et al., 1999). Interestingly, another metalloprotease that cleaves TNF-α, TACE (also called ADAM17), was also suggested to be an APP a-secretase (Buxbaum et al., 1998), however another work suggested that ADAM17 is distinct from the APP α-secretase (Parvathy et al., 1998). In any event, it appears likely that the secretion of soluble factors such as TNF-α and sAPPα involves the catalytic activity of metalloproteases from the ADAM family. We now report an interaction between ADAM10 and the zinc finger protein 198 (ZNF198, also called FIM). This is a large protein (1377 aa) encoded by a gene that consistently undergoes the chromosomal translocation t(8;13)(p11;q12) with the FGF receptor-1 gene in myeloproliferative syndrome (Reiter et al., 1998). The function of ZNF198 is unknown, but its very name (zinc finger protein) suggests that it might be involved in transcription activation or protein-protein interactions. In this respect, it makes sense that ZNF198 interacts with ADAM10, another zinc-binding protein.

We also report an interaction between ADAM10 and the enzyme 3′-phosphoadenosine 5′-phosphosulfate (PAPS) synthase 1 (PAPSS1). PAPS is the only source of sulfate used in the sulfate assimilation pathway and in the sulfation of proteins, carbohydrates, and lipids. PAPS synthesis involves two steps: the transfer of a sulfate group to ATP (generating adenosine 5′-phosphosulfate, APS), and the transfer of a phosphate group from ATP to APS (generating PAPS). In plants, bacteria, fungi, and yeast, the ATP sulfurylase and APS kinase activities are found on separate polypeptides. In mammals, both activities are elicited by a single bifunctional enzyme, PAPSS1. Although there is no direct evidence that ADAM10 is sulfated, it contains several tyrosine residues that are preceded or followed by acidic residues, thus representing potential sulfation sites. In addition, a novel member of the ADAM family, ADAMTS-1, was shown to bind to the extracellular matrix (ECM) through sulfated glycosaminoglycans such as heparan sulfate (Kuno, Matsushima, 1998). Thus, the interaction between ADAM10 and PAPSS1 suggests that ADAM10 might also interact with the ECM through sulfated glycosaminoglycans. In this respect, it is interesting that alterations of ECM-associated ADAM proteins have been observed in the hippocampus of AD patients (Gerst et al., 2000).

We also report an interaction between ADAM10 and the KIAA0356 protein. The KDRI reports a cDNA sequence of 5371 bp containing an ORF of 3168 bp coding for 1056 aa. Analysis of the amino acid sequence revealed the presence of one leucine-zipper motif (aa 166 to 187), two Plekstrin Homology (PH) domains (aa 537 to 627 and aa 748 to 779), one protein kinase C terminal domain (aa 841 to 849), and one PHD finger domain (aa 1002 to 1048). The mRNA for KIAA0356 (analyzed by rt-PCR) is found at medium levels in placenta, liver, and ovary, followed by kidney and lung. Low levels are found in brain, thymus, prostate and testis. The message is undetected in other tissues examined (heart, skeletal muscle, pancreas, and spleen).

GIPC is a protein that we reported as an interactor with δ-catenin (see U.S. patent application Ser. No. 09/466,139 filed 21 Dec. 1999 and published PCT application WO 00/37483). GIPC contains a PDZ domain and also interacts with the C-terminus of a protein called RGS-GAIP, which is a GTPase activating protein for Gαi heterotrimeric G-proteins (De Vries et al., 1998b). GAIP was recently shown to be located on clathrin-coated vesicles (De Vries et al., 1998a). Therefore, when considering the interactions between PS1 and δ-catenin (Zhou et al., 1997b; Tanahashi and Tabira, 1999; Kosik, 1998) and between PS1 and rab11 (see U.S. patent application Ser. No. 09/466,139 filed 21 Dec. 1999 and published PCT application WO 00/37483), the pieces of a complex puzzle come together: the GAIP-GIPC complex (involved in GTPase activation) could be brought into the proximity of a potential GTPase target like rab11A through interactions of GIPC with δ-catenin, δ-catenin with PS1, and PS1 with rab11A. It is also remarkable that both GAIP and PS1 have been located in clathrin-coated vesicles (De Vries et al., 1998a; Efthimiopoulos et al., 1998), and that we found δ-catenin to interact with clathrin. When PS1 was first discovered (and first named S1182), its physiological function was unknown, although it was speculated that PS1 was involved in protein trafficking (Hardy, 1997). The pattern of interactions that is now taking shape around PS1 fully supports this original speculation. The interactions of PS1 and δ-catenin with rab11A, GIPC, and clathrin suggest a crucial role in the control of intracellular vesicle trafficking. Because APP is also found in rab11-positive clathrin-coated vesicles, the control of vesicle trafficking is important in determining the ultimate fate of the APP molecules leading to Aβ release or secretion of neurotrophic/protective sAPP. It should also be pointed out that a mouse homolog of GIPC was cloned and described in GenBank. In the first entry, the mouse GIPC is named synactin (GenBank entry AF104358), a protein that interacts with syndecan, a cell surface heparin-sulfate proteoglycan that links the cytoskeleton to the extracellular matrix. In another entry, mouse GIPC is called Semcapl (GenBank entry AF061263), which stands for “semaphorin F cytoplasmic domain associated protein 1”. Thus, GIPC is also thought to interact with semaphorin F, and therefore, it is possibly involved in axonal outgrowth and guidance. The interaction pattern of GIPC puts it at the heart of the control of vesicle trafficking and membrane fusion, with direct consequences on the metabolism of proteins such as APP, PS1, δ-catenin, and AChE.

To gain more insight into the role of GIPC in APP metabolism and Alzheimer's disease, we searched for GIPC interactor. We now report an interaction between GIPC and ADAM17. Also called TACE (TNF-α Converting Enzyme), ADAM17, was also suggested to be an APP α-secretase (Buxbaum et al., 1998), although these results have been challenged (Parvathy et al., 1998). As mentioned above, ADAM10, another metalloprotease that cleaves pro-TNF-α to release soluble TNF-α (Rosendahl et al., 1997), was also proposed to be an APP α-secretase (Lammich et al., 1999). Thus, it appears likely that the secretion of soluble factors such as TNF-α and sAPPα involves the catalytic activity of a common metalloprotease from the ADAM family. The interaction between ADAM17 and GIPC certainly bring ADAM17 close to APP, by virtue of the δ-catenin connection. Along those lines, one could argue that ADAM17 may have α-secretase activity only in brain, because of the brain-selective nature of δ-catenin. We also report an interaction between GIPC and the receptor for the glucagon-like peptide 2 (GLP2R). This protein of 553 aa has the typical structure of a G-protein coupled receptor, probably linked to Gs, as its activation by the glucagon-like peptide 2 (GLP-2) results in increased cAMP production. GLP2R is expressed primarily in the gastrointestinal tract, although we isolated our interacting clone from a hippocampus library. Our data suggest that GIPC brings a Gs coupled receptor in close proximity to PS1 and APP (through the d-catenin connection). Considering that APP interacts with BAT3 (see U.S. patent application Ser. No. 09/466,139 filed 21 Dec. 1999 and published PCT application WO 00/37483), which in turn associates with the adenylyl cyclase-associated protein CAP (Hubberstey et al., 1996), we suggest that alteration of the metabolism of Alzheimer proteins such as APP and PS1 might impair the function of GLP2R and CAP, and eventually reduce the levels of cAMP, an essential component of LTP in the hippocampus (Villacres et al., 1998; Davis and Laroche, 1998).

We report an interaction between KIAA0443 and PPP2R2B, the β isoform of the regulatory subunit B of protein phosphatase 2A (PP2A). The KIAA0443 protein was originally reported as a δ-catenin interactor (see U.S. patent application Ser. No. 09/466,139 filed 21 Dec. 1999 and published PCT application WO 00/37483) and was subsequently found to bind the phosphatidylinositol 4-kinase (P14K) and the serotonin receptor 2A (5HT-2A) (see U.S. patent application Ser. No. 60/240,790 filed 17 Oct. 2000). The Kazusa DNA research Institute (KDRI) report for KIAA0443 shows a cDNA sequence of 5190 base pairs (bp) with an open reading frame of 4212 bp coding for 1404 amino acids. The protein contains four lipocalin domains scattered between amino acids 409 and 1017, suggesting that this KIAA protein might be involved in the transport of small lipophilic molecules. An ATP binding site is located between amino acids 1385 to 1392. The message for KIAA0443 is at highest levels in brain, kidney, prostate, and ovary. Medium levels are found in thymus and small intestine. Low levels are found in placenta, lung, pancreas, heart, and testis. In liver, skeletal muscle, and spleen, the message is barely detectable. PP2A is composed of a heterodimeric core (the 36 kDa catalytic subunit and the 65 kDa constant regulatory subunit A) that associates with a variety of regulatory subunits B. While the 6 isoform is expressed ubiquitously (Tanabe et al., 1996), the β isoform reported here appears to be neuron-specific (Mayer et al. 1991). As the regulatory B subunit of PP2A is supposed to confer substrate specificity, and because KIAA0443 is a δ-catenin interactor, one can speculate that δ-catenin might be phosphorylated (as β-catenin is), and its level of phosphorylation might be controlled by PP2A containing the P regulatory subunit B (while PP2A containing the 6 regulatory subunit B controls the phosphorylation of β-catenin).

We also report an interaction between the KIAA0443 and MAP1b (microtubule-associated protein 1b). MAP1b is a neuronal cytoskeletal protein involved in neurite outgrowth and stabilization (Shea and Beermann, 1994). The phosphorylated form of MAP1b is particularly important for neurite extension in the hippocampus (Boyne et al, 1995). Defects in axonal elongation and neuronal migration have been reported in mice with disrupted tau and MAP1b genes (Shea and Beermann, 1994). The interactions of δ-catenin with ERAB and with NACP (reported in U.S. patent application Ser. No. 09/466,139 filed 21 Dec. 1999 and published PCT application WO 00/37483) suggest that it might be part of a complex which contains APP, a protein that is well known to stimulate neurite extension (Saitoh et al., 1994; Roch et al., 1993; Saitoh and Roch, 1995; Mattson et al., 1999; Mattson and Duan, 1999). The interaction described above of KIAA0443 with the beta isoform of the regulatory subunit B of the protein phosphatase 2A (a component of the wingless pathway) suggests that it might play a role in the phosphorylation state of MAP1b. In fact MAP1b was shown to be phosphorylated by both cyclin-dependent kinases and by the glycogen synthase kinase 3 (another component of the wingless pathway) (Garcia-Perez et al., 1998). It should also be noted that MAP1b was recently located in Lewy bodies and was found to bind α-synuclein (NACP) filaments (Jensen et al., 2000). Thus, KIAA0443 interacts with two proteins (δ-catenin and MAP1b) that both interact with NACP, a protein associated with the pathological features of both Alzheimer's and Parkinson's diseases. This interaction pattern suggests that KIAA0443 and δ-catenin might be central components of neurodegenerative diseases with neurofibrillary pathology. We suggest that pharmacological modulation of the KIAA0443-PPP2R2B interaction and the KIAA0443-MAP1b interaction is a new therapeutic avenue against Alzheimer's disease and other neurodegenerative conditions.

We also report an interaction between the KIAA0443 protein and the tetraspanin NET-7 protein. The cDNA for NET-7 was identified by sequence analysis of EST databases (Serru et al., 2000). These authors found seven new members of the tetraspanin family which they called NET-1 to NET-7 (for New EST Tetraspanin). The tetraspanin family contains nearly 20 members, all of which are cell-surface proteins that span the membrane four times, forming two extracellular loops. Some members of the family are found in virtually all tissues, whereas others are highly restricted. The EST profile for NET-7 suggests a widely distributed expression pattern. In terms of function, tetraspanins are involved in diverse processes such as cell activation and proliferation, adhesion and motility, differentiation, and cancer. The particular function of NET-7 is unknown. The structure and function of the tetraspanin family has recently been reviewed (Maecker et al., 1997). The KIAA0443 bait used in the search was form amino acids 901 to 1200, a domain of the protein that contains the third and fourth lipocalin domains. We found one clone in a brain library encoding amino acids 1 to 219 of NET-7. This region of the protein contains three of the four transmembrane domains, and parts of the protein found on either side of the membrane.

We report an interaction between CASK and the T-lymphoma invasion and metastasis inducing protein 1 (TIAM1). CASK is a member of the MAGUK family (membrane associated guanylate kinase) and contains one PDZ, one SH3, and one guanylate kinase (GK) domains. CASK also contains a protein kinase domain (aa 12 to 276), which makes it a MAGUK protein with direct signalling potential. CASK interacts with Mint1 (Borg et al., 1998; Borg et al., 1999), which in turns interact with APP. The Mint/CASK complex is thought to regulate APP metabolism, as well as APP and Aβ secretion (Mueller et al., 2000). TIAM1 is a large protein (1591 aa) that was first identified in mouse as the product of a gene involved in metastasis (Habets et al., 1994). The same group later cloned the human gene (Habets et al., 1995). TIAM1 contains several biologically active domains (identified by HMMER Pfam): a RhoGEF (guanine nucleotide exchange factor) was found from aa 1044 to 1233, two Plekstrin homology (PH) domains were found from aa 434 to 547 and from aa 1317 to 1395, one PDZ domain was found from aa 845 to 927, and an RBD domain was found from aa 765 to 832. TIAM1 acts as a GDP dissociation stimulator protein that stimulates the GDP-GTP exchange activity of Rho-like GTPases, and thus activates them. Recently, TIAM1 was shown to activate human Rac1 (Bollag et al., 2000; Fleming et al., 2000).

The CASK bait used in the search was from aa 1 to 325, a region of the protein that contains the protein kinase domain. We found five clones encoding aa 1450 to 1591 (C-terminus) of TIAM1 in a brain library, and one clone encoding aa 1470 to 1591 in a hippocampus library. TIAM1 was initially identified by its activity as a metastasis-inducing protein, and most subsequent studies have focused on its cancer-related activity (Michiels and Collard, 1999). However, another work (Leeuwen et al., 1997) showed that the GEF (guanine nucleotide exchange factor) activity of TIAM1 also affects neuronal morphology. Specifically, the authors showed that through opposing effects on Rho and Rac, TIAM1 stimulates neurite outgrowth. This observation is interesting because CASK also interacts with APP (Borg et al., 1998; Borg et al., 1999; Mueller et al., 2000) and the neurite extension activity of APP are well documented (Saitoh et al., 1994; Roch et al., 1993; Saitoh and Roch, 1995; Mattson et al., 1999; Mattson and Duan, 1999). Thus, CASK (a post-synaptic density protein) interacts with two proteins (APP and TIAM1) which are known to stimulate neurite extension. In addition, we previously reported that APP interacts with KIAA0351, a protein that contains two GEF domains (one of which is 32% identical and 47% similar to TIAM1), and one Plekstrin homology (PH) domain (see U.S. patent application Ser. No. 09/466,139 filed 21 Dec. 1999 and published PCT application WO 00/37483). We suggest that pharmacological modulation of the CASK-TIAM1 interaction is a new therapeutic avenue against Alzheimer's disease and other neurodegenerative condition.

We report a interaction between the discs large 2 protein (DLG2) and the huntingtin-associated protein interacting protein (HAPIP). DLG2 is a member of the MAGUK family (membrane associated guanylate kinase) and contains three PDZ domains, one SH3 domain, and one guanylate kinase (GK) domain (Wheal et al., 1998; Dimitratos et al., 1999). As most MAGUK proteins, DLG2 is found at the post-synaptic density where it interacts with other synaptic scaffolding proteins and with synaptic signalling proteins such as neurotransmitter receptors and channels (e.g. NMDA receptors, potassium channels) (Nagano et al., 1998; Makino et al., 1997; Muller et al., 1996). As its name implies, HAPIP was first identified by its binding to HAP1, the Huntingtin-associated protein 1 (Colomer et al., 1997). HAPIP is also called Duo because its structure resembles that of Trio, although it is shorter. These authors mention that Duo contains a guanine nucleotide exchange factor (GEF) domain that is likely to be rac1-specific, a Plekstrin homology (PH) domain and spectrin-like repeat units. The Duo mRNA is brain-specific, and among various brain regions, the cerebral cortex, amygdala, hippocampus, and caudate nucleus show the highest levels of expression, while the brain stem and cerebellum show the lowest levels.

The DLG2 bait used in the search was from aa 411 to 616, a region of the protein that contains the third PDZ domain and the SH3 domain. We found two clones encoding aa 1592 to 1663 (C-terminus) of Duo in a hippocampus library. The last three residues of Duo are TYV-COOH, which is a PDZ-binding motif. Thus, we suggest that the third PDZ domain of CASK interacts with the C-terminus of Duo. Duo was initially identified as an interactor with the Huntingtin-associated protein 1 (Colomer et al., 1997). Because Duo contains a number of biologically active domains, including a rac1 GEF domain (guanine nucleotide exchange factor), the authors suggested that huntingtin might play a role in the ras-related signalling pathway. However, they stopped short of saying that disruption of the ras pathway might underlie Huntington's disease pathology. Indeed, a more recent work (Bertaux et al., 1998) showed that the huntingtin-HAP1 interactions do not contribute to Huntington's disease pathology in transgenic mice, suggesting that Duo is not involved in Huntington's disease pathology either. The DLG2-Duo interaction suggests that PDZ interactions can mediate clustering of GEF proteins by the MAGUKs. Many of the interactions that we released recently with MAGUK baits appeared to be mediated by the PDZ domains, or by the SH3-GK domains. The fact that the same preys were found with different MAGUK baits suggest that these interactions reflect affinity of the SH3 domain for the GK domain and affinity of PDZ-binding domains for PDZ domains, rather that specific protein-protein interactions.

On the other hand, the two interactions reported here (CASK-TIAM1 and DLG2-HAPIP) appear to be different because neither TIAM1 nor HAPIP (Duo) were found by more than one bait protein. It is interesting though, that two MAGUK proteins (CASK and DLG2) interact with two different but closely related proteins such as TIAM1 and Duo. Both contain GEF and PH domains. Furthermore, APP (a CASK interactor) also interacts with KIAA0351 (see U.S. patent application Ser. No. 09/466,139 filed 21 Dec. 1999 and published PCT application WO 00/37483), a protein that contains two GEF domains (one of which is 32% identical and 47% similar to TIAM1), and one Plekstrin homology (PH) domain, and which also interacts with Trio. Thus, we suggest that APP and two MAGUK proteins (CASK and DLG2) are components of signalling complexes that include GEF proteins such as KIAA0351, TIAM1, TRIO, and Duo. It should also be noted that TRIO was initially identified as an interactor for LAR (Debant et al., 1996), a transmembrane receptor with tyrosine phosphatase activity. The same study also reports that TRIO contains an Ig-like domain (close to the kinase domain in the C-terminal region), and four spectrin repeats (in the N-terminal region). KIAA0351 itself contains a PH domain and a ras GEF domain and was identified as an APP interactor. Thus, APP interacts directly with a transmembrane receptor tyrosine phosphatase, PTPZ (see U.S. patent application Ser. No. 09/466,139 filed 21 Dec. 1999 and published PCT application WO 00/37483), and indirectly (through the KIAA0351 and TRIO connection) with another transmembrane receptor tyrosine phosphatase, LAR. The neurotrophic and neuroprotective effects of sAPP are well documented (Roch et al., 1993; Saitoh and Roch, 1995; Roch and Puttfarcken, 1996; Mattson et al., 1999; Mattson and Duan, 1999). In this respect, it is important to note that Abl, TRIO, LAR, and other associated proteins are involved in axonal development (Lanier and Gertler, 2000). A more recent study also showed that downregulation of LAR activity prevents apoptosis and increases NGF-induced neurite outgrowth (Tisi et al., 2000). Together with the recent observation that pleiotrophin binding to PTPZ inhibits its activity (Meng et al., 2000), these results suggest that inhibition of receptor tyrosine phosphatase activity is a key element underlying the neurotrophic or neuroprotective effects of secreted factors such as sAPP.

We report an interaction between the calcium and integrin binding protein (CIB) and the zinc finger protein 127 (ZNF127). As its name indicates, CIB was initially identified for its calcium and integrin binding properties (Naik et al., 1997). CIB contains two EF-hands and is 58% similar to calcineurin B (regulatory subunit) and 56% similar to calmodulin. The authors suggested that CIB might be the regulatory subunit of a novel (yet to be discovered) calcium-dependent phosphatase. Also known as calmyrin, CIB was reported as an interactor with both presenilins, although the interaction with PS2 is stronger than with PS1 (Stabler et al., 1999). Because mutations in both PS1 (Chan et al., 2000) and PS2 (Leissring et al., 1999) disrupt calcium homeostasis, CIB might be involved in the neuronal apoptotic cascade elicited by the presenilin mutations. ZNF127 was identified as a protein of 507 amino acids with a RING (C3HC4) zinc finger motif and multiple C3H zinc finger motifs (Jong et al., 1999). Northern blot analysis revealed that the ZNF127 gene is expressed ubiquitously as an approximately 3-kb transcript. Interestingly, the entire coding sequence overlaps a second gene, ZNF127AS, which is transcribed from the antisense strand with a different transcript size and pattern of expression. Allele-specific analysis showed that the ZNF127 gene is expressed only from the paternal allele. Another work (Nicholls et al., 1998) showed that the ZNF127 gene (on chromosome 15) is part of a coordinately regulated imprinted domain affected in Prader-Willi syndrome, a disorder characterized by diminished fetal activity, obesity, muscular hypotonia, mental retardation, short stature, hypogonadotropic hypogonadism, and small hands and feet. ZNF127 is also known as Makorin 3 (MKRN3), a member of the Makorin family of proteins featuring RING and C3H zinc-finger motifs with abundant expression in developing brain and nervous system (Gray et al., 2000). The bait used in the search was from amino acids 1 to 191 of CIB (full-length protein). We found one clone in a brain library encoding amino acids 313 to 507 of ZNF127, a region of the protein that contains the RING domain.

A number of genetic studies showed that proteins of the low density lipoprotein (LDL) receptor (LDLR) family are involved in Alzheimer's disease (AD) (Lendon et al., 1997; Wavrant-DeVrieze et al., 1997; Lambert et al., 1998; Kang et al., 1997). In addition to LDLR itself, these proteins include the LDLR related proteins 1 and 2 (LRP1 and LRP2), the LRP associated protein 1 (LRPAP1), and the LDLR related protein deleted in tumor (LRPDIT). Complementing the genetic evidence, biochemical studies have shown that proteins of the LDLR family interact with APP and proteins of the Fe65 network. These interactions modulate APP metabolism and Aβ production (Trommsdorff et al., 1998; Kounnas et al., 1995; Howell et al., 1999; Willnow et al., 1995). Furthermore, LDLR and related proteins are also involved in neurite extension (Narita et al., 1997), probably through an APP-mediated mechanism. Other studies recently showed that LRP6 is involved in the wingless pathway (Pinson et al., 2000; Tamai et al., 2000). This pathway includes the adaptor proteins axin and β-catenin, and signalling molecules such as the protein phosphatase 2A (PP2A) and the glycogen synthase kinase 3β (GSK3) (Pinson et al., 2000; Tamai et al., 2000), which was also proposed to be a tau kinase that associates with PS1 (Takashima et al., 1998). Because PS1 was found in β-catenin complexes (Kang et al., 1999), the double connection between the wingless pathway and the APP network (LRP protein family with axin and PS1 with β-catenin) could be the common denominator to amyloid and tangle pathology. We now report a number of interactions that strengthen this concept.

In U.S. patent application Ser. No. 60/240,790 filed 17 Oct. 2000, we reported interactions between BAT3 and LRP2 and LRPAP1. BAT3 is an APP interactor (see U.S. patent application Ser. No. 09/466,139 filed 21 Dec. 1999 and published PCT application WO 00/37483). We now report that axin, the central component of the wingless pathway, interacts with LRP1, LRP2, and LRPDIT. Thus, proteins of the LRP family appear to interact with both axin and BAT3. We also report interactions between axin and the novel protein PN9113 and between BAT3 and the novel protein PN9113. The sequence of this novel protein is provided below. Although PN9113 does not show any sequence similarity with the LRP proteins, it is another common interactor with BAT3 and axin, thus potentially generating another link between APP and the wingless pathway. Finally, we also report an interaction between BAT3 and the protein Dickkopf 3 (DKK-3). This interaction is to be considered in the light of the recent work showing that LRP6 interacts with the DKK family (Mao et al., 2001). Thus, DKK proteins are common interactors between BAT3 and the LRP proteins. Our interaction network suggests that BAT3 and axin are important components of protein complexes that bring together APP and the wingless pathway. Because GSK3β was proposed to be a tau kinase (Takashima et al., 1998), and because APP and LRP interactors such as Fe65 and X11 can recruit kinases such as Abl and Src that can also act as tau kinases (Russo et al., 1998; Trommsdorff et al., 1998), our data suggest that BAT3 and axin are core components of protein complexes that modulate APP metabolism and tau phosphorylation. Thus, pharmacological modulation of these interactions open novel therapeutic avenues against the cellular mechanisms at the crossroads of the amyloid and tangle pathology observed in AD.

APP metabolism is a critical event in the pathogenesis of Alzheimer's, because it leads to the release of either toxic (Aβ) or trophic (sAPP) metabolites (Cummings et al., 1998; Roch and Puttfarcken, 1996). In this respect, it is very important to identify proteins involved in the intracellular trafficking of APP. Proteins that interact with the cytosolic C-terminal region of APP play a major role in this process. The interaction of APP with Fe65, with Fe65 L, with Mint1, and with Mint2 have been well documented (Russo et al., 1998; Sastre et al., 1998). In turn, Fe65 was shown to interact with Mena, the mammalian homolog of the Drosophila enabled protein (Ermekova et al., 1997), and with the transcription factor LSF (Zambrano et al., 1998). The functional significance of those interactions have been discussed (Russo et al., 1998). Basically, this interaction network centered around the cytosolic tail of APP is proposed to be involved in 1) endocytosis and intracellular trafficking of APP, and 2) intracellular signalling events mediated by APP. Another work has shown that Fe65 and the mammalian homolog of the Drosophila disabled protein (DAB) bind to the cytosolic tail of several proteins involved in AD, like the LDL receptor, the LDL receptor related protein (LRP), and APP (Trommsdorff et al., 1998). The authors proposed that Fe65 and DAB can serve as molecular scaffold that bring together components of intracellular signalling complexes, including non-receptor tyrosine kinases such as the Abl protein. Thus, searching for interactors for LRP, Abl, and other proteins involved in this network may yield novel drug targets and therapeutic opportunities.

Abl is a cytoplasmic and nuclear protein tyrosine kinase that has been implicated in processes of cell differentiation, cell division, cell adhesion, and stress response (Mauro and Druker, 2001; Fernandez-Luna, 2000). The Abl protein contains 1130 amino acids (Fainstein et al., 1989) with an SH3 domain from amino acids 61 to 121, and SH2 domain from amino acids 127 to 217, and a protein kinase domain from amino acids 242 to 493. This kinase domain contains an ATP binding site from amino acids 248 to 271 and a tyrosine kinase active site from amino acids 359 to 371. The SH3 domain negatively regulates the tyrosine kinase activity, while deletion of the SH3 domain from Abl results in a protein with constitutive kinase activity. Furthermore, a very recent report (Zambrano et al., 2001) showed that, in cells expressing a constitutively active form of Abl, the cyto-tail of the amyloid protein precursor (APP) is tyrosine phosphorylated. Thus, Abl activity could modulate APP interaction with proteins such as Fe65, Fe65L, Mint1, and Mint2, which have been implicated in APP metabolism and Aβ production. There is also ample evidence that apoptosis is a major factor responsible for the neuronal loss observed in AD and other neurodegenerative conditions (Cotman et al., 1994; Smale et al., 1995; Shimohama, 2000; Behl, 2000; Offen et al., 2000). In particular, the role of the Bax protein in amyloid-induced neurotoxicity has been established (Selznick et al., 2000). Likewise, caspase-3 was shown to be activated in apoptotic neurons in response to exposure to the Aβ peptide (Harada and Sugimoto, 1999; Robertson et al., 2000). In addition, APP and the presenilins are targets of caspase-3 (Tesco et al., 1998; Weidemann et al., 1999; Gervais et al., 1999; Walter et al., 1998). Thus, Bax and caspase-3 interactors are also worth searching for. The protein CASK was identified as an interactor with the X11 (Mint1) protein (Borg et al., 1998; Borg et al., 1999). CASK is a member of the membrane-associated guanylate kinase (MAGUK) family. Proteins from this family typically contain one to three PDZ domains, an SH3 domain, and a guanylate kinase (GK) domain. In addition, CASK contains a serine/threonine protein kinase domain in the N-terminal region. MAGUK proteins are mainly localized at the synapses and function as synaptic scaffolding and clustering molecules for signalling proteins such as NMDA receptors and potassium channels (Kornau et al., 1995; Nagano et al., 1998; Muller et al., 1996; Nehring et al., 2000). Because of the effect of the Mint 1-CASK complex on APP trafficking and metabolism (Tomita et al., 1999; Mueller et al., 2000), searching for CASK interactors may also yield drug target opportunities that could modulate amyloid production. Likewise, searching for novel MAGUK interactors is also a strategy to find new signalling proteins involved in synaptic function and that represent therapeutic opportunities against neurodegeneration. Members of the MAGUK family that we used in searches include the post-synaptic density protein 95 (PSD95), the discs-large protein 1 (DLG1), the discs-large protein 2 (DLG2), the discs-large protein 3 (DLG3), and the neuroendocrine discs-large protein (NE-dlg).

We report an interaction between the estrogen-related receptor gamma (ERR-γ) and the Ran binding protein 2 (RANBP2). ERR-γ is an orphan nuclear receptor that was identified by its similarity with the human estrogen-related receptor 2 (hERR2, also called hERR-β) using bioinformatics (EST database mining) and inverse PCR (Chen et al., 1999). The hERR2 gene itself was identified by cloning cDNAs coding for proteins related to steroid hormone receptors (Giguere et al., 1988). Subsequent studies showed that hERR2 and hERR1 are constitutive activators of the classic estrogen response element (ERE) as well as the palindromic thyroid hormone response element [TRE(pal)] but not the glucocorticoid response element (GRE) (Xie et al., 1999). These authors also showed that both orphan receptors binds a number of transcription coactivators (including the steroid receptor coactivator 1 SRC-1, also known as NCOA1) in a ligand-independent manner. Furthermore, hERR1 and hERR2 were shown to bind DNA as monomers, while classic steroid and retinoid receptors associate with DNA as homo- or heterodimers (Sem et al., 1997). The Ras-related nuclear protein Ran is a small GTPase that has been implicated in nuclear transport. RANBP2 was first identified as a “giant nucleopore protein” that binds Ran (Yokoyama et al., 1995). In an independent study looking for Ran interactors, RANBP2 was identified under the name Nup358 (nucleopore protein of 358 kDa (Wu et al., 1995). RANBP2 contains an amino-terminal 700-residue leucine-rich region, four domains with high similarity to RANBP1, eight zinc-finger motifs similar to those of Nup153, and a carboxy terminus with high homology to cyclophilin. The ERR-γ bait used in the search was from amino acids 160 to 436, a region of the protein that contains the second zinc-finger domain, and the ligand-binding domain. We found one clone in a hippocampus library encoding amino acids 812 to 1155 of RANBP2, a region of the protein that does not contain any of the domains mentioned above.

We report an interaction between the estrogen-related receptor gamma (ERR-γ) and the G19 protein (G19P1). ERR-γ is an orphan nuclear receptor that was identified by its similarity with the human estrogen-related receptor 2 (hERR2, also called hERR-β) using bioinformatics (EST database mining) and inverse PCR (Chen et al. 1999). The hERR2 gene itself was identified by cloning cDNAs coding for proteins related to steroid hormone receptors (Giguere et al., 1988). Subsequent studies showed that hERR2 and hERR1 are constitutive activators of the classic estrogen response element (ERE) as well as the palindromic thyroid hormone response element [TRE(pal)] but not the glucocorticoid response element (GRE) (Xie et al., 1999). These authors also showed that both orphan receptors binds a number of transcription coactivators (including the steroid receptor coactivator 1 SRC-1, also known as NCOA1) in a ligand-independent manner. Furthermore, hERR1 and hERR2 were shown to bind DNA as monomers, while classic steroid and retinoid receptors associate with DNA as homo- or heterodimers (Sem et al., 1997). G19P1 is a gene coding for a protein of 80 kDa that was first identified as a protein kinase C substrate (Sakai et al., 1989) in fibroblasts and epidermal carcinoma cells. The protein has 527 amino acids and contains an extremely Glu-rich region (residues 313 to 336). The G19P1 gene is on chromosome 19. The ERR-γ bait used in the search was from amino acids 160 to 436, a region of the protein that contains the second zinc-finger domain, and the ligand-binding domain. We found five clones in a hippocampus library coding for amino acids 1 to 225 of the G19P1 protein. The amino-terminal 15 residues of G19P1 are very hydrophobic, possibly representing a signal peptide, suggesting that the protein could be secreted. However, its interaction with ERR-γ, and its identification as a substrate for PKC (an intracellular enzyme) suggest that G19P1 is indeed intracellular.

We report an interaction between the estrogen-related receptor gamma (ERR-γ) and the apoptosis inhibitor 2 (AIP2). ERR-γ is an orphan nuclear receptor that was identified by its similarity with the human estrogen-related receptor 2 (hERR2, also called hERR-β) using bioinformatics (EST database mining) and inverse PCR (Chen et al., 1999). The hERR2 gene itself was identified by cloning cDNAs coding for proteins related to steroid hormone receptors (Giguere et al., 1988). Subsequent studies showed that hERR2 and hERR1 are constitutive activators of the classic estrogen response element (ERE) as well as the palindromic thyroid hormone response element [TRE(pal)] but not the glucocorticoid response element (GRE) (Xie et al., 1999). These authors also showed that both orphan receptors binds a number of transcription coactivators (including the steroid receptor coactivator 1 SRC-1, also known as NCOA1) in a ligand-independent manner. Furthermore, hERR1 and hERR2 were shown to bind DNA as monomers, while classic steroid and retinoid receptors associate with DNA as homo- or heterodimers (Sem et al., 1997). AIP2 was identified as an interactor with the tumor necrosis factor receptor-associated factors TRAF1 and TRAF2, suggesting that AIP2 inhibits apoptosis by regulating signals required for activation of ICE-like proteases (Uren et al., 1996). This interaction between ERRg and AIP2 suggests that the neuroprotective effects of estrogen (Diaz Brinton et al., 2000) might be mediated by AIP2.

We report an interaction between the estrogen-related receptor gamma (ERR-γ) and the homeotic protein Prox1. ERR-γ is an orphan nuclear receptor that was identified by its similarity with the human estrogen-related receptor 2 (hERR2, also called hERR-β) using bioinformatics (EST database mining) and inverse PCR (Chen et al., 1999). The hERR2 gene itself was identified by cloning cDNAs coding for proteins related to steroid hormone receptors (Giguere et al., 1988). Subsequent studies showed that hERR2 and hERR1 are constitutive activators of the classic estrogen response element (ERE) as well as the palindromic thyroid hormone response element [TRE(pal)] but not the glucocorticoid response element (GRE) (Xie et al., 1999). These authors also showed that both orphan receptors binds a number of transcription coactivators (including the steroid receptor coactivator 1 SRC-1, also known as NCOA1) in a ligand-independent manner. Furthermore, hERR1 and hERR2 were shown to bind DNA as monomers, while classic steroid and retinoid receptors associate with DNA as homo- or heterodimers (Sem et al., 1997). Prox1 is a transcription factor that is essential for normal development, as a recent study showed that homozygous Prox1-null mice die at mid-gestation from multiple developmental defects (Wigle et al., 1999). This interaction suggests that Prox1 mediates some of the transcriptional activation elicited by estrogen and ERR-γ.

We report an interaction between caspase-3 (casp-3) and neurocalcin delta. Neurocalcin is a novel member of the neural calcium-sensor protein family, defined by the photoreceptor cell-specific protein recoverin, that has been proposed to be involved in the regulation of calcium-dependent phosphorylation in signal transduction pathways. Originally isolated from bovine brain (Terasawa et al., 1992), neurocalcin exists in five isoforms designated alpha, beta, gamma 1, gamma 2, and delta. The human gene for neurocalcin was recently cloned (GenBank entry NM_{—}032041). Neurocalcin contains 3 EF hands and is 45% identical to recoverin, a calcium-binding protein expressed specifically in the retina and pineal gland. While recoverin is found predominantly in photoreceptor cells and is involved in the calcium-dependent inhibition of rhodopsin phosphorylation, neurocalcin is found throughout the brain, including the inner ear (Iino et al., 1995b), the olfactory bulb (Porteros et al. 1996) and the vomeronasal organ (Tino et al., 1995a). This expression pattern suggests that neurocalcin is involved in the calcium-dependent regulation of signals elicited by a variety of receptors. Neurocalcin is also found in GABAergic hippocampal interneurons (Martinez-Guijarro et al., 1998). In the temporal cortex, neurocalcin localization is very similar to that of synaptophysin (Shimohama et al., 1996). This finding suggests that the reduced levels of neurocalcin observed in AD brains compared to controls reflect a biochemical deficit related to the synaptic degeneration in AD. Neurocalcin is also 87% identical to another calcium binding protein expressed specifically in the hippocampus, named hippocalcin (GenBank entry NM_{—}002143). As our interacting neurocalcin clones code for a domain (aa 130 to 193) that is 94% identical to the corresponding domain of hippocalcin, we suggest that casp-3 might also interact with hippocalcin. In addition to its hippocampus-specific expression profile, hippocalcin is interesting because it interacts with the mixed lineage kinase 2 (MLK2) (Nagata Ki et al., 1998) and the mixed lineage kinase 3 (MLK3). As we reported in U.S. patent application Ser. No. 60/240,790 filed 17 Oct. 2000, MLK2 interacts with CIB, another calcium-binding proteins that interacts with the presenilins (Stabler et al., 1999). As the MLK2 signaling cascade is activated by a mutant form of huntingtin (polyglutamine-expanded) (Liu et al., 2000), and as this mutant form of huntingtin is cleaved by casp-3 (Petersen et al., 1999; Goldberg et al., 1996), neurocalcin and/or hippocalcin appear to be adaptor proteins generating a link between MLK2 and casp-3, both enzymes involved in the neurodegenerative process observed in Huntington's disease. Likewise, MLK2 and casp-3 are also involved in neurodegeneration observed in Alzheimer's disease: MLK2 interacts with CIB, and casp-3 cleaves APP during apoptosis (Gervais et al., 1999), mediates Aβ toxicity (Harada and Sugimoto, 1999), and cleaves both PS1 and PS2 (van de Craen et al., 1999). While this later work suggests that FAD mutations in PS1 do not alter caspase-dependent processing, a more recent study (Kovacs et al., 1999) showed that some FAD PS1 mutations do activate casp-3. Moreover, another work (Begley et al., 1999) showed that FAD PS1 mutations alter calcium homeostasis and activate casp-3 in cortical synaptic compartments. Thus, an intriguing concept is emerging, in which caps-3 and MLK2 are central players involved in neurodegeneration, interacting with calcium binding proteins (CIB, hippocalcin, and/or neurocalcin). Mutations in the presenilins or in Huntingtin trigger a cascade of biochemical reactions leading to Alzheimer or Huntington disease. We suggest that modulation of the interaction between caspase-3 and neurocalcin or hippocalcin is a novel therapeutic avenue against Alzheimer's diseases and other neurodegenerative disorders.

We report an interaction between caspase-3 (casp-3) and calcineurin B, the regulatory subunit of a calcium-dependent phosphatase, which we already reported as an Abl interactor (see above). Caspase-3 involvement in Alzheimer and other neurodegenerative diseases has been widely described (Robertson et al., 2000). Several caspases, including casp-3, cleave APP during apoptosis (Gervais et al., 1999). The major proteolytic site of caspase-3 is within the cytoplasmic tail of APP, and cleavage at this site occurs in hippocampal neurons in vivo following acute excitotoxic or ischemic brain injury. In addition, casp-3 also mediates Aβ toxicity (Harada and Sugimoto, 1999). Casp-3 also cleaves and activates caspases-6, -7, and -9, and cleaves a mutant form of huntingtin associated with Huntington disease (Goldberg et al., 1996). Furthermore, a recent work (Takuma et al., 1999) showed that apoptosis in Ca2+ reperfusion injury of cultured astrocytes is mediated by caspase-3 and is blocked by FK506, a calcineurin inhibitor, suggesting that casp-3 activity is influenced by its phosphorylation state. Another study showed that high levels of calcineurin activity induce neuronal apoptosis mediated by casp-3, and this phenomenon is blocked by FK506 (Takuma et al., 1999). Together, these observations suggest that dephosphorylation of casp-3 by calcineurin might activate casp-3 and the initiation of apoptosis. The interaction that we report between casp-3 and calcineurin further supports this concept. We suggest that modulation of the interaction between caspase-3 and calcineurin is a novel therapeutic avenue against Alzheimer's diseases and other neurodegenerative disorders mediated by apoptosis.

We also report an interaction between FAK2 and the polyglutamine tract-binding protein 1 (PQBP1). FAK2 is a tyrosine kinase involved in neurite extension and neuronal differentiation and survival (Park et al., 2000; Menegon et al., 1999). We reported that FAK2 interacts with δ-catenin (see U.S. patent application Ser. No. 09/466,139 filed 21 Dec. 1999 and published PCT application WO 00/37483), thus suggesting that FAK2 might be involved in Alzheimer's disease. Our interacting clone for PQBP1 codes for aa 1 to 167, in which a WW domain was identified (aa 46 to 80). The complete PQBP1 proteins contains 265 aa and several functional domains, including a putative nuclear localization signal sequence, a C2 domain implicated in Ca2+-dependent phospholipid signaling, and the WWP/WW domain which binds to proline-rich motifs in other proteins (Waragai et al., 1999). PQBP1 interacts with the polyglutamine tract of the brain-specific transcription factor Brn-2, thereby inhibiting its activity. It also interacts with the polyglutamine tract of triplet repeat disease gene products (Waragai et al., 2000). PQBP-1 is a ubiquitous protein expressed primarily in neurons throughout the brain, with abundant levels in hippocampus, cerebellar cortex and olfactory bulb. Its interaction with polyglutamine tracts under physiological and pathological conditions affects neuronal survival. This interaction between FAK2 and PQBP1 suggests that either PQBP1 is a FAK2 substrate, or that PQBP1 might be an adaptor molecule that brings together FAK2 and its possible targets such as the polyglutamine tract-containing proteins. In any event, pharmacological modulation of the FAK2-PQBP1 interaction is a new therapeutic avenue against Alzheimer's disease and other neurodegenerative conditions.

APP metabolism is a critical event in the pathogenesis of Alzheimer's, because it leads to the release of either toxic (Aβ) or trophic (sAPP) metabolites (Cummings et al., 1998; Roch and Puttfarcken, 1996). In this respect, it is very important to identify proteins involved in the intracellular trafficking of APP. Proteins that interact with the cytosolic C-terminal region of APP play a major role in this process. The interaction of APP with Fe65, with Fe65 L, with Mint1, and with Mint2 have been well documented (Russo et al., 1998; Sastre et al., 1998). In turn, Fe65 was shown to interact with Mena, the mammalian homolog of the Drosophila enabled protein (Ermekova et al., 1997), and with the transcription factor LSF (Zambrano et al., 1998). The functional significance of those interactions have been discussed (Russo et al., 1998). Basically, this interaction network centered around the cytosolic tail of APP is proposed to be involved in 1) endocytosis and intracellular trafficking of APP, and 2) intracellular signalling events mediated by APP. Another work has shown that Fe65 and the mammalian homolog of the Drosophila disabled protein (DAB) bind to the cytosolic tail of several proteins involved in AD, like the LDL receptor, the LDL receptor related protein (LRP), and APP (Trommsdorff et al., 1998). The authors proposed that Fe65 and DAB can serve as molecular scaffold that bring together components of intracellular signalling complexes, including non-receptor tyrosine kinases such as the Abl protein. Thus, searching for interactors for LRP, Abl, and other proteins involved in this network may yield novel drug targets and therapeutic opportunities.

Abl is a cytoplasmic and nuclear protein tyrosine kinase that has been implicated in processes of cell differentiation, cell division, cell adhesion, and stress response (Mauro and Druker, 2001; Fernandez-Luna, 2000). The Abl protein contains 1130 amino acids (Fainstein et al., 1989) with an SH3 domain from amino acids 61 to 121, and SH2 domain from amino acids 127 to 217, and a protein kinase domain from amino acids 242 to 493. This kinase domain contains an ATP binding site from amino acids 248 to 271 and a tyrosine kinase active site from amino acids 359 to 371. The SH3 domain negatively regulates the tyrosine kinase activity, while deletion of the SH3 domain from Abl results in a protein with constitutive kinase activity. Furthermore, a very recent report (Zambrano et al., 2001) showed that, in cells expressing a constitutively active form of Abl, the cyto-tail of the amyloid protein precursor (APP) is tyrosine phosphorylated. Thus, Abl activity could modulate APP interaction with proteins such as Fe65, Fe65L, Mint1, and Mint2, which have been implicated in APP metabolism and Aβ production. There is also ample evidence that apoptosis is a major factor responsible for the neuronal loss observed in AD and other neurodegenerative conditions (Cotman et al., 1994; Smale et al., 1995; Shimohama, 2000; Behl, 2000; Offen et al., 2000). In particular, the role of the Bax protein in amyloid-induced neurotoxicity has been established (Selznick et al., 2000). Likewise, caspase-3 was shown to be activated in apoptotic neurons in response to exposure to the Aβ peptide (Harada and Sugimoto, 1999; Robertson et al., 2000). In addition, APP and the presenilins are targets of caspase-3 (Tesco et al., 1998; Weidemann et al., 1999; Gervais et al., 1999; Walter et al., 1998). Thus, Bax and caspase-3 interactors are also worth searching for. The protein CASK was identified as an interactor with the X11 (Mint1) protein (Borg et al., 1998; Borg et al., 1999). CASK is a member of the membrane-associated guanylate kinase (MAGUK) family. Proteins from this family typically contain one to three PDZ domains, an SH3 domain, and a guanylate kinase (GK) domain. In addition, CASK contains a serine/threonine protein kinase domain in the N-terminal region. MAGUK proteins are mainly localized at the synapses and function as synaptic scaffolding and clustering molecules for signalling proteins such as NMDA receptors and potassium channels (Kornau et al., 1995; Nagano et al., 1998; Muller et al., 1996; Nehring et al., 2000). Because of the effect of the Mint1-CASK complex on APP trafficking and metabolism (Tomita et al., 1999; Mueller et al., 2000), searching for CASK interactors may also yield drug target opportunities that could modulate amyloid production. Likewise, searching for novel MAGUK interactors is also a strategy to find new signalling proteins involved in synaptic function and that represent therapeutic opportunities against neurodegeneration. Members of the MAGUK family that we used in searches include the post-synaptic density protein 95 (PSD95), the discs-large protein 1 (DLG1), the discs-large protein 2 (DLG2), the discs-large protein 3 (DLG3), and the neuroendocrine discs-large protein (NE-dlg).

Using a fragment of Abl from amino acids 1 to 252 we found three interacting proteins that may be involved in the same biological pathway: the protein KIAA0410, the nucleoporin p62 and the suppressor of cytokine signaling 2 (SOCS2). Proteins of the KIAA series were identified by random cDNA cloning at the Kazusa DNA Research Institute (KDRI) in Japan. The report from the KDRI for KIAA0410 shows a complete sequence of 6356 bp with an ORF of 1455 bp coding for 485 aa. The putative ATG initiation codon is in a fair Kozak environment (G in −3), and preceded by an upstream in-frame STOP codons. The mRNA expression profile, performed at the KDRI by rt-PCR, shows very low KIAA0410 mRNA levels in kidney and ovary, and barely detectable levels in placenta, lung, liver, thymus, prostate, and testis. In all other tissues examined (including brain), mRNA levels are below the detection threshold. Nevertheless, we isolated 4 identical interacting clones, all from a hippocampus library. KIAA0410 is 87% identical to the rat nucleoporin p58 and we suggest that KIAA0410 is probably the human homolog of rat nucleoporin p58. This protein is a member of the p62 pore complex (Hu et al., 1996), an assembly of nuclear pore complex glycoproteins that includes p62, p58, p54, and p45 nucleoporins. The p58 and p45 proteins are generated by alternative splicing of a unique message. All four proteins of the complex are present on both side of the nuclear membrane, thus making the interactions with Abl (cytoplasmic) topologically possible. The Abl bait used in the search, from aa 1 to 252, interacts with an N-terminal domain of KIAA0410 (p58, aa 41 to 389).

In the same search, we identified a C-terminal domain of p62 (aa 439 to 505) as an interactor. Protein sequence analysis shows that p62 and KIAA0410 share very limited similarity, and only in their N-terminal domains. Thus, it is remarkable that Abl interacts with two proteins from the same complex, through domains that do not share any similarity with each other. The functional significance of those two interactions becomes clear in the light of the interaction of Abl with SOCS2.

Cytokines are secreted proteins that elicit cellular responses mediated by specific receptors, and activation of the “JAK-STAT” pathway. Upon binding of the cytokines to their respective receptors, members of the Janus Kinase (JAK) family are activated. In turn, members of the STAT family of transcription factors are phosphorylated, dimerize, and increase the transcription of genes with STAT recognition sites in their promoters. Termination of the signal elicited by cytokines is brought about by intracellular proteins of the SOCS (suppressor of cytokine signaling) family. The first three members of the family, SOCS-1, SOCS-2, and SOCS-3, were cloned for their ability to inhibit cellular signals elicited by IL-6 (Starr et al., 1997; Starr and Hilton, 1998). In another study (Dey et al. 1998), SOCS-1 and SOCS-2 were shown to interact with the cytosolic domain of the insulin-like growth factor I receptor (IGF-IR) and inhibit its signaling. The SOCS genes are expressed in many tissues and cell types, including brain (Polizzotto et al., 2000). While SOCS-1 and SOCS-3 display a low and widespread expression pattern, SOCS-2 is expressed at high levels and is found exclusively in neurons. SOCS-2 expression is switched on at the time of neuronal differentiation. Structurally, the SOCS proteins contain a variable amino-terminal region, a central Src-homology 2 (SH2) domain and a novel conserved carboxy-terminal motif termed the SOCS box (Starr et al., 1997). In addition to the amyloid cascade, the involvement of an inflammatory component in AD neurodegeneration is well documented (Eikelenboom et al., 2001; Halliday et al., 2001). Very recently (Luterman et al., 2001), elevated levels of IL-6 and TGF-beta 1 mRNA were observed in the entorhinal cortex of patients with severe AD-like dementia compared to non-demented age-matched controls, but not in patients in earlier disease stages. Moreover, IL-6 mRNA levels correlate with the number of tangles, but not senile plaques.

Recently, nucleoporin p62 was shown to be involved in the translocation of activated STAT3 from the cytoplasm into the nucleus in brain neurons (Lu et al., 1998). Although activation of the STATs usually occurs through phosphorylation by a tyrosine kinase from the JAK family, STAT proteins can also be phosphorylated by other tyrosine kinases such as Src and Abl (Bromberg and Darnell, 2000). Thus, STAT-3 phosphorylation by Abl might happen in the vicinity of the nuclear pore, just before its translocation into the nucleus. In the light of these studies, the interactions that we report here suggest that Abl might be involved in the modulation of the JAK-STAT pathway, through its interactions with SOCS-2, KIAA0410 (nucleoporin p58), and nucleoporin p62. We suggest that pharmacological modulation of Abl activity and interactions with KIAA0410 (nucleoporin p58), and nucleoporin p62 could provide novel therapeutic opportunities for the regulation of the JAK-STAT pathway and the inflammation component of Alzheimer's disease.

We also report an interaction between Abl and the AF6 protein. Most acute leukemia in infancy show abnormalities of chromosome band 11q23. In these cases, translocation results in fusion of a gene at 11q23, variously called ALL1, MLL, and the human homolog of fly trithorax, with genes on chromosome 4, 9, or 19. Prasad et al (1993) described the cloning and characterization of the “partner gene” involved in a fourth common translocation involving 11q23, t(6;11)(q27;q23). The gene, designated AF6, was found to be expressed in a variety of cell types and to encode a protein of 1,612 amino acids. The protein contains short stretches rich in proline, charged amino acids, serines, or glutamines. In addition, the AF6 protein contains the GLGF motif shared with several proteins thought to be involved in signal transduction at special cell-cell junctions.

We also report an interaction between Abl and calcineurin B, which is the regulatory subunit of a calcium-dependent, calmodulin-activated phosphatase. As the nomenclature of phosphatases has recently been changed, calcineurin B is also known as protein phosphatase 3. As calcineurin B and CIB are 58% similar (28% identical), this connection between calcineurin B and Abl is not unexpected: Abl is a substrate of the DNA-dependent protein kinase (DNA-PK) (Kharbanda et al., 1997), a protein which is a known CIB interactor (Wu and Lieber, 1997) (CIB is called KIP in this particular study). The similarity between CIB and calcineurin suggests the possibility that Abl might interact with CIB, and that DNA-PK might interact with calcineurin B. Interestingly, Abl also interacts with break point cluster (Pendergast et al., 1991), which is a 6-catenin interactor. In addition to its involvement in cancerous cell transformation, Abl also participates, together with DNA-PK, in the determination of cell fate in response to DNA damage, i.e. growth arrest and repair or induction of apoptosis (Kharbanda et al., 1998). Thus, CIB and/or calcineurin B may be regulatory subunits of DNA-PK and be involved in the control of apoptotic processes through the interaction between DNA-PK and Abl. Another possibility is the potential regulatory role of calcineurin B in the signaling cascade modulated by Abl in response to ligand binding to receptors of the LDL receptor family. Fe65 and DAB bind to the cytoplasmic tails of LRP, the LDL receptor, and APP, where they can potentially serve as molecular scaffolds for the assembly of cytosolic multiprotein complexes (Trommsdorff et al., 1998). FE65 contains two distinct protein interaction domains that interact with LRP and APP, respectively, raising the possibility that LRP can modulate the intracellular trafficking of APP (Russo et al., 1998). Tyrosine-phosphorylated DAB can recruit nonreceptor tyrosine kinases, such as Abl, to the cytoplasmic tails of the proteins to which it binds, suggesting a molecular pathway by which receptor/ligand interaction on the cell surface could generate an intracellular signal, modulated by regulatory proteins such as calcineurin B. Finally, there is a possible link between the Abl-calcineurin B connection and apoptosis due to disruption of calcium homeostasis, resulting from mutations in the presenilins. The effect of PS1 mutations on the ryanodine receptor and calcium homeostasis are well documented (Mattson et al., 2000; Chan et al., 2000). A recent work by Pack-Chung et al (2000) shows that PS2 also modulates the activity of the ryanodine receptor, through its interaction with sorcin, and that sorcin is localized in the same subcellular compartment as calcineurin B. Thus, Abl might be involved in apoptosis through its interaction with calcineurin B and disruption of calcium homeostasis.

We also report an interaction between Abl and KIAA0269 (WAVE1). The KDRI report shows a full-length sequence of 2625 bp with an ORF coding for 559 aa, in which a leucine zipper domain was identified from aa 69 to 91 (the protein sequence from the KDRI contains 567 aa because their program translates mRNA sequence into amino acids not from the ATG initiation codon, but from the first codon following the first in-frame stop codon upstream of the ATG initiation codon). Northern blot data from the KDRI shows that the KIAA0269 mRNA is found at high levels in testis and brain and low levels in all other tissues examined. KIAA0269 is also known as WAVE1, a WASP-family protein involved in actin clustering induced by Rac (Miki et al., 1998). A very recent paper shows that WAVE1 functions through two distinct mechanisms, Arp2/3 complex-dependent and -independent (Sasaki et al., 2000). Another recent paper shows that the Arp2/3 complex and Mena act together in the stimulation of actin polymerization in growth cones by nerve growth factor (Goldberg et al., 2000). As Abl associates with several proteins involved in axonal growth, including proteins from the Ena family, it is possible that Abl is involved in an Arp2/3 complex-independent mechanism with effects on actin polymerization.

We also report an interaction between Abl and KIAA0779. The report from the KDRI shows a partial sequence of 3743 bp with an ORF of 960 bp coding for 320 aa, representing the C-terminal region of the protein. The Kyte-Doolittle hydropathy profile shows two very hydrophobic domains (index 3 or above) that might potentially cross the membrane. Indeed, the domain analysis performed at the KDRI reports two transmembrane domains, from aa 257 to 279, and from aa 287 to 309. No other well characterized protein domain was identified in KIAA0779. The tissue distribution profile performed at the KDRI (by rt-PCR-ELISA) shows highest levels in brain and ovary, followed by testis, liver, lung, heart, and kidney, and lowest levels in pancreas and spleen. Our results indicate that Abl might interact with the cytosolic C-terminal domain of a transmembrane protein, thus potentially transducing the signal elicited by a novel receptor.

We also report an interaction between Abl and KIAA0820. The report from the KDRI shows a partial sequence of 5804 bp with an ORF of 2106 bp coding for 702 aa. The mRNA expression profile performed at the KDRI by rt-PCR, shows very high KIAA0820 mRNA levels in brain and medium to very low levels in all other tissues examined. Within the brain, all regions (including hippocampus and amygdala) have very high levels of KIAA0820 mRNA, with slightly lower levels in the spinal cord and the corpus callosum. The domain analysis performed at the KDRI reveals the presence of two domains of the dynamin family, and one pleckstrin homology (PH) domain. KIAA0820 is 96% identical to rat dynamin 3, a protein of 848 aa said to be expressed specifically in testis (GenBank entry D14076). Because KIAA0820 is found at much higher levels in brain than testis, it is unclear whether KIAA0820 is the human homolog of rat dynamin 3. In any event, Abl appears to interact with a protein that mediates the microtubule-associated transport of intracellular vesicles.

We also report an interaction between Abl and KIAA0846. The report from the KDRI shows a complete sequence of 4204 bp with an ORF of 2067 bp coding for 689 aa. The putative ATG initiation codon is in an excellent Kozak environment (A in −3 and G in +4), and is preceded by several upstream in-frame STOP codons. The expression profile performed at the KDRI by rt-PCR shows medium to high levels of KIAA0846 mRNA in heart, brain, lung, and kidney. Medium levels are observed in liver, spleen, and ovary. Medium to low levels are found in skeletal muscle, pancreas, and testis. The domain analysis performed at the KDRI reveals the presence of two calcium-binding EF hands between aa 400 and 500, a RasGEF domain (Guanine nucleotide Exchange Factor) between aa 148 and 334, and a phorbol esters/diacylglycerol (DAG) binding domain between aa 496 and 545 (also known as the Protein kinase C conserved region 1 (C1) domain). KIAA0846 is 53% identical to a human protein known as “calcium and DAG-regulated guanine nucleotide exchange factor II”. Therefore, we suggest that Abl interacts with a novel human protein that activates Ras (or other GTPases) upon calcium and DAG binding.

We also report an interaction between Abl and Sox-2, a transcription factor of the Sox family which consists of a large number of embryonically expressed genes containing a 79-amino-acid DNA-binding domain known as the HMG (High Mobility Group) box. Sox-2 is expressed in many tissues and was shown to activate the crystallin gene in the developing lens in chicken and mouse (Kamachi et al., 1995), and is involved in neural induction (Mizuseki et al., 1998). Sox-2 was also shown to repress the transcriptional activation mediated by the POU transcription factor Oct-4, expressed specifically in the germ line (Botquin et al., 1998). The interaction suggests that Abl might control the transcriptional events mediated by Sox-2, either by direct phosphorylation of Sox-2 or the phosphorylation of proteins that associate with Sox-2 (such as POU transcription factors). In support of this hypothesis, Abl was shown to control the activity of the STAT3 transcription factor by direct phosphorylation and induction of its nuclear translocation (Danial and Rothman, 2000; Bromberg and Darnell, 2000).

We also first report an interaction between Abl and adenylyl cyclase associated protein 2 (CAP2). This protein is 64% identical to CAP1 (Yu et al. 1994), with which it heterodimerizes (Hubberstey et al., 1996). CAP1 in turn was shown to bind to BAT3 (Hubberstey et al., 1996), an APP interactor. Like BAT3, CAP proteins contain a central proline-rich region (Freeman et al., 1996) that interacts with proteins containing SH3 domains, such as Abl. In fact, CAP1 was found to bind strongly to Abl (Freeman et al., 1996). Thus, both CAP1 and CAP2 bind to Abl. Because cAMP and CREB-mediated transcription are essential for long-term potentiation (LTP) in the hippocampus (Walton et al., 1999), the interactions between Abl and CAP1 and Abl and CAP2 suggest that Abl might also play a role in hippocampal LTP. However, although Abl is expressed in the hippocampus, Grant et al (1992) showed that Abl is not necessary for LTP in this brain region. This suggests that the CAP proteins might also interact with other proteins necessary for LTP.

We also report an interaction between Abl and the centrosomal protein kendrin. Also called pericentrin-B (Li et al., 2001), kendrin is a large protein (3321 amino acids) that was identified as a component of the centrosome (Flory et al., 2000). The C-terminal region of kendrin binds to calmodulin, and the N-terminal region shares 61% identity (75% similarity) with pericentrin. The Abl bait used in the search was from amino acids 1 to 252, a region of the protein that contains the SH3 domain. We identified three clones in a brain library encoding amino acids 1984 to 2708 and one clone in a hippocampus library encoding amino acids 1707 to 2133 of kendrin. Thus, we suggest that the domain of kendrin that interacts with Abl is from amino acids 1984 to 2133. The centrosome is a structure that is essential for microtubule nucleation, a process involved in the mitotic spindle formation necessary for chromosome segregation during mitosis. Through direct interaction of centrosomal components (such as pericentrin) with microtubules, the centrosome is also important for the general organization and structure of the cytoskeleton. In neurons, the centrosome plays an important role in the generation of neuritic cytoskeleton (Baas, 1996; Baas, 1998). Beside amyloid plaques, cytoskeletal structures known as neurofibrillary tangles are an invariable pathological feature of Alzheimer's disease (AD). The major component of these tangles is a hyperphosphorylated form of the microtubule-associated protein tau (Mesulam, 2000). In this respect, it is interesting that tau and tau-like proteins have been found to associate with the centrosome (Cross et al., 1996). The interaction of Abl with the centrosomal protein kendrin suggests that Abl could be involved in tau phosphorylation. Reciprocally, the kinase activity of Abl was shown to be stimulated by microtubule damage, leading to cell death (Nehme et al., 2000). Furthermore, a very recent report (Zambrano et al., 2001) showed that, in cells expressing a constitutively active form of Abl, the cyto-tail of the amyloid protein precursor (APP) is tyrosine phosphorylated. Thus, Abl activity could modulate APP interaction with proteins such as Fe65, Fe65L, Mint1, and Mint2, which have been implicated in APP metabolism and Aβ production. In brief, the interaction between Abl and kendrin suggests that Abl might be at the crossroads between the amyloidogenic pathway and neurofibrillary tangle formation. Finally, as the presenilins (PS1 and PS2) are clearly involved in amyloidogenesis, it is worth noting that the presenilins have been detected in the centrosome (Li et al., 1997b).

We also report an interaction between Abl and KIAA0886. The report from the KDRI shows a sequence of 4053 bp with an ORF of 3699 bp coding for 1233 aa. The expression profile performed at the KDRI by rt-PCR shows very high levels of KIAA0886 mRNA in all tissues examined, with the exception of pancreas (high) and spleen (medium). Domain analysis by the KDRI reports 4 transmembrane domains in the C-terminal region, from aa 1006 to 1157. Our interacting clone codes for aa 1024 to 1192 (C-terminus), a domain that is on both sides of the membrane. KIAA0886 shares extensive similarity and identity with a number of other proteins. Smaller proteins are 100% identical to some KIAA0886 domains, suggesting the existence of several splice variants. First, the foocen-m cDNA codes for a protein of 373 aa which is 100% identical to KIAA0886 over a domain from aa 1 to 185 and a second domain from aa 1005 to 1192 (C-terminus). Basically, foocen-m appears to be a truncated version of KIAA0886, missing aa 186 to 1004. Except for the GenBank entry (AF132047) that claims that foocen-m is a member of the reticulon family, no information is available on this protein. Second, the ASY cDNA codes for a protein of 373 aa, which is 99.5% identical to foocen-m (371 aa out of 373), and thus represents the same KIAA0886 splice variant. Although no article was published on the function of this ASY protein, the GenBank entry (AB015639) describes it as a “cell-death inducing” protein. Third, the Neuroendocrine-Specific Protein type C (NSP-C) cDNA codes for a protein of 199 aa that is 100% identical to the 188 C-terminal aa of ASY, foocen-m, and KIAA0886, and has a distinct 11 aa N-terminus (GenBank entry AF077050). Basically, this protein represent the C-terminal half of ASY or foocen-m. KIAA0886 is also 76% identical (with long stretches of perfect identity) to the rat Nogo-A protein (Chen et al. 2000). We suggest that KIAA0886 is either the human homolog of rat Nogo-A, or a Nogo-A-like protein. The Nogo-A protein was recently shown to be an inhibitor of neurite outgrowth present in CNS myelin and synthesized by oligodendrocytes (Chen et al., 2000). On the other hand, the expression of NSP-C (basically identical to the C-terminal 188 aa of Nogo-A) is correlated with neuronal differentiation (Hens et al. 1998). Thus, it appears that alternative splicing of the same primary transcript can give rise to proteins having opposite effects on neuronal differentiation. Because the ASY protein is described in GenBank as a cell death-inducing protein, we suggest that the N-terminal region of Nogo-A inhibits neurite outgrowth, while the C-terminal region correlates with neuronal differentiation. The Abl-interacting domain is present in all forms of KIAA0886 related proteins. In particular, KIAA0886 and rat Nogo-A are virtually identical over the KIAA0886 domain interacting with Abl. Thus, the interaction between Abl and the Nogo-A related proteins might control neuronal differentiation and neurite outgrowth.

We also report that Bax and FAK2 interact with a domain of KIAA0886 (aa 556 to 990) that is present in Nogo-A and KIAA0886, but not in the smaller splice variants, ASY and NSP-C. We suggest that a complex pattern of interaction between the KIAA0886-related proteins and Abl, Bax, and FAK2 might control neuronal differentiation and neurite outgrowth. Because Bax is an apoptotic protein (Martin, 2001; Korsmeyer et al., 1999), and because FAK2 is involved in neurite extension and neuronal differentiation (Park et al., 2000; Menegon et al., 1999), we suggest that the interaction between Bax and KIAA0886 or Nogo-A leads to inhibition of neurite outgrowth and neuronal cell death, while the interaction between FAK2 and KIAA0886 or Nogo-A leads to neurite outgrowth and neuronal differentiation. The function of Abl in this complex might be to phosphorylate KIAA0886 or Nogo-A, thus modulating their interaction with Bax or FAK2.

We also report an interaction between Abl and the transcription factor ZFM1 The gene for ZFM1 was initially isolated as a candidate tumor suppressor (Toda et al., 1994) which was supposedly involved in multiple endocrine neoplasia type 1 (MEN1). However, a more recent work showed that ZFM1 is excluded as a candidate gene for MEN1 (Lloyd et al., 1997). The ZFM1 gene encodes a protein of 623 amino acids containing domains with interesting structural properties including a nuclear transport domain, a metal binding motif, and glutamine- and proline-rich regions (Wrehlke et al., 1997). ZFM1 was suggested to be a new member of a protein family that combines features of signal transduction and RNA activation. An interesting study by Covini et al (Covini et al., 1999) showed a transient elevation of the ZFM1 message in the brain following global ischemia. The ZFM1 mRNA was induced in the dentate gyrus as early as 4 hours post-ischemia. Expression peaked at 2 days in the whole hippocampus and cortex, and then progressively decreased towards control levels. By day 4, expression had disappeared almost entirely from the cells in the CA 1 region of the hippocampus, concomitant with the degeneration of pyramidal neurons. Because ZFM1 was shown to be induced by p53-mediated apoptosis (Amson et al., 1996), Covini et al suggested that ZFM1 may represent a relevant link between p53 and the neuroprotective or neurodegenerative processes which follow cerebral ischemia. Its interaction with Abl suggests that its effects might be mediated by phosphorylation events.

We also report an interaction between LSF and the transcription factor ZFM1. Because LSF interact with APP, PS1, and Fe65, a disruption of the transcriptional activity mediated by ZFM1 might be involved in AD related neurodegeneration. In support of this hypothesis, a very recent study identified LSF as a new Alzheimer susceptibility gene, located on chromosome 12 (Lambert et al., 2000).

We report an interaction between BAX and the enzyme bleomycin hydrolase. Bax is an apoptotic protein (Martin, 2001; Korsmeyer et al., 1999) that was suggested to mediate amyloid toxicity (Selznick et al., 2000). Originally isolated as a protease that confers chemotherapy resistance to cancer cells (it cleaves the glycopeptide bleomycin) (Ferrando et al. 1996), bleomycin hydrolase is expressed in all tissues and is thought to have a general function in protein degradation and turnover. While some studies suggested a genetic association between bleomycin hydrolase and Alzheimer's disease (Montoya et al., 1998; Shastry and Giblin, 1999), these results have been challenged by others (Farrer et al., 1998; Namba et al., 1999a; Thome et al., 1999). Most recently however, the association between bleomycin hydrolase genotype and Alzheimer's disease was confirmed (Papassotiropoulos et al., 2000). This association was more pronounced in ApoE4 carriers. Moreover, bleomycin hydrolase might be involved in AD pathology in response to oxidative stress. It is also worth noting that bleomycin hydrolase has been localized in senile plaques (Raina et al. 1999), and very recently, bleomycin hydrolase was shown to interact with and regulate the secretion of APP (Namba et al., 1999b; Lefterov et al., 2000) (both sAPPα and sAPPβ, as well as the Aβ peptide). Bleomycin hydrolase protects cancer cells from apoptosis by cleaving bleomycin, however its interaction with BAX suggests an involvement in the regulation of apoptosis that is independent from its bleomycin-cleaving activity. Because its normal function (still not clearly elucidated) might be in protein degradation and turnover, it is tempting to speculate that bleomycin hydrolase is somehow involved in Ab clearance. Its reported interaction with APP and its localization in senile plaques support this hypothesis. Mutations in the bleomycin hydrolase gene that reduce the activity of the enzyme might account for the elevated AD risk factor described for particular bleomycin hydrolase genotypes.

The proteins disclosed in the present invention were found to interact with APP or other proteins involved in AD. Because of the involvement of these proteins in AD, the proteins disclosed herein also participate in the pathogenesis of AD. Therefore, the present invention provides a list of uses of those proteins and DNA encoding those proteins for the development of diagnostic and therapeutic tools against AD.

### 2.2. Protein Complexes

The present invention comprises complexes formed between full-length proteins, as well as fragments of these full-length proteins that interact in a manner analogous to the polypeptides disclosed in the tables above. Importantly, in most cases in the tables above, for each interacting protein pair, exemplary fragments of bait and prey proteins are provided that interact with each other to form a protein complex. It is widely accepted in the field of protein-protein interactions that interactions between fragments of full-length proteins are generally indicative of interactions formed between corresponding full-length proteins containing such fragments. Thus, in view of the disclosure provided herein, an ordinarily skilled person in the art, apprised of the interacting polypeptides disclosed in the tables above, would immediately envisage protein complexes comprising the corresponding full-length proteins, as well as numerous other complexes comprised of alternative fragments of the bait and prey proteins, interacting in the same manner. For example, such other fragments may include bait or prey protein fragments containing the relevant amino acid residues defined by the coordinates provided in the tables, but with additional amino acid residues flanking the defined amino acid residues at either or both ends.

Practically, one of skill in the art, using the exact species of interacting polypeptides disclosed in the tables above as a starting point, would understand that the genus of interacting polypeptides encompassed by the instant invention includes alternative polypeptide fragments, either shorter or longer, that include the necessary interaction domains required to form the protein-protein interactions, which result in the formation of the protein complexes of the present invention. Routine experimentation, optionally directed by analysis of multiple sequence alignments to identify conserved amino acid residues and stretches of contiguous identical amino acid residues, can be used to define the minimal interacting fragments of the polypeptides in the tables. The routine experimentation needed to define such minimal interacting fragments can be readily practiced by one of skill in the art of molecular biology using the polymerase chain reaction to selectively amplify nucleic acids encoding smaller and smaller portions of the polypeptides disclosed-portions of the interacting proteins disclosed in the tables. The amplified nucleic acids can then be incorporated into expression cassettes in expression vectors that can be used to direct the expression of shorter polypeptides. Such routine experimentation often involves systematically constructing expression vectors encoding progressively shorter portions of one of the two interacting polypeptides described in the tables. For example, a fixed number of nucleotides (i.e., about 15, 30, 45, or 60), encoding a fixed number of amino acid residues (i.e., about 5, 10, 15, or 20, respectively) from either end of the interacting fragment, can be excluded from the coding sequence that is inserted into the cassette that directs the expression of the recombinant polypeptide fragment. The terminally-shortened recombinant polypeptides so expressed are then tested for their ability to interact with the partner protein disclosed in the tables. Reiterative rounds of amplification and cloning of shorter and shorter nucleic acids, followed by the expression of shorter and shorter fragments of the interacting proteins, and subsequent testing to determine whether such truncated fragments still interact, will predictably lead to the elucidation of minimal fragments still capable of interacting with the interacting partner identified in the table.

Additionally, using the species of interacting polypeptides described in the tables above as a starting point, one of skill in the art would also understand that the genus of interacting polypeptides can include polypeptides that are longer than those disclosed, as long as they contain the necessary domain responsible for the interactions. Practically, one of skill of molecular biology, using routine experimentation, can extend the length of the polypeptides described in the tables, from either or both ends, to ultimately encompass full-length (e.g., native) proteins, or even fusion proteins, which interact to form a protein complex representing the naturally-occurring complex found in vivo.

Furthermore, one of skill in the art of molecular biology and protein-protein interactions, using routine experimentation, can introduce amino acid sequence variations in the polypeptides disclosed in the tables, or in polypeptides both longer or shorter than those disclosed. Optionally directed by analysis of multiple sequence alignments to identify variable amino acid residues, one of average skill in the art can use site-directed mutagenesis to introduce specific changes in the amino acid sequence of the interacting partner polypeptides, and thereby produce “synthetic homologues” of the bait or prey proteins, or any interacting fragments thereof. For example, one of average skill in the art could introduce changes in the codons encoding specific amino acid residues found to vary in orthologous proteins. Similarly, one of average skill in the art could introduce changes in the codons encoding specific amino acid residues that result in conservative substitutions of those amino acid residues. For example, using such methods, one of skill in the art could introduce a site-specific mutation that changes a “CTT” codon to an “ATT” codon, thereby causing a leucine residue in the native polypeptide to be replaced by an isoleucine residue in the synthetic homologue. To a first approximation, such a conservative substitution in the expressed polypeptide would not be expected to abrogate the ability of the synthetic homologue to interact with its partner protein, and it may in fact increase the affinity of the interaction (see Graversen et al., J. Biol. Chem. 275:37390-37396 (2000)).

As a typical example of the relative skill in the art of (a) identifying and cloning orthologous proteins from divergent species, (b) identifying conserved regions of such orthologous proteins, and (c) further identifying orthologous interactions made by such proteins in the formation of multiprotein complexes, the work of Queimado and colleagues (Queimado et al., Nucleic Acids Res. 29:1884-1891 (2001)) is incorporated herein by reference in its entirety. Queimado and coworkers used the amino acid sequence of the yeast Mms19 protein to screen public databases for orthologous human and murine proteins. Upon identifying likely candidate coding sequences in cDNA libraries, they rapidly amplified cDNAs corresponding to the mRNAs expressed from the human and murine MMS19 genes. The amplified cDNAs were cloned, sequenced, and used to express the respective Mms19 proteins, which were tested for their ability to functionally complement yeast cells with deleted MMS19 genes. The identification, cloning and sequencing of MMS19 genes from divergent species allowed Queimado and colleagues to identify conserved HEAT repeat domains, known to be responsible for the assembly of multiprotein complexes required for nucleotide excision repair and transcription by RNA Polymerase II. (See: Queimado et al., Nucleic Acids Res. 29:1884-1891 (2001).)

As a typical example of the relative skill in the art of using site-specific mutations to introduce amino acid substitutions of specific residues in a protein, and subsequently determining the effect of such substitutions on the ability of the “synthetic homologous proteins” (i.e., site-specific mutant proteins) to interact with a binding partner, the work of Graversen and colleagues (Graversen et al., J. Biol. Chem. 275:37390-37396 (2000)) is incorporated herein by reference in its entirety. Graversen and colleagues introduced over a dozen specific amino acid changes in the C-type lectin domain of tetranectin at four locations, and then determined the effect these single amino acid substitutions had on the affinity of binding of C-type lectin domain for plasminogen kringle 4—the natural binding partner of tetranectin—using surface plasmon resonance binding and isothermal titration calorimetry binding analyses.

Given the fact that routine experimentation can yield polypeptides that are either shorter or longer than those specific polypeptides disclosed in the tables above, or, alternatively, are synthetic homologues of the disclosed interacting polypeptides; and given the fact that many such alternative fragments or synthetic homologues of these interacting polypeptides can still interact to form a protein complex—and can be readily confirmed as interacting with partner proteins—hundreds, if not thousands, of alternative species of protein complexes corresponding to each protein pair disclosed in the tables above are intrinsically disclosed herein to one of skill in the art apprised of the specific protein-protein interactions disclosed in the tables. Further, to a skilled artisan, such alternative species of protein complexes can not only be immediately envisaged and derived from the complexes identified in the tables above, but can be created and verified, using routine experimentation.

Fragments of the native full-length interacting proteins should contain interacting domains, and can interact to form a protein complex of the present invention. Such interacting fragments typically contain a contiguous span of anywhere from 5 to 10 amino acid residues up to several hundred amino acid residues. In most instances, however, the interacting fragments contain a contiguous span of about 20, 25, 30, 35, 40, 45, 50, 60, 70, 80, 90, 100, 120, 140, 160, 180, 200, or more, amino acid residues, from the native full-length interacting proteins. Interacting fragments can be identified by routine experimentation, as described herein.

The present invention also provides for protein complexes comprising homologues of the interacting proteins disclosed in the tables, and for fragments of such homologous proteins. Such protein complexes may contain any combination of homologous proteins, including naturally occurring homologues or, alternatively, “synthetic homologues” specifically engineered from naturally occurring proteins.

For example, the homologous proteins encompassed in the present invention include orthologous proteins, and fragments thereof, that can interact in a manner consistent with the interactions disclosed in the tables. Examples of such orthologous proteins include orthologous proteins from eukaryotic species, including plant, fungal, and animal orthologs, particularly mammalian orthologs. Especially useful orthologs are those orthologous proteins from species of ape, monkey, mouse, rat, rabbit, guinea pig, hamster, gerbil, cat, dog, pig, cow, sheep, goat, horse, chicken, duck, turkey, or fish, etc. The protein complexes encompassed by the present invention can comprise interacting proteins from the same species, or from different species.

When the protein complexes of the present invention comprise fragments of full-length proteins, the fragments of such homologous proteins can consist of about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, or more, contiguous amino acid residues. Such fragments can be about 50%, 60%, or 70%, preferably about 80% or 85%, more preferably 90%, 91%, 92%, 93%, 94%, and even more preferably 95%, 96%, 97%, 98%, or 99% identical to a corresponding fragment of the human homologue. Corresponding fragments can be identified by pairwise alignment of the homologous fragment with the full-length human sequence using BLASTP program, as described above. Once a corresponding fragment is identified by alignment, the percent identity between the homologous fragment and corresponding fragment of a human protein can be calculated.

Importantly, when protein complexes of the present invention comprise homologous proteins, protein fragments, or fragments of homologous proteins, such proteins comprise an interaction domain that facilitates the interaction between the protein, homologous protein, protein fragment, or fragment of a homologous protein and its binding partner.

Thus, for example, one interacting partner in a protein complex can be a complete native BAT3, a BAT3 homologue capable of interacting with, e.g., PN9113, a BAT3 derivative, a derivative of the BAT3 homologue, a BAT3 fragment capable of interacting with PN9113 (BAT3 fragment(s) containing the coordinates shown in the tables above), a homologue or derivative of the BAT3 fragment, or a fusion protein containing (1) complete native BAT3, (2) a BAT3 homologue capable of interacting with PN9113 or (3) a BAT3 fragment capable of interacting with PN9113. Besides native PN9113, useful interacting partners for BAT3 or a homologue or derivative or fragment thereof also include homologues of PN9113 capable of interacting with BAT3, derivatives of the native or homologue PN9113 capable of interacting with BAT3, fragments of the PN9113 capable of interacting with BAT3 (e.g., a fragment containing the identified interacting regions shown in the tables above), derivatives of the PN9113 fragments, or fusion proteins containing (1) a complete PN9113, (2) a PN9113 homologue capable of interacting with BAT3 or (3) a PN9113 fragment capable of interacting with BAT3.

PN9113 fragments capable of interacting with BAT3 can be identified by the combination of molecular engineering of a PN9113-encoding nucleic acid and a method for testing protein-protein interaction. For example, the coordinates in the tables above can be used as starting points and various PN9113 fragments falling within the coordinates can be generated by deletions from either or both ends of the coordinates. The resulting fragments can be tested for their ability to interact with BAT3 using any methods known in the art for detecting protein-protein interactions (e.g., yeast two-hybrid method). Alternatively, various PN9113 fragments can be made by chemical synthesis. The PN9113 fragments can then be tested for their ability to interact with BAT3 using any method known in the art for detecting protein-protein interactions. Examples of such methods include protein affinity chromatography, affinity blotting, in vitro binding assays, yeast two-hybrid assays, surface plasmon resonance and isothermal titration calorimetry binding analyses, and the like. Likewise, BAT3 fragments capable of interacting with PN9113 can also be identified in a similar manner.

Other protein complexes can be formed in a similar manner based on other interactions provided in the tables.

In a specific embodiment of the protein complex of the present invention, two or more interacting partners are directly fused together, or covalently linked together through a peptide linker, forming a hybrid protein having a single unbranched polypeptide chain. Thus, the protein complex may be formed by “intramolecular” interactions between two portions of the hybrid protein. Again, one or both of the fused or linked interacting partners in this protein complex may be a native protein or a homologue, derivative or fragment of a native protein.

The protein complexes of the present invention can also be in a modified form. For example, an antibody selectively immunoreactive with the protein complex can be bound to the protein complex. In another example, a non-antibody modulator capable of enhancing the interaction between the interacting partners in the protein complex may be included. Alternatively, the protein members in the protein complex may be cross-linked for purposes of stabilization. Various crosslinking methods may be used. For example, a bifunctional reagent in the form of R-S-S-R′ may be used in which the R and R′ groups can react with certain amino acid side chains in the protein complex forming covalent linkages. See e.g., Traut et al., in Creighton ed., Protein Function: A Practical Approach, IRL Press, Oxford, 1989; Baird et al., J. Biol. Chem., 251:6953-6962 (1976). Other useful crosslinking agents include, e.g., Denny-Jaffee reagent, a heterbiofunctional photoactivable moiety cleavable through an azo linkage (See Denny et al, Proc. Natl. Acad. Sci. USA, 81:5286-5290 (1984)), and ¹²⁵I-{S-[N-(3-iodo-4-azidosalicyl)cysteaminyl]-2-thiopyridine}, a cysteine-specific photocrosslinking reagent (see Chen et al., Science, 265:90-92 (1994)).

The above-described protein complexes may further include any additional components, e.g., other proteins, nucleic acids, lipid molecules, monosaccharides or polysaccharides, ions, etc.

### 2.3. Methods of Preparing Protein Complexes

The protein complex of the present invention can be prepared by a variety of methods. Specifically, a protein complex can be isolated directly from an animal tissue sample, preferably a human tissue sample containing the protein complex. Alternatively, a protein complex can be purified from host cells that recombinantly express the members of the protein complex. As will be apparent to a skilled artisan, a protein complex can be prepared from a tissue sample or recombinant host cells by coimmunoprecipitation using an antibody immunoreactive with an interacting protein partner, or preferably an antibody selectively immunoreactive with the protein complex as will be discussed in detail below.

The antibodies can be monoclonal or polyclonal. Communoprecipitation is a commonly used method in the art for isolating or detecting bound proteins. In this procedure, generally a serum sample or tissue or cell lysate is admixed with a suitable antibody. The protein complex bound to the antibody is precipitated and washed. The bound protein complexes are then eluted.

Alternatively, immunoaffinity chromatography and immunoblotting techniques may also be used in isolating the protein complexes from native tissue samples or recombinant host cells using an antibody immunoreactive with an interacting protein partner, or preferably an antibody selectively immunoreactive with the protein complex. For example, in protein immunoaffinity chromatography, the antibody is covalently or non-covalently coupled to a matrix (e.g., Sepharose), which is then packed into a column. Extract from a tissue sample, or lysate from recombinant cells is passed through the column where it contacts the antibodies attached to the matrix. The column is then washed with a low-salt solution to wash away the unbound or loosely (non-specifically) bound components. The protein complexes that are retained in the column can be then eluted from the column using a high-salt solution, a competitive antigen of the antibody, a chaotropic solvent, or sodium dodecyl sulfate (SDS), or the like. In immunoblotting, crude proteins samples from a tissue sample extract or recombinant host cell lysate are fractionated by polyacrylamide gel electrophoresis (PAGE) and then transferred to a membrane, e.g., nitrocellulose. Components of the protein complex can then be located on the membrane and identified by a variety of techniques, e.g., probing with specific antibodies.

In another embodiment, individual interacting protein partners may be isolated or purified independently from tissue samples or recombinant host cells using similar methods as described above. The individual interacting protein partners are then combined under conditions conducive to their interaction thereby forming a protein complex of the present invention. It is noted that different protein-protein interactions may require different conditions. As a starting point, for example, a buffer having 20 mM Tris-HCl, pH 7.0 and 500 mM NaCl may be used. Several different parameters may be varied, including temperature, pH, salt concentration, reducing agent, and the like. Some minor degree of experimentation may be required to determine the optimum incubation condition, this being well within the capability of one skilled in the art once apprised of the present disclosure.

In yet another embodiment, the protein complex of the present invention may be prepared from tissue samples or recombinant host cells or other suitable sources by protein affinity chromatography or affinity blotting. That is, one of the interacting protein partners is used to isolate the other interacting protein partner(s) by binding affinity thus forming protein complexes. Thus, an interacting protein partner prepared by purification from tissue samples or by recombinant expression or chemical synthesis may be bound covalently or non-covalently to a matrix, e.g., Sepharose, which is then packed into a chromatography column. The tissue sample extract or cell lysate from the recombinant cells can then be contacted with the bound protein on the matrix. A low-salt solution is used to wash off the unbound or loosely bound components, and a high-salt solution is then employed to elute the bound protein complexes in the column. In affinity blotting, crude protein samples from a tissue sample or recombinant host cell lysate can be fractionated by polyacrylamide gel electrophoresis (PAGE) and then transferred to a membrane, e.g., nitrocellulose. The purified interacting protein member is then bound to its interacting protein partner(s) on the membrane forming protein complexes, which are then isolated from the membrane.

It will be apparent to skilled artisans that any recombinant expression methods may be used in the present invention for purposes of expressing the protein complexes or individual interacting proteins. Generally, a nucleic acid encoding an interacting protein member can be introduced into a suitable host cell. For purposes of forming a recombinant protein complex within a host cell, nucleic acids encoding two or more interacting protein members should be introduced into the host cell.

Typically, the nucleic acids, preferably in the form of DNA, are incorporated into a vector to form expression vectors capable of directing the production of the interacting protein member(s) once introduced into a host cell. Many types of vectors can be used for the present invention. Methods for the construction of an expression vector for purposes of this invention should be apparent to skilled artisans apprised of the present disclosure. See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.

Generally, the expression vectors include an expression cassette having a promoter operably linked to a DNA encoding an interacting protein member. The promoter can be a native promoter, i.e., the promoter found in naturally occurring cells to be responsible for the expression of the interacting protein member in the cells. Alternatively, the expression cassette can be a chimeric one, i.e., having a heterologous promoter that is not the native promoter responsible for the expression of the interacting protein member in naturally occurring cells. The expression vector may further include an origin of DNA replication for the replication of the vectors in host cells. Preferably, the expression vectors also include a replication origin for the amplification of the vectors in, e.g., E. coli, and selection marker(s) for selecting and maintaining only those host cells harboring the expression vectors. Additionally, the expression cassettes preferably also contain inducible elements, which function to control the transcription from the DNA encoding an interacting protein member. Other regulatory sequences such as transcriptional enhancer sequences and translation regulation sequences (e.g., Shine-Dalgarno sequence) can also be operably included in the expression cassettes. Termination sequences such as the polyadenylation signals from bovine growth hormone, SV40, lacZ and AcMNPV polyhedral protein genes may also be operably linked to the DNA encoding an interacting protein member in the expression cassettes. An epitope tag coding sequence for detection and/or purification of the expressed protein can also be operably linked to the DNA encoding an interacting protein member such that a fusion protein is expressed. Examples of useful epitope tags include, but are not limited to, influenza virus hemagglutinin (HA), Simian Virus 5 (V5), polyhistidine (6×His), c-myc, lacZ, GST, and the like. Proteins with polyhistidine tags can be easily detected and/or purified with Ni affinity columns, while specific antibodies immunoreactive with many epitope tags are generally commercially available. The expression vectors may also contain components that direct the expressed protein extracellularly or to a particular intracellular compartment. Signal peptides, nuclear localization sequences, endoplasmic reticulum retention signals, mitochondrial localization sequences, myristoylation signals, palmitoylation signals, and transmembrane sequences are examples of optional vector components that can determine the destination of expressed proteins. When it is desirable to express two or more interacting protein members in a single host cell, the DNA fragments encoding the interacting protein members may be incorporated into a single vector or different vectors.

The thus constructed expression vectors can be introduced into the host cells by any techniques known in the art, e.g., by direct DNA transformation, microinjection, electroporation, viral infection, lipofection, gene gun, and the like. The expression of the interacting protein members may be transient or stable. The expression vectors can be maintained in host cells in an extrachromosomal state, i.e., as self-replicating plasmids or viruses. Alternatively, the expression vectors can be integrated into chromosomes of the host cells by conventional techniques such as selection of stable cell lines or site-specific recombination. In stable cell lines, at least the expression cassette portion of the expression vector is integrated into a chromosome of the host cells.

The vector construct can be designed to be suitable for expression in various host cells, including but not limited to bacteria, yeast cells, plant cells, insect cells, and mammalian and human cells. Methods for preparing expression vectors for expression in different host cells should be apparent to a skilled artisan.

Homologues and fragments of the native interacting protein members can also be easily expressed using the recombinant methods described above. For example, to express a protein fragment, the DNA fragment incorporated into the expression vector can be selected such that it only encodes the protein fragment. Likewise, a specific hybrid protein can be expressed using a recombinant DNA encoding the hybrid protein. Similarly, a homologue protein may be expressed from a DNA sequence encoding the homologue protein. A homologue-encoding DNA sequence may be obtained by manipulating the native protein-encoding sequence using recombinant DNA techniques. For this purpose, random or site-directed mutagenesis can be conducted using techniques generally known in the art. To make protein derivatives, for example, the amino acid sequence of a native interacting protein member may be changed in predetermined manners by site-directed DNA mutagenesis to create or remove consensus sequences for, e.g., phosphorylation by protein kinases, glycosylation, ribosylation, myristolation, palmytoylation, ubiquitination, and the like. Alternatively, non-natural amino acids can be incorporated into an interacting protein member during the synthesis of the protein in recombinant host cells. For example, photoreactive lysine derivatives can be incorporated into an interacting protein member during translation by using a modified lysyl-tRNA. See, e.g., Wiedmann et al., Nature, 328:830-833 (1989); Musch et al., Cell, 69:343-352 (1992). Other photoreactive amino acid derivatives can also be incorporated in a similar manner. See, e.g., High et al., J. Biol. Chem., 368:28745-28751 (1993). Indeed, the photoreactive amino acid derivatives thus incorporated into an interacting protein member can function to cross-link the protein to its interacting protein partner in a protein complex under predetermined conditions.

In addition, derivatives of the native interacting protein members of the present invention can also be prepared by chemically linking certain moieties to amino acid side chains of the native proteins.

If desired, the homologues and derivatives thus generated can be tested to determine whether they are capable of interacting with their intended partners to form protein complexes. Testing can be conducted by e.g., the yeast two-hybrid system or other methods known in the art for detecting protein-protein interaction.

A hybrid protein as described above having any interacting pair of the proteins described in the tables, or a homologue, derivative, or fragment thereof covalently linked together by a peptide bond or a peptide linker can be expressed recombinantly from a chimeric nucleic acid, e.g., a DNA or mRNA fragment encoding the fusion protein. Accordingly, the present invention also provides a nucleic acid encoding the hybrid protein of the present invention. In addition, an expression vector having incorporated therein a nucleic acid encoding the hybrid protein of the present invention is also provided. The methods for making such chimeric nucleic acids and expression vectors containing them will be apparent to skilled artisans apprised of the present disclosure.

### 2.4. Protein Microchip

In accordance with another embodiment of the present invention, a protein microchip or microarray is provided having one or more of the protein complexes and/or antibodies selectively immunoreactive with the protein complexes of the present invention. Protein microarrays are becoming increasingly important in both proteomics research and protein-based detection and diagnosis of diseases. The protein microarrays in accordance with this embodiment of the present invention will be useful in a variety of applications including, e.g., large-scale or high-throughput screening for compounds capable of binding to the protein complexes or modulating the interactions between the interacting protein members in the protein complexes.

The protein microarray of the present invention can be prepared in a number of methods known in the art. An example of a suitable method is that disclosed in MacBeath and Schreiber, Science, 289:1760-1763 (2000). Essentially, glass microscope slides are treated with an aldehyde-containing silane reagent (SuperAldehyde Substrates purchased from TeleChem International, Cupertino, Calif.). Nanoliter volumes of protein samples in a phosphate-buffered saline with 40% glycerol are then spotted onto the treated slides using a high-precision contact-printing robot. After incubation, the slides are immersed in a bovine serum albumin (BSA)-containing buffer to quench the unreacted aldehydes and to form a BSA layer that functions to prevent non-specific protein binding in subsequent applications of the microchip. Alternatively, as disclosed in MacBeath and Schreiber, proteins or protein complexes of the present invention can be attached to a BSA-NHS slide by covalent linkages. BSA-NHS slides are fabricated by first attaching a molecular layer of BSA to the surface of glass slides and then activating the BSA with N,N′-disuccinimidyl carbonate. As a result, the amino groups of the lysine, aspartate, and glutamate residues on the BSA are activated and can form covalent urea or amide linkages with protein samples spotted on the slides. See MacBeath and Schreiber, Science, 289:1760-1763 (2000).

Another example of a useful method for preparing the protein microchip of the present invention is that disclosed in PCT Publication Nos. WO 00/4389A2 and WO 00/04382, both of which are assigned to Zyomyx and are incorporated herein by reference. First, a substrate or chip base is covered with one or more layers of thin organic film to eliminate any surface defects, insulate proteins from the base materials, and to ensure uniform protein array. Next, a plurality of protein-capturing agents (e.g., antibodies, peptides, etc.) are arrayed and attached to the base that is covered with the thin film. Proteins or protein complexes can then be bound to the capturing agents forming a protein microarray. The protein microchips are kept in flow chambers with an aqueous solution.

The protein microarray of the present invention can also be made by the method disclosed in PCT Publication No. WO 99/36576 assigned to Packard Bioscience Company, which is incorporated herein by reference. For example, a three-dimensional hydrophilic polymer matrix, i.e., a gel, is first dispensed on a solid substrate such as a glass slide. The polymer matrix gel is capable of expanding or contracting and contains a coupling reagent that reacts with amine groups. Thus, proteins and protein complexes can be contacted with the matrix gel in an expanded aqueous and porous state to allow reactions between the amine groups on the protein or protein complexes with the coupling reagents thus immobilizing the proteins and protein complexes on the substrate. Thereafter, the gel is contracted to embed the attached proteins and protein complexes in the matrix gel.

Alternatively, the proteins and protein complexes of the present invention can be incorporated into a commercially available protein microchip, e.g., the ProteinChip System from Ciphergen Biosystems Inc., Palo Alto, Calif. The ProteinChip System comprises metal chips having a treated surface, which interact with proteins. Basically, a metal chip surface is coated with a silicon dioxide film. The molecules of interest such as proteins and protein complexes can then be attached covalently to the chip surface via a silane coupling agent.

The protein microchips of the present invention can also be prepared with other methods known in the art, e.g., those disclosed in U.S. Pat. Nos. 6,087,102, 6,139,831, 6,087,103; PCT Publication Nos. WO 99/60156, WO 99/39210, WO 00/54046, WO 00/53625, WO 99/51773, WO 99/35289, WO 97/42507, WO 01/01142, WO 00/63694, WO 00/61806, WO 99/61148, WO 99/40434, all of which are incorporated herein by reference.

### 3. Antibodies

In accordance with another aspect of the present invention, an antibody immunoreactive against a protein complex of the present invention is provided. In one embodiment, the antibody is selectively immunoreactive with a protein complex of the present invention. Specifically, the phrase “selectively immunoreactive with a protein complex” as used herein means that the immunoreactivity of the antibody of the present invention with the protein complex is substantially higher than that with the individual interacting members of the protein complex so that the binding of the antibody to the protein complex is readily distinguishable from the binding of the antibody to the individual interacting member proteins based on the strength of the binding affinities. Preferably, the binding constants differ by a magnitude of at least 2 fold, more preferably at least 5 fold, even more preferably at least 10 fold, and most preferably at least 100 fold. In a specific embodiment, the antibody is not substantially immunoreactive with the interacting protein members of the protein complex.

The antibodies of the present invention can be readily prepared using procedures generally known in the art. See, e.g., Harlow and Lane, Antibodies: A Laboratory Manual, Cold Spring Harbor Press, 1988. Typically, the protein complex against which an immunoreactive antibody is desired is used as the antigen for producing an immune response in a host animal. In one embodiment, the protein complex used consists of the native proteins. Preferably, the protein complex includes only protein fragments containing interacting regions provided in the tables. As a result, a greater portion of the total antibodies may be selectively immunoreactive with the protein complexes. The interaction domains can be selected from, e.g., those regions summarized in the tables above. In addition, various techniques known in the art for predicting epitopes may also be employed to design antigenic peptides based on the interacting protein members in a protein complex of the present invention to increase the possibility of producing an antibody selectively immunoreactive with the protein complex. Suitable epitope-prediction computer programs include, e.g., MacVector from International Biotechnologies, Inc. and Protean from DNAStar.

In a specific embodiment, a hybrid protein as described above in Section 2.1 is used as an antigen which has a first protein that is any one of the proteins described in the tables, or a homologue, derivative, or fragment thereof covalently linked by a peptide bond or a peptide linker to a second protein which is the interacting partner of the first protein, or a homologue, derivative, or fragment of the second protein. In a preferred embodiment, the hybrid protein consists of two interacting domains selected from the regions identified in a table above, or homologues or derivatives thereof, covalently linked together by a peptide bond or a linker molecule.

The antibody of the present invention can be a polyclonal antibody to a protein complex of the present invention. To produce the polyclonal antibody, various animal hosts can be employed, including, e.g., mice, rats, rabbits, goats, guinea pigs, hamsters, etc. A suitable antigen which is a protein complex of the present invention or a derivative thereof as described above can be administered directly to a host animal to illicit immune reactions. Alternatively, it can be administered together with a carrier such as keyhole limpet hemocyanin (KLH), bovine serum albumin (BSA), ovalbumin, and Tetanus toxoid. Optionally, the antigen is conjugated to a carrier by a coupling agent such as carbodiimide, glutaraldehyde, and MBS. Any conventional adjuvants may be used to boost the immune response of the host animal to the protein complex antigen. Suitable adjuvants known in the art include but are not limited to Complete Freund's Adjuvant (which contains killed mycobacterial cells and mineral oil), incomplete Freund's Adjuvant (which lacks the cellular components), aluminum salts, MF59 from Chiron (Emeryville, Calif.), monophospholipid, synthetic trehalose dicorynomycolate (TDM) and cell wall skeleton (CWS) both from Corixa Corp. (Seattle, Wash.), non-ionic surfactant vesicles (NISV) from Proteus International PLC (Cheshire, U.K.), and saponins. The antigen preparation can be administered to a host animal by subcutaneous, intramuscular, intravenous, intradermal, or intraperitoneal injection, or by injection into a lymphoid organ.

The antibodies of the present invention may also be monoclonal. Such monoclonal antibodies may be developed using any conventional techniques known in the art. For example, the popular hybridoma method disclosed in Kohler and Milstein, Nature, 256:495-497 (1975) is now a well-developed technique that can be used in the present invention. See U.S. Pat. No. 4,376,110, which is incorporated herein by reference. Essentially, B-lymphocytes producing a polyclonal antibody against a protein complex of the present invention can be fused with myeloma cells to generate a library of hybridoma clones. The hybridoma population is then screened for antigen binding specificity and also for immunoglobulin class (isotype). In this manner, pure hybridoma clones producing specific homogenous antibodies can be selected. See generally, Harlow and Lane, Antibodies: A Laboratory Manual, Cold Spring Harbor Press, 1988. Alternatively, other techniques known in the art may also be used to prepare monoclonal antibodies, which include but are not limited to the EBV hybridoma technique, the human N-cell hybridoma technique, and the trioma technique.

In addition, antibodies selectively immunoreactive with a protein complex of the present invention may also be recombinantly produced. For example, cDNAs prepared by PCR amplification from activated B-lymphocytes or hybridomas may be cloned into an expression vector to form a cDNA library, which is then introduced into a host cell for recombinant expression. The cDNA encoding a specific desired protein may then be isolated from the library. The isolated cDNA can be introduced into a suitable host cell for the expression of the protein. Thus, recombinant techniques can be used to produce specific native antibodies, hybrid antibodies capable of simultaneous reaction with more than one antigen, chimeric antibodies (e.g., the constant and variable regions are derived from different sources), univalent antibodies that comprise one heavy and light chain pair coupled with the Fc region of a third (heavy) chain, Fab proteins, and the like. See U.S. Pat. No. 4,816,567; European Patent Publication No. 0088994; Munro, Nature, 312:597 (1984); Morrison, Science, 229:1202 (1985); Oi et al., BioTechniques, 4:214 (1986); and Wood et al., Nature, 314:446-449 (1985), all of which are incorporated herein by reference. Antibody fragments such as Fv fragments, single-chain Fv fragments (scFv), Fab′ fragments, and F(ab′)₂ fragments can also be recombinantly produced by methods disclosed in, e.g., U.S. Pat. No. 4,946,778; Skerra & Plückthun, Science, 240:1038-1041 (1988); Better et al., Science, 240:1041-1043 (1988); and Bird, et al., Science, 242:423-426 (1988), all of which are incorporated herein by reference.

In a preferred embodiment, the antibodies provided in accordance with the present invention are partially or fully humanized antibodies. For this purpose, any methods known in the art may be used. For example, partially humanized chimeric antibodies having V regions derived from the tumor-specific mouse monoclonal antibody, but human C regions are disclosed in Morrison and Oi, Adv. Immunol., 44:65-92 (1989). In addition, fully humanized antibodies can be made using transgenic non-human animals. For example, transgenic non-human animals such as transgenic mice can be produced in which endogenous immunoglobulin genes are suppressed or deleted, while heterologous antibodies are encoded entirely by exogenous immunoglobulin genes, preferably human immunoglobulin genes, recombinantly introduced into the genome. See e.g., U.S. Pat. Nos. 5,530,101; 5,545,806; 6,075,181; PCT Publication No. WO 94/02602; Green et. al., Nat. Genetics, 7: 13-21 (1994); and Lonberg et al., Nature 368: 856-859 (1994), all of which are incorporated herein by reference. The transgenic non-human host animal may be immunized with suitable antigens such as a protein complex of the present invention or one or more of the interacting protein members thereof to illicit specific immune response thus producing humanized antibodies. In addition, cell lines producing specific humanized antibodies can also be derived from the immunized transgenic non-human animals. For example, mature B-lymphocytes obtained from a transgenic animal producing humanized antibodies can be fused to myeloma cells and the resulting hybridoma clones may be selected for specific humanized antibodies with desired binding specificities. Alternatively, cDNAs may be extracted from mature B-lymphocytes and used in establishing a library that is subsequently screened for clones encoding humanized antibodies with desired binding specificities.

In yet another embodiment, a bifunctional antibody is provided that has two different antigen binding sites, each being specific to a different interacting protein member in a protein complex of the present invention. The bifunctional antibody may be produced using a variety of methods known in the art. For example, two different monoclonal antibody-producing hybridomas can be fused together. One of the two hybridomas may produce a monoclonal antibody specific against an interacting protein member of a protein complex of the present invention, while the other hybridoma generates a monoclonal antibody immunoreactive with another interacting protein member of the protein complex. The thus formed new hybridoma produces different antibodies including a desired bifunctional antibody, i.e., an antibody immunoreactive with both of the interacting protein members. The bifunctional antibody can be readily purified. See Milstein and Cuello, Nature, 305:537-540 (1983).

Alternatively, a bifunctional antibody may also be produced using heterobifunctional crosslinkers to chemically link two different monoclonal antibodies, each being immunoreactive with a different interacting protein member of a protein complex. Therefore, the aggregate will bind to two interacting protein members of the protein complex. See Staerz et al, Nature, 314:628-631 (1985); Perez et al, Nature, 316:354-356 (1985).

In addition, bifunctional antibodies can also be produced by recombinantly expressing light and heavy chain genes in a hybridoma that itself produces a monoclonal antibody. As a result, a mixture of antibodies including a bifunctional antibody is produced. See DeMonte et al, Proc. Natl. Acad. Sci., USA, 87:2941-2945 (1990); Lenz and Weidle, Gene, 87:213-218 (1990).

Preferably, a bifunctional antibody in accordance with the present invention is produced by the method disclosed in U.S. Pat. No. 5,582,996, which is incorporated herein by reference. For example, two different Fabs can be provided and mixed together. The first Fab can bind to an interacting protein member of a protein complex, and has a heavy chain constant region having a first complementary domain not naturally present in the Fab but capable of binding a second complementary domain. The second Fab is capable of binding another interacting protein member of the protein complex, and has a heavy chain constant region comprising a second complementary domain not naturally present in the Fab but capable of binding to the first complementary domain. Each of the two complementary domains is capable of stably binding to the other but not to itself. For example, the leucine zipper regions of c-fos and c-jun oncogenes may be used as the first and second complementary domains. As a result, the first and second complementary domains interact with each other to form a leucine zipper thus associating the two different Fabs into a single antibody construct capable of binding to two antigenic sites.

Other suitable methods known in the art for producing bifunctional antibodies may also be used, which include those disclosed in Holliger et al., Proc. Nat'l Acad. Sci. USA, 90:6444-6448 (1993); de Kruif et al., J. Biol. Chem., 271:7630-7634 (1996); Coloma and Morrison, Nat. Biotechnol., 15:159-163 (1997); Muller et al, FEBS Lett., 422:259-264 (1998); and Muller et al., FEBS Lett., 432:45-49 (1998), all of which are incorporated herein by reference.

### 4. Methods of Detecting Protein Complexes

Another aspect of the present invention relates to methods for detecting the protein complexes of the present invention, particularly for determining the concentration of a specific protein complex in a patient sample.

In one embodiment, the concentration of a protein complex of the present invention is determined in cells, tissue, or an organ of a patient. For example, the protein complex can be isolated or purified from a patient sample obtained from cells, tissue, or an organ of the patient and the amount thereof is determined. As described above, the protein complex can be prepared from cells, tissue or organ samples by coimmunoprecipitation using an antibody immunoreactive with an interacting protein member, a bifunctional antibody that is immunoreactive with two or more interacting protein members of the protein complex, or preferably an antibody selectively immunoreactive with the protein complex. When bifunctional antibodies or antibodies immunoreactive with only free interacting protein members are used, individual interacting protein members not complexed with other proteins may also be isolated along with the protein complex containing such individual proteins. However, they can be readily separated from the protein complex using methods known in the art, e.g., size-based separation methods such as gel filtration, or by subtracting the protein complex from the mixture using an antibody specific against another individual interacting protein member. Additionally, proteins in a sample can be separated in a gel such as polyacrylamide gel and subsequently immunoblotted using an antibody immunoreactive with the protein complex.

Alternatively, the concentration of the protein complex can be determined in a sample without separation, isolation or purification. For this purpose, it is preferred that an antibody selectively immunoreactive with the specific protein complex is used in an immunoassay. For example, immunocytochemical methods can be used. Other well known antibody-based techniques can also be used including, e.g., enzyme-linked immunosorbent assay (ELISA), radioimmunoassay (RIA), immunoradiometric assays (IRMA), fluorescent immunoassays, protein A immunoassays, and immunoenzymatic assays (IEMA). See e.g., U.S. Pat. Nos. 4,376,110 and 4,486,530, both of which are incorporated herein by reference.

In addition, since a specific protein complex is formed from its interacting protein members, if one of the interacting protein members is at a relatively low concentration in a patient, it may be reasonably expected that the concentration of the protein complex in the patient may also be low. Therefore, the concentration of an individual interacting protein member of a specific protein complex can be determined in a patient sample which can then be used as a reasonably accurate indicator of the concentration of the protein complex in the sample. For this purpose, antibodies against an individual interacting protein member of a specific complex can be used in any one of the methods described above. In a preferred embodiment, the concentration of each of the interacting protein members of a protein complex is determined in a patient sample and the relative concentration of the protein complex is then deduced.

In addition, the relative protein complex concentration in a patient can also be determined by determining the concentration of the mRNA encoding an interacting protein member of the protein complex. Preferably, each interacting protein member's mRNA concentration in a patient sample is determined. For this purpose, methods for determining mRNA concentration generally known in the art may all be used. Examples of such methods include, e.g., Northern blot assay, dot blot assay, PCR assay (preferably quantitative PCR assay), in situ hybridization assay, and the like.

As discussed above, each interaction between members of an interacting protein pair of the present invention suggests that the proteins and/or the protein complexes formed by such proteins may be involved in common biological processes and disease pathways. In addition, the interactions under physiological conditions may lead to the formation of protein complexes in vivo. The protein complexes are expected to mediate the functions and biological activities of the interacting members of the protein complexes. Thus, aberrations in the protein complexes or the individual proteins and the degree of the aberration may be indicators for the diseases or disorders. These aberrations may be used as parameters for classifying and/or staging one of the above-described diseases. In addition, they may also be indicators for patients' response to a drug therapy.

Association between a physiological state (e.g., physiological disorder, predisposition to the disorder, a disease state, response to a drug therapy, or other physiological phenomena or phenotypes) and a specific aberration in a protein complex of the present invention or an individual interacting member thereof can be readily determined by comparative analysis of the protein complex and/or the interacting members thereof in a normal population and an abnormal or affected population. Thus, for example, one can study the concentration, localization and distribution of a particular protein complex, mutations in the interacting protein members of the protein complex, and/or the binding affinity between the interacting protein members in both a normal population and a population affected with a particular physiological disorder described above. The study results can be compared and analyzed by statistical means. Any detected statistically significant difference in the two populations would indicate an association. For example, if the concentration of the protein complex is statistically significantly higher in the affected population than in the normal population, then it can be reasonably concluded that higher concentration of the protein complex is associated with the physiological disorder.

Thus, once an association is established between a particular type of aberration in a particular protein complex of the present invention or in an interacting protein member thereof and a physiological disorder or disease or predisposition to the physiological disorder or disease, then the particular physiological disorder or disease or predisposition to the physiological disorder or disease can be diagnosed or detected by determining whether a patient has the particular aberration.

Accordingly, the present invention also provides a method for diagnosing in a patient a disease or physiological disorder, or a predisposition to the disease or disorder, such as Alzheimer's disease (including mild cognitive impairment), Huntington's disease, Parkinson's disease, schizophrenia, depression, dementia and other neurodegenerative diseases, as well as diabetes, obesity and coronary artery disease by determining whether there is any aberration in the patient with respect to a protein complex identified according to the present invention. The same protein complex is analyzed in a normal individual and is compared with the results obtained in the patient. In this manner, any protein complex aberration in the patient can be detected. As used herein, the term “aberration” when used in the context of protein complexes of the present invention means any alterations of a protein complex including increased or decreased concentration of the protein complex in a particular cell or tissue or organ or the total body, altered localization of the protein complex in cellular compartments or in locations of a tissue or organ, changes in binding affinity of an interacting protein member of the protein complex, mutations in an interacting protein member or the gene encoding the protein, and the like. As will be apparent to a skilled artisan, the term “aberration” is used in a relative sense. That is, an aberration is relative to a normal condition.

As used herein, the term “diagnosis” means detecting a disease or disorder or determining the stage or degree of a disease or disorder. The term “diagnosis” also encompasses detecting a predisposition to a disease or disorder, determining the therapeutic effect of a drug therapy, or predicting the pattern of response to a drug therapy or xenobiotics. The diagnosis methods of the present invention may be used independently, or in combination with other diagnosing and/or staging methods known in the medical art for a particular disease or disorder.

Thus, in one embodiment, the method of diagnosis is conducted by detecting, in a patient, the concentrations of one or more protein complexes of the present invention using any one of the methods described above, and determining whether the patient has an aberrant concentration of the protein complexes.

The diagnosis may also be based on the determination of the concentrations of one or more interacting protein members (at the protein, cDNA or mRNA level) of a protein complex of the present invention. An aberrant concentration of an interacting protein member may indicate a physiological disorder or a predisposition to a physiological disorder.

In another embodiment, the method of diagnosis comprises determining, in a patient, the cellular localization, or tissue or organ distribution of a protein complex of the present invention and determining whether the patient has an aberrant localization or distribution of the protein complex. For example, immunocytochemical or immunohistochemical assays can be performed on a cell, tissue or organ sample from a patient using an antibody selectively immunoreactive with a protein complex of the present invention. Antibodies immunoreactive with both an individual interacting protein member and a protein complex containing the protein member may also be used, in which case it is preferred that antibodies immunoreactive with other interacting protein members are also used in the assay. In addition, nucleic acid probes may also be used in in situ hybridization assays to detect the localization or distribution of the mRNAs encoding the interacting protein members of a protein complex. Preferably, the mRNA encoding each interacting protein member of a protein complex is detected concurrently.

In yet another embodiment, the method of diagnosis of the present invention comprises detecting any mutations in one or more interacting protein members of a protein complex of the present invention. In particular, it is desirable to determine whether the interacting protein members have any mutations that will lead to, or are associated with, changes in the functional activity of the proteins or changes in their binding affinity to other interacting protein members in forming a protein complex of the present invention. Examples of such mutations include but are not limited to, e.g., deletions, insertions and rearrangements in the genes encoding the protein members, and nucleotide or amino acid substitutions and the like. In a preferred embodiment, the domains of the interacting protein members that are responsible for the protein-protein interactions, and lead to protein complex formation, are screened to detect any mutations therein. For example, genomic DNA or cDNA encoding an interacting protein member can be prepared from a patient sample, and sequenced. The thus obtained sequence may be compared with known wild-type sequences to identify any mutations. Alternatively, an interacting protein member may be purified from a patient sample and analyzed by protein sequencing or mass spectrometry to detect any amino acid sequence changes. Any methods known in the art for detecting mutations may be used, as will be apparent to skilled artisans apprised of the present disclosure.

In another embodiment, the method of diagnosis includes determining the binding constant of the interacting protein members of one or more protein complexes. For example, the interacting protein members can be obtained from a patient by direct purification or by recombinant expression from genomic DNAs or cDNAs prepared from a patient sample encoding the interacting protein members. Binding constants represent the strength of the protein-protein interaction between the interacting protein members in a protein complex. Thus, by measuring binding constants, subtle aberrations in binding affinity may be detected.

A number of methods known in the art for estimating and determining binding constants in protein-protein interactions are reviewed in Phizicky and Fields, et al., Microbiol. Rev., 59:94-123 (1995), which is incorporated herein by reference. For example, protein affinity chromatography may be used. First, columns are prepared with different concentrations of an interacting protein member, which is covalently bound to the columns. Then a preparation of an interacting protein partner is run through the column and washed with buffer. The interacting protein partner bound to the interacting protein member linked to the column is then eluted. A binding constant is then estimated based on the concentrations of the bound protein and the eluted protein. Alternatively, the method of sedimentation through gradients monitors the rate of sedimentation of a mixture of proteins through gradients of glycerol or sucrose. At concentrations above the binding constant, proteins can sediment as a protein complex. Thus, binding constant can be calculated based on the concentrations. Other suitable methods known in the art for estimating binding constant include but are not limited to gel filtration column such as nonequilibrium “small-zone” gel filtration columns (See e.g., Gill et al., J. Mol. Biol., 220:307-324 (1991)), the Hummel-Dreyer method of equilibrium gel filtration (See e.g., Hummel and Dreyer, Biochim. Biophys. Acta, 63:530-532 (1962)) and large-zone equilibrium gel filtration (See e.g., Gilbert and Kellett, J. Biol. Chem., 246:6079-6086 (1971)), sedimentation equilibrium (See e.g., Rivas and Minton, Trends Biochem., 18:284-287 (1993)), fluorescence methods such as fluorescence spectrum (See e.g., Otto-Bruc et al, Biochemistry, 32:8632-8645 (1993)) and fluorescence polarization or anisotropy with tagged molecules (See e.g., Weiel and Hershey, Biochemistry, 20:5859-5865 (1981)), solution equilibrium measured with immobilized binding protein (See e.g., Nelson and Long, Biochemistry, 30:2384-2390 (1991)), and surface plasmon resonance (See e.g., Panayotou et al., Mol. Cell. Biol., 13:3567-3576 (1993)).

In another embodiment, the diagnosis method of the present invention comprises detecting protein-protein interactions in functional assay systems such as the yeast two-hybrid system. Accordingly, to determine the protein-protein interaction between two interacting protein members that normally form a protein complex in normal individuals, cDNAs encoding the interacting protein members can be isolated from a patient to be diagnosed. The thus cloned cDNAs or fragments thereof can be subcloned into vectors for use in yeast two-hybrid systems. Preferably a reverse yeast two-hybrid system is used such that failure of interaction between the proteins may be positively detected. The use of yeast two-hybrid systems or other systems for detecting protein-protein interactions is known in the art and is described below in Section 5.3.1.

A kit may be used for conducting the diagnosis methods of the present invention. Typically, the kit should contain, in a carrier or compartmentalized container, reagents useful in any of the above-described embodiments of the diagnosis method. The carrier can be a container or support, in the form of, e.g., bag, box, tube, rack, and is optionally compartmentalized. The carrier may define an enclosed confinement for safety purposes during shipment and storage. In one embodiment, the kit includes an antibody selectively immunoreactive with a protein complex of the present invention. In addition, antibodies against individual interacting protein members of the protein complexes may also be included. The antibodies may be labeled with a detectable marker such as radioactive isotopes, or enzymatic or fluorescence markers. Alternatively secondary antibodies such as labeled anti-IgG and the like may be included for detection purposes. Optionally, the kit can include one or more of the protein complexes of the present invention prepared or purified from a normal individual or an individual afflicted with a physiological disorder associated with an aberration in the protein complexes or an interacting protein member thereof. In addition, the kit may further include one or more of the interacting protein members of the protein complexes of the present invention prepared or purified from a normal individual or an individual afflicted with a physiological disorder associated with an aberration in the protein complexes or an interacting protein member thereof. Suitable oligonucleotide primers useful in the amplification of the genes or cDNAs for the interacting protein members may also be provided in the kit. In particular, in a preferred embodiment, the kit includes a first oligonucleotide selectively hybridizable to the mRNA or cDNA encoding one member of an interacting pair of proteins and a second oligonucleotide selectively hybridizable to the mRNA or cDNA encoding the other of the interacting pair. Additional oligonucleotides hybridizing to a region of the genes encoding an interacting pair of proteins may also be included. Such oligonucleotides may be used as PCR primers for, e.g., quantitative PCR amplification of mRNAs encoding the interacting proteins, or as hybridizing probes for detecting the mRNAs. The oligonucleotides may have a length of from about 8 nucleotides to about 100 nucleotides, preferably from about 12 to about 50 nucleotides, and more preferably from about 15 to about 30 nucleotides. In addition, the kit may also contain oligonucleotides that can be used as hybridization probes for detecting the cDNAs or mRNAs encoding the interacting protein members. Preferably, instructions for using the kit or reagents contained therein are also included in the kit.

### 5. Use of Protein Complexes or Interacting Protein Members Thereof in Screening Assays for Modulators

The protein complexes of the present invention and interacting members thereof can also be used in screening assays to identify modulators of the protein complexes, and/or the interacting proteins. In addition, homologues, derivatives or fragments of the interacting proteins provided in this invention may also be used in such screening assays. As used herein, the term “modulator” encompasses any compounds that can cause any form of alteration of the biological activities or functions of the proteins or protein complexes, including, e.g., enhancing or reducing their biological activities, increasing or decreasing their stability, altering their affinity or specificity to certain other biological molecules, etc. In addition, the term “modulator” as used herein also includes any compounds that simply bind any of the proteins described in the tables, and/or the proteins complexes of the present invention. For example, a modulator can be an “interaction antagonist” capable of interfering with or disrupting or dissociating protein-protein interaction between an interacting pair of proteins identified in the tables, or homologues, fragments or derivatives thereof. A modulator can also be an “interaction agonist” that initiates or strengthens the interaction between the protein members of a protein complex of the present invention, or homologues, fragments or derivatives thereof.

In addition, the discovery of protein ligands of the present invention allows the use of screening assays to identify modulators of individual proteins of the protein complexes. Typical high-throughput screening assays involve measuring the modulation of the enzymatic activity of a protein. However, typical high-throughput screening assays are not applicable to proteins that exhibit little or no measurable enzymatic activity. The present discovery of novel ligands of proteins allows a screen to be setup that does not utilize enzymatic activity measurements. Consequently, the present invention enables a non-enzymatic high-throughput assay to be performed for modulators of individual proteins and/or protein complexes described in the tables.

Accordingly, the present invention provides screening methods for selecting modulators of any of the proteins described in the tables, or a mutant form thereof, or a protein-protein interaction between an interacting pair of proteins provided in the present invention, or homologues, fragments or derivatives thereof.

The selected compounds can be tested for their ability to modulate (interfere with or strengthen) the interaction between the interacting partners within the protein complexes of the present invention. In addition, the compounds can also be further tested for their ability to modulate (inhibit or enhance) cellular functions such as neurotransmission, regulation of APP production and APP metabolism, regulation of Aβ production and Aβ metabolism, processing of proteins destined for secretion, regulation of the wingless pathway, and modulation of thyroid receptor signaling in cells as well as their effectiveness in treating diseases such as Alzheimer's disease (including mild cognitive impairment), Huntington's disease, Parkinson's disease, schizophrenia, depression, dementia and other neurodegenerative diseases, as well as diabetes, obesity and coronary artery disease.

The modulators selected in accordance with the screening methods of the present invention can be effective in modulating the functions or activities of individual interacting proteins, or the protein complexes of the present invention. For example, compounds capable of binding to the protein complexes may be capable of modulating the functions of the protein complexes. Additionally, compounds that interfere with, weaken, dissociate or disrupt, or alternatively, initiate, facilitate or stabilize the protein-protein interaction between the interacting protein members of the protein complexes can also be effective in modulating the functions or activities of the protein complexes. Thus, the compounds identified in the screening methods of the present invention can be made into therapeutically or prophylactically effective drugs for preventing or ameliorating diseases, disorders or symptoms caused by or associated with a protein complex or an interacting member thereof. Alternatively, they may be used as leads to aid the design and identification of therapeutically or prophylactically effective compounds for diseases, disorders or symptoms caused by or associated with the protein complex or interacting protein members thereof. The protein complexes and/or interacting protein members thereof in accordance with the present invention can be used in any of a variety of drug screening techniques. Drug screening can be performed as described herein or using well-known techniques, such as those described in U.S. Pat. Nos. 5,800,998 and 5,891,628, both of which are incorporated herein by reference.

### 5.1. Test Compounds

Any test compounds may be screened in the screening assays of the present invention to select modulators of the protein complexes or interacting members thereof. By the term “selecting” or “select” compounds it is intended to encompass both (a) choosing compounds from a group previously unknown to be modulators of a protein complex or interacting protein members thereof, and (b) testing compounds that are known to be capable of binding, or modulating the functions and activities of, a protein complex or interacting protein members thereof. Both types of compounds are generally referred to herein as “test compounds.” The test compounds may include, by way of example, proteins (e.g., antibodies, small peptides, artificial or natural proteins), nucleic acids, and derivatives, mimetics and analogs thereof, and small organic molecules having a molecular weight of no greater than 10,000 daltons, more preferably less than 5,000 daltons. Preferably, the test compounds are provided in library formats known in the art, e.g., in chemically synthesized libraries, recombinantly expressed libraries (e.g., phage display libraries), and in vitro translation-based libraries (e.g., ribosome display libraries).

For example, the screening assays of the present invention can be used in the antibody production processes described in Section 3 to select antibodies with desirable specificities. Various forms of antibodies or derivatives thereof may be screened, including but not limited to, polyclonal antibodies, monoclonal antibodies, bifunctional antibodies, chimeric antibodies, single chain antibodies, antibody fragments such as Fv fragments, single-chain Fv fragments (scFv), Fab′ fragments, and F(ab′)₂ fragments, and various modified forms of antibodies such as catalytic antibodies, and antibodies conjugated to toxins or drugs, and the like. The antibodies can be of any types such as IgG, IgE, IgA, or IgM. Humanized antibodies are particularly preferred. Preferably, the various antibodies and antibody fragments may be provided in libraries to allow large-scale high throughput screening. For example, expression libraries expressing antibodies or antibody fragments may be constructed by a method disclosed, e.g., in Huse et al., Science, 246:1275-1281 (1989), which is incorporated herein by reference. Single-chain Fv (scFv) antibodies are of particular interest in diagnostic and therapeutic applications. Methods for providing antibody libraries are also provided in U.S. Pat. Nos. 6,096,551; 5,844,093; 5,837,460; 5,789,208; and 5,667,988, all of which are incorporated herein by reference.

Peptidic test compounds may be peptides having L-amino acids and/or D-amino acids, phosphopeptides, and other types of peptides. The screened peptides can be of any size, but preferably have less than about 50 amino acids. Smaller peptides are easier to deliver into a patient's body. Various forms of modified peptides may also be screened. Like antibodies, peptides can also be provided in, e.g., combinatorial libraries. See generally, Gallop et al., J. Med. Chem., 37:1233-1251 (1994). Methods for making random peptide libraries are disclosed in, e.g., Devlin et al, Science, 249:404-406 (1990). Other suitable methods for constructing peptide libraries and screening peptides therefrom are disclosed in, e.g., Scott and Smith, Science, 249:386-390 (1990); Moran et al., J. Am. Chem. Soc., 117:10787-10788 (1995) (a library of electronically tagged synthetic peptides); Stachelhaus et al., Science, 269:69-72 (1995); U.S. Pat. Nos. 6,156,511; 6,107,059; 6,015,561; 5,750,344; 5,834,318; 5,750,344, all of which are incorporated herein by reference. For example, random-sequence peptide phage display libraries may be generated by cloning synthetic oligonucleotides into the gene III or gene VIII of an E. coli filamentous phage. The thus generated phage can propagate in E. coli. and express peptides encoded by the oligonucleotides as fusion proteins on the surface of the phage. Scott and Smith, Science, 249:368-390 (1990). Alternatively, the “peptides on plasmids” method may also be used to form peptide libraries. In this method, random peptides may be fused to the C-terminus of the E. coli. Lac repressor by recombinant technologies and expressed from a plasmid that also contains Lac repressor-binding sites. As a result, the peptide fusions bind to the same plasmid that encodes them.

Small organic or inorganic non-peptide non-nucleotide compounds are preferred test compounds for the screening assays of the present invention. They too can be provided in a library format. See generally, Gordan et al. J. Med. Chem., 37:1385-1401 (1994). For example, benzodiazepine libraries are provided in Bunin and Ellman, J. Am. Chem. Soc., 114:10997-10998 (1992), which is incorporated herein by reference. Methods for constructing and screening peptoid libraries are disclosed in Simon et al., Proc. Natl. Acad. Sci. USA, 89:9367-9371 (1992). Methods for the biosynthesis of novel polyketides in a library format are described in McDaniel et al, Science, 262:1546-1550 (1993) and Kao et al., Science, 265:509-512 (1994). Various libraries of small organic molecules and methods of construction thereof are disclosed in U.S. Pat. Nos. 6,162,926 (multiply-substituted fullerene derivatives); 6,093,798 (hydroxamic acid derivatives); 5,962,337 (combinatorial 1,4-benzodiazepin-2,5-dione library); 5,877,278 (Synthesis of N-substituted oligomers); 5,866,341 (compositions and methods for screening drug libraries); 5,792,821 (polymerizable cyclodextrin derivatives); 5,766,963 (hydroxypropylamine library); and 5,698,685 (morpholino-subunit combinatorial library), all of which are incorporated herein by reference.

Other compounds such as oligonucleotides and peptide nucleic acids (PNA), and analogs and derivatives thereof may also be screened to identify clinically useful compounds. Combinatorial libraries of oligonucleotides are also known in the art. See Gold et al., J. Biol. Chem., 270:13581-13584 (1995).

### 5.2. In vitro Screening Assays

The test compounds may be screened in an in vitro assay to identify compounds capable of binding the protein complexes or interacting protein members thereof in accordance with the present invention. For this purpose, a test compound is contacted with a protein complex or an interacting protein member thereof under conditions and for a time sufficient to allow specific interaction between the test compound and the target components to occur, thereby resulting in the binding of the compound to the target, and the formation of a complex. Subsequently, the binding event is detected.

Various screening techniques known in the art may be used in the present invention. The protein complexes and the interacting protein members thereof may be prepared by any suitable methods, e.g., by recombinant expression and purification. The protein complexes and/or interacting protein members thereof (both are referred to as “target” hereinafter in this section) may be free in solution. A test compound may be mixed with a target forming a liquid mixture. The compound may be labeled with a detectable marker. Upon mixing under suitable conditions, the binding complex having the compound and the target may be co-immunoprecipitated and washed. The compound in the precipitated complex may be detected based on the marker on the compound.

In a preferred embodiment, the target is immobilized on a solid support or on a cell surface. Preferably, the target can be arrayed into a protein microchip in a method described in Section 2.3. For example, a target may be immobilized directly onto a microchip substrate such as glass slides or onto multi-well plates using non-neutralizing antibodies, i.e., antibodies capable of binding to the target but do not substantially affect its biological activities. To affect the screening, test compounds can be contacted with the immobilized target to allow binding to occur to form complexes under standard binding assay conditions. Either the targets or test compounds are labeled with a detectable marker using well-known labeling techniques. For example, U.S. Pat. No. 5,741,713 discloses combinatorial libraries of biochemical compounds labeled with NMR active isotopes. To identify binding compounds, one may measure the formation of the target-test compound complexes or kinetics for the formation thereof. When combinatorial libraries of organic non-peptide non-nucleic acid compounds are screened, it is preferred that labeled or encoded (or “tagged”) combinatorial libraries are used to allow rapid decoding of lead structures. This is especially important because, unlike biological libraries, individual compounds found in chemical libraries cannot be amplified by self-amplification. Tagged combinatorial libraries are provided in, e.g., Borchardt and Still, J. Am. Chem. Soc., 116:373-374 (1994) and Moran et al., J. Am. Chem. Soc., 117:10787-10788 (1995), both of which are incorporated herein by reference.

Alternatively, the test compounds can be immobilized on a solid support, e.g., forming a microarray of test compounds. The target protein or protein complex is then contacted with the test compounds. The target may be labeled with any suitable detection marker. For example, the target may be labeled with radioactive isotopes or fluorescence marker before binding reaction occurs. Alternatively, after the binding reactions, antibodies that are immunoreactive with the target and are labeled with radioactive materials, fluorescence markers, enzymes, or labeled secondary anti-Ig antibodies may be used to detect any bound target thus identifying the binding compound. One example of this embodiment is the protein probing method. That is, the target provided in accordance with the present invention is used as a probe to screen expression libraries of proteins or random peptides. The expression libraries can be phage display libraries, in vitro translation-based libraries, or ordinary expression cDNA libraries. The libraries may be immobilized on a solid support such as nitrocellulose filters. See e.g., Sikela and Hahn, Proc. Natl. Acad. Sci. USA, 84:3038-3042 (1987). The probe may be labeled with a radioactive isotope or a fluorescence marker. Alternatively, the probe can be biotinylated and detected with a streptavidin-alkaline phosphatase conjugate. More conveniently, the bound probe may be detected with an antibody.

In one embodiment, the proteins identified in the tables are used as targets in an assay to select modulators of the proteins in the tables. In a specific embodiment, a screening assay for modulators of BAT3 is performed by using PN9113 as a ligand for BAT3. For example, in this screen, BAT3 can be immobilized on a solid support and is contacted with test compounds. PN9113 can be labeled with a detectable marker such as radioactive materials or fluorescence markers using label techniques known in the art. The labeled PN9113 is allowed to contact the immobilized BAT3 and levels of BAT3-PN9113 protein complex formed are detected by washing away unbound PN9113. The ability of the test compounds to modulate BAT3 is determined by comparing the level of BAT3-PN9113 complex formed when BAT3 is contacted with test compounds to the level formed in the absence of test compounds. Alternatively, as will be apparent to skilled artisans, the PN9113 protein can be detected with labeled antibody against PN9113, or by an antibody specific to a polypeptide that is fused to PN9113.

In yet another embodiment, the protein complexes identified in the tables are used as a target in the assay. In a specific embodiment, a protein complex used in the screening assay includes a hybrid protein as described in Section 2.1, which is formed by fusion of two interacting protein members or fragments or interaction domains thereof. The hybrid protein may also be designed such that it contains a detectable epitope tag fused thereto. Suitable examples of such epitope tags include sequences derived from, e.g., influenza virus hemagglutinin (HA), Simian Virus 5 (V5), polyhistidine (6×His), c-myc, lacZ, GST, and the like.

In addition, a known ligand capable of binding to the target can be used in competitive binding assays. Complexes between the known ligand and the target can be formed and then contacted with test compounds. The ability of a test compound to interfere with the interaction between the target and the known ligand is measured. One exemplary ligand is an antibody capable of specifically binding the target. Particularly, such an antibody is especially useful for identifying peptides that share one or more antigenic determinants of the target protein complex or interacting protein members thereof.

In a specific preferred embodiment, the target is one member of an interacting pair of proteins disclosed according the present invention, or a homologue, derivative or fragment thereof, and the competitive ligand is the other member of the interacting pair of proteins, or a homologue, derivative or fragment thereof. Preferably, either the target or the ligand or both are labeled with or detectable marker. Alternatively, either the target or the ligand or both are fusion proteins that contain a detectable epitope tag having one or more sequences derived from, e.g., influenza virus hemagglutinin (HA), Simian Virus 5 (V5), polyhistidine (6×His), c-myc, lacZ, GST, and the like.

Thus, for example, the target can be immobilized to a solid support. The ligand can be a fusion protein having a fragment of an interactor of the target protein fused to an epitope tag, e.g., c-myc. The ligand can be contacted with the target in the presence or absence of one or more test compounds. Both ligand molecules associated with the immobilized target and ligand molecules not associated with the target can be detected with, e.g., an antibody against the c-myc tag. As a result, test compounds capable of binding the target or ligand, or disrupting the protein-protein interaction between the target and ligand can be identified or selected.

Test compounds may also be screened in an in vitro assay to identify compounds capable of dissociating the protein complexes identified in the tables above. Thus, for example, any one of the interacting pairs of proteins described in the tables above can be contacted with a test compound and the integrity of the protein complex can be assessed. Conversely, test compounds may also be screened to identify compounds capable of enhancing the interactions between the constituent members of the protein complexes formed by the interactions described in the tables. The assays can be conducted in a manner similar to the binding assays described above. For example, the presence or absence of a particular pair of interacting proteins can be detected by an antibody selectively immunoreactive with the protein complex formed by those two proteins. Thus, after incubation of the protein complex with a test compound, an immunoprecipitation assay can be conducted with the antibody. If the test compound disrupts the protein complex, then the amount of immunoprecipitated protein complex in this assay will be significantly less than that in a control assay in which the same protein complex is not contacted with the test compound. Similarly, two proteins—the interaction between which is to be enhanced—may be incubated together with a test compound. Thereafter, a protein complex formed by the two interacting proteins may be detected by the selectively immunoreactive antibody. The amount of protein complex may be compared to that formed in the absence of the test compound. Various other detection methods may be suitable in the dissociation assay, as will be apparent to a skilled artisan apprised of the present disclosure.

In another embodiment, fluorescent resonance energy transfer (FRET) is used to screen for modulators of interacting proteins of the protein complexes of the present invention. FRET assays measure the energy transfer of a fluorescent label to another fluorescent label. Fluorescent labels absorb light preferentially at one wavelength and emit light preferentially at a second wavelength. FRET assays utilize this characteristic by selecting a fluorescent label, called a donor fluorophore, that emits light preferentially at the wavelength a second label, called the acceptor fluorophore, preferentially absorbs light. The proximity of the donor and acceptor fluorophore can be determined by measuring the energy transfer from the donor fluorophore to the acceptor fluorophore. Measuring the energy transfer is performed by shining light on a solution containing acceptor and donor fluorophores at the wavelength the donor fluorophore absorbs light and measuring fluorescence at the wavelength the acceptor fluorophore emits light. The amount of fluorescence of the acceptor fluorophore indicates the proximity of the donor and acceptor fluorophores.

For example, FRET assays can be used to screen for modulators of BAT3 by labeling BAT3 or an antibody to BAT3 with an acceptor fluorophore and labeling a BAT3 substrate or interactor (e.g., PN9113) or an antibody to a BAT3 substrate/interactor with an acceptor fluorophore. If the test compound is a BAT3 modulator it will decrease the fluorescence of the acceptor fluorophore because the acceptor and donor fluorphore will not be as close to each other.

In a specific embodiment of a FRET assay, TP³⁺ is attached to an antibody to BAT3, and BODIPY-TMR is attached to an antibody to an interactor (e.g., PN9113). The fluorescently labeled antibodies, BAT3, and BAT3 substrates are put in solution together. Light at the wavelength that TP³⁺ preferentially absorbs light is shined on the solution and the fluorescence of the solution is measured at the wavelength that BODIPY-TMR preferentially emits light. A test compound is then added to the solution and light at the wavelength that TP³⁺ preferentially absorbs light is shined on the solution and the fluorescence of the solution is measured at the wavelength that BODIPY-TMR preferentially emits light. If the fluorescence of the solution with the test compound decreases compared to the fluorescence of the solution without the test compound then the test compound is a BAT3 modulator.

### 5.3. In Vivo Screening Assays

Test compounds can also be screened in any in vivo assays to select modulators of the protein complexes or interacting protein members thereof in accordance with the present invention. For example, any in vivo assays known in the art to be useful in identifying compounds capable of strengthening or interfering with the stability of the protein complexes of the present invention may be used.

In a specific example, a screening assay for modulators of a BAT3 is performed by using PN9113 as a ligand for BAT3. In this screen, BAT3 is contacted with test compounds in the presence of PN9113 and the levels of BAT3-PN9113 protein complex formed when BAT3 is contacted with the test compound in the presence of PN9113 is detected. The ability of the test compounds to modulate BAT3 is determined by comparing the level of BAT3-PN9113 complex formed when BAT3 is contacted with test compounds to the level formed in the absence of test compounds. If the level of BAT3-PN9113 protein complex formed when BAT3 is contacted with the test compound then the test compound is a modulator of BAT3.

To screen peptidic compounds for modulators of BAT3, the two-hybrid systems described in Section 4 may be used in the screening assays in which the BAT3 protein is expressed in, e.g., a bait fusion protein and the peptidic test compounds are expressed in, e.g., prey fusion proteins. Screening peptidic compounds for modulators of the proteins identified in the tables can also be performed using the two-hybrid systems described in Section 4 by expressing the proteins identified in the tables in, e.g., a bait fusion protein and expressing the peptidic test compounds in e.g., prey fusion proteins.

To screen for modulators of the protein-protein interaction between BAT3 and a BAT3-interacting protein, the methods of the present invention typically comprise contacting the BAT3 protein with the BAT3-interacting protein in the presence of a test compound, and determining the interaction between the BAT3 protein and the BAT3-interacting protein. In a preferred embodiment, a two-hybrid system, e.g., a yeast two-hybrid system as described in detail in Section 4 is employed.

### 5.3.1. Two-Hybrid Assays

In a preferred embodiment, one of the yeast two-hybrid systems or their analogous or derivative forms is used. Examples of suitable two-hybrid systems known in the art include, but are not limited to, those disclosed in U.S. Pat. Nos. 5,283,173; 5,525,490; 5,585,245; 5,637,463; 5,695,941; 5,733,726; 5,776,689; 5,885,779; 5,905,025; 6,037,136; 6,057,101; 6,114,111; and Bartel and Fields, eds., The Yeast Two-Hybrid System, Oxford University Press, New York, N.Y., 1997, all of which are incorporated herein by reference.

Typically, in a classic transcription-based two-hybrid assay, two chimeric genes are prepared encoding two fusion proteins: one contains a transcription activation domain fused to an interacting protein member of a protein complex of the present invention or an interaction domain or fragment of the interacting protein member, while the other fusion protein includes a DNA binding domain fused to another interacting protein member of the protein complex or a fragment or interaction domain thereof. For the purpose of convenience, the two interacting protein members, fragments or interaction domains thereof are referred to as “bait fusion protein” and “prey fusion protein,” respectively. The chimeric genes encoding the fusion proteins are termed “bait chimeric gene” and “prey chimeric gene,” respectively. Typically, a “bait vector” and a “prey vector” are provided for the expression of a bait chimeric gene and a prey chimeric gene, respectively.

### 5.3.1.1. Vectors

Many types of vectors can be used in a transcription-based two-hybrid assay. Methods for the construction of bait vectors and prey vectors should be apparent to skilled artisans in the art apprised of the present disclosure. See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Rothstein in DNA Cloning: A Practical Approach, Vol. 11, Ed. DM Glover, IRL Press, Wash., D.C., 1986.

Generally, the bait and prey vectors include an expression cassette having a promoter operably linked to a chimeric gene for the transcription of the chimeric gene. The vectors may also include an origin of DNA replication for the replication of the vectors in host cells and a replication origin for the amplification of the vectors in, e.g., E. coli, and selection marker(s) for selecting and maintaining only those host cells harboring the vectors. Additionally, the expression cassette preferably also contains inducible elements, which function to control the expression of a chimeric gene. Making the expression of the chimeric genes inducible and controllable is especially important in the event that the fusion proteins or components thereof are toxic to the host cells. Other regulatory sequences such as transcriptional enhancer sequences and translation regulation sequences (e.g., Shine-Dalgarno sequence) can also be included in the expression cassette. Termination sequences such as the bovine growth hormone, SV40, lacZ and AcMNPV polyhedral polyadenylation signals may also be operably linked to a chimeric gene in the expression cassette. An epitope tag coding sequence for detection and/or purification of the fusion proteins can also be operably linked to the chimeric gene in the expression cassette. Examples of useful epitope tags include, but are not limited to, influenza virus hemagglutinin (HA), Simian Virus 5 (V5), polyhistidine (6×His), c-myc, lacZ, GST, and the like. Proteins with polyhistidine tags can be easily detected and/or purified with Ni affinity columns, while specific antibodies to many epitope tags are generally commercially available. The vectors can be introduced into the host cells by any techniques known in the art, e.g., by direct DNA transformation, microinjection, electroporation, viral infection, lipofection, gene gun, and the like. The bait and prey vectors can be maintained in host cells in an extrachromosomal state, i.e., as self-replicating plasmids or viruses. Alternatively, one or both vectors can be integrated into chromosomes of the host cells by conventional techniques such as selection of stable cell lines or site-specific recombination.

The in vivo assays of the present invention can be conducted in many different host cells, including but not limited to bacteria, yeast cells, plant cells, insect cells, and mammalian cells. A skilled artisan will recognize that the designs of the vectors can vary with the host cells used. In one embodiment, the assay is conducted in prokaryotic cells such as Escherichia coli, Salmonella, Klebsiella, Pseudomonas, Caulobacter, and Rhizobium. Suitable origins of replication for the expression vectors useful in this embodiment of the present invention include, e.g., the ColE1, pSC101, and M13 origins of replication. Examples of suitable promoters include, for example, the T7 promoter, the lacZ promoter, and the like. In addition, inducible promoters are also useful in modulating the expression of the chimeric genes. For example, the lac operon from bacteriophage lambda plac5 is well known in the art and is inducible by the addition of IPTG to the growth medium. Other known inducible promoters useful in a bacteria expression system include pL of bacteriophage λ, the trp promoter, and hybrid promoters such as the tac promoter, and the like.

In addition, selection marker sequences for selecting and maintaining only those prokaryotic cells expressing the desirable fusion proteins should also be incorporated into the expression vectors. Numerous selection markers including auxotrophic markers and antibiotic resistance markers are known in the art and can all be useful for purposes of this invention. For example, the bla gene, which confers ampicillin resistance, is the most commonly used selection marker in prokaryotic expression vectors. Other suitable markers include genes that confer neomycin, kanamycin, or hygromycin resistance to the host cells. In fact, many vectors are commercially available from vendors such as Invitrogen Corp. of Carlsbad, Calif., Clontech Corp. of Palo Alto, Calif., and Stratagene Corp. of La Jolla, Calif., and Promega Corp. of Madison, Wis. These commercially available vectors, e.g., pBR322, pSPORT, pBluescriptIISK, pcDNAI, and pcDNAII all have a multiple cloning site into which the chimeric genes of the present invention can be conveniently inserted using conventional recombinant techniques. The constructed expression vectors can be introduced into host cells by various transformation or transfection techniques generally known in the art.

In another embodiment, mammalian cells are used as host cells for the expression of the fusion proteins and detection of protein-protein interactions. For this purpose, virtually any mammalian cells can be used including normal tissue cells, stable cell lines, and transformed tumor cells. Conveniently, mammalian cell lines such as CHO cells, Jurkat T cells, NIH 3T3 cells, HEK-293 cells, CV-1 cells, COS-1 cells, HeLa cells, VERO cells, MDCK cells, WI38 cells, and the like are used. Mammalian expression vectors are well known in the art and many are commercially available. Examples of suitable promoters for the transcription of the chimeric genes in mammalian cells include viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Inducible promoters can also be used. Suitable inducible promoters include, for example, the tetracycline responsive element (TRE) (See Gossen et al., Proc. Natl. Acad. Sci. USA, 89:5547-5551 (1992)), metallothionein IIA promoter, ecdysone-responsive promoter, and heat shock promoters. Suitable origins of replication for the replication and maintenance of the expression vectors in mammalian cells include, e.g., the Epstein Barr origin of replication in the presence of the Epstein Barr nuclear antigen (see Sugden et al., Mole. Cell. Biol., 5:410-413 (1985)) and the SV40 origin of replication in the presence of the SV40 T antigen (which is present in COS-1 and COS-7 cells) (see Margolskee et al., Mole. Cell. Biol., 8:2837 (1988)). Suitable selection markers include, but are not limited to, genes conferring resistance to neomycin, hygromycin, zeocin, and the like. Many commercially available mammalian expression vectors may be useful for the present invention, including, e.g., pCEP4, pcDNAI, pIND, pSecTag2, pVAX1, pcDNA3.1, and pBI-EGFP, and pDisplay. The vectors can be introduced into mammalian cells using any known techniques such as calcium phosphate precipitation, lipofection, electroporation, and the like. The bait vector and prey vector can be co-transformed into the same cell or, alternatively, introduced into two different cells which are subsequently fused together by cell fusion or other suitable techniques.

Viral expression vectors, which permit introduction of recombinant genes into cells by viral infection, can also be used for the expression of the fusion proteins. Viral expression vectors generally known in the art include viral vectors based on adenovirus, bovine papilloma virus, murine stem cell virus (MSCV), MFG virus, and retrovirus. See Sarver, et al., Mol. Cell. Biol., 1: 486 (1981); Logan & Shenk, Proc. Natl. Acad. Sci. USA, 81:3655-3659 (1984); Mackett, et al, Proc. Natl. Acad. Sci. USA, 79:7415-7419 (1982); Mackett, et al., J. Virol., 49:857-864 (1984); Panicali, et al., Proc. Natl. Acad. Sci. USA, 79:4927-4931 (1982); Cone & Mulligan, Proc. Natl. Acad. Sci. USA, 81:6349-6353 (1984); Mann et al, Cell, 33:153-159 (1993); Pear et al., Proc. Natl. Acad. Sci. USA, 90:8392-8396 (1993); Kitamura et al., Proc. Natl. Acad. Sci. USA, 92:9146-9150 (1995); Kinsella et al., Human Gene Therapy, 7:1405-1413 (1996); Hofmann et al., Proc. Natl. Acad. Sci. USA, 93:5185-5190 (1996); Choate et al., Human Gene Therapy, 7:2247 (1996); WO 94/19478; Hawley et al, Gene Therapy, 1:136 (1994) and Rivere et al., Genetics, 92:6733 (1995), all of which are incorporated by reference.

Generally, to construct a viral vector, a chimeric gene according to the present invention can be operably linked to a suitable promoter. The promoter-chimeric gene construct is then inserted into a non-essential region of the viral vector, typically a modified viral genome. This results in a viable recombinant virus capable of expressing the fusion protein encoded by the chimeric gene in infected host cells. Once in the host cell, the recombinant virus typically is integrated into the genome of the host cell. However, recombinant bovine papilloma viruses typically replicate and remain as extrachromosomal elements.

In another embodiment, the detection assays of the present invention are conducted in plant cell systems. Methods for expressing exogenous proteins in plant cells are well known in the art. See generally, Weissbach & Weissbach, Methods for Plant Molecular Biology, Academic Press, NY, 1988; Grierson & Corey, Plant Molecular Biology, 2d Ed., Blackie, London, 1988. Recombinant virus expression vectors based on, e.g., cauliflower mosaic virus (CaMV) or tobacco mosaic virus (TMV) can all be used. Alternatively, recombinant plasmid expression vectors such as Ti plasmid vectors and Ri plasmid vectors are also useful. The chimeric genes encoding the fusion proteins of the present invention can be conveniently cloned into the expression vectors and placed under control of a viral promoter such as the 35S RNA and 19S RNA promoters of CaMV or the coat protein promoter of TMV, or of a plant promoter, e.g., the promoter of the small subunit of RUBISCO and heat shock promoters (e.g., soybean hsp17.5-E or hsp17.3-B promoters).

In addition, the in vivo assay of the present invention can also be conducted in insect cells, e.g., Spodoptera frugiperda cells, using a baculovirus expression system. Expression vectors and host cells useful in this system are well known in the art and are generally available from various commercial vendors. For example, the chimeric genes of the present invention can be conveniently cloned into a non-essential region (e.g., the polyhedrin gene) of an Autographa californica nuclear polyhedrosis virus (AcNPV) vector and placed under control of an AcNPV promoter (e.g., the polyhedrin promoter). The non-occluded recombinant viruses thus generated can be used to infect host cells such as Spodoptera frugiperda cells in which the chimeric genes are expressed. See U.S. Pat. No. 4,215,051.

In a preferred embodiment of the present invention, the fusion proteins are expressed in a yeast expression system using yeasts such as Saccharomyces cerevisiae, Hansenula polymorpha, Pichia pastoris, and Schizosaccharomyces pombe as host cells. The expression of recombinant proteins in yeasts is a well-developed field, and the techniques useful in this respect are disclosed in detail in The Molecular Biology of the Yeast Saccharomyces, Eds. Strathern et al., Vols. I and II, Cold Spring Harbor Press, 1982; Ausubel et al., Current Protocols in Molecular Biology, New York, Wiley, 1994; and Guthrie and Fink, Guide to Yeast Genetics and Molecular Biology, in Methods in Enzymology, Vol. 194, 1991, all of which are incorporated herein by reference. Sudbery, Curr. Opin. Biotech., 7:517-524 (1996) reviews the successes in the art of expressing recombinant proteins in various yeast species; the entire content and references cited therein are incorporated herein by reference. In addition, Bartel and Fields, eds., The Yeast Two-Hybrid System, Oxford University Press, New York, N.Y., 1997 contains extensive discussions of recombinant expression of fusion proteins in yeasts in the context of various yeast two-hybrid systems, and cites numerous relevant references. These and other methods known in the art can all be used for purposes of the present invention. The application of such methods to the present invention should be apparent to a skilled artisan apprised of the present disclosure.

Generally, each of the two chimeric genes is included in a separate expression vector (bait vector and prey vector). Both vectors can be co-transformed into a single yeast host cell. As will be apparent to a skilled artisan, it is also possible to express both chimeric genes from a single vector. In a preferred embodiment, the bait vector and prey vector are introduced into two haploid yeast cells of opposite mating types, e.g., a-type and α-type, respectively. The two haploid cells can be mated at a desired time to form a diploid cell expressing both chimeric genes.

Generally, the bait and prey vectors for recombinant expression in yeast include a yeast replication origin such as the 2p origin or the ARSH4 sequence for the replication and maintenance of the vectors in yeast cells. Preferably, the vectors also have a bacteria origin of replication (e.g., ColE1) and a bacteria selection marker (e.g., amp^{R} marker, i.e., bla gene). Optionally, the CEN6 centromeric sequence is included to control the replication of the vectors in yeast cells. Any constitutive or inducible promoters capable of driving gene transcription in yeast cells may be employed to control the expression of the chimeric genes. Such promoters are operably linked to the chimeric genes. Examples of suitable constitutive promoters include but are not limited to the yeast ADH1, PGK1, TEF2, GPD1, HIS3, and CYC1 promoters. Examples of suitable inducible promoters include but are not limited to the yeast GAL1 (inducible by galactose), CUP1 (inducible by Cu⁺⁺), and FUS1 (inducible by pheromone) promoters; the AOX/MOX promoter from H. polymorpha and P. pastoris (repressed by glucose or ethanol and induced by methanol); chimeric promoters such as those that contain LexA operators (inducible by LexA-containing transcription factors); and the like. Inducible promoters are preferred when the fusion proteins encoded by the chimeric genes are toxic to the host cells. If it is desirable, certain transcription repressing sequences such as the upstream repressing sequence (URS) from SPO13 promoter can be operably linked to the promoter sequence, e.g., to the 5′ end of the promoter region. Such upstream repressing sequences function to fine-tune the expression level of the chimeric genes.

Preferably, a transcriptional termination signal is operably linked to the chimeric genes in the vectors. Generally, transcriptional termination signal sequences derived from, e.g., the CYC1 and ADH1 genes can be used.

Additionally, it is preferred that the bait vector and prey vector contain one or more selectable markers for the selection and maintenance of only those yeast cells that harbor one or both chimeric genes. Any selectable markers known in the art can be used for purposes of this invention so long as yeast cells expressing the chimeric gene(s) can be positively identified or negatively selected. Examples of markers that can be positively identified are those based on color assays, including the lacZ gene (which encodes β-galactosidase), the firefly luciferase gene, secreted alkaline phosphatase, horseradish peroxidase, the blue fluorescent protein (BFP), and the green fluorescent protein (GFP) gene (see Cubitt et al., Trends Biochem. Sci., 20:448-455 (1995)). Other markers allowing detection by fluorescence, chemiluminescence, UV absorption, infrared radiation, and the like can also be used. Among the markers that can be selected are auxotrophic markers including, but not limited to, URA3, HIS3, TRP1, LEU2, LYS2, ADE2, and the like. Typically, for purposes of auxotrophic selection, the yeast host cells transformed with bait vector and/or prey vector are cultured in a medium lacking a particular nutrient. Other selectable markers are not based on auxotrophies, but rather on resistance or sensitivity to an antibiotic or other xenobiotic. Examples of such markers include but are not limited to chloramphenicol acetyl transferase (CAT) gene, which confers resistance to chloramphenicol; CAN1 gene, which encodes an arginine permease and thereby renders cells sensitive to canavanine (see Sikorski et al., Meth. Enzymol., 194:302-318 (1991)); the bacterial kanamycin resistance gene (kan^{R}), which renders eukaryotic cells resistant to the aminoglycoside G418 (see Wach et al., Yeast, 10: 1793-1808 (1994)); and CYH2 gene, which confers sensitivity to cycloheximide (see Sikorski et al., Meth. Enzymol., 194:302-318 (1991)). In addition, the CUP1 gene, which encodes metallothionein and thereby confers resistance to copper, is also a suitable selection marker. Each of the above selection markers may be used alone or in combination. One or more selection markers can be included in a particular bait or prey vector. The bait vector and prey vector may have the same or different selection markers. In addition, the selection pressure can be placed on the transformed host cells either before or after mating the haploid yeast cells.

As will be apparent, the selection markers used should complement the host strains in which the bait and/or prey vectors are expressed. In other words, when a gene is used as a selection marker gene, a yeast strain lacking the selection marker gene (or having mutation in the corresponding gene) should be used as host cells. Numerous yeast strains or derivative strains corresponding to various selection markers are known in the art. Many of them have been developed specifically for certain yeast two-hybrid systems. The application and optional modification of such strains with respect to the present invention will be apparent to a skilled artisan apprised of the present disclosure. Methods for genetically manipulating yeast strains using genetic crossing or recombinant mutagenesis are well known in the art. See e.g., Rothstein, Meth. Enzymol., 101:202-211 (1983). By way of example, the following yeast strains are well known in the art, and can be used in the present invention upon necessary modifications and adjustment:

L40 strain which has the genotype MATa his3Δ200 trp1-901 leu2-3,112 ade2 LYS2::(lexAop)4-HIS3 URA3::(lexAop)8-lacZ;

EGY48 strain which has the genotype MATa trp1 his3 ura3 6ops-LEU2; and

MaV103 strain which has the genotype MATa ura3-52 leu2-3,112 trp1-901 his3Δ200 ade2-101 gal4Δ gal80Δ SPAL10::URA3 GAL1::HIS3::lys2 (see Kumar et al., J. Biol. Chem. 272:13548-13554 (1997); Vidal et al., Proc. Natl. Acad. Sci. USA, 93:10315-10320 (1996)). Such strains are generally available in the research community, and can also be obtained by simple yeast genetic manipulation. See, e.g., The Yeast Two-Hybrid System, Bartel and Fields, eds., pages 173-182, Oxford University Press, New York, N.Y., 1997.

In addition, the following yeast strains are commercially available:

Y190 strain which is available from Clontech, Palo Alto, Calif. and has the genotype MATa gal4 gal80 his3Δ200 trp1-901 ade2-101 ura3-52 leu2-3, 112 URA3::GAL1-lacZ LYS2::GAL1-HIS3 cyh^{r}; and

YRG-2 Strain which is available from Stratagene, La Jolla, Calif. and has the genotype MATα ura3-52 his3-200 ade2-101 lys2-801 trp1-901 leu2-3, 112 gal4-542 gal80-538 LYS2::GAL1-HIS3 URA3::GAL1/CYC1-lacZ.

In fact, different versions of vectors and host strains specially designed for yeast two-hybrid system analysis are available in kits from commercial vendors such as Clontech, Palo Alto, Calif. and Stratagene, La Jolla, Calif., all of which can be modified for use in the present invention.

### 5.3.1.2. Reporters

Generally, in a transcription-based two-hybrid assay, the interaction between a bait fusion protein and a prey fusion protein brings the DNA-binding domain and the transcription-activation domain into proximity forming a functional transcriptional factor that acts on a specific promoter to drive the expression of a reporter protein. The transcription activation domain and the DNA-binding domain may be selected from various known transcriptional activators, e.g., GAL4, GCN4, ARD1, the human estrogen receptor, E. coli LexA protein, herpes simplex virus VP16 (Triezenberg et al., Genes Dev. 2:718-729 (1988)), the E. coli B42 protein (acid blob, see Gyuris et al., Cell, 75:791-803 (1993)), NF-kB p65, and the like. The reporter gene and the promoter driving its transcription typically are incorporated into a separate reporter vector. Alternatively, the host cells are engineered to contain such a promoter-reporter gene sequence in their chromosomes. Thus, the interaction or lack of interaction between two interacting protein members of a protein complex can be determined by detecting or measuring changes in the assay system's reporter. Although the reporters and selection markers can be of similar types and used in a similar manner in the present invention, the reporters and selection markers should be carefully selected in a particular detection assay such that they are distinguishable from each other and do not interfere with each other's function.

Many different types of reporters are useful in the screening assays. For example, a reporter protein may be a fusion protein having an epitope tag fused to a protein. Commonly used and commercially available epitope tags include sequences derived from, e.g., influenza virus hemagglutinin (HA), Simian Virus 5 (V5), polyhistidine (6×His), c-myc, lacZ, GST, and the like. Antibodies specific to these epitope tags are generally commercially available. Thus, the expressed reporter can be detected using an epitope-specific antibody in an immunoassay.

In another embodiment, the reporter is selected such that it can be detected by a color-based assay. Examples of such reporters include, e.g., the lacZ protein (β-galactosidase), the green fluorescent protein (GFP), which can be detected by fluorescence assay and sorted by flow-activated cell sorting (FACS) (See Cubitt et al., Trends Biochem. Sci., 20:448-455 (1995)), secreted alkaline phosphatase, horseradish peroxidase, the blue fluorescent protein (BFP), and luciferase photoproteins such as aequorin, obelin, mnemiopsin, and berovin (See U.S. Pat. No. 6,087,476, which is incorporated herein by reference).

Alternatively, an auxotrophic factor is used as a reporter in a host strain deficient in the auxotrophic factor. Thus, suitable auxotrophic reporter genes include, but are not limited to, URA3, HIS3, TRP1, LEU2, LYS2, ADE2, and the like. For example, yeast cells containing a mutant URA3 gene can be used as host cells (Ura⁻ phenotype). Such cells lack URA3-encoded functional orotidine-5′-phosphate decarboxylase, an enzyme required by yeast cells for the biosynthesis of uracil. As a result, the cells are unable to grow on a medium lacking uracil. However, wild-type orotidine-5′-phosphate decarboxylase catalyzes the conversion of a non-toxic compound 5-fluoroorotic acid (5-FOA) to a toxic product, 5-fluorouracil. Thus, yeast cells containing a wild-type URA3 gene are sensitive to 5-FOA and cannot grow on a medium containing 5-FOA. Therefore, when the interaction between the interacting protein members in the fusion proteins results in the expression of active orotidine-5′-phosphate decarboxylase, the Ura⁻ (Foa^{R}) yeast cells will be able to grow on a uracil deficient medium (SC-Ura plates). However, such cells will not survive on a medium containing 5-FOA. Thus, protein-protein interactions can be detected based on cell growth.

Additionally, antibiotic resistance reporters can also be employed in a similar manner. In this respect, host cells sensitive to a particular antibiotic are used. Antibiotic resistance reporters include, for example, the chloramphenicol acetyl transferase (CAT) gene and the kan^{R} gene, which confer resistance to G418 in eukaryotes, and kanamycin in prokaryotes, respectively.

### 5.3.1.3. Screening Assays for Interaction Antagonists

The screening assays of the present invention are useful for identifying compounds capable of interfering with, disrupting, or dissociating the protein-protein interactions formed between members of the interacting protein pairs disclosed in the tables above, or between mutant and wild type, or mutant and mutant forms of these proteins. Since the protein complexes of the present invention are associated with Alzheimer's disease (including mild cognitive impairment), Huntington's disease, Parkinson's disease, schizophrenia, depression, dementia and other neurodegenerative diseases, as well as diabetes, obesity and coronary artery disease (either directly through their known cellular roles or functions or through the association of mutant forms of these proteins with the disease, or indirectly—through their interactions with other proteins known to be linked to Alzheimer's disease (including mild cognitive impairment), Huntington's disease, Parkinson's disease, schizophrenia, depression, dementia and other neurodegenerative diseases, as well as diabetes, obesity and coronary artery disease), disruption or dissociation of particular protein-protein interactions may be desirable to ameliorate the disease condition, or to alleviate disease symptoms. Alternatively, if the disease or disorder is associated with increased expression of any of the proteins presented in the tables, or with expression of a mutant form, or forms, of these proteins, then the disease or disorder may be ameliorated, or symptoms reduced, by weakening or dissociating the interaction between the interacting proteins in patients. Also, if a disease or disorder is associated with a mutant form of an interacting protein that form stronger protein-protein interactions with its protein partner than its wild type counterpart, then the disease or disorder may be treated with a compound that weakens, disrupts or interferes with the interaction between the mutant protein and its interacting partner.

In a screening assay for an interaction antagonist, a first protein, which is a protein selected from any of the protein pairs described in the tables (or a homologue, fragment or derivative thereof), or a mutant form of the first protein (or a homologue, fragment or derivative thereof), and a second protein, which is the interacting partner of the first protein identified in the tables above (or a homologue, fragment or derivative thereof), or a mutant form of the second protein (or a homologue, fragment or derivative thereof), are used as test proteins expressed in the form of fusion proteins as described above for purposes of a two-hybrid assay. The fusion proteins are expressed in a host cell and allowed to interact with each other in the presence of one or more test compounds.

In a preferred embodiment, a counterselectable marker is used as a reporter such that a detectable signal (e.g., appearance of color or fluorescence, or cell survival) is present only when the test compound is capable of interfering with the interaction between the two test proteins. In this respect, the reporters used in various “reverse two-hybrid systems” known in the art may be employed. Reverse two-hybrid systems are disclosed in, e.g., U.S. Pat. Nos. 5,525,490; 5,733,726; 5,885,779; Vidal et al., Proc. Natl. Acad. Sci. USA, 93:10315-10320 (1996); and Vidal et al., Proc. Natl. Acad. Sci. USA, 93:10321-10326 (1996), all of which are incorporated herein by reference.

Examples of suitable counterselectable reporters useful in a yeast system include the URA3 gene (encoding orotidine-5′-decarboxylase, which converts 5-fluoroorotic acid (5-FOA) to the toxic metabolite 5-fluorouracil), the CAN1 gene (encoding arginine permease, which transports the toxic arginine analog canavanine into yeast cells), the GAL1 gene (encoding galactokinase, which catalyzes the conversion of 2-deoxygalactose to toxic 2-deoxygalactose-1-phosphate), the LYS2 gene (encoding α-aminoadipate reductase, which renders yeast cells unable to grow on a medium containing α-aminoadipate as the sole nitrogen source), the MET15 gene (encoding O-acetylhomoserine sulfhydrylase, which confers on yeast cells sensitivity to methyl mercury), and the CYH2 gene (encoding L29 ribosomal protein, which confers sensitivity to cycloheximide). In addition, any known cytotoxic agents including cytotoxic proteins such as the diphtheria toxin (DTA) catalytic domain can also be used as counterselectable reporters. See U.S. Pat. No. 5,733,726. DTA causes the ADP-ribosylation of elongation factor-2 and thus inhibits protein synthesis and causes cell death. Other examples of cytotoxic agents include ricin, Shiga toxin, and exotoxin A of Pseudomonas aeruginosa.

For example, when the URA3 gene is used as a counterselectable reporter gene, yeast cells containing a mutant URA3 gene can be used as host cells (Ura⁻ Foa^{R} phenotype) for the in vivo assay. Such cells lack URA3-encoded functional orotidine-5′-phosphate decarboxylase, an enzyme required for the biosynthesis of uracil. As a result, the cells are unable to grow on media lacking uracil. However, because of the absence of a wild-type orotidine-5′-phosphate decarboxylase, the yeast cells cannot convert non-toxic 5-fluoroorotic acid (5-FOA) to a toxic product, 5-fluorouracil. Thus, such yeast cells are resistant to 5-FOA and can grow on a medium containing 5-FOA. Therefore, for example, to screen for a compound capable of disrupting interactions between BAT3 (or a homologue, fragment or derivative thereof), or a mutant form of BAT3 (or a homologue, fragment or derivative thereof), and PN9113 (or a homologue, fragment or derivative thereof), or a mutant form of PN9113 (or a homologue, fragment or derivative thereof), BAT3 (or a homologue, fragment or derivative thereof) is expressed as a fusion protein with a DNA-binding domain of a suitable transcription activator while PN9113 (or a homologue, fragment or derivative thereof) is expressed as a fusion protein with a transcription activation domain of a suitable transcription activator. In the host strain, the reporter URA3 gene may be operably linked to a promoter specifically responsive to the association of the transcription activation domain and the DNA-binding domain. After the fusion proteins are expressed in the Ura⁻ Foa^{R} yeast cells, an in vivo screening assay can be conducted in the presence of a test compound with the yeast cells being cultured on a medium containing uracil and 5-FOA. If the test compound does not disrupt the interaction between BAT3 and PN9113, active URA3 gene product, i.e., orotidine-5′-decarboxylase, which converts 5-FOA to toxic 5-fluorouracil, is expressed. As a result, the yeast cells cannot grow. On the other hand, when the test compound disrupts the interaction between BAT3 and PN9113, no active orotidine-5′-decarboxylase is produced in the host yeast cells. Consequently, the yeast cells will survive and grow on the 5-FOA-containing medium. Therefore, compounds capable of interfering with or dissociating the interaction between BAT3 and PN9113 can thus be identified based on colony formation.

As will be apparent, the screening assay of the present invention can be applied in a format appropriate for large-scale screening. For example, combinatorial technologies can be employed to construct combinatorial libraries of small organic molecules or small peptides. See generally, e.g., Kenan et al., Trends Biochem. Sc., 19:57-64 (1994); Gallop et al, J. Med. Chem., 37:1233-1251 (1994); Gordon et al, J. Med. Chem., 37:1385-1401 (1994); Ecker et al., Biotechnology, 13:351-360 (1995). Such combinatorial libraries of compounds can be applied to the screening assay of the present invention to isolate specific modulators of particular protein-protein interactions. In the case of random peptide libraries, the random peptides can be co-expressed with the fusion proteins of the present invention in host cells and assayed in vivo. See e.g., Yang et al., Nucl. Acids Res., 23:1152-1156 (1995). Alternatively, they can be added to the culture medium for uptake by the host cells.

Conveniently, yeast mating is used in an in vivo screening assay. For example, haploid cells of a-mating type expressing one fusion protein as described above are mated with haploid cells of α-mating type expressing the other fusion protein. Upon mating, the diploid cells are spread on a suitable medium to form a lawn. Drops of test compounds can be deposited onto different areas of the lawn. After culturing the lawn for an appropriate period of time, drops containing a compound capable of modulating the interaction between the particular test proteins in the fusion proteins can be identified by stimulation or inhibition of growth in the vicinity of the drops.

The screening assays of the present invention for identifying compounds capable of modulating protein-protein interactions can also be fine-tuned by various techniques to adjust the thresholds or sensitivity of the positive and negative selections. Mutations can be introduced into the reporter proteins to adjust their activities. The uptake of test compounds by the host cells can also be adjusted. For example, yeast high uptake mutants such as the erg6 mutant strains can facilitate yeast uptake of the test compounds. See Gaber et al., Mol. Cell. Biol., 9:3447-3456 (1989). Likewise, the uptake of the selection compounds such as 5-FOA, 2-deoxygalactose, cycloheximide, α-aminoadipate, and the like can also be fine-tuned.

Generally, a control assay is performed in which the above screening assay is conducted in the absence of the test compound. The result of this assay is then compared with that obtained in the presence of the test compound.

### 5.3.1.4. Screening Assays for Interaction Agonists

The screening assays of the present invention can also be used to identify compounds that trigger or initiate, enhance or stabilize the protein-protein interactions formed between members of the interacting protein pairs disclosed in the tables above, or between combinations of mutant and wild type forms of such proteins, or pairs of mutant proteins. For example, if a disease or disorder is associated with the decreased expression of any one of the individual proteins, or one of the protein pairs selected from the tables, then the disease or disorder may be treated by strengthening or stabilizing the interactions between the interacting partner proteins in patients. Alternatively, if a disease or disorder is associated with a mutant form, or forms, of the interacting proteins that exhibit weakened or abolished interactions with their binding partner(s), then the disease or disorder may be treated with a compound that initiates or stabilizes the interaction between the mutant form, or forms, of the interacting proteins.

Thus, a screening assay can be performed in the same manner as described above, except that a positively selectable marker is used. For example, a first protein, which is any protein selected from the proteins described in the tables (or a homologue, fragment, or derivative thereof), or a mutant form of the first protein (or a homologue, fragment, or derivative thereof), and a second protein, which is an interacting partner of the first protein (or a homologue, fragment, or derivative thereof), or a mutant form of the second protein (or a homologue, fragment, or derivative thereof), are used as test proteins expressed in the form of fusion proteins as described above for purposes of a two-hybrid assay. The fusion proteins are expressed in host cells and are allowed to interact with each other in the presence of one or more test compounds.

A gene encoding a positively selectable marker such as β-galatosidase may be used as a reporter gene such that when a test compound enables, enhances or strengthens the interaction between a first protein, (or a homologue, fragment, or derivative thereof), or a mutant form of the first protein (or a homologue, fragment, or derivative thereof), and a second protein (or a homologue, fragment, or derivative thereof), or a mutant form of the second (or a homologue, fragment, or derivative thereof), β-galatosidase is expressed. As a result, the compound may be identified based on the appearance of a blue color when the host cells are cultured in a medium containing X-Gal.

Generally, a control assay is performed in which the above screening assay is conducted in the absence of the test compound. The result of this assay is then compared with that obtained in the presence of the test compound.

### 5.4. Optimization of the Identified Compounds

Once test compounds are selected that are capable of modulating the interaction between the interacting protein pairs of proteins described in the tables, or modulating the activity or intracellular levels of their constituent proteins, a secondary assay can be performed to confirm the specificity and effect of the compounds selected in the primary screens. Exemplary secondary assays are cell-based assays or animal based assays.

In addition, once test compounds are selected that are capable of modulating the proteins in the tables or the interaction between the interacting protein pairs of proteins described in the tables, or modulating the activity or intracellular levels of their constituent proteins, a data set including data defining the identity or characteristics of the test compounds can be generated. The data set may include information relating to the properties of a selected test compound, e.g., chemical structure, chirality, molecular weight, melting point, etc. Alternatively, the data set may simply include assigned identification numbers understood by the researchers conducting the screening assay and/or researchers receiving the data set as representing specific test compounds. The data or information can be cast in a transmittable form that can be communicated or transmitted to other researchers, particularly researchers in a different country. Such a transmittable form can vary and can be tangible or intangible. For example, the data set defining one or more selected test compounds can be embodied in texts, tables, diagrams, molecular structures, photographs, charts, images or any other visual forms. The data or information can be recorded on a tangible media such as paper or embodied in computer-readable forms (e.g., electronic, electromagnetic, optical or other signals). The data in a computer-readable form can be stored in a computer usable storage medium (e.g., floppy disks, magnetic tapes, optical disks, and the like) or transmitted directly through a communication infrastructure. In particular, the data embodied in electronic signals can be transmitted in the form of email or posted on a website on the Internet or Intranet. In addition, the information or data on a selected test compound can also be recorded in an audio form and transmitted through any suitable media, e.g., analog or digital cable lines, fiber optic cables, etc., via telephone, facsimile, wireless mobile phone, Internet phone and the like.

Thus, the information and data on a test compound selected in a screening assay described above or by virtual screening as discussed below can be produced anywhere in the world and transmitted to a different location. For example, when a screening assay is conducted offshore, the information and data on a selected test compound can be generated and cast in a transmittable form as described above. The data and information in a transmittable form thus can be imported into the U.S. or transmitted to any other countries, where the data and information may be used in further testing the selected test compound and/or in modifying and optimizing the selected test compound to develop lead compounds for testing in clinical trials.

Compounds can also be selected based on structural models of the target protein or protein complex and/or test compounds. In addition, once an effective compound is identified, structural analogs or mimetics thereof can be produced based on rational drug design with the aim of improving drug efficacy and stability, and reducing side effects. Methods known in the art for rational drug design can be used in the present invention. See, e.g., Hodgson et al., Bio/Technology, 9:19-21 (1991); U.S. Pat. Nos. 5,800,998 and 5,891,628, all of which are incorporated herein by reference. An example of rational drug design is the development of HIV protease inhibitors. See Erickson et al., Science, 249:527-533 (1990).

In this respect, structural information on the target protein or protein complex is obtained. Preferably, atomic coordinates defining a three-dimensional structure of the target protein or protein complex can be obtained. For example, each of the interacting pairs can be expressed and purified. The purified interacting protein pairs are then allowed to interact with each other in vitro under appropriate conditions. Optionally, the interacting protein complex can be stabilized by crosslinking or other techniques. The interacting complex can be studied using various biophysical techniques including, e.g., X-ray crystallography, NMR, computer modeling, mass spectrometry, and the like. Likewise, structural information can also be obtained from protein complexes formed by interacting proteins and a compound that initiates or stabilizes the interaction of the proteins. Methods for obtaining such atomic coordinates by X-ray crystallography, NMR, and the like are known in the art and the application thereof to the target protein or protein complex of the present invention should be apparent to skilled persons in the art of structural biology. See Smyth and Martin, Mol. Pathol., 53:8-14 (2000); Oakley and Wilce, Clin. Exp. Pharmacol. Physiol., 27(3):145-151 (2000); Ferentz and Wagner, Q. Rev. Biophys., 33:29-65 (2000); Hicks, Curr. Med. Chem., 8(6):627-650 (2001); and Roberts, Curr. Opin. Biotechnol., 10:42-47 (1999).

In addition, understanding of the interaction between the proteins of interest in the presence or absence of a modulator can also be derived by mutagenic analysis using a yeast two-hybrid system or other methods for detecting protein-protein interactions. In this respect, various mutations can be introduced into the interacting proteins and the effect of the mutations on protein-protein interaction examined by a suitable method such as the yeast two-hybrid system.

Various mutations including amino acid substitutions, deletions and insertions can be introduced into a protein sequence using conventional recombinant DNA technologies. Generally, it is particularly desirable to decipher the protein binding sites. Thus, it is important that the mutations introduced only affect protein-protein interactions and cause minimal structural disturbances. Mutations are preferably designed based on knowledge of the three-dimensional structure of the interacting proteins. Preferably, mutations are introduced to alter charged amino acids or hydrophobic amino acids exposed on the surface of the proteins, since ionic interactions and hydrophobic interactions are often involved in protein-protein interactions. Alternatively, the “alanine scanning mutagenesis” technique is used. See Wells, et al., Methods Enzymol., 202:301-306 (1991); Bass et al., Proc. Natl. Acad. Sci. USA, 88:4498-4502 (1991); Bennet et al., J. Biol. Chem., 266:5191-5201 (1991); Diamond et al., J. Virol., 68:863-876 (1994). Using this technique, charged or hydrophobic amino acid residues of the interacting proteins are replaced by alanine, and the effect on the interaction between the proteins is analyzed using e.g., the yeast two-hybrid system. For example, the entire protein sequence can be scanned in a window of five amino acids. When two or more charged or hydrophobic amino acids appear in a window, the charged or hydrophobic amino acids are changed to alanine using standard recombinant DNA techniques. The thus-mutated proteins are used as “test proteins” in the above-described two-hybrid assays to examine the effect of the mutations on protein-protein interaction. Preferably, the mutational analyses are conducted both in the presence and in the absence of an identified modulator compound. In this manner, the domains or residues of the proteins important to protein-protein interaction and/or the interaction between the modulator compound and the interacting proteins can be identified.

Based on the information obtained, structural relationships between the interacting proteins, as well as between the identified modulators and the interacting proteins are elucidated. For example, for the identified modulators (i.e., lead compounds), the three-dimensional structure and chemical moieties critical to their modulating effect on the interacting proteins are revealed. Using this information and various techniques known in the art of molecular modeling (i.e., simulated annealing), medicinal chemists can then design analog compounds that might be more effective modulators of the protein-protein interactions of the present invention. For example, the analog compounds might show more specific or tighter binding to their targets, and thereby might exhibit fewer side effects, or might have more desirable pharmacological characteristics (e.g., greater solubility).

In addition, if the lead compound is a peptide, it can also be analyzed by the alanine scanning technique and/or the two-hybrid assay to determine the domains or residues of the peptide important to its modulating effect on particular protein-protein interactions. The peptide compound can be used as a lead molecule for rational design of small organic molecules or peptide mimetics. See Huber et al., Curr. Med. Chem., 1:13-34 (1994).

The domains, residues or moieties critical to the modulating effect of the identified compound constitute the active region of the compound known as its “pharmacophore.” Once the pharmacophore has been elucidated, a structural model can be established by a modeling process that may incorporate data from NMR analysis, X-ray diffraction data, alanine scanning, spectroscopic techniques and the like. Various techniques including computational analysis (e.g., molecular modeling and simulated annealing), similarity mapping and the like can all be used in this modeling process. See e.g., Perry et al., in OSAR: Quantitative Structure-Activity Relationships in Drug Design, pp. 189-193, Alan R. Liss, Inc., 1989; Rotivinen et al., Acta Pharmaceutical Fennica, 97:159-166 (1988); Lewis et al., Proc. R. Soc. Lond., 236:125-140 (1989); McKinaly et al., Annu. Rev. Pharmacol. Toxiciol., 29:111-122 (1989). Commercial molecular modeling systems available from Polygen Corporation, Waltham, Mass., include the CHARMm program, which performs energy minimization and molecular dynamics functions, and QUANTA program, which performs construction, graphic modeling and analysis of molecular structure. Such programs allow interactive construction, modification, and visualization of molecules. Other computer modeling programs are also available from BioDesign, Inc. (Pasadena, Calif.), Hypercube, Inc. (Cambridge, Ontario), and Allelix, Inc. (Mississauga, Ontario, Canada).

A template can be formed based on the established model. Various compounds can then be designed by linking various chemical groups or moieties to the template. Various moieties of the template can also be replaced. In addition, in the case of a peptide lead compound, the peptide or mimetics thereof can be cyclized, e.g., by linking the N-terminus and C-terminus together, to increase its stability. These rationally designed compounds are further tested. In this manner, pharmacologically acceptable and stable compounds with improved efficacy and reduced side effects can be developed. The compounds identified in accordance with the present invention can be incorporated into a pharmaceutical formulation suitable for administration to an individual.

In addition, the structural models or atomic coordinates defining a three-dimensional structure of the target protein or protein complex can also be used in virtual screen to select compounds capable of modulating the target protein or protein complex. Various methods of computer-based virtual screen using atomic coordinates are generally known in the art. For example, U.S. Pat. No. 5,798,247 (which is incorporated herein by reference) discloses a method of identifying a compound (specifically, an interleukin converting enzyme inhibitor) by determining binding interactions between an organic compound and binding sites of a binding cavity within the target protein. The binding sites are defined by atomic coordinates.

The compounds designed or selected based on rational drug design or virtual screen can be tested for their ability to modulate (interfere with or strengthen) the interaction between the interacting partners within the protein complexes of the present invention. In addition, the compounds can also be further tested for their ability to modulate (inhibit or enhance) cellular functions such as neurotransmission, regulation of APP production and APP metabolism, regulation of Aβ production and Aβ metabolism, processing of proteins destined for secretion, regulation of the wingless pathway, and modulation of thyroid receptor signaling in cells as well as their effectiveness in treating diseases such as Alzheimer's disease (including mild cognitive impairment), Huntington's disease, Parkinson's disease, schizophrenia, depression, dementia and other neurodegenerative diseases, as well as diabetes, obesity and coronary artery disease.

Following the selection of desirable compounds according to the methods disclosed above, the methods of the present invention further provide for the manufacture of the selected compounds. Compounds found to desirably modulate the interaction between the interacting protein pairs of proteins of the present invention, or to desirably modulate the activity or intracellular levels of their constituent proteins, can be manufactured for further experimental studies, or for therapeutic use.

### 6. Therapeutic Applications

As described above, the interactions between the interacting pairs of proteins of the present invention suggest that these proteins and/or the protein complexes formed by them may be involved in common biological processes and disease pathways. The protein complexes may mediate the functions of the individual proteins of each interacting protein pair, or of the interacting pairs themselves, in the biological processes or disease pathways. Thus, one may modulate such biological processes or treat diseases by modulating the functions and activities of any of the individual proteins described in the tables, and/or a protein complex comprising some combination of these proteins. As used herein, modulating a protein selected from the tables, or a protein complex comprising some combination of these proteins means altering (enhancing or reducing) the intracellular concentrations or activities of the proteins or protein complexes, e.g., increasing the concentrations of a particular protein described in the tables, or a protein complex comprising some combination of these proteins, enhancing or reducing their biological activities, increasing or decreasing their stability, altering their affinity or specificity to certain other biological molecules, etc. For example, a pair of interacting proteins listed in the tables may be involved in neurotransmission, regulation of APP production and APP metabolism, regulation of Aβ production and Aβ metabolism, processing of proteins destined for secretion, regulation of the wingless pathway, and modulation of thyroid receptor signaling. Thus, assays such as those described in Section 4 may be used in determining the effect of an aberration in a particular protein complex or an interacting member thereof on neurotransmission, regulation of APP production and APP metabolism, regulation of Aβ production and Aβ metabolism, processing of proteins destined for secretion, regulation of the wingless pathway, and modulation of thyroid receptor signaling. In addition, it is also possible to determine, using the same assay methods, the presence or absence of an association between a protein complex of the present invention or an interacting member thereof and a physiological disorder or disease such as Alzheimer's disease (including mild cognitive impairment), Huntington's disease, Parkinson's disease, schizophrenia, depression, dementia and other neurodegenerative diseases, as well as diabetes, obesity and coronary artery disease or predisposition to a physiological disorder or disease.

Once such associations are established, the diagnostic methods as described in Section 4 can be used in diagnosing the disease or disorder, or a patient's predisposition to it. In addition, various in vitro and in vivo assays may be employed to test the therapeutic or prophylactic efficacies of the various therapeutic approaches described in Sections 6.2 and 6.3 that are aimed at modulating the functions and activities of a particular protein complex of the present invention, or an interacting member thereof. Similar assays can also be used to test whether the therapeutic approaches described in Sections 6.2 and 6.3 result in the modulation of neurotransmission, regulation of APP production and APP metabolism, regulation of Aβ production and Aβ metabolism, processing of proteins destined for secretion, regulation of the wingless pathway, and modulation of thyroid receptor signaling. The cell model or transgenic animal model described in Section 7 may be employed in the in vitro and in vivo assays.

In accordance with this aspect of the present invention, methods are provided for modulating (promoting or inhibiting) a protein complex of the present invention formed by the interactions described in the tables. The human cells can be in in vitro cell or tissue cultures. The methods are also applicable to human cells in a patient.

In one embodiment, the concentration of a protein complex formed by the interactions described in the tables is reduced in the cells. Various methods can be employed to reduce the concentration of the protein complex. For example, the protein complex concentration can be reduced by interfering with the interactions between the interacting protein partners. Hence, compounds capable of interfering with interactions between interacting pairs of proteins identified in the tables can be administered to the cells in vitro or in vivo in a patient. Such compounds can be compounds capable of binding specific proteins listed in the tables. They can also be antibodies immunoreactive with specific proteins identified in the tables. Also, the compounds can be small peptides derived from a first interacting protein of the present invention, or a mimetic thereof, that are capable of binding a second protein of the present invention, the second protein being a binding partner of the first protein as shown in the tables above.

In another embodiment, the method of modulating the protein complex includes inhibiting the expression of any of the individual proteins described in the tables. The inhibition can be at the transcriptional, translational, or post-translational level. For example, antisense compounds and ribozyme compounds can be administered to human cells in cultures or in human bodies. In addition, RNA interference technologies may also be employed to administer to cells double-stranded RNA or RNA hairpins capable of “knocking down” the expression of any of the interacting proteins of the present invention.

In the various embodiments described above, preferably the concentrations or activities of both partners in an interacting pair of proteins of the present invention are reduced or inhibited, or the concentration or activitie of a single constituent protein of a protein complex formed by the interactions described in the tables is reduced or inhibited.

In yet another embodiment, an antibody selectively immunoreactive with a pair of interacting proteins identified in the tables is administered to cells in vitro or in human bodies to inhibit the protein complex activities and/or reduce the concentration of the protein complex in the cells or patient.

Further provided by the present invention is a method of treatment of a disease or disorder comprising identifying a patient that has a particular disease or disorder, shows symptoms of having a particular disease or disorder, is predisposed to, or at risk of developing a particular disease or disorder, and treating the disease or disorder by modulating a protein or protein-protein interaction according to the present invention.

### 6.1. Applicable Diseases

Alzheimer's Disease (AD) is a neurodegenerative disease characterized by a progressive decline of cognitive functions, including loss or declarative and procedural memory, decreased learning ability, reduced attention span, and severe impairment in thinking ability, judgment, and decision making. Mood disorders and depression are also often observed in AD patients. It is estimated that AD affects about 4 million people in the USA and 20 million people worldwide. Because AD is an age-related disorder (with an average onset at 65 years), the incidence of the disease in industrialized countries is expected to rise dramatically as the population of these countries is aging.

AD is characterized by the following neuropathological features:
a massive loss of neurons and synapses in the brain regions involved in higher cognitive functions (association cortex, hippocampus, amygdala). Cholinergic neurons are particularly affected.
neuritic (senile) plaques that are composed of a core of amyloid material surrounded by a halo of dystrophic neurites, reactive type I astrocytes, and numerous microglial cells (Selkoe, 1994b; Selkoe, 1994c; Dickson, 1997; Hardy, Gwinn-Hardy, 1998; Selkoe, 1996a). The major component of the core is a peptide of 39 to 42 amino acids called the amyloid 0 protein, or Aβ. Although the Aβ protein is produced by the intracellular processing of its precursor, APP, the amyloid deposits forming the core of the plaques are extracellular. Studies have shown that the longer form of Aβ (Aβ42) is much more amyloidogenic than the shorter forms (Aβ40 or Aβ39).
neurofibrillary tangles that are composed of paired-helical filaments (PHF) (Ray et al., 1998; Brion, 1998). Biochemical analyses revealed that the main component of PHF is a hyper-phosphorylated form of the microtubule-associated protein T. These tangles are intracellular structures, found in the cell body of dying neurons, as well as some dystrophic neurites in the halo surrounding neuritic plaques.

Both plaques and tangles are found in the same brain regions affected by neuronal and synaptic loss.

Although the neuronal and synaptic loss is universally recognized as the primary cause of the decline of cognitive functions, the cellular, biochemical, and molecular events responsible for this neuronal and synaptic loss are subject to fierce controversy. The number of tangles shows a better correlation than the amyloid load with the cognitive decline (Albert, 1996). On the other hand, a number of studies showed that amyloid can be directly toxic to neurons (Iversen et al. 1995; Weiss et al. 1994; Lorenzo, Yankner, 1996; Storey, Cappai, 1999), resulting in behavioral impairment (Ma et al., 1996). It has also been shown that the toxicity of some compounds (amyloid or tangles) could be aggravated by activation of the complement cascade (Rogers et al. 1992b; Rozemuller et al. 1992; Rogers et al. 1992a; Webster et al. 1997), suggesting the possible involvement of inflammatory process in the neuronal death (Fagarasan and Aisen, 1996; Kalaria et al. 1996b; Kalaria et al. 1996a; Farlow, 1998).

Genetic and molecular studies of some familial forms of AD (FAD) have recently provided evidence that boosted the amyloid hypothesis (Ii, 1995; Price et al., 1995; Hardy, 1997; Selkoe, 1996b). The assumption is that since the deposition of Aβ in the core of senile plaques is observed in all Alzheimer cases, if Aβ is the primary cause of AD, then mutations that are linked to FAD should induce changes that, in one way or another, foster Aβ deposition. There are 3 FAD genes known so far (Hardy, Gwinn-Hardy, 1998; Ray et al., 1998), and the activity of all of them results in increased Aβ deposition, a very compelling argument in favor of the amyloid hypothesis.

The first of the 3 FAD genes codes for the Aβ precursor, APP (Selkoe, 1996b). Mutations in the APP gene are very rare, but all of them cause AD with 100% penetrance and result in elevated production of either total Aβ or Aβ42, both in vitro (transfected cells) and in vivo (transgenic animals). The other two FAD genes code for presenilin 1 and 2 (PS1, PS2) (Hardy, 1997). The presenilins contain 8 transmembrane domains and several lines of evidence suggest that they are involved in intracellular protein trafficking, although their exact function is still unknown. Mutations in the presenilin genes are more common than in the APP genes, and all of them also cause FAD with 100% penetrance. In addition, in vitro and in vivo studies have demonstrated that PS1 and PS2 mutations shift APP metabolism, resulting in elevated Aβ42 production. For a recent review on the genetics of AD, see Lippa (1999).

In spite of these compelling genetic data, it is still unclear whether Aβ generation and amyloid deposition are the primary cause of neuronal death and synaptic loss observed in AD. Moreover, the biochemical events leading to Aβ production, the relationship between APP and the presenilins, and between amyloid and neurofibrillary tangles are poorly understood. Thus, the picture of interactions between the major Alzheimer proteins is very incomplete, and it is clear that a large number of novel proteins are yet to be discovered. To this end, we have initiated a systematic study looking at proteins interacting with various domains of the major Alzheimer proteins (see below). The results from these experiments provide a more complete understanding of the protein-protein interactions involved in AD pathogenesis, and thus will greatly help in the identification of a drug target. Because AD is a neurodegenerative disease, it is also expected that this project will identify novel proteins involved in neuronal survival, neurite outgrowth, and maintenance of synaptic structures, thus opening opportunities into potentially any pathological condition in which the integrity of neurons and synapses is threatened.

The methods for modulating the functions and activities of a protein complex of the present invention, or an interacting member thereof, may be employed to modulate neurotransmission and APP metabolism.

In addition, the methods of the present invention can also be useful in treating or preventing neurodegenerative disorders including, but not limited to, Alzheimer's disease (including mild cognitive impairment), frontotemporal dementia, Parkinson's disease, Huntington's disease, brain trauma, infarction, hemorrhage, amytrophic lateral sclerosis/Lou Gehrig's disease (ALS), inherited ataxias such as olivopontocerebellar atrophy (spinocerebellar ataxia type 1), and Machado-Joseph disease (spinocerebellar ataxia type 3).

The methods for modulating the functions and activities of a protein complex of the present invention, or an interacting member thereof, may be employed to modulate neurotransmission, regulation of APP production and APP metabolism, regulation of Aβ production and Aβ metabolism, processing of proteins destined for secretion, regulation of the wingless pathway, and modulation of thyroid receptor signaling. In addition, the methods may also be used in the treatment or prevention of diseases and disorders such as Alzheimer's disease (including mild cognitive impairment), Huntington's disease, Parkinson's disease, schizophrenia, depression, dementia and other neurodegenerative diseases, as well as diabetes, obesity and coronary artery disease.

Thus, the picture of interactions between the major AD proteins is very incomplete, and it is clear that a number of novel proteins are yet to be discovered. Although a number of molecules have been identified as possibly involved in the disease progression, no particular protein (or set of proteins) has been identified as primarily responsible for the loss of neurons and synapses. More importantly, none of the various components identified so far in the cascade of events leading to AD is a confirmed drug target.

There continues to be a need in the art for the discovery of additional proteins interacting with various domains of the major Alzheimer proteins, including APP and the presenilins. There continues to be a need in the art also to identify the protein-protein interactions that are involved in AD pathogenesis, and to thus identify drug targets.

In a specific embodiment, a method for treating or preventing schizophrenia is provided which includes modulating the protein complexes of the present invention, or the interacting protein members thereof. Other various diseases involving abnormal neuronal growth or injuries may also be treated. Examples of such disorders include various disorders caused by supratentorial mass lesions, subtentorial mass or destructive lesions, or metabolic brain diseases, lesions of the peripheral common motor, sensory and autonomic pathways. The method for modulating the protein complexes or interacting protein members thereof may also be used in treating diseases and disorders such as delirium, dementia, Korsakoff’ syndrome, manic-depressive psychosis, anxiety, depression, and hysteria.

### 6.2. Inhibiting Protein Complex or Interacting Protein Members Thereof

In one aspect of the present invention, methods are provided for reducing in cells or tissue the concentration and/or activity of a protein complex identified in accordance with the present invention that comprises one or more of the interacting pairs of proteins described in the tables. In addition, methods are also provided for reducing in cells or tissue the concentration and/or activity of any of the individual proteins identified in the tables. By reducing the concentration of a protein complex and/or one or more of the protein constituents of the protein complex and/or inhibiting the functional activities of the protein complex and/or one or more of the protein constituents of the protein complex, the diseases involving such a protein complex or protein constituents of the protein complex may be treated or prevented.

### 6.2.1. Antibody Therapy

In one embodiment, an antibody may be administered to cells or tissue in vitro or to patients. The antibody administered may be immunoreactive with any of the individual proteins described in the tables, or with one of the protein complexes of the present invention. Suitable antibodies may be monoclonal or polyclonal that fall within any antibody class, e.g., IgG, IgM, IgA, IgE, etc. The antibody suitable for this invention may also take a form of various antibody fragments including, but not limited to, Fab and F(ab′)₂, single-chain fragments (scFv), and the like. In another embodiment, an antibody selectively immunoreactive with the protein complex formed from at least one of the interacting pairs of proteins described in the tables, is administered to cells or tissue in vitro or in to patient. In yet another embodiment, an antibody specific to an individual protein selected from any of the tables is administered to cells or tissue in vitro or in a patient. Methods for making the antibodies of the present invention should be apparent to a person of skill in the art, especially in view of the discussions in Section 3 above. The antibodies can be administered in any suitable form via any suitable route as described in Section 8 below. Preferably, the antibodies are administered in a pharmaceutical composition together with a pharmaceutically acceptable carrier.

Alternatively, the antibodies may be delivered by a gene-therapy approach. That is, nucleic acids encoding the antibodies, particularly single-chain fragments (scFv), may be introduced into cells or tissue in vitro or in a patient such that desirable antibodies may be produced recombinantly in vivo from the nucleic acids. For this purpose, the nucleic acids with appropriate transcriptional and translation regulatory sequences can be directly administered into the patient. Alternatively, the nucleic acids can be incorporated into a suitable vector as described in Sections 2.2 and 5.3.1.1 and delivered into cells or tissue in vitro or in a patient along with the vector. The expression vector containing the nucleic acids can be administered directly to cells or tissue in vitro or in a patient. It can also be introduced into cells, preferably cells derived from a patient to be treated, and subsequently delivered into the patient by cell transplantation. See Section 6.3.2 below.

### 6.2.2. siRNA Therapy

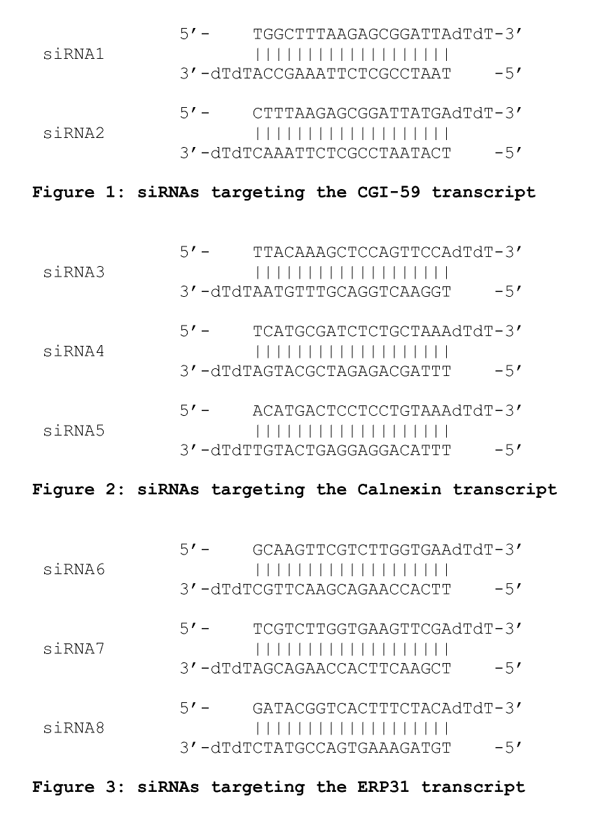

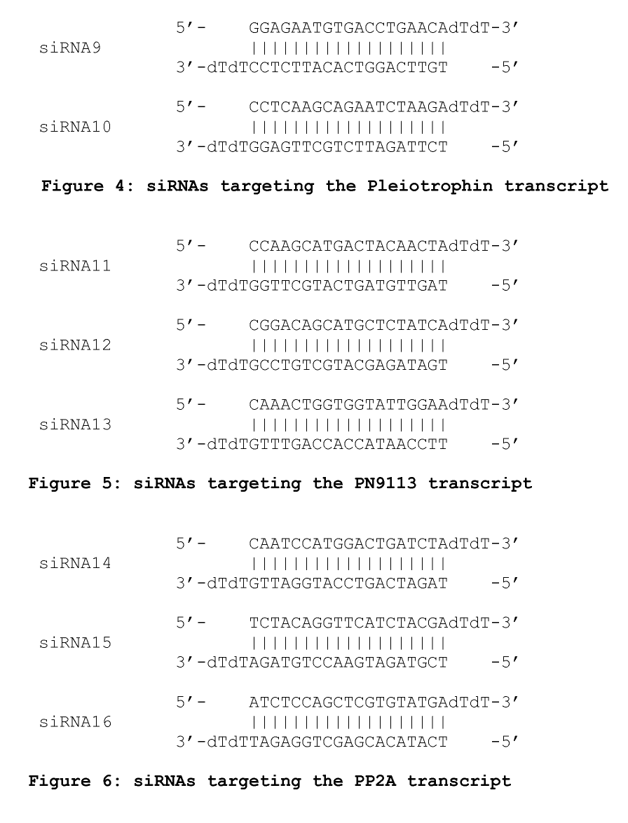

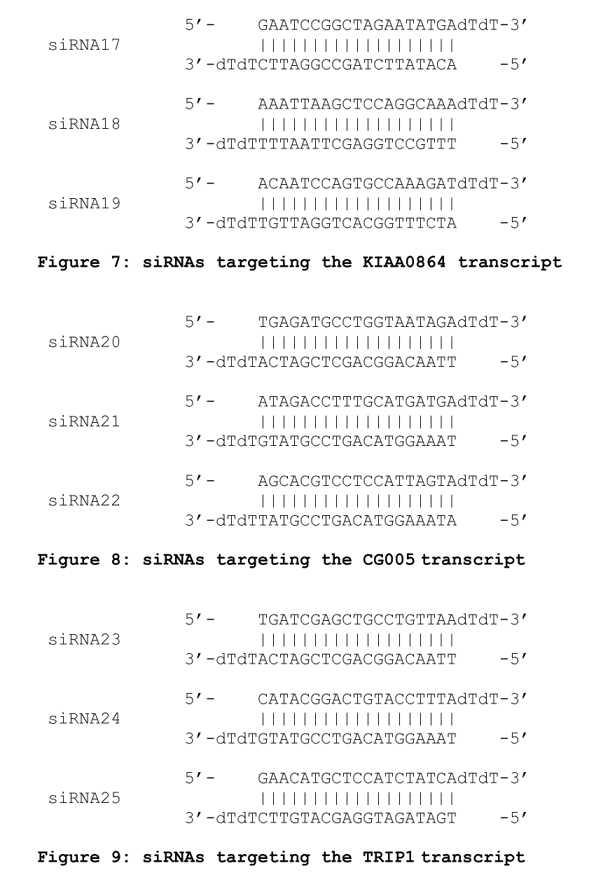

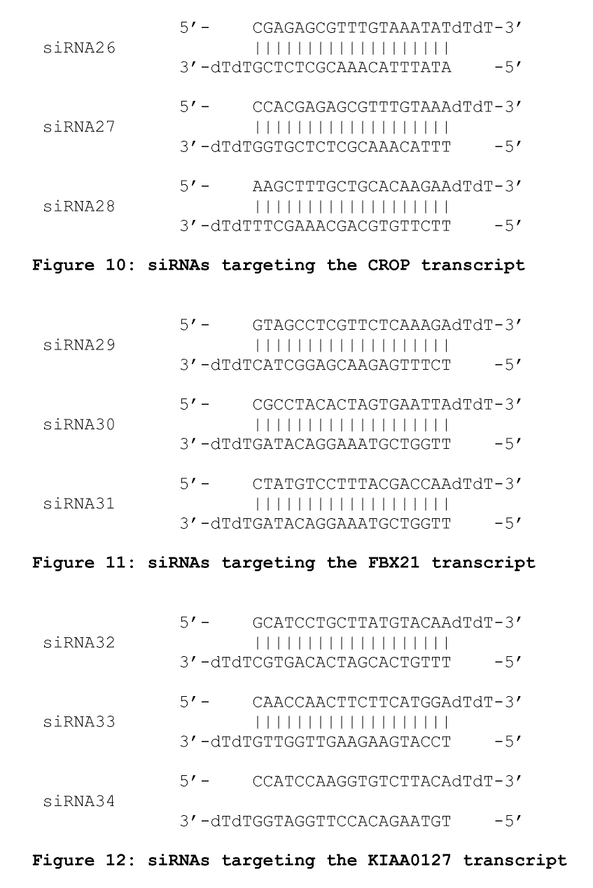

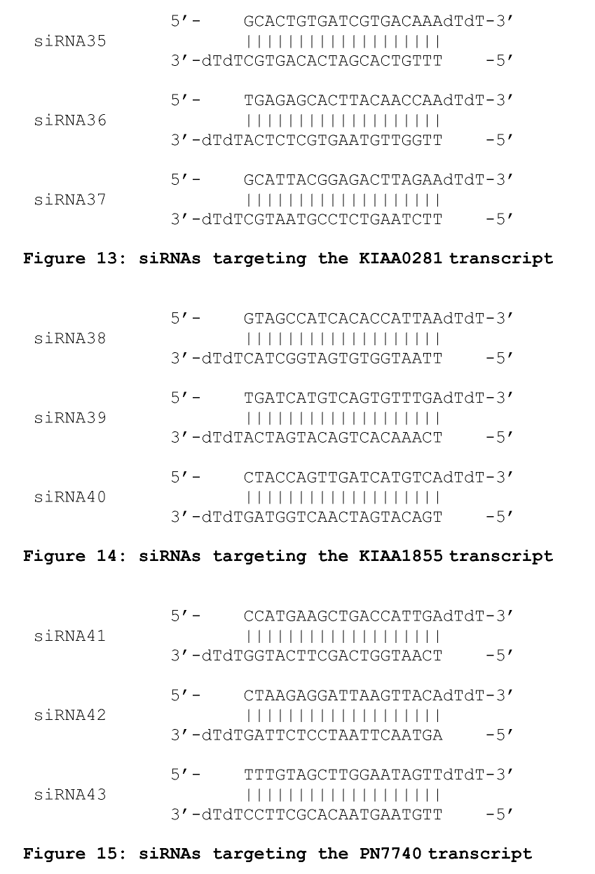

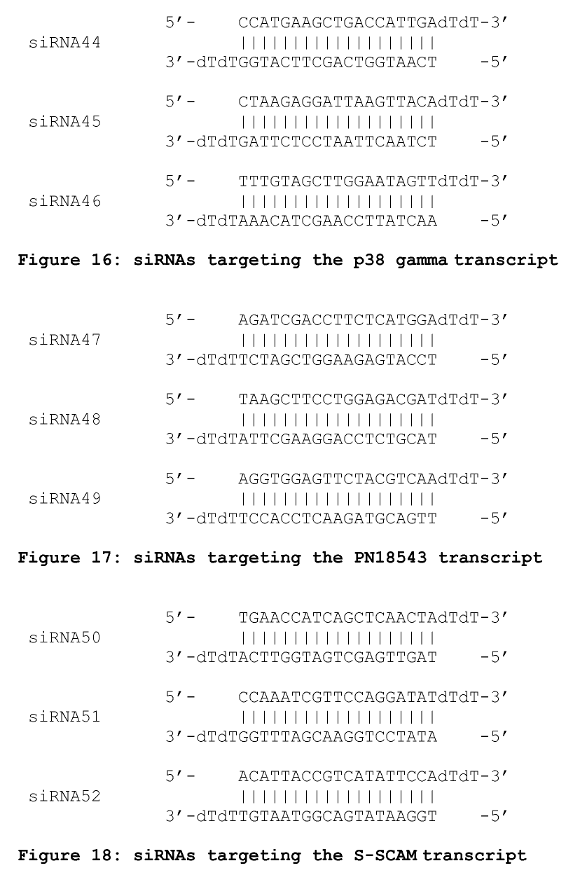

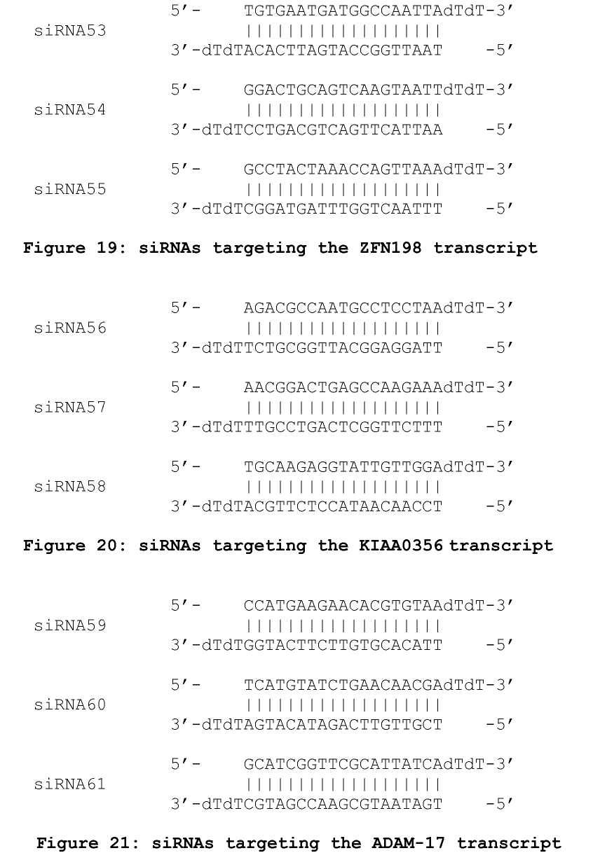

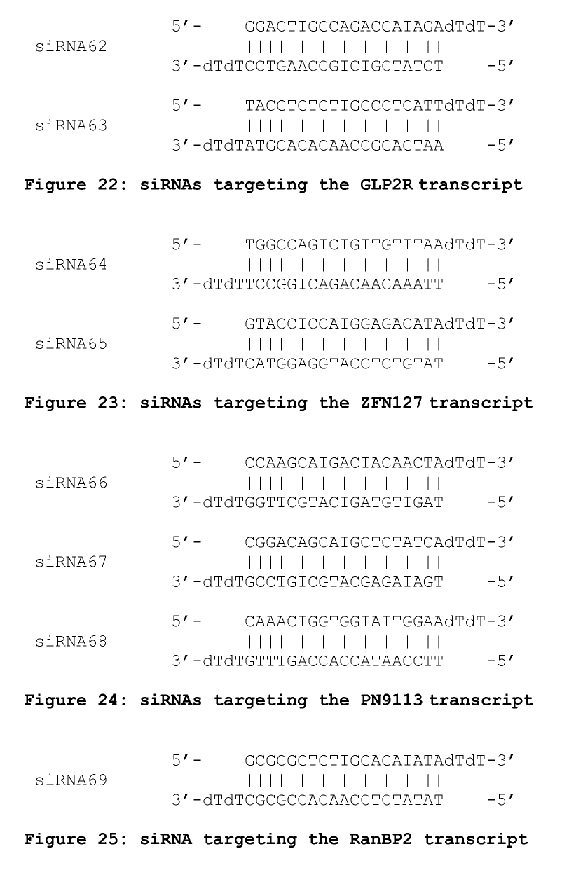

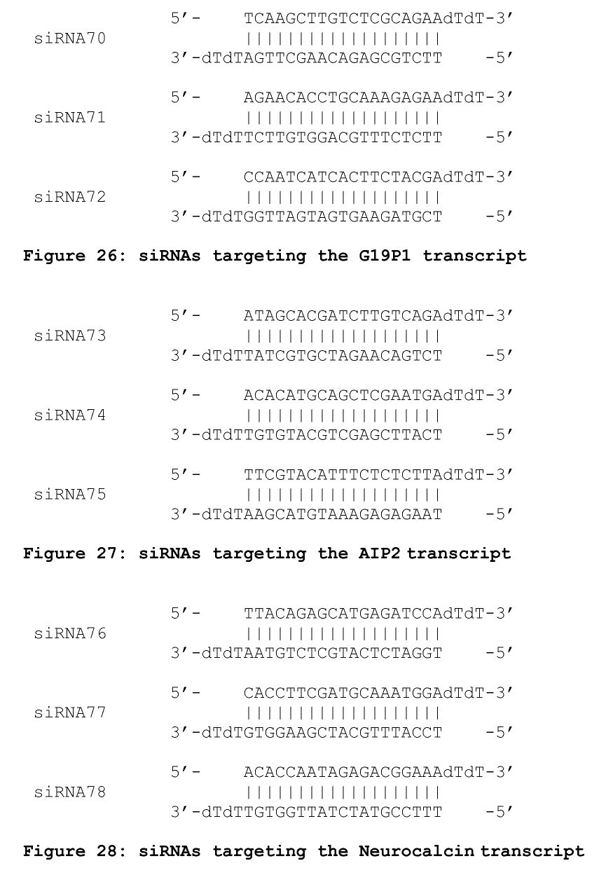

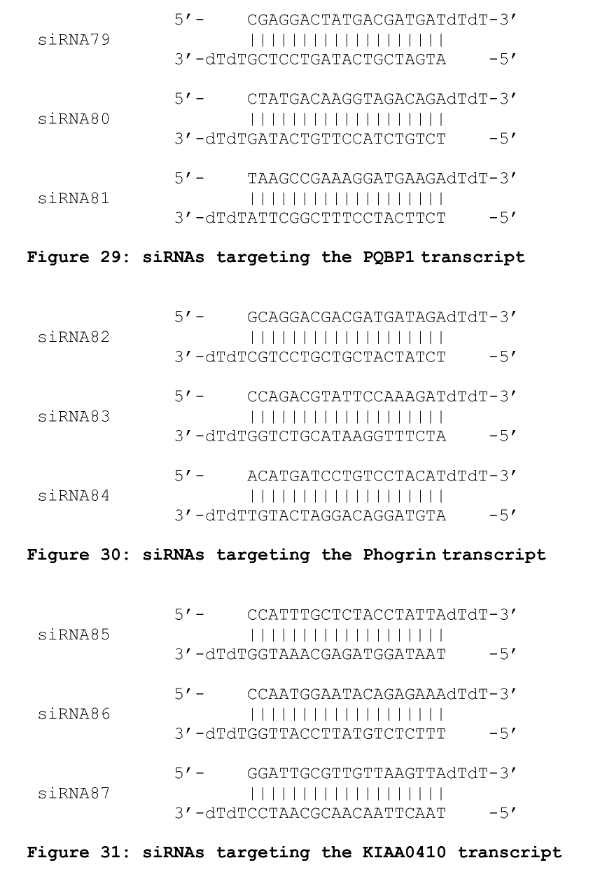

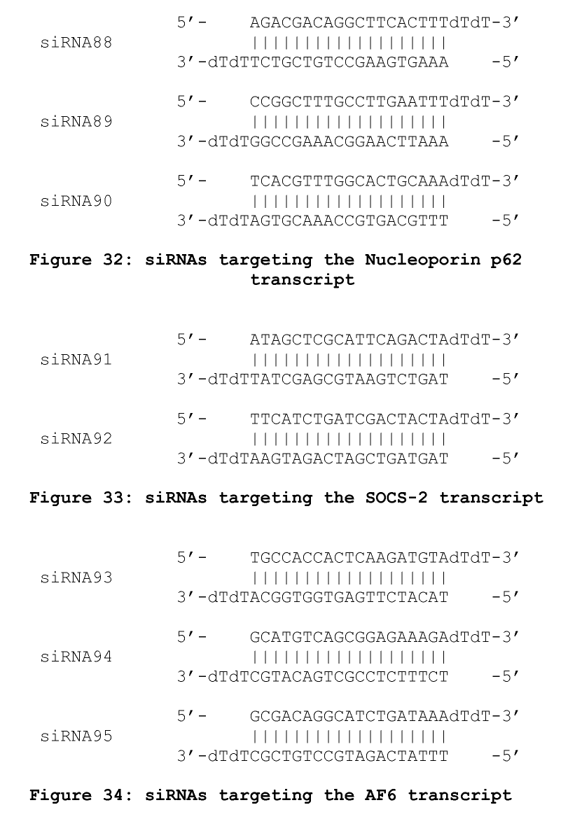

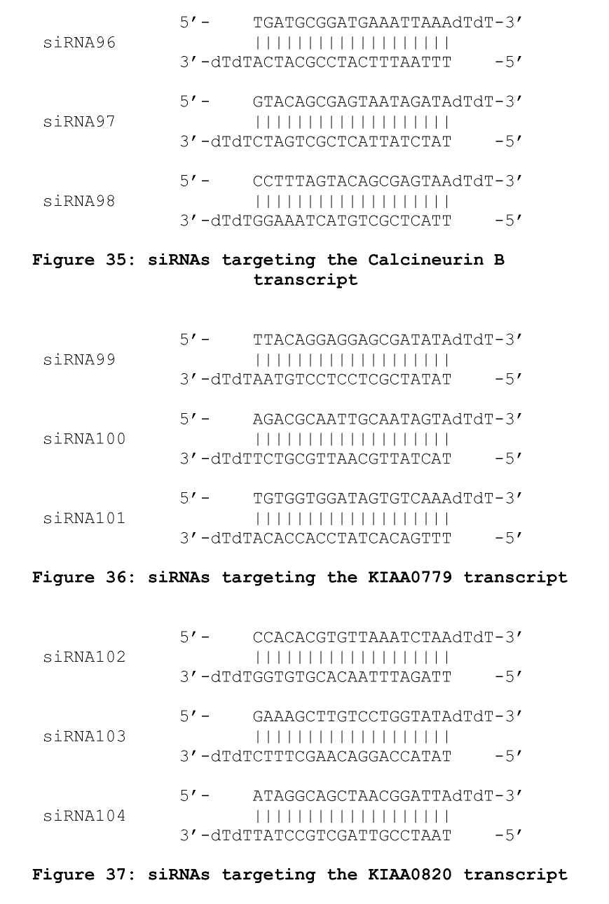

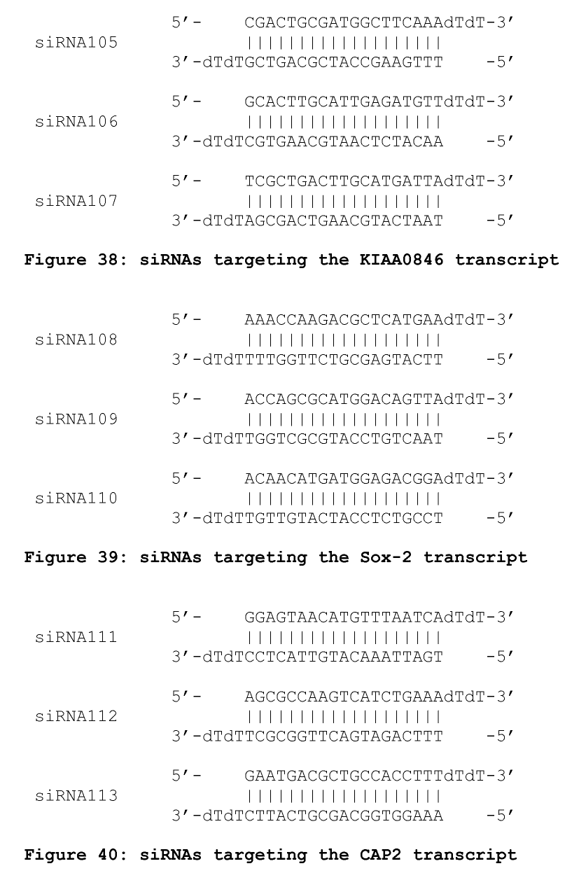

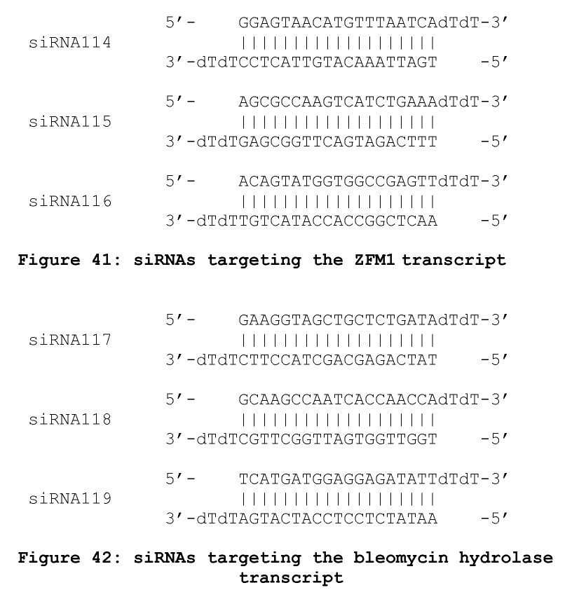

In another embodiment, double-stranded small interfering RNA (siRNA) compounds specific to nucleic acids encoding one or more interacting protein members of a protein complex identified in the present invention are administered to cells or tissue in vitro or in a patient to be therapeutically or prophylactically treated. Exemplary siRNA molecules for the targets identified in these studies are provided in FIGS. 1-42.

As is generally known in the art now, siRNA compounds are RNA duplexes comprising two complementary single-stranded RNAs of 21 nucleotides that form 19 base pairs and possess 3′ overhangs of two nucleotides. See Elbashir et al., Nature 411:494-498 (2001); and PCT Publication Nos. WO 00/44895; WO 01/36646; WO 99/32619; WO 00/01846; WO 01/29058; WO 99/07409; and WO 00/44914. When appropriately targeted via its nucleotide sequence to a specific mRNA in cells, an siRNA can specifically suppress gene expression through a process known as RNA interference (RNAi). See e.g., Zamore & Aronin, Nature Medicine, 9:266-267 (2003). siRNAs can reduce the cellular level of specific mRNAs, and decrease the level of proteins coded by such mRNAs. siRNAs utilize sequence complementarity to target an mRNA for destruction, and are sequence-specific. Thus, they can be highly target-specific, and in mammals have been shown to target mRNAs encoded by different alleles of the same gene. Because of this precision, side effects typically associated with traditional drugs can be reduced or eliminated. In addition, they are relatively stable, and like antisense and ribozyme molecules, they can also be modified to achieve improved pharmaceutical characteristics, such as increased stability, deliverability, and ease of manufacture. Moreover, because siRNA molecules take advantage of a natural cellular pathway, i.e., RNA interference, they are highly efficient in destroying targeted mRNA molecules. As a result, it is relatively easy to achieve a therapeutically effective concentration of an siRNA compound in patients. Thus, siRNAs are a promising new class of drugs being actively developed by pharmaceutical companies.

Indeed, in vivo inhibition of specific gene expression by RNAi has been achieved in various organisms including mammals. For example, Song et al., Nature Medicine, 9:347-351 (2003) discloses that intravenous injection of Fas siRNA compounds into laboratory mice with autoimmune hepatitis specifically reduced Fas mRNA levels and expression of Fas protein in mouse liver cells. The gene silencing effect persisted without diminution for 10 days after the intravenous injection. The injected siRNA was effective in protecting the mice from liver failure and fibrosis. Song et al., Nature Medicine, 9:347-351 (2003). Several other approaches for delivery of siRNA into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002).

The siRNA compounds provided according to the present invention can be synthesized using conventional RNA synthesis methods. For example, they can be chemically synthesized using appropriately protected ribonucleoside phosphoramidites and a conventional DNA/RNA synthesizer. Various applicable methods for RNA synthesis are disclosed in, e.g., Usman et al., J. Am. Chem. Soc., 109:7845-7854 (1987) and Scaringe et al., Nucleic Acids Res., 18:5433-5441 (1990). Custom siRNA synthesis services are available from commercial vendors such as Ambion (Austin, Tex., USA), Dharmacon Research (Lafayette, Colo., USA), Pierce Chemical (Rockford, Ill., USA), ChemGenes (Ashland, Mass., USA), Proligo (Hamburg, Germany), and Cruachem (Glasgow, UK).

The siRNA compounds can also be various modified equivalents of the siRNA structures. As used herein, “modified equivalent” means a modified form of a particular siRNA compound having the same target-specificity (i.e., recognizing the same mRNA molecules that complement the unmodified particular siRNA compound). Thus, a modified equivalent of an unmodified siRNA compound can have modified ribonucleotides, that is, ribonucleotides that contain a modification in the chemical structure of an unmodified nucleotide base, sugar and/or phosphate (or phosphodiester linkage). As is known in the art, an “unmodified ribonucleotide” has one of the bases adenine, cytosine, guanine, and uracil joined to the 1′ carbon of beta-D-ribo-furanose.

Preferably, modified siRNA compounds contain modified backbones or non-natural internucleoside linkages, e.g., modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. See e.g., U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Examples of the non-phosphorous containing backbones described above are disclosed in, e.g., U.S. Pat. Nos. 5,034,506; 5,185,444; 5,214,134; 5,216,141; 5,235,033; 5,264,562; 5,264,564; 5,405,938; 5,434,257; 5,470,967; 5,489,677; 5,541,307; 5,561,225; 5,596,086; 5,610,289; 5,602,240; 5,608,046; 5,610,289; 5,618,704; 5,623,070; 5,663,312; 5,677,437; and 5,677,439, each of which is herein incorporated by reference.

Modified forms of siRNA compounds can also contain modified nucleosides (nucleoside analogs), i.e., modified purine or pyrimidine bases, e.g., 5-substituted pyrimidines, 6-azapyrimidines, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), 2-thiouridine, 4-thiouridine, 5-(carboxyhydroxymethyl)uridine, 5′-carboxymethylaminomethyl-2-thiouridine, 5-carboxymethylaminomethyluridine, 5-methoxyaminomethyl-2-thiouridine, 5-methylaminomethyluridine, 5-methylcarbonylmethyluridine, 5-methyloxyuridine, 5-methyl-2-thiouridine, 4-acetylcytidine, 3-methylcytidine, propyne, quesosine, wybutosine, wybutoxosine, beta-D-galactosylqueosine, N-2, N-6 and O-substituted purines, inosine, 1-methyladenosine, 1-methylinosine, 2,2-dimethylguanosine, 2-methyladenosine, 2-methylguanosine, N6-methyladenosine, 7-methylguanosine, 2-methylthio-N-6-isopentenyladenosine, beta-D-mannosylqueosine, uridine-5-oxyacetic acid, 2-thiocytidine, threonine derivatives, and the like. See e.g., U.S. Pat. Nos. 3,687,808; 4,845,205; 5,130,302; 5,175,273; 5,367,066; 5,432,272; 5,459,255; 5,484,908; 5,502,177; 5,525,711; 5,587,469; 5,594,121; 5,596,091; 5,681,941; and 5,750,692, PCT Publication No. WO 92/07065; PCT Publication No. WO 93/15187; and Limbach et al., Nucleic Acids Res., 22:2183 (1994), each of which is incorporated herein by reference in its entirety.

In addition, modified siRNA compounds can also have substituted or modified sugar moieties, e.g., 2′-O-methoxyethyl sugar moieties. See e.g., U.S. Pat. Nos. 4,981,957; 5,118,800; 5,319,080; 5,393,878; 5,446,137; 5,466,786; 5,514,785; 5,567,811; 5,576,427; 5,591,722; 5,610,300; 5,627,0531 5,639,873; 5,646,265; 5,658,873; 5,670,633; and 5,700,920, each of which is herein incorporated by reference.

Modified siRNA compounds may be synthesized by the methods disclosed in, e.g., U.S. Pat. No. 5,652,094; International Publication Nos. WO 91/03162; WO 92/07065 and WO 93/15187; European Patent Application No. 92110298.4; Perrault et al., Nature, 344:565 (1990); Pieken et al., Science, 253:314 (1991); and Usman and Cedergren, Trends in Biochem. Sci., 17:334 (1992).

Preferably, the 3′ overhangs of the siRNAs of the present invention are modified to provide resistance to cellular nucleases. In one embodiment the 3′ overhangs comprise 2′-deoxyribonucleotides. In a preferred embodiment (depicted in FIG. 1) these 3′ overhangs comprise a dinucleotide made of two 2′-deoxythymine residues (i.e., dTdT) linked by a 5′-3′ phosphodiester linkage.

siRNA compounds may be administered to mammals by various methods through different routes. For example, they can be administered by intravenous injection. See Song et al., Nature Medicine, 9:347-351 (2003). They can also be delivered directly to a particular organ or tissue by any suitable localized administration methods. Several other approaches for delivery of siRNA into animals have also proved to be successful. See e.g., McCaffery et al, Nature, 418:38-39 (2002); Lewis et al., Nature Genetics, 32:107-108 (2002); and Xia et al., Nature Biotech., 20:1006-1010 (2002). Alternatively, they may be delivered encapsulated in liposomes, by iontophoresis, or by incorporation into other vehicles such as hydrogels, cyclodextrins, biodegradable nanocapsules, and bioadhesive microspheres.

In addition, they may also be delivered by a gene therapy approach, using a DNA vector from which siRNA compounds in, e.g., small hairpin form (shRNA), can be transcribed directly. Recent studies have demonstrated that while double-stranded siRNAs are very effective at mediating RNAi, short, single-stranded, hairpin-shaped RNAs can also mediate RNAi, presumably because they fold into intramolecular duplexes that are processed into double-stranded siRNAs by cellular enzymes. Sui et al., Proc. Natl. Acad. Sci. U.S.A., 99:5515-5520 (2002); Yu et al., Proc. Natl. Acad. Sci. U.S.A., 99:6047-6052 (2002); and Paul et al., Nature Biotech., 20:505-508 (2002)). This discovery has significant and far-reaching implications, since the production of such shRNAs can be readily achieved in vivo by transfecting cells or tissues with DNA vectors bearing short inverted repeats separated by a small number of (e.g., 3 to 9) nucleotides that direct the transcription of such small hairpin RNAs. Additionally, if mechanisms are included to direct the integration of the transcription cassette into the host cell genome, or to ensure the stability of the transcription vector, the RNAi caused by the encoded shRNAs, can be made stable and heritable. Not only have such techniques been used to “knock down” the expression of specific genes in mammalian cells, but they have now been successfully employed to knock down the expression of exogenously expressed transgenes, as well as endogenous genes in the brain and liver of living mice. See generally Hannon, Nature. 418:244-251 (2002) and Shi, Trends Genet., 19:9-12 (2003); see also Xia et al., Nature Biotech., 20:1006-1010 (2002).

Additional siRNA compounds targeted at different sites of the nucleic acids encoding one or more interacting protein members of a protein complex identified in the present invention may also be designed and synthesized according to general guidelines provided herein and generally known to skilled artisans. See e.g., Elbashir, et al. Nature 411: 494-498 (2001). For example, guidelines have been compiled into “The siRNA User Guide” which is available at the website of the Tuschl Lab of The Rockefeller University, New York, N.Y.

Additionally, to assist in the design of siRNAs for the efficient RNAi-mediated silencing of any target gene, several siRNA supply companies maintain web-based design tools that utilize these general guidelines for “picking” siRNAs when presented with the mRNA or coding DNA sequence of the target gene. Examples of such tools can be found at the web sites of Dharmacon, Inc. (Lafayette, Colo.), Ambion, Inc. (Austin, Tex.), and Qiagen, Inc. (Valencia, Calif.), among others. Generally speaking, when provided with an mRNA or coding DNA sequence, these design tools scan the sequence for potential siRNA targets, using several distinct criteria. For example, the design tools may scan for an open reading frame and limit further scanning to that region of sequence. They may then scan for a particular dinucleotide, the most desirable of which being AA, or alternatively CA, GA or TA. Upon finding one of these dinucleotides, they will then examine the dinucleotide and the 19 nucleotides immediately 3′ of it for G/C content, nucleotide triplets (esp. GGG & CCC), and, using a BLAST algorithm search, for whether or not the 19 nucleotide sequence is unique to a specific target gene in the human genome. The features that make for an “ideal” target sequence are: (1) a 5′-most dinucleotide sequence of AA, or, less preferably, CA, GA or TA; (2) a G/C content of approximately 30-50%; (3) lack of trinucleotide repeats, especially GGG and CCC, and (4) being unique to the target gene (i.e., sequences that share no significant homology with genes other than the one being targeted), so that other genes are not inadvertently targeted by the same siRNA designed for this particular target sequence. Another criteria to be considered is whether or not the target sequence includes a known polymorphic site. If so, siRNAs designed to target one particular allele may not effectively target another allele, since single base mismatches between the target sequence and its complementary strand in a given siRNA can greatly reduce the effectiveness of RNAi induced by that siRNA. Given that target sequence and such design tools and design criteria, an ordinarily skilled artisan apprised of the present disclosure should be able to design and synthesized additional siRNA compounds useful in reducing the mRNA level and therefore protein level of one or more interacting protein members of a protein complex identified in the present invention.

### 6.2.3. Antisense Therapy

In another embodiment, antisense compounds specific to nucleic acids encoding one or more interacting protein members of a protein complex identified in the present invention are administered to cells or tissue in vitro or in a patient to be therapeutically or prophylactically treated. The antisense compounds should specifically inhibit the expression of the one or more interacting protein members. As is known in the art, antisense drugs generally act by hybridizing to a particular target nucleic acid thus blocking gene expression. Methods for designing antisense compounds and using such compounds in treating diseases are well known and well developed in the art. For example, the antisense drug Vitravene® (fomivirsen), a 21-base long oligonucleotide, has been successfully developed and marketed by Isis Pharmaceuticals, Inc. for treating cytomegalovirus (CMV)-induced retinitis. Exemplary antisense molecules directed towards the targets identified as a result of these studies are given in SEQ ID NOS. 161-283.

Any methods for designing and making antisense compounds may be used for the purpose of the present invention. See generally, Sanghvi et al, eds., Antisense Research and Applications, CRC Press, Boca Raton, 1993. Typically, antisense compounds are oligonucleotides designed based on the nucleotide sequence of the mRNA or gene of one or more target proteins, e.g., the interacting protein members of a particular protein complex of the present invention. In particular, antisense compounds can be designed to specifically hybridize to a particular region of the gene sequence or mRNA of one or more of the interacting protein members to modulate (increase or decrease) replication, transcription, or translation. As used herein, the term “specifically hybridize” or paraphrases thereof means a sufficient degree of complementarity or pairing between an antisense oligo and a target DNA or mRNA such that stable and specific binding occurs therebetween. In particular, 100% complementary or pairing is not required. Specific hybridization takes place when sufficient hybridization occurs between the antisense compound and its intended target nucleic acids in the substantial absence of non-specific binding of the antisense compound to non-target sequences under predetermined conditions, e.g., for purposes of in vivo treatment, preferably under physiological conditions. Preferably, specific hybridization results in the interference with normal expression of the target DNA or mRNA.

For example, antisense oligonucleotides can be designed to specifically hybridize to target genes, in regions critical for regulation of transcription; to pre-mRNAs, in regions critical for correct splicing of nascent transcripts; and to mature mRNAs, in regions critical for translation initiation or mRNA stability and localization.

As is generally known in the art, commonly used oligonucleotides are oligomers or polymers of ribonucleotides or deoxyribonucleotides, that are composed of a naturally-occurring nitrogenous base, a sugar (ribose or deoxyribose) and a phosphate group. In nature, the nucleotides are linked together by phosphodiester bonds between the 3′ and 5′ positions of neighboring sugar moieties. However, it is noted that the term “oligonucleotides” also encompasses various non-naturally occurring mimetics and derivatives, i.e., modified forms, of naturally occurring oligonucleotides as described below. Typically an antisense compound of the present invention is an oligonucleotide having from about 6 to about 200, and preferably from about 8 to about 30 nucleoside bases.

The antisense compounds preferably contain modified backbones or non-natural internucleoside linkages, including but not limited to, modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. See e.g., U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Examples of the non-phosphorous containing backbones described above are disclosed in, e.g., U.S. Pat. Nos. 5,034,506; 5,185,444; 5,214,134; 5,216,141; 5,235,033; 5,264,562; 5,264,564; 5,405,938; 5,434,257; 5,470,967; 5,489,677; 5,541,307; 5,561,225; 5,596,086; 5,610,289; 5,602,240; 5,608,046; 5,610,289; 5,618,704; 5,623,070; 5,663,312; 5,677,437; and 5,677,439, each of which is herein incorporated by reference.

Another useful modified oligonucleotide is peptide nucleic acid (PNA), in which the sugar-backbone of an oligonucleotide is replaced with an amide containing backbone, e.g., an aminoethylglycine backbone. See U.S. Pat. Nos. 5,539,082 and 5,714,331; and Nielsen et al., Science, 254, 1497-1500 (1991), all of which are incorporated herein by reference. PNA antisense compounds are resistant to RNase H digestion and thus exhibit longer half-life. In addition, various modifications may be made in PNA backbones to impart desirable drug profiles such as better stability, increased drug uptake, higher affinity to target nucleic acid, etc.

Alternatively, the antisense compounds are oligonucleotides containing modified nucleosides, i.e., modified purine or pyrimidine bases, e.g., 5-substituted pyrimidines, 6-azapyrimidines, and N-2, N-6 and O-substituted purines, and the like. See e.g., U.S. Pat. Nos. 3,687,808; 4,845,205; 5,130,302; 5,175,273; 5,367,066; 5,432,272; 5,459,255; 5,484,908; 5,502,177; 5,525,711; 5,587,469; 5,594,121; 5,596,091; 5,681,941; and 5,750,692, each of which is incorporated herein by reference in its entirety.

In addition, oligonucleotides with substituted or modified sugar moieties may also be used. For example, an antisense compound may have one or more 2′-O-methoxyethyl sugar moieties. See e.g., U.S. Pat. Nos. 4,981,957; 5,118,800; 5,319,080; 5,393,878; 5,446,137; 5,466,786; 5,514,785; 5,567,811; 5,576,427; 5,591,722; 5,610,300; 5,627,0531 5,639,873; 5,646,265; 5,658,873; 5,670,633; and 5,700,920, each of which is herein incorporated by reference.

Other types of oligonucleotide modifications are also useful including linking an oligonucleotide to a lipid, phospholipid or cholesterol moiety, cholic acid, thioether, aliphatic chain, polyamine, polyethylene glycol (PEG), or a protein or peptide. The modified oligonucleotides may exhibit increased uptake into cells, and improved stability, i.e., resistance to nuclease digestion and other biodegradations. See e.g., U.S. Pat. No. 4,522,811; Burnham, Am. J. Hosp. Pharm., 15:210-218 (1994).

Antisense compounds can be synthesized using any suitable methods known in the art. In fact, antisense compounds may be custom made by commercial suppliers. Alternatively, antisense compounds may be prepared using DNA synthesizers available commercially from various vendors, e.g., Applied Biosystems Group of Norwalk, Conn.

The antisense compounds can be formulated into a pharmaceutical composition with suitable carriers and administered into cells or tissue in vitro or in a patient using any suitable route of administration. Alternatively, the antisense compounds may also be used in a “gene-therapy” approach. That is, the oligonucleotide is subcloned into a suitable vector and transformed into human cells. The antisense oligonucleotide is then produced in vivo through transcription. Methods for gene therapy are disclosed in Section 6.3.2 below.

### 6.2.4. Ribozyme Therapy

In another embodiment, an enzymatic RNA or ribozyme is designed to target the nucleic acids encoding one or more of the interacting protein members of the protein complexes of the present invention. Ribozymes are RNA molecules possessing enzymatic activity. One class of ribozymes is capable of repeatedly cleaving other separate RNA molecules into two or more pieces in a nucleotide base sequence specific manner. See Kim et al., Proc. Natl. Acad. of Sci. USA, 84:8788 (1987); Haseloff and Gerlach, Nature, 334:585 (1988); and Jefferies et al., Nucleic Acid Res., 17:1371 (1989). Such ribozymes typically have two functional domains: a catalytic domain and a binding sequence that guides the binding of ribozymes to a target RNA through complementary base-pairing. Once a specifically-designed ribozyme is bound to a target mRNA, it enzymatically cleaves the target mRNA, typically reducing its stability and destroying its ability to direct translation of an encoded protein. After a ribozyme has cleaved its RNA target, it is released from that target RNA and thereafter can bind and cleave another target. That is, a single ribozyme molecule can repeatedly bind and cleave new targets. Therefore, one advantage of ribozyme treatment is that a lower amount of exogenous RNA is required as compared to conventional antisense therapies. In addition, ribozymes exhibit less affinity to mRNA targets than DNA-based antisense oligonucleotides, and therefore are less prone to bind to unintended targets.

In accordance with the present invention, a ribozyme may target any portion of the mRNA encoding one or more interacting protein members of the protein complexes formed by the interactions described in the tables. Methods for selecting a ribozyme target sequence and designing and making ribozymes are generally known in the art. See e.g., U.S. Pat. Nos. 4,987,071; 5,496,698; 5,525,468; 5,631,359; 5,646,020; 5,672,511; and 6,140,491, each of which is incorporated herein by reference in its entirety. For example, suitable ribozymes may be designed in various configurations such as hammerhead motifs, hairpin motifs, hepatitis delta virus motifs, group I intron motifs, or RNase P RNA motifs. See e.g., U.S. Pat. Nos. 4,987,071; 5,496,698; 5,525,468; 5,631,359; 5,646,020; 5,672,511; and 6,140,491; Rossi et al., AIDS Res. Human Retroviruses 8:183 (1992); Hampel and Tritz, Biochemistry 28:4929 (1989); Hampel et al., Nucleic Acids Res., 18:299 (1990); Perrotta and Been, Biochemistry 31:16 (1992); and Guerrier-Takada et al., Cell, 35:849 (1983).

Ribozymes can be synthesized by the same methods used for normal RNA synthesis. For example, such methods are disclosed in Usman et al., J. Am. Chem. Soc., 109:7845-7854 (1987) and Scaringe et al., Nucleic Acids Res., 18:5433-5441 (1990). Modified ribozymes may be synthesized by the methods disclosed in, e.g., U.S. Pat. No. 5,652,094; International Publication Nos. WO 91/03162; WO 92/07065 and WO 93/15187; European Patent Application No. 92110298.4; Perrault et al., Nature, 344:565 (1990); Pieken et al., Science, 253:314 (1991); and Usman and Cedergren, Trends in Biochem. Sci., 17:334 (1992).

Ribozymes of the present invention may be administered to cells by any known methods, e.g., disclosed in International Publication No. WO 94/02595. For example, they can be administered directly to cells or tissue in vitro or in a patient through any suitable route, e.g., intravenous injection. Alternatively, they may be delivered encapsulated in liposomes, by iontophoresis, or by incorporation into other vehicles such as hydrogels, cyclodextrins, biodegradable nanocapsules, and bioadhesive microspheres. In addition, they may also be delivered by a gene therapy approach, using a DNA vector from which the ribozyme RNA can be transcribed directly. Gene therapy methods are disclosed in detail below in Section 6.3.2.

### 6.2.5. Other Methods

The in-patient concentrations and activities of the protein complexes and interacting proteins of the present invention may also be altered by other methods. For example, compounds identified in accordance with the methods described in Section 5 that are capable of interfering with or dissociating protein-protein interactions between the interacting protein members of a protein complex may be administered to cells or tissue in vitro or in a patient. Compounds identified in in vitro binding assays described in Section 5.2 that bind to the protein complexes of the present invention, or the interacting members thereof, may also be used in the treatment. Compounds identified in in vitro binding assays described in Section 5.2 that bind to the protein complexes of the present invention, or the interacting members thereof, may also be used in the treatment.

In addition, potentially useful agents also include incomplete proteins, i.e., fragments of the interacting protein members that are capable of binding to their respective binding partners in a protein complex but are defective with respect to their normal cellular functions. For example, binding domains of the interacting member proteins of a protein complex may be used as competitive inhibitors of the activities of the protein complex. As will be apparent to skilled artisans, derivatives or homologues of the binding domains may also be used. Binding domains can be easily identified using molecular biology techniques, e.g., mutagenesis in combination with yeast two-hybrid assays. Preferably, the protein fragment used is a fragment of an interacting protein member having a length of less than 90%, 80%, more preferably less than 75%, 65%, 50%, or less than 40% of the full length of the protein member. In one embodiment, a fragment of a protein identified in the tables above is administered. In a specific embodiment, one or more of the interaction domains of a protein identified in the tables, within the regions listed in the tables, is administered to cells or tissue in vitro, or are administered to a patient in need of such treatment. For example, suitable protein fragments can include polypeptides having a contiguous span of 4, 5, 6, 7, 8, 9, 10, 11, 12, 13, 14, 15, 18, 20 or 25, preferably from 4 to 30, 40 or 50 amino acids or more of the sequence of a first protein identified in the tables, that are capable of interacting with a second protein described in the tables. Also, suitable protein fragments can include peptides capable of binding one or more of the proteins described in the tables, and having an amino acid sequence of from 4 to 30 amino acids that is at least 75%, 80%, 82%, 85%, 87%, 90%, 95% or more identical to a contiguous span of amino acids of a protein described in the tables. Alternatively, a polypeptide capable of interacting with a first protein of an interacting pair of proteins of the present invention, and having a contiguous span of 4, 5, 6, 7, 8, 9, 10, 11, 12, 13, 14, 15, 18, 20 or 25, preferably from 4 to 30, 40 or 50 or more amino acids of the amino acid sequence of a second protein of the same interacting pair of proteins, may be administered. Also, other examples of suitable compounds include a peptide capable of binding a first interacting partner of a pair of interacting proteins of the present invention and having an amino acid sequence of from 4 to 30, 40, 50 or more amino acids that is at least 75%, 80%, 82%, 85%, 87%, 90%, 92%, 95% or more identical to a contiguous span of amino acids from a second interacting partner of a pair of interacting proteins of the present invention. In addition, the administered compounds can be an antibody or antibody fragment, preferably a single-chain antibody immunoreactive with any of the proteins listed in the tables, or a protein complex of the present invention. Exemplary interfering protein fragments with respect to the targets identified in these studies are given in SEQ ID NOS. 7-160.

The protein fragments suitable as competitive inhibitors can be delivered into cells by direct cell internalization, receptor mediated endocytosis, or via a “transporter.” It is noted that when the target proteins or protein complexes to be modulated reside inside cells, the compound administered to cells in vitro or in vivo in the method of the present invention preferably is delivered into the cells in order to achieve optimal results. Thus, preferably, the compound to be delivered is associated with a transporter capable of increasing the uptake of the compound by cells harboring the target protein or protein complex. As used herein, the term “transporter” refers to an entity (e.g., a compound or a composition or a physical structure formed from multiple copies of a compound or multiple different compounds) that is capable of facilitating the uptake of a compound of the present invention by animal cells, particularly human cells. That is, the cell uptake of a compound of the present invention in the presence of a “transporter” is at least 50% higher than the cell uptake of the compound in the absence of the “transporter.” Preferably, a “transporter” is selected such that the cell uptake of a compound of the present invention in the presence of a “transporter” is at least 75% higher, preferably at least 100% or 200% higher, and more preferably at least 300%, 400% or 500% higher than the cell uptake of the compound in the absence of the “transporter.” Methods of assaying cell uptake of a compound should be apparent to skilled artisans. For example, the compound to be delivered can be labeled with a radioactive isotope or another detectable marker (e.g., a fluorescence marker), and added to cultured cells in the presence or absence of a transporter, and incubated for a time period sufficient to allow maximal uptake. Cells can then be separated from the culture medium and the detectable signal (e.g., radioactivity) caused by the compound inside the cells can be measured. The result obtained in the presence of a transporter can be compared to that obtained in the absence of a transporter.

Many molecules and structures known in the art can be used as “transporters.” In one embodiment, a penetratin is used as a transporter. For example, the homeodomain of Antennapedia, a Drosophila transcription factor, can be used as a transporter to deliver a compound of the present invention. Indeed, any suitable member of the penetratin class of peptides can be used to carry a compound of the present invention into cells. Penetratins are disclosed in, e.g., Derossi et al, Trends Cell Biol., 8:84-87 (1998), which is incorporated herein by reference. Penetratins transport molecules attached thereto across cytoplasmic membranes or nuclear membranes efficiently, in a receptor-independent, energy-independent, and cell type-independent manner. Methods for using a penetratin as a carrier to deliver oligonucleotides and polypeptides are also disclosed in U.S. Pat. No. 6,080,724; Pooga et al., Nat. Biotech., 16:857 (1998); and Schutze et al., J. Immunol., 157:650 (1996), all of which are incorporated herein by reference. U.S. Pat. No. 6,080,724 defines the minimal requirements for a penetratin peptide as a peptide of 16 amino acids with 6 to 10 of which being hydrophobic. The amino acid at position 6 counting from either the N- or C-terminus is tryptophan, while the amino acids at positions 3 and 5 counting from either the N- or C-terminus are not both valine. Preferably, the helix 3 of the homeodomain of Drosophila Antennapedia is used as a transporter. More preferably, a peptide having a sequence of amino acid residues 43-58 of the homeodomain Antp is employed as a transporter. In addition, other naturally occurring homologs of the helix 3 of the homeodomain of Drosophila Antennapedia can be used. For example, homeodomains of Fushi-tarazu and Engrailed have been shown to be capable of transporting peptides into cells.

See Han et al., Mol. Cells, 10:728-32 (2000). As used herein, the term “penetratin” also encompasses peptoid analogs of the penetratin peptides. Typically, the penetratin peptides and peptoid analogs thereof are covalently linked to a compound to be delivered into cells thus increasing the cellular uptake of the compound.

In another embodiment, the HIV-1 tat protein or a derivative thereof is used as a “transporter” covalently linked to a compound according to the present invention. The use of HIV-1 tat protein and derivatives thereof to deliver macromolecules into cells has been known in the art. See Green and Loewenstein, Cell, 55:1179 (1988); Frankel and Pabo, Cell, 55:1189 (1988); Vives et al., J. Biol. Chem., 272:16010-16017 (1997); Schwarze et al., Science, 285:1569-1572 (1999). It is known that the sequence responsible for cellular uptake consists of the highly basic region, amino acid residues 49-57. See e.g., Vives et al., J. Biol. Chem., 272:16010-16017 (1997); Wender et al., Proc. Nat'l Acad. Sci. USA, 97:13003-13008 (2000). The basic domain is believed to target the lipid bilayer component of cell membranes. It causes a covalently linked protein or nucleic acid to cross cell membrane rapidly in a cell type-independent manner. Proteins ranging in size from 15 to 120 kD have been delivered with this technology into a variety of cell types both in vitro and in vivo. See Schwarze et al., Science, 285:1569-1572 (1999). Any HIV tat-derived peptides or peptoid analogs thereof capable of transporting macromolecules such as peptides can be used for purposes of the present invention. For example, any native tat peptides having the highly basic region, amino acid residues 49-57 can be used as a transporter by covalently linking it to the compound to be delivered. In addition, various analogs of the tat peptide of amino acid residues 49-57 can also be useful transporters for purposes of this invention. Examples of various such analogs are disclosed in Wender et al., Proc. Nat'l Acad. Sci. USA, 97:13003-13008 (2000) (which is incorporated herein by reference) including, e.g., d-Tat₄₉₋₅₇, retro-inverso isomers of l- or d-Tat₄₉₋₅₇ (i.e., l-Tat₅₇₋₄₉ and d-Tat₅₇₋₄₉), L-arginine oligomers, D-arginine oligomers, L-lysine oligomers, D-lysine oligomers, L-histine oligomers, D-histine oligomers, L-ornithine oligomers, D-ornithine oligomers, and various homologues, derivatives (e.g., modified forms with conjugates linked to the small peptides) and peptoid analogs thereof. Preferably, arginine oligomers are preferred to the other oligomers, since arginine oligomers are much more efficient in promoting cellular uptake. As used herein, the term “oligomer” means a molecule that includes a covalently linked chain of amino acid residues of the same amino acids having a large enough number of such amino acid residues to confer transporter activities on the molecule. Typically, an oligomer contains at least 6, preferably at least 7, 8, or 9 such amino acid residues. In one embodiment, the transporter is a peptide that includes at least six contiguous amino acid residues that are a combination of two or more of L-arginine, D-arginine, L-lysine, D-lysine, L-histidine, D-histine, L-ornithine, and D-ornithine.

Other useful transporters known in the art include, but are not limited to, short peptide sequences derived from fibroblast growth factor (See Lin et al., J. Biol. Chem., 270:14255-14258 (1998)), Galparan (See Pooga et al., FASEB J. 12:67-77 (1998)), and HSV-1 structural protein VP22 (See Elliott and O'Hare, Cell, 88:223-233 (1997)).

As the above-described various transporters are generally peptides, fusion proteins can be conveniently made by recombinant expression to contain a transporter peptide covalently linked by a peptide bond to a competitive protein fragment. Alternatively, conventional methods can be used to chemically synthesize a transporter peptide or a peptide of the present invention or both.

The hybrid peptide can be administered to cells or tissue in vitro or to a patient in a suitable pharmaceutical composition as provided in Section 8.

In addition to peptide-based transporters, various other types of transporters can also be used, including but not limited to cationic liposomes (see Rui et al., J. Am. Chem. Soc., 120:11213-11218 (1998)), dendrimers (Kono et al., Bioconjugate Chem., 10:1115-1121 (1999)), siderophores (Ghosh et al., Chem. Biol., 3:1011-1019 (1996)), etc. In a specific embodiment, the compound according to the present invention is encapsulated into liposomes for delivery into cells.

Additionally, when a compound according to the present invention is a peptide, it can be administered to cells by a gene therapy method. That is, a nucleic acid encoding the peptide can be administered to in vitro cells or to cells in vivo in a human or animal body. Any suitable gene therapy methods may be used for purposes of the present invention. Various gene therapy methods are well known in the art and are described in Section 6.3.2. below. Successes in gene therapy have been reported recently. See e.g., Kay et al., Nature Genet., 24:257-61 (2000); Cavazzana-Calvo et al., Science, 288:669 (2000); and Blaese et al., Science, 270: 475 (1995); Kantoff, et al., J. Exp. Med., 166:219 (1987).

In yet another embodiment, the gene therapy methods discussed in Section 6.3.2 below are used to “knock out” the gene encoding an interacting protein member of a protein complex, or to reduce the gene expression level. For example, the gene may be replaced with a different gene sequence or a non-functional sequence or simply deleted by homologous recombination. In another gene therapy embodiment, the method disclosed in U.S. Pat. No. 5,641,670, which is incorporated herein by reference, may be used to reduce the expression of the genes for the interacting protein members. Essentially, an exogenous DNA having at least a regulatory sequence, an exon and a splice donor site can be introduced into an endogenous gene encoding an interacting protein member by homologous recombination such that the regulatory sequence, the exon and the splice donor site present in the DNA construct become operatively linked to the endogenous gene. As a result, the expression of the endogenous gene is controlled by the newly introduced exogenous regulatory sequence. Therefore, when the exogenous regulatory sequence is a strong gene expression repressor, the expression of the endogenous gene encoding the interacting protein member is reduced or blocked. See U.S. Pat. No. 5,641,670.

### 6.3. Activation of Protein Complex or Interacting Protein Members Thereof

The present invention also provides methods for increasing in cells or tissue in vitro or in a patient the concentration and/or activity of a protein complex, or of an individual protein member thereof, identified in accordance with the present invention. Such methods can be particularly useful in instances where a reduced concentration and/or activity of a protein complex, or a protein member thereof, is associated with a particular disease or disorder to be treated, or where an increased concentration and/or activity of a protein complex, or a protein member thereof, would be beneficial to the improvement of a cellular function or disease state. By increasing the concentration of the protein complex, or a protein member thereof, and/or stimulating the functional activities of the protein complex or a protein member thereof, the disease or disorder may be treated or prevented.

### 6.3.1. Administration of Protein Complex or Protein Members Thereof

Where the concentration or activity of a particular protein complex of the present invention, or any individual protein constituent of a protein complex in cells or tissue in vitro or in a patient is determined to be low or is desired to be increased, the protein complex, or an individual constituent protein of the protein complex may be administered directly to the patient to increase the concentration and/or activity of the protein complex, or the individual constituent protein. For this purpose, protein complexes prepared by any one of the methods described in Section 2.2 may be administered to the patient, preferably in a pharmaceutical composition as described below. Alternatively, one or more individual interacting protein members of the protein complex may also be administered to the patient in need of treatment. For example, one or more of the individual proteins or the interacting pairs of proteins described in the tables may be given to cells or tissue in vitro or to a patient. Proteins isolated or purified from normal individuals or recombinantly produced can all be used in this respect. Preferably, two or more interacting protein members of a protein complex are administered. The proteins or protein complexes may be administered to a patient needing treatment using any of the methods described in Section 8.

### 6.3.2. Gene Therapy

In another embodiment, the concentration and/or activity of a particular protein complex comprising one or more of the interacting pairs of proteins described in the tables or an individual constituent protein of a protein complex of the present invention is increased or restored in patients, tissue or cells by a gene therapy approach. For example, nucleic acids encoding one or more protein members of a protein complex of the present invention, or portions or fragments thereof are introduced into patients, tissue, or cells such that the protein(s) are expressed from the introduced nucleic acids. For these purposes, nucleic acids encoding one or more of the proteins described in the tables, or fragments, homologues or derivatives thereof can be used in the gene therapy in accordance with the present invention. For example, if a disease-causing mutation exists in one of the protein members in cells or tissue in vitro or in a patient, then a nucleic acid encoding a wild-type protein can be introduced into tissue cells of the patient. The exogenous nucleic acid can be used to replace the corresponding endogenous defective gene by, e.g., homologous recombination. See U.S. Pat. No. 6,010,908, which is incorporated herein by reference. Alternatively, if the disease-causing mutation is a recessive mutation, the exogenous nucleic acid is simply used to express a wild-type protein in addition to the endogenous mutant protein. In another approach, the method disclosed in U.S. Pat. No. 6,077,705 may be employed in gene therapy. That is, the patient is administered both a nucleic acid construct encoding a ribozyme and a nucleic acid construct comprising a ribozyme resistant gene encoding a wild type form of the gene product. As a result, undesirable expression of the endogenous gene is inhibited and a desirable wild-type exogenous gene is introduced. In yet another embodiment, if the endogenous gene is of wild-type and the level of expression of the protein encoded thereby is desired to be increased, additional copies of wild-type exogenous genes may be introduced into the patient by gene therapy, or alternatively, a gene activation method such as that disclosed in U.S. Pat. No. 5,641,670 may be used.

Various gene therapy methods are well known in the art. Successes in gene therapy have been reported recently. See e.g., Kay et al., Nature Genet., 24:257-61 (2000); Cavazzana-Calvo et al., Science, 288:669 (2000); and Blaese et al., Science, 270: 475 (1995); Kantoff, et al., J. Exp. Med. 166:219 (1987).

Any suitable gene therapy methods may be used for the purposes of the present invention. Generally, a nucleic acid encoding a desirable protein (e.g., one selected from any of the tables) is incorporated into a suitable expression vector and is operably linked to a promoter in the vector. Suitable promoters include but are not limited to viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Where tissue-specific expression of the exogenous gene is desirable, tissue-specific promoters may be operably linked to the exogenous gene. In addition, selection markers may also be included in the vector for purposes of selecting, in vitro, those cells that contain the exogenous gene. Various selection markers known in the art may be used including, but not limited to, e.g., genes conferring resistance to neomycin, hygromycin, zeocin, and the like.

In one embodiment, the exogenous nucleic acid (gene) is incorporated into a plasmid DNA vector. Many commercially available expression vectors may be useful for the present invention, including, e.g., pCEP4, pcDNAI, pIND, pSecTag2, pVAX1, pcDNA3.1, and pBI-EGFP, and pDisplay.

Various viral vectors may also be used. Typically, in a viral vector, the viral genome is engineered to eliminate the disease-causing capability of the virus, e.g., the ability to replicate in the host cells. The exogenous nucleic acid to be introduced into cells or tissue in vitro or in a patient may be incorporated into the engineered viral genome, e.g., by inserting it into a viral gene that is non-essential to the viral infectivity. Viral vectors are convenient to use as they can be easily introduced into cells, tissues and patients by way of infection. Once in the host cell, the recombinant virus typically is integrated into the genome of the host cell. In rare instances, the recombinant virus may also replicate and remain as extrachromosomal elements.

A large number of retroviral vectors have been developed for gene therapy. These include vectors derived from oncoretroviruses (e.g., MLV), lentiviruses (e.g., HIV and SIV) and other retroviruses. For example, gene therapy vectors have been developed based on murine leukemia virus (See, Cepko, et al., Cell, 37:1053-1062 (1984), Cone and Mulligan, Proc. Natl. Acad. Sci. U.S.A., 81:6349-6353 (1984)), mouse mammary tumor virus (See, Salmons et al., Biochem. Biophys. Res. Commun., 159:1191-1198 (1984)), gibbon ape leukemia virus (See, Miller et al., J. Virology, 65:2220-2224 (1991)), HIV, (See Shimada et al., J. Clin. Invest., 88:1043-1047 (1991)), and avianretroviruses (See Cosset et al., J. Virology, 64:1070-1078 (1990)). In addition, various retroviral vectors are also described in U.S. Pat. Nos. 6,168,916; 6,140,111; 6,096,534; 5,985,655; 5,911,983; 4,980,286; and 4,868,116, all of which are incorporated herein by reference.

Adeno-associated virus (AAV) vectors have been successfully tested in clinical trials. See e.g., Kay et al., Nature Genet. 24:257-61 (2000). AAV is a naturally occurring defective virus that requires other viruses such as adenoviruses or herpes viruses as helper viruses. See Muzyczka, Curr. Top. Microbiol. Immun., 158:97 (1992). A recombinant AAV virus useful as a gene therapy vector is disclosed in U.S. Pat. No. 6,153,436, which is incorporated herein by reference.

Adenoviral vectors can also be useful for purposes of gene therapy in accordance with the present invention. For example, U.S. Pat. No. 6,001,816 discloses an adenoviral vector, which is used to deliver a leptin gene intravenously to a mammal to treat obesity. Other recombinant adenoviral vectors may also be used, which include those disclosed in U.S. Pat. Nos. 6,171,855; 6,140,087; 6,063,622; 6,033,908; and 5,932,210, and Rosenfeld et al., Science, 252:431-434 (1991); and Rosenfeld et al., Cell, 68:143-155 (1992).

Other useful viral vectors include recombinant hepatitis viral vectors (See, e.g., U.S. Pat. No. 5,981,274), and recombinant entomopox vectors (See, e.g., U.S. Pat. Nos. 5,721,352 and 5,753,258).

Other non-traditional vectors may also be used for purposes of this invention. For example, International Publication No. WO 94/18834 discloses a method of delivering DNA into mammalian cells by conjugating the DNA to be delivered with a polyelectrolyte to form a complex. The complex may be microinjected into or taken up by cells.

The exogenous gene fragment or plasmid DNA vector containing the exogenous gene may also be introduced into cells by way of receptor-mediated endocytosis. See e.g., U.S. Pat. No. 6,090,619; Wu and Wu, J. Biol. Chem., 263:14621 (1988); Curiel et al., Proc. Natl. Acad. Sci. USA, 88:8850 (1991). For example, U.S. Pat. No. 6,083,741 discloses introducing an exogenous nucleic acid into mammalian cells by associating the nucleic acid to a polycation moiety (e.g., poly-L-lysine having 3-100 lysine residues), which is itself coupled to an integrin receptor binding moiety (e.g., a cyclic peptide having the sequence Arg-Gly-Asp).

Alternatively, the exogenous nucleic acid or vectors containing it can also be delivered into cells via amphiphiles. See e.g., U.S. Pat. No. 6,071,890. Typically, the exogenous nucleic acid or a vector containing the nucleic acid forms a complex with the cationic amphiphile. Mammalian cells contacted with the complex can readily take it up.

The exogenous gene can be introduced into cells or tissue in vitro or in a patient for purposes of gene therapy by various methods known in the art. For example, the exogenous gene sequences alone or in a conjugated or complex form described above, or incorporated into viral or DNA vectors, may be administered directly by injection into an appropriate tissue or organ of a patient. Alternatively, catheters or like devices may be used to deliver exogenous gene sequences, complexes, or vectors into a target organ or tissue. Suitable catheters are disclosed in, e.g., U.S. Pat. Nos. 4,186,745; 5,397,307; 5,547,472; 5,674,192; and 6,129,705, all of which are incorporated herein by reference.

In addition, the exogenous gene or vectors containing the gene can be introduced into isolated cells using any known techniques such as calcium phosphate precipitation, microinjection, lipofection, electroporation, biolystics, receptor-mediated endocytosis, and the like. Cells expressing the exogenous gene may be selected and redelivered back to the patient by, e.g., injection or cell transplantation. The appropriate amount of cells delivered to a patient will vary with patient conditions, and desired effect, which can be determined by a skilled artisan. See e.g., U.S. Pat. Nos. 6,054,288; 6,048,524; and 6,048,729. Preferably, the cells used are autologous, i.e., cells obtained from the patient being treated.

### 6.4. Small Organic Compounds

Diseases or disorders in cells or tissue in vitro, or in a patient, associated with the decreased concentration or activity of a protein complex of the present invention, or an individual protein constituent of a protein complex identified in accordance with the present invention, can also be ameliorated by administering to the patient a compound identified by the methods described in Sections 5.3.1.4, 5.2, and Section 5.4, which is capable of modulating the functions or intracellular levels of the protein complex or a constituent protein, e.g., by triggering or initiating, enhancing or stabilizing protein-protein interaction between the interacting protein members of the protein complex, or the mutant forms of such interacting protein members found in the patient.

### 7. Cell and Animal Models

In another aspect of the present invention, cell and animal models are provided in which one or more of the constituent proteins of the interacting pairs of proteins described in the tables, exhibit aberrant function, activity, or concentration when compared with wild type cells and animals (e.g., increased or decreased concentration, altered interactions between protein complex constituents due to mutations in interaction domains, and/or altered distribution or localization of the proteins in organs, tissues, cells, or cellular compartments). Such cell and animal models are useful tools for studying cellular functions and biological processes associated with the proteins identified in the tables. Such cell and animal models are also useful tools for studying disorders and diseases associated with the proteins identified in the tables, and for testing various methods for modulating the cellular functions, and for treating the diseases and disorders, associated with aberrations in these proteins. For example, a cell or animal model may be used to determine if BAT3 exhibits aberrant function, activity, or concentration when compared with wild type cells or animals. In another example, a cell or animal model may be used to determine if PN9113 exhibits aberrant function, activity, or concentration when compared with wild type cells or animals.

### 7.1. Cell Models

Cell models having an aberrant form of one or more of the proteins or protein complexes identified in the tables are provided in accordance with the present invention.

The cell models may be established by isolating, from a patient, cells having an aberrant form of one or more of the protein complexes of the present invention. The isolated cells may be cultured in vitro as a primary cell culture. Alternatively, the cells obtained from the primary cell culture or directly from the patient may be immortalized to establish a human cell line. Any methods for constructing immortalized human cell lines may be used in this respect. See generally Yeager and Reddel, Curr. Opini. Biotech., 10:465-469 (1999). For example, the human cells may be immortalized by transfection of plasmids expressing the SV40 early region genes (See e.g., Jha et al., Exp. Cell Res., 245:1-7 (1998)), introduction of the HPV E6 and E7 oncogenes (See e.g., Reznikoff et al., Genes Dev., 8:2227-2240 (1994)), and infection with Epstein-Barr virus (See e.g., Tahara et al., Oncogene, 15:1911-1920 (1997)). Alternatively, the human cells may be immortalized by recombinantly expressing the gene for the human telomerase catalytic subunit hTERT in the human cells. See Bodnar et al., Science, 279:349-352 (1998).

In alternative embodiments, cell models are provided by recombinantly manipulating appropriate host cells. The host cells may be bacteria cells, yeast cells, insect cells, plant cells, animal cells, and the like. Preferably, the cells are derived from mammals, most preferably humans. The host cells may be obtained directly from an individual, or a primary cell culture, or preferably an immortal stable human cell line. In a preferred embodiment, human embryonic stem cells or pluripotent cell lines derived from human stem cells are used as host cells. Methods for obtaining such cells are disclosed in, e.g., Shamblott, et al., Proc. Natl. Acad. Sci. USA, 95:13726-13731 (1998) and Thomson et al., Science, 282:1145-1147 (1998).

In one embodiment, a cell model is provided by recombinantly expressing one or more of the proteins or protein complexes identified in the tables in cells that do not normally express such protein complexes. For example, cells that do not contain a particular protein or protein complex may be engineered to express the protein or protein complex. In a specific embodiment, a particular human protein complex is expressed in non-human cells. The cell model may be prepared by introducing into host cells nucleic acids encoding all interacting protein members required for the formation of a particular protein complex, and expressing the protein members in the host cells. For this purpose, the recombinant expression methods described in Section 2.2 may be used. In addition, the methods for introducing nucleic acids into host cells disclosed in the context of gene therapy in Section 6.3.2 may also be used.

In another embodiment, a cell model over-expressing one or more of the proteins or protein complexes identified in the tables. The cell model may be established by increasing the expression level of one or more of the interacting protein members of the protein complexes. In a specific embodiment, all interacting protein members of a particular protein complex are over-expressed. The over-expression may be achieved by introducing into host cells exogenous nucleic acids encoding the proteins to be over-expressed, and selecting those cells that over-express the proteins. The expression of the exogenous nucleic acids may be transient or, preferably stable. The recombinant expression methods described in Section 2.2, and the methods for introducing nucleic acids into host cells disclosed in the context of gene therapy in Section 6.3.2 may be used. Alternatively, the gene activation method disclosed in U.S. Pat. No. 5,641,670 can be used. Any host cells may be employed for establishing the cell model. Preferably, human cells lacking a protein or protein complex to be over-expressed, or having a normal concentration of the protein or protein complex, are used as host cells. The host cells may be obtained directly from an individual, or a primary cell culture, or preferably a stable immortal human cell line. In a preferred embodiment, human embryonic stem cells or pluripotent cell lines derived from human stem cells are used as host cells. Methods for obtaining such cells are disclosed in, e.g., Shamblott, et al., Proc. Natl. Acad. Sci. USA, 95:13726-13731 (1998), and Thomson et al., Science, 282:1145-1147 (1998).

In yet another embodiment, a cell model expressing an abnormally low level of one or more of the proteins or protein complexes identified in the tables is provided. Typically, the cell model is established by genetically manipulating cells that express a normal and detectable level of a protein or protein complex identified in the tables. Generally the expression level of one or more of the interacting protein members of the protein complex is reduced by recombinant methods. In a specific embodiment, the expression of all interacting protein members of a particular protein complex is reduced. The reduced expression may be achieved by “knocking out” the genes encoding one or more interacting protein members. Alternatively, mutations that can cause reduced expression level (e.g., reduced transcription and/or translation efficiency, and decreased mRNA stability) may also be introduced into the gene by homologous recombination. A gene encoding a ribozyme, antisense, or siRNA compound specific to the mRNA encoding an interacting protein member may also be introduced into the host cells, preferably stably integrated into the genome of the host cells. In addition, a gene encoding an antibody or fragment thereof specific to an interacting protein member may also be introduced into the host cells. The recombinant expression methods described in Sections 2.2, 6.1 and 6.2 can all be used for purposes of manipulating the host cells.

In a specific embodiment, an siRNA compound specific to the mRNA encoding BAT3 is introduced into a host cell in order to decrease the expression level of BAT3. In another specific embodiment, an siRNA compound specific to the mRNA encoding PN9113 is introduced into a host cell in order to decrease the expression level of PN9113.

The present invention also contemplates a cell model provided by recombinant DNA techniques that exhibits aberrant interactions between the interacting protein members of a protein complex identified in the present invention. For example, variants of the interacting protein members of a particular protein complex exhibiting altered protein-protein interaction properties and the nucleic acid variants encoding such variant proteins may be obtained by random or site-directed mutagenesis in combination with a protein-protein interaction assay system, particularly the yeast two-hybrid system described in Section 5.3.1. Essentially, the genes encoding one or more interacting protein members of a particular protein complex may be subject to random or site-specific mutagenesis and the mutated gene sequences are used in yeast two-hybrid system to test the protein-protein interaction characteristics of the protein variants encoded by the gene variants. In this manner, variants of the interacting protein members of the protein complex may be identified that exhibit altered protein-protein interaction properties in forming the protein complex, e.g., increased or decreased binding affinity, and the like. The nucleic acid variants encoding such protein variants may be introduced into host cells by the methods described above, preferably into host cells that normally do not express the interacting proteins.

### 7.2. Cell-Based Assays

The cell models of the present invention containing an aberrant form of a protein or protein complex identified in the tables are useful in screening assays for identifying compounds useful in treating diseases and disorders involving neurotransmission, regulation of APP production and APP metabolism, regulation of Aβ production and Aβ metabolism, processing of proteins destined for secretion, regulation of the wingless pathway, and modulation of thyroid receptor signaling such as Alzheimer's disease (including mild cognitive impairment), Huntington's disease, Parkinson's disease, schizophrenia, depression, dementia and other neurodegenerative diseases, as well as diabetes, obesity and coronary artery disease. In addition, they may also be used in in vitro pre-clinical assays for testing compounds, such as those identified in the screening assays of the present invention.

For example, cells may be treated with compounds to be tested and assayed for the compound's activity. A variety of parameters relevant to particularly physiological disorders or diseases may be analyzed.

### 7.3. Transgenic Animals

In another aspect of the present invention, transgenic non-human animals are created expressing an aberrant form of one or more of the protein complexes of the present invention. Animals of any species may be used to generate the transgenic animal models, including but not limited to, mice, rats, hamsters, sheep, pigs, rabbits, guinea pigs, preferably non-human primates such as monkeys, chimpanzees, baboons, and the like.

In one embodiment, transgenic animals are made to over-express one or more protein complexes formed from a first protein, which is any one of the proteins described in the tables, or a derivative, fragment or homologue thereof (including the animal counterpart of the first protein, i.e., an orthologue) and a second protein, which is any of the proteins described in the tables that interacts with the first protein, or derivatives, fragments or homologues thereof (including orthologues). Over-expression may be directed in a tissue or cell type that normally expresses animal counterparts of such protein complexes. Consequently, the concentration of the protein complex(es) will be elevated to higher levels than normal. Alternatively, the one or more protein complexes are expressed in tissues or cells that do not normally express such proteins and hence do not normally contain the protein complexes of the present invention. In a specific embodiment, a first protein, which is any one of the proteins described in the tables which is a human protein and a second protein, which is any of the proteins described in the tables that interacts with the first protein and is a human protein, are expressed in the transgenic animals.

To achieve over-expression in transgenic animals, the transgenic animals are made such that they contain and express exogenous, orthologous genes encoding a first protein, which is any of the proteins identified in the tables or a homologue, derivative or mutant form thereof, and one or more second proteins, which are any of the proteins described in the tables that interact with the first protein, or homologues, derivatives or mutant forms thereof. Preferably, the exogenous genes are human genes. Such exogenous genes may be operably linked to a native or non-native promoter, preferably a non-native promoter. For example, an exogenous gene encoding one of the proteins described in the tables may be operably linked to a promoter that is not the native promoter of that protein. If the expression of the exogenous gene is desired to be limited to a particular tissue, an appropriate tissue-specific promoter may be used.

Over-expression may also be achieved by manipulating the native promoter to create mutations that lead to gene over-expression, or by a gene activation method such as that disclosed in U.S. Pat. No. 5,641,670 as described above.

In another embodiment, the transgenic animal expresses an abnormally low concentration of the complex comprising at least one of the interacting pairs of proteins described in the tables. In a specific embodiment, the transgenic animal is a “knockout” animal wherein the endogenous gene encoding the animal orthologue of a first protein, which is any of the proteins described in the tables, and/or an endogenous gene encoding an animal orthologue of a second protein, which is any of the proteins identified in the tables that interacts with the first protein, are knocked out. In a specific embodiment, the expression of the animal orthologues of both the first and second proteins are reduced or knocked out. The reduced expression may be achieved by knocking out the genes encoding one or both interacting protein members, typically by homologous recombination. Alternatively, mutations that can cause reduced expression (e.g., reduced transcription and/or translation efficiency, or decreased mRNA stability) may also be introduced into the endogenous genes by homologous recombination. Genes encoding ribozymes or antisense compounds specific to the mRNAs encoding the interacting protein members may also be introduced into the transgenic animal. In addition, genes encoding antibodies or fragments thereof specific to the interacting protein members may also be introduced into the transgenic animal.

In an alternate embodiment, transgenic animals are made in which the endogenous genes encoding the animal orthologues of any of the proteins described in the tables are replaced with orthologous human genes.

In yet another embodiment, the transgenic animal of this invention expresses specific mutant forms of any of the proteins described in the tables that exhibit aberrant interactions. For this purpose, variants of any of the proteins described in the tables exhibiting altered protein-protein interaction properties, and the nucleic acid variants encoding such variant proteins, may be obtained by random or site-specific mutagenesis in combination with a protein-protein interaction assay system, particularly the yeast two-hybrid system described in Section 5.3.1. For example, variants of BAT3 and PN9113 exhibiting increased, decreased or abolished binding affinity to each other may be identified and isolated. The transgenic animal of the present invention may be made to express such protein variants by modifying the endogenous genes. Alternatively, the nucleic acid variants may be introduced exogenously into the transgenic animal genome to express the protein variants therein. In a specific embodiment, the exogenous nucleic acid variants are derived from orthologous human genes and the corresponding endogenous genes are knocked out.

Any techniques known in the art for making transgenic animals may be used for purposes of the present invention. For example, the transgenic animals of the present invention may be provided by methods described in, e.g., Jaenisch, Science, 240:1468-1474 (1988); Capecchi, et al., Science, 244:1288-1291 (1989); Hasty et al., Nature, 350:243 (1991); Shinkai et al., Cell, 68:855 (1992); Mombaerts et al., Cell, 68:869 (1992); Philpott et al., Science, 256:1448 (1992); Snouwaert et al., Science, 257:1083 (1992); Donehower et al., Nature, 356:215 (1992); Hogan et al., Manipulating the Mouse Embryo; A Laboratory Manual, 2^{nd} edition, Cold Spring Harbor Laboratory Press, 1994; and U.S. Pat. Nos. 4,873,191; 5,800,998; 5,891,628, all of which are incorporated herein by reference.

Generally, the founder lines may be established by introducing appropriate exogenous nucleic acids into, or modifying an endogenous gene in, germ lines, embryonic stem cells, embryos, or sperm which are then used in producing a transgenic animal. The gene introduction may be conducted by various methods including those described in Sections 2.2, 6.1 and 6.2. See also, Van der Putten et al., Proc. Natl. Acad. Sci. USA, 82:6148-6152 (1985); Thompson et al., Cell, 56:313-321 (1989); Lo, Mol. Cell. Biol., 3:1803-1814 (1983); Gordon, Transgenic Animals, Intl. Rev. Cytol. 115:171-229 (1989); and Lavitrano et al., Cell, 57:717-723 (1989). In a specific embodiment, the exogenous gene is incorporated into an appropriate vector, such as those described in Sections 2.2 and 6.2, and is transformed into embryonic stem (ES) cells. The transformed ES cells are then injected into a blastocyst. The blastocyst with the transformed ES cells is then implanted into a surrogate mother animal. In this manner, a chimeric founder line animal containing the exogenous nucleic acid (transgene) may be produced.

Preferably, site-specific recombination is employed to integrate the exogenous gene into a specific predetermined site in the animal genome, or to replace an endogenous gene or a portion thereof with the exogenous sequence. Various site-specific recombination systems may be used including those disclosed in Sauer, Curr. Opin. Biotechnol., 5:521-527 (1994); Capecchi, et al, Science, 244:1288-1291 (1989); and Gu et al., Science, 265:103-106 (1994). Specifically, the Cre/lox site-specific recombination system known in the art may be conveniently used which employs the bacteriophage P1 protein Cre recombinase and its recognition sequence loxP. See Rajewsky et al., J. Clin. Invest., 98:600-603 (1996); Sauer, Methods, 14:381-392 (1998); Gu et al., Cell, 73:1155-1164 (1993); Araki et al., Proc. Natl. Acad. Sci. USA, 92:160-164 (1995); Lakso et al., Proc. Natl. Acad. Sci. USA, 89:6232-6236 (1992); and Orban et al., Proc. Natl. Acad. Sci. USA, 89:6861-6865 (1992).

The transgenic animals of the present invention may be transgenic animals that carry a transgene in all cells or mosaic transgenic animals carrying a transgene only in certain cells, e.g., somatic cells. The transgenic animals may have a single copy or multiple copies of a particular transgene.

The founder transgenic animals thus produced may be bred to produce various offsprings. For example, they can be inbred, outbred, and crossbred to establish homozygous lines, heterozygous lines, and compound homozygous or heterozygous lines.

### 8. Pharmaceutical Compositions and Formulations

In another aspect of the present invention, pharmaceutical compositions are also provided containing one or more of the therapeutic agents provided in the present invention as described in Section 6. The compositions are prepared as a pharmaceutical formulation suitable for administration into a patient. Accordingly, the present invention also extends to pharmaceutical compositions, medicaments, drugs or other compositions containing one or more of the therapeutic agent in accordance with the present invention.

For example, such therapeutic agents include, but are not limited to, (1) small organic compounds selected based on the screening methods of the present invention capable of interfering with the interaction between a first protein which is any of the interacting proteins described in the tables and a second protein which is any of the proteins identified in the tables that interacts with the first protein, (2) antisense compounds specifically hybridizable to nucleic acids (gene or mRNA) encoding the first protein (3) antisense compounds specific to the gene or mRNA encoding the second protein, (4) ribozyme compounds specific to nucleic acids (gene or mRNA) encoding the first protein, (5) ribozyme compounds specific to the gene or mRNA encoding the second protein, (6) antibodies immunoreactive with the first protein or the second protein, (7) antibodies selectively immunoreactive with a protein complex of the present invention, (8) small organic compounds capable of binding a protein complex of the present invention, (9) small peptide compounds as described above (optionally linked to a transporter) capable of interacting with the first protein or the second protein, (10) nucleic acids encoding the antibodies or peptides, (11) siRNA compounds specific to the gene or mRNA encoding the first protein, (12) siRNA compounds specific to the gene or mRNA encoding the second protein, etc.

The compositions are prepared as a pharmaceutical formulation suitable for administration into a patient. Accordingly, the present invention also extends to pharmaceutical compositions, medicaments, drugs or other compositions containing one or more of the therapeutic agent in accordance with the present invention.

In the pharmaceutical composition, an active compound identified in accordance

with the present invention can be in any pharmaceutically acceptable salt form. As used herein, the term “pharmaceutically acceptable salts” refers to the relatively non-toxic, organic or inorganic salts of the compounds of the present invention, including inorganic or organic acid addition salts of the compound. Examples of such salts include, but are not limited to, hydrochloride salts, sulfate salts, bisulfate salts, borate salts, nitrate salts, acetate salts, phosphate salts, hydrobromide salts, laurylsulfonate salts, glucoheptonate salts, oxalate salts, oleate salts, laurate salts, stearate salts, palmitate salts, valerate salts, benzoate salts, naphthylate salts, mesylate salts, tosylate salts, citrate salts, lactate salts, maleate salts, succinate salts, tartrate salts, fumarate salts, and the like. See, e.g., Berge, et al., J. Pharm. Sci., 66:1-19 (1977).

For oral delivery, the active compounds can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetate, citrate or phosphate buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network that swells in water to form a gel like material. Preferably, hydrogels is biodegradable or biosorbable. For purposes of this invention, hydrogels made of polyethylene glycols, collagen, or poly(glycolic-co-L-lactic acid) may be useful. See, e.g., Phillips et al., J. Pharmaceut. Sci. 73:1718-1720 (1984).

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active compounds can also be administered in combination with another active agent that synergistically treats or prevents the same symptoms or is effective for another disease or symptom in the patient treated so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

Generally, the toxicity profile and therapeutic efficacy of the therapeutic agents can be determined by standard pharmaceutical procedures in cell models or animal models, e.g., those provided in Section 7. As is known in the art, the LD₅₀ represents the dose lethal to about 50% of a tested population. The ED₅₀ is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but significantly below the LD₅₀ obtained from cell or animal models.

It will be apparent to skilled artisans that therapeutically effective amount for each active compound to be included in a pharmaceutical composition of the present invention can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like. The amount of administration can also be adjusted as the various factors change over time.

### EXAMPLES

### 1. Yeast Two-Hybrid System

The principles and methods of the yeast two-hybrid system have been described in detail in The Yeast Two-Hybrid System, Bartel and Fields, eds., pages 183-196, Oxford University Press, New York, N.Y., 1997. The following is thus a description of the particular procedure that we used to identify the interactions of the present invention.

The cDNA encoding the bait protein was generated by PCR from cDNA prepared from a desired tissue. The cDNA product was then introduced by recombination into the yeast expression vector pGBT.Q, which is a close derivative of pGBT.C (See Bartel et al., Nat. Genet., 12:72-77 (1996)) in which the polylinker site has been modified to include M13 sequencing sites. The new construct was selected directly in the yeast strain PNY200 for its ability to drive tryptophane synthesis (genotype of this strain: MATαtrp1-901 leu2-3,112 ura3-52 his3-200 ade2 gal4Δgal80). In these yeast cells, the bait was produced as a C-terminal fusion protein with the DNA binding domain of the transcription factor Gal4 (amino acids 1 to 147). Prey libraries were transformed into the yeast strain BK100 (genotype of this strain: MATαtrp1-901 leu2-3,112 ura3-52 his3-200 gal4Δgal80LYS2::GAL-HIS3 GAL2-ADE2 met2::GAL7-lacZ), and selected for the ability to drive leucine synthesis. In these yeast cells, each cDNA was expressed as a fusion protein with the transcription activation domain of the transcription factor Gal4 (amino acids 768 to 881) and a 9 amino acid hemagglutinin epitope tag. PNY200 cells (MATα mating type), expressing the bait, were then mated with BK100 cells (MATa mating type), expressing prey proteins from a prey library. The resulting diploid yeast cells expressing proteins interacting with the bait protein were selected for the ability to synthesize tryptophan, leucine, histidine, and adenine. DNA was prepared from each clone, transformed by electroporation into E. coli strain KC8 (Clontech KC8 electrocompetent cells, Catalog No. C2023-1), and the cells were selected on ampicillin-containing plates in the absence of either tryptophane (selection for the bait plasmid) or leucine (selection for the library plasmid). DNA for both plasmids was prepared and sequenced by the dideoxynucleotide chain termination method. The identity of the bait cDNA insert was confirmed and the cDNA insert from the prey library plasmid was identified using the BLAST program to search against public nucleotide and protein databases. Plasmids from the prey library were then individually transformed into yeast cells together with a plasmid driving the synthesis of lamin and 5 other test proteins, respectively, fused to the Gal4 DNA binding domain. Clones that gave a positive signal in the β-galactosidase assay were considered false-positives and discarded. Plasmids for the remaining clones were transformed into yeast cells together with the original bait plasmid. Clones that gave a positive signal in the β-galactosidase assay were considered true positives.

Bait sequences indicated in the tables were used in the yeast two-hybrid system described above. The isolated prey sequences are summarized in the tables. The GenBank Accession Nos. for the bait and prey proteins are also provided in the tables, upon which the bait and prey sequences are aligned.

### 2. Production of Antibodies Selectively Immunoreactive with Protein Complex

The BAT3-interacting region of PN9113 and the PN9113-interacting region of BAT3 are indicated in the tables. Both regions, or fragments thereof, are recombinantly-expressed in E. coli. and isolated and purified. Mixing the two purified interacting regions forms a protein complex. A protein complex is also formed by mixing recombinantly expressed intact complete BAT3 and PN9113. The two protein complexes are used as antigens in immunizing a mouse. mRNA is isolated from the immunized mouse spleen cells, and first-strand cDNA is synthesized using the mRNA as a template. The V_{H} and V_{K} genes are amplified from the thus synthesized cDNAs by PCR using appropriate primers.

The amplified V_{H} and V_{K} genes are ligated together and subcloned into a phagemid vector for the construction of a phage display library. E. coli. cells are transformed with the ligation mixtures, and thus a phage display library is established. Alternatively, the ligated V_{H} and Vₖ genes are subcloned into a vector suitable for ribosome display in which the V_{H}-Vₖ sequence is under the control of a T7 promoter. See Schaffitzel et al., J. Immun. Meth., 231:119-135 (1999).

The libraries are screened for their ability to bind BAT3-PN9113 complex and BAT3 or PN9113, alone. Several rounds of screening are generally performed. Clones corresponding to scFv fragments that bind the BAT3-PN9113 complex, but not isolated BAT3 or PN9113 are selected and purified. A single purified clone is used to prepare an antibody selectively immunoreactive with the complex comprising BAT3 and PN9113. The antibody is then verified by an immunochemistry method such as RIA and ELISA.

In addition, the clones corresponding to scFv fragments that bind the complex comprising BAT3 and PN9113, and also bind isolated BAT3 and/or PN9113 may be selected. The scFv genes in the clones are diversified by mutagenesis methods such as oligonucleotide-directed mutagenesis, error-prone PCR (See Lin-Goerke et al., Biotechniques, 23:409 (1997)), dNTP analogues (See Zaccolo et al., J. Mol. Biol., 255:589 (1996)), and other methods. The diversified clones are further screened in phage display or ribosome display libraries. In this manner, scFv fragments selectively immunoreactive with the complex comprising BAT3 and PN9113 may be obtained.

### 3. Yeast Screen to Identify Small Molecule Inhibitors of the Interaction Between BAT3 and PN9113

Beta-galactosidase is used as a reporter enzyme to signal the interaction between yeast two-hybrid protein pairs expressed from plasmids in Saccharomyces cerevisiae. Yeast strain MY209 (ade2 his3 leu2 trp1 cyh2 ura3::GAL1p-lacZ gal4 gal80 lys2::GAL1p-HIS3) bearing one plasmid with the genotype of LEU2 CEN4 ARS1 ADH1p-SV40NLS-GAL4 (768-881)—PN9113-PGK1t AmpR ColE1_ori, and another plasmid having a genotype of TRP1 CEN4 ARS ADH1p-GAL4(1-147)-BAT3-ADH1t AmpR ColE1_ori is cultured in synthetic complete media lacking leucine and tryptophan (SC-Leu-Trp) overnight at 30° C. The BAT3 and PN9113 nucleic acids in the plasmids can code for the full-length BAT3 and PN9113 proteins, respectively, or fragments thereof. This culture is diluted to 0.01 OD₆₃₀ units/ml using SC-Leu-Trp media. The diluted MY209 culture is dispensed into 96-well microplates. Compounds from a library of small molecules are added to the microplates; the final concentration of test compounds is approximately 60 μM. The assay plates are incubated at 30° C. overnight.

The following day an aliquot of concentrated substrate/lysis buffer is added to each well and the plates incubated at 37° C. for 1-2 hours. At an appropriate time an aliquot of stop solution is added to each well to halt the beta-galactosidase reaction. For all microplates an absorbance reading is obtained to assay the generation of product from the enzyme substrate. The presence of putative inhibitors of the interaction between BAT3 and PN9113 results in inhibition of the beta-galactosidase signal generated by MY209. Additional testing eliminates compounds that decreased expression of beta-galactosidase by affecting yeast cell growth and non-specific inhibitors that affected the beta-galactosidase signal generated by the interaction of an unrelated protein pair.

Once a hit, i.e., a compound which inhibits the interaction between the interacting proteins, is obtained, the compound is identified and subjected to further testing wherein the compounds are assayed at several concentrations to determine an IC₅₀ value, this being the concentration of the compound at which the signal seen in the two-hybrid assay described in this Example is 50% of the signal seen in the absence of the inhibitor.

### 4. Enzyme-Linked Immunosorbent Assay (ELISA)

pGEX5X-2 (Amersham Biosciences; Uppsala, Sweden) is used for the expression of a GST-PN9113 fusion protein. The pGEX5X-2-PN9113 construct is transfected into Escherichia coli strain DH5a (Invitrogen; Carlsbad, Calif.) and fusion protein is prepared by inducing log phase cells (O.D. 595=0.4) with 0.2 mM isopropyl-β-D-thiogalactopyranoside (IPTG). Cultures are harvested after approximately 4 hours of induction, and cells pelleted by centrifugation. Cell pellets are resuspended in lysis buffer (1% nonidet P-40 [NP-40], 150 mM NaCl, 10 mM Tris pH 7.4, 1 mM ABESF [4-(2-aminoethyl) benzenesulfonyl fluoride]), lysed by sonication and the lysate cleared of insoluble materials by centrifugation. Cleared lysate is incubated with Glutathione Sepharose beads (Amersham Biosciences; Uppsala, Sweden) followed by thorough washing with lysis buffer. The GST-PN9113 fusion protein is then eluted from the beads with 5 mM reduced glutathione. Eluted protein is dialyzed against phosphate buffer saline (PBS) to remove the reduced glutathione.

A stable Drosophila Schneider 2 (S2) myc-BAT3 expression cell line is generated by transfecting S2 cells with pCoHygro (Invitrogen; Carlsbad, Calif.) and an expression vector that directs the expression of the myc-BAT3 fusion protein. Briefly, S2 cells are washed and re-suspended in serum free Express Five media (Invitrogen; Carlsbad, Calif.). Plasmid/liposome complexes are then added (NovaFECTOR, Venn Nova; Pompano Beach, Fla.) and allowed to incubate with cells for 12 hours under standard growth conditions (room temperature, no CO₂ buffering). Following this incubation period fetal bovine serum is added to a final concentration of 20% and cells are allowed to recover for 24 hours. The media is replaced and cells are grown for an additional 24 hours. Transfected cells are then selected in 350 μg/ml hygromycin for three weeks. Expression of myc-BAT3 is confirmed by Western blotting. This cell line is referred to as S2-myc-BAT3.

GST-PN9113 fusion protein is immobilized to wells of an ELISA plate as follows:

Nunc Maxisorb 96 well ELISA plates (Nalge Nunc International; Rochester, N.Y.) are incubated with 100 μl of 10 μg/ml of GST-PN9113 in 50 mM carbonate buffer (pH 9.6) and stored overnight at 4° Celsius. This plate is referred to as the ELISA plate.

A compound dilution plate is generated in the following manner. In a 96 well polypropylene plate (Greiner, Germany) 50 μl of DMSO is pipetted into columns 2-12. In the same polypropylene plate pipette, 10 μl of each compound being tested for its ability to modulate protein-protein interactions is plated in the wells of column 1 followed by 90 μl of DMSO (final volume of 100 μl). Compounds selected from primary screens or from virtual screening, or designed based on the primary screen hits are then serially diluted by removing 50 μl from column 1 and transferring it to column 2 (50:50 dilution). Serial dilutions are continued until column 10. This plate is termed the compound dilution plate.

Next, 12 μl from each well of the compound dilution plate is transferred into its corresponding well in a new polypropylene plate. 108 μl of S2-myc-BAT3-containing lysate (1×10⁶ cell equivalents/ml) in phosphate buffered saline is added to all wells of columns 1-11. 108 μl of phosphate buffered saline without lysate is added into all wells of column 12. The plate is then mixed on a shaker for 15 minutes. This plate is referred to as the compound preincubation plate.

The ELISA plate is emptied of its contents and 400 μl of Superblock (Pierce Endogen; Rockford, Ill.) is added to all the wells and allowed to sit for 1 hour at room temperature. 100 μl from all columns of the compound preincubation plate are transferred into the corresponding wells of the ELISA binding plate. The plate is then covered and allowed to incubate for 1.5 hours room temperature.

The interaction of the myc-tagged BAT3 with the immobilized GST-PN9113 is detected by washing the ELISA plate followed by an incubation with 100 μl/well of 1 μg/ml of mouse anti-myc IgG (clone 9E10; Roche Applied Science; Indianapolis, Ind.) in phosphate buffered saline. After 1 hour at room temperature, the plates are washed with phosphate buffered saline and incubated with 100 μl/well of 250 ng/ml of goat anti-mouse IgG conjugated to horseradish peroxidase in phosphate buffer saline. Plates are then washed again with phosphate buffered saline and incubated with the fluorescent substrate solution Quantiblu (Pierce Endogen; Rockford, Ill.). Horseradish peroxidase activity is then measured by reading the plates in a fluorescent plate reader (325 nm excitation, 420 nm emission).

### 5. Effects of Antisense Inhibitors on Protein Expression

The effects of antisense inhibitors on protein expression can be measured by a variety of methods known in the art. A preferred method is to measure mRNA levels using real-time quantitative polymerase chain reaction (PCR) methods. Real-time PCR can be performed using the ABI PRISM™ 7700 Sequence Detection System according to the manufacturer's instructions. The ABI PRISM™ 7700 Sequence Detection System is available from PE-APPLIED Biosystems, Foster City, Calif.

Other methods of measuring mRNA levels may also be used to determine the effects of anitisense inhibitors on proteins. For example competitive PCR and Northern blot analysis are well known in the art and may be performed to determine mRNA levels. Specifically, methods of RNA isolation and Northern blot analysis may be performed according to Ausubel, F. M. et al., Current Protocols in Molecular Biology, Volume 1, pp. 4.1.1-4.2.9 and 4.5.1-4.5.3, John Wiley & Sons, Inc., 1993.

The effects of antisense inhibitors on protein expression may also be determined by measuring protein levels of the proteins of interest. Various methods known in the art may be used, such as immunoprecipitation, Western blot analysis, ELISA, or fluorescence-activated cell sorting (FACS). Antibodies to the proteins of interest are often commercially available, and may be found by such sources as the MSRS catalogue of antibodies (Aerie Corporation, Birmingham, Mich.). Antibodies can also be prepared through conventional antibody generation methods, such as found in Ausubel, F. M. et al., Current Protocols in Molecular Biology, Volume 2, pp. 11.12.1-11.12.9 and 11.4.1-11.11.5 John Wiley & Sons, Inc., 1997. Furthermore, immunoprecipitation analysis can be performed according to Ausubel, F. M. et al., Current Protocols in Molecular Biology, Volume 2, pp. 10.16.1-10.16.11, John Wiley & Sons, Inc., 1997, and ELISA can be performed according to Ausubel, F. M. et al., Current Protocols in Molecular Biology, Volume 2, pp. 11.1.1-11.2.22, John Wiley & Sons, Inc., 1997 or as described in Example 4, above.

Although the foregoing invention has been described in some detail by way of illustration and example for purposes of clarity of understanding, it will be obvious that certain changes and modifications may be practiced within the scope of the appended claims.

In various parts of this disclosure, certain publications or patents are discussed or cited. The mere discussion of, or reference to, such publications or patents is not intended as admission that they are prior art to the present invention.

What is claimed is:

1. An isolated protein complex comprising a first protein interacting with a second protein, said first protein being:
(a) a bait protein identified in any one of Tables 1 through 84,
(b) a homologue of (a) that interacts with said second protein and has an amino acid sequence at least 85% identical to that of (a),
(c) a fragment of (a) or (b) that interacts with said second protein, or
(d) a fusion protein comprising (a), (b), or (c); and
said second protein being:
(i) an interacting prey protein identified in the same table as said bait protein,
(ii) a homologue of (i) that interacts with said first protein and has an amino acid sequence at least 85% identical to that of (i),
(iii) a fragment of (i) or (ii) that interacts with said first protein, or
(iv) a fusion protein comprising (i), (ii), or (iii).

2. The isolated protein complex of claim 1 wherein said first protein and said second protein are both fusion proteins.

3. A method of making the isolated protein complex of claim 1 comprising:
providing said first protein and said second protein, and
contacting said first and second proteins under conditions that allow said first and second proteins to interact to form said isolated protein complex.

4. A method for selecting modulators of a protein complex of claim 1, comprising:
providing said first protein and said second protein;
contacting said first protein and said second protein in the presence and absence of a test compound; and
detecting the protein complex formed by the interaction between said first protein and said second protein in the presence and absence of said test compound.

5. The method of claim 4, wherein said detecting step comprises measuring the amount of the protein complex formed.

6. The method of claim 4, further comprising a step of generating a data set defining one or more selected test compounds, said data set being embodied in a transmittable form.

7. The method of claim 4, wherein at least one of said first and second proteins is a fusion protein having a detectable tag.

8. The method of claim 4, wherein said contacting step is conducted in a substantially cell free environment.

9. The method of claim 4, wherein the interaction between said first protein and said second protein occurs within a host cell.

10. The method of claim 9, wherein said host cell is a yeast cell.

11. A method for identifying modulators of an isolated protein complex of claim 1, comprising:
providing said isolated protein complex;
contacting said isolated protein complex with a test compound; and
determining whether the amount of said isolated protein complex is altered in the presence of said test compound, relative to the absence of said test compound.

12. A method for modulating, in a host cell, a protein complex of claim 1, comprising:
administering to said cell an siRNA or antisense oligonucleotide that causes the reduction of expression of said bait protein or said interacting prey protein.

13. A protein microarray comprising an isolated protein complex according to claim 1.

14. A method of treating Alzheimer's disease, mild cognitive impairment, Huntington's disease, Parkinson's disease, schizophrenia, depression, dementia, diabetes, obesity or coronary artery disease, comprising:
identifying a patient in need of treatment; and
administering to a patient in need of such treatment a compound that modulates the interaction between a first protein, which is a bait protein of one of Tables 1 through 84, and a second protein, which is the corresponding prey protein from said one of Tables 1 through 84.

15. The method of claim 14 wherein said compound that modulates the interaction between said first and second proteins binds to said first protein or to said second protein.

16. The method of claim 15 wherein said compound that modulates the interaction between said first and second proteins disrupts or interferes with the interaction of said first protein with said second protein.

17. A method of treating Alzheimer's disease, mild cognitive impairment, Huntington's disease, Parkinson's disease, schizophrenia, depression, dementia, diabetes, obesity or coronary artery disease, comprising:
identifying a patient in need of treatment; and
administering to a patient in need of such treatment an siRNA or antisense oligonucleotide that reduces the concentration of one of the protein complexes of Tables 1 through 84, by causing the reduction of expression of the bait protein or the interacting prey protein that interact to form said one of the protein complexes of Tables 1 through 84.

18. A method of detecting an alteration associated with a disease or disorder selected from Alzheimer's disease, mild cognitive impairment, Huntington's disease, Parkinson's disease, schizophrenia, depression, dementia, diabetes, obesity or coronary artery disease, comprising:
identifying a patient with said disease or disorder,
obtaining a sample from said patient, and
assaying said sample for an alteration in the nucleotide sequence of the gene or genes encoding one or both of the proteins identified in one of Tables 1 through 84, or an alteration in the level of expression of the gene or genes encoding one or both of the proteins identified in one of the Tables 1 through 84, as compared to patients without said disease or disorder;
wherein the detection of said alteration in said sample identifies said alteration as being associated with said disease or disorder.

19. The method of claim 18, wherein said alteration is an alteration in the nucleotide sequence of the gene or genes encoding one or both of the proteins identified in one of the Tables 1 through 84.

20. The method of claim 18, wherein said alteration is an alteration in the level of expression of one or both of the proteins identified in one of the Tables 1 through 84.

21. The method of claim 18 further comprising the step of determining if said alteration has been inherited.

22. A method of genotyping an individual with a disease or disorder selected from Alzheimer's disease, mild cognitive impairment, Huntington's disease, Parkinson's disease, schizophrenia, depression, dementia, diabetes, obesity or coronary artery disease, comprising:
identifying an individual with said disease or disorder, and
determining if said individual has alteration in the nucleotide sequence of the gene or genes encoding one or both of the proteins identified in one of the Tables 1 through 84, or an alteration in the level of expression of the gene or genes encoding one or both of the proteins identified in one of the Tables 1 through 84, as compared to patients without said disease or disorder.

23. The method of claim 22, wherein said alteration is an alteration in the nucleotide sequence of the gene or genes encoding one or both of the proteins identified in one of the Tables 1 through 84.

24. The method of claim 22, wherein said alteration is an alteration in the level of expression of one or both of the proteins identified in one of the Tables 1 through 84.

25. The method of claim 21 further comprising the step of determining if said alteration has been inherited.


SEQUENCE LISTING (283 sequences)

SEQ ID NO: 1 - DNA, 2740 bp, Homo sapiens
cgagaatttccagcaggcaaggcagtggccgctttgactgcttgcttcggagatccgagacgacggagaaggcactcttatttaccgaccaagaaagctcctcccccgtcctccgttagctaattaaaacatttttcagggacgtagccatccagagacattccattattgttccattgacctttccctcatcactgagtcctttggagctgagttatgtcaacagctgccttaattactttggtcagaagtggtgggaaccaggtgagaaggagagtgctgctaagctcccgcctgctgcaggacgacaggcgggtgacacccacgtgccacagctccacttcagagcctaggtgttctcggtttgacccagatggtagtgggagtccagctacctgggacaattttgggatctgggataaccgcattgatgagccaattctgctgccacccagcattaagtatggcaagccaattcccaaaatcagcttggaaaatgtggggtgcgcctcacagattggcaaacggaaagagaatgaagatcggtttgacttcgctcagctgacagatgaggtcctgtactttgcagtgtatgatggacacggtggacctgcagcagctgatttctgtcatacccacatggagaaatgtattatggatttgcttcctaaggagaagaacttggaaactctgttgaccttggcttttctagaaatagataaagccttttcgagtcatgcccgcctgtctgctgatgcaactcttctgacctctgggactactgcaacagtagccctattgcgagatggtattgaactggttgtagccagtgttggggacagccgggctattttgtgtagaaaaggaaaacccatgaagctgaccattgaccatactccagaaagaaaagatgaaaaagaaaggatcaagaaatgtggtggttttgtagcttggaatagtttggggcagcctcacgtaaatggcaggcttgcaatgacaagaagtattggagatttggaccttaagaccagtggtgtcatagcagaacctgaaactaagaggattaagttacatcatgctgatgacagcttcctggtcctcaccacagatggaattaacttcatggtgaatagtcaagagatttgtgactttgtcaatcagtgccatgatcccaacgaagcagcccatgcggtgactgaacaggcaatacagtacggtactgaggataacagtactgcagtagtagtgccttttggtgcctggggaaaatataagaactctgaaatcaacttctcattcagcagaagctttgcctccagtggacgatgggcctgattaccagctgggacttagagtttctgtgcaacagtttttcactgagcatgtcaagaaactgataagatcaaaaaggtctcctaactcactagatcagcgcacaagtcagtgtaaaccacttagatagtagttttttcataaatgctcatcatatttatgttccgctgtacatgttcagtataaatatatgtgtagtgaagctactgtgagtctttaaatggaaagagcaaatgagaagtggtttggatacacttgatgagagatgagagtgtcacattaataatttttaagactcttaggcagctatgggtttcttttgatcatttttgttctttattcatttgaacacgtttttgaagttcttcaaaactagtcagtttgaattttgacagctattcaatatgtgatctccaagtttaaaaaaatttttttccagacttccctaatcctaaaatgcgagtttttatttttaataactgtaccaaggaataagtatgaaaacagttctctgttaccatattttgtattctggaccacttactggtgaaagcaaccatgcaaaagaaattaatttggccaggcacagtggctcatgcctgtaatcccaaattgctgggattacagcactgtgccctcctaggaaattattttttaagtgaaattttatttttattttttttaggattttggtagagaatgagtaggcctactcatcaatatcaaacaggacatttagtttctttccttagaacagacataaatttaatttcatggtaatatgataataagaaaatgcttctatttttctttagcacctccatggttctcatatacccatgtctgtaaaaagtgacatgagaattttgttgggttacattttattgtatttattagattcgcttatatagatgacttaggcagaaataaagtcatgtctttagaaggtgaacaagccaacttgtgatggcctgccttttgcttttggcagttgggatgagaacaattgactctcccattggttgttagatagttgaaatggtgcgttggtggtcatacttagtgttctaggctgtgaaatcatggagttcttccacttccaagaatgactcatttgctgttggattctagtacagaatttagcagcctgatgtgtccccaaactgatttaatttctactgaagtgcccttgtgtacatttgttttgtaatttaccaaagtactacctgagtgtataatgactcctgcagtgagttaatgtaattgctgctttgaccattgttttaaatctgtgtactagagtaactgtgagcagaatgaaatcacattatctcagtgttcaaaatatcattctaataaagtacatgcattaaacaattttaaaaaaaacaaaaaaaaaaaaaaaaaa

SEQ ID NO: 2 - protein, 372 aa, Homo sapiens
MSTAALITLVRSGGNQVRRRVLLSSRLLQDDRRVTPTCHSSTSEPRCSRFDPDGSGSPATWDNFGIWDNRIDEPILLPPSIKYGKPIPKISLENVGCASQIGKRKENEDRFDFAQLTDEVLYFAVYDGHGGPAAADFCHTHMEKCIMDLLPKEKNLETLLTLAFLEIDKAFSSHARLSADATLLTSGTTATVALLRDGIELVVASVGDSRAILCRKGKPMKLTIDHTPERKDEKERIKKCGGFVAWNSLGQPHVNGRLAMTRSIGDLDLKTSGVIAEPETKRIKLHHADDSFLVLTTDGINFMVNSQEICDFVNQCHDPNEAAHAVTEQAIQYGTEDNSTAVVVPFGAWGKYKNSEINFSFSRSFASSGRWA

SEQ ID NO: 3 - DNA, 4173 bp, Homo sapiens
atggcgcgggccaagctgccgcgctcgccgtccgagggcaaggcgggcccggggggcgccccagccggcgccgcagcccccgaggagcctcacgggctcagcccgctgctgccggcccgcggcgggggctccgtgggcagcgacgtgggccagaggcttcctgtagaagatttcagcctggactcctccctgtctcaggtccaggtggagttctacgtcaacgagaacaccttcaaggagcggctcaagctgttcttcatcaaaaaccaaagatcgagcctgaggatccggctgttcaacttctccctgaagctgctcacctgcctgctctacattgtgcgcgtcctgctcgatgacccggccctgggcatcggatgctggggctgcccaaagcagaactactccttcaatgactcgtcctccgagatcaactgggctcctattctgtgggtggagagaaagatgacactgtgggcgatccaggtcatcgtggccataataagcttcctggagacgatgcttctcatctacctcagctacaaaggcaacatctgggagcagatcttccgcgtgtccttcgtcctggagatgatcaacactctgcccttcatcatcacgatcttctggccgccgctgcggaacctgttcatccccgtctttctgaactgctggctggccaagcacgcgctggaaaacatgattaatgacttccaccgtgccatcctgcggacacagtcagccatgttcaaccaggtcctcatcctcttctgcaccctgctgtgcctcgttttcacggggacctgcggcatccagcacctggagcgggcgggcgagaacctgtccctcctgacctccttctacttctgcatcgtcaccttctccaccgtgggctacggtgacgtcacgcccaagatctggccatcgcagctgctggtggtcatcatgatctgcgtggccctcgtggtgctcccactgcagttcgaggagctcgtctacctctggatggagcggcagaagtcagggggcaactacagccgccaccgtgcgcagacggagaagcacgtggtcctgtgtgtcagctccctcaagatcgaccttctcatggacttcctgaacgagttctacgcccacccccggctccaggactattacgtggtcatcctgtgccccacggagatggatgtccaggtgcgcagagtcctgcagatccctctgtggtcccagcgggtcatctacctccagggctctgcactcaaagaccaggacctcatgcgagccaagatggacaatggggaggcctgcttcatcctcagcagcaggaacgaggtggaccgcacggctgcagaccaccagaccatcctgcgcgcctgggccgtgaaggacttcgcccccaactgccccctctacgtccagatcctcaaacctgaaaacaagtttcacgtcaagtttgctgaccacgtggtgtgtgaggaggagtgcaagtacgccatgctggcgctgaactgcatctgcccggcgacctccaccctcatcaccctgctggtgcacacgtcccgcggccaggagggacaggagtctccggagcagtggcagcgcatgtatgggcgctgctccggcaacgaggtgtaccacatccgcatgggtgacagcaagttcttccgcgagtacgagggcaagagcttcacctacgcggccttccacgcccacaagaagtatggcgtgtgcctcatcgggctgaagcgggaggacaacaagagcatcctgctgaacccggggccccggcacatcctggccgcctctgacacctgcttctacatcaacatcaccaaggaggagaactcggccttcatcttcaagcaggaggagaagcggaagaagagggccttctcggggcaggggctgcacgagggtccggcccgcctgcccgtgcacagcatcatcgcctccatggggacagtggccatggacctgcagggcacagagcaccggcctacgcagagcggcggtgggggcgggggcagcaagctggcactgcccacggagaacggctcgggcagccggcggcccagcatcgcgcccgtcctggaactggccgacagctcagccctgctgccctgcgacctgctgagcgaccagtcggaggatgaggtgacgccgtcggacgacgaggggctctccgtggtagagtatgtgaagggctaccctcccaactcgccctacatcggcagctccccaaccctgtgccacctcctgcctgtgaaagcccccttctgctgcctgcggctggacaagggctgcaagcacaacagctatgaagacgccaaggcctacgggttcaagaacaagctgatcatcgtctcggcagagacggccggcaatgggctgtacaacttcatcgtgccactgcgggcctactacagatcccgcaaggagctgaaccccatcgtgctgctgctggacaacaagcccgaccaccacttcctggaagccatctgctgcttccccatggtctactacatggagggctctgtggacaacctggacagcctgctgcagtgtggcatcatctatgcggacaacctggtggtggtggacaaggagagcaccatgagcgccgaggaggactacatggcggacgccaagaccatcgtcaacgtgcagaccatgttccggctcttccccagcctcagcatcaccacggagctcacccacccttccaacatgcgcttcatgcagttccgcgccaaggacagctactctctggctctttccaaactagaaaagagggagcgagagaatggctccaacctggccttcatgttccgcctgccgttcgccgccggccgcgtcttcagcatcagcatgttggacacactgctctaccagtccttcgtgaaggactacatgatcaccatcacccggctgctgctgggcctggacaccacgccgggctcggggtacctctgtgccatgaaaatcaccgagggcgacctgtggatccgcacgtacggccgcctcttccagaagctctgctcctccagcgccgagatccccattggcatctaccggacagagagccacgtcttctccacctcggagccccacgacctcagagcccagtcccagatctcggtgaacgtggaggactgtgaggacacacgggaagtgaaggggccctggggctcccgcgctggcaccggaggcagctcccagggccgccacacgggcggcggtgaccccgcagagcacccactgctacggcgcaagagcctgcagtgggcccggaggctgagccgcaaggcgcccaagcaggcaggccgggcggcggccgcggagtggatcagccagcagcgcctcagcctgtaccggcgctctgagcgccaggagctctccgagctggtgaagaaccgcatgaagcacctggggctgcccaccaccggctacgaggacgtagcaaatttaacagccagtgatgtcatgaatcgggtaaacctgggatatttgcaagacgagatgaacgaccaccagaacaccctctcctacgtcctcatcaaccctccgcccgacacgaggctggagcccagtgacattgtctatctcatccgctccgaccccctggctcacgtggccagcagctcccagagccggaagagcagctgcagccacaagctgtcgtcctgcaaccccgagactcgcgacgagacacagctctgagccagccctgcacggagctcaggccaccaagcccggggtcctcaggaaggacgtggaggagcgtgtgaggacacggtggcactagcgtgaccctggggatggcacactctactcaccatggctcctgggactccaccctggaaaggagcccctcatgcggggggagggccagctcacccctgggcacctgcaggctagtgaggagagttttttaacctatttttacacgtcgatgcagtccacttctctttacacagatgtaccgcaactcgtgaccagggctggctgggagggcaacgcagggactggacgccctacagggccgagcccaggctgtgctggagggtggggctggggtgcatggggaggggagcagaacccagaacccaggagccccgcgtggccacacccaactcagagccggcctgagcgttcacggccaggcagcctcgcttcctt

SEQ ID NO: 4 - protein, 1228 aa, Homo sapiens
MARAKLPRSPSEGKAGPGGAPAGAAAPEEPHGLSPLLPARGGGSVGSDVGQRLPVEDFSLSSLSQVQVEFYVNENTFKERLKLFFIKNQRSSLRIRLFNFSLKLLTCLLYIVRVLLDDPALGIGCWGCPKQNYSFNDSSSEINWAPILWVERKMTLWAIQVIVAIISFLETMLLIYLSYGNIWEQIFRVSFVLEMINTLPFIITIFWPPLRNLFIPVFLNCWLAKHALENMINDFHRAILRTQSAMFNQVLILFCTLLCLVFTGTCGIQHLERAGENLSLLTSFYFCIVTFSTVGYGDTPKIWPSQLLVVIMICVALVVLPLQFEELVYLWMERQKSGGNYSRHRAQTEKHVVLCVSSLKIDLLMDFLNEFYAHPRLQDYYVVILCPTEMDVQVRRVLQIPLWSQRVIYLQGSALKDDLMRAKMDNGEACFILSSRNEVDRTAADHQTILRAWAVKDFAPNCPLYVQILKPENKFHVKFADHVVCEEECKYAMLALNCICPATSTLITLLVHTSRGQEGQESPEQWQRMYGRCSGNVYHIRMGDSKFFREYEGKSFTYAAFHAHKKYGVCLIGLKREDNKSILLNPGPRHILAASDTCFYINITKEENSAFIFKQEEKRKKRAFSGQGLHEGPARLPVHSIIASMGTVAMDLQGTHRPTQSGGGGGGSKLALPTENGSGSRRPSIAPVLELADSSALLPCDLLSDQSEDEVTPSDDEGLSVVEYVKGYPPNSPYIGSSPTLCHLLPVKAPFCCLRLDKGCKHNSYEDAKAYGFKKLIIVSAETAGNGLYNFIVPLRAYYRSRKELNPIVLLLDNKPDHHFLEAICCFPMVYYMEGSVDNLDSLLQCGIIYADNLVVVDKESTMSAEEDYMADAKTIVNVQTMFRLFPSLSITTLTHPSNMRFMQFRAKDSYSLALSKLEKRERENGSNLAFMFRLPFAAGRVFSISMLDTLLYQSFVKDYMITITRLLLGLDTTPGSGYLCAMKITEGDLWIRTYGRLFQKLCSSSAEIPIGIYRTESHVFSTSEPHDLRAQSQISVNVEDCEDTREVKGPWGSRAGTGGSSQGRHTGGGDPAEHPLLRRKSLQWARRLSRKAPKQAGRAAAAEWISQQRLSLYRRSERQELSELVKNRMKHGLPTTGYEDVANLTASDVMNRVNLGYLQDEMNDHQNTLSYVLINPPPDTRLEPSDIVYLIRSDPLAHVASSSQSRKSSCSHKLSSCNPETRDETQL

SEQ ID NO: 5 - DNA, 4060 bp, Homo sapiens
gactgaatcatatatgggggaaaccacttctcaggcagccagctggttctcaggtcagcaagagacgatccatttttatcggaagctttatagtagcaataatgctaatacccagcactcgggctccacaatgtagaggaaatggcatcgcctggcagtgacagctatattgtgcgtgtcaaggctgtggttatgaccagagatgactccagcgggggatggttcccacaggaaggaggcgggatcagtcgcgtcggggtctgtaaggtcatgcaccccgaaggcaatggacgaagcggctttctcatccatggtgaccgacagaaagacaaactggtggtattggaatgctatgtaagaaaggacttggtctacaccaaagccaatccaacgtttcatcactggaaggtcgataataggaagtttggacttactttccaaagccctgctgatgcccgagcctttgacaggggagtaaggaaagcaatcgaagaccttatagaaggttcaacaacgtcatcttccaccatccataatgaagctgagcttggcgatgatgacgtttttacaacagctacagacagttcttctaattcctctcagaagagagagcaacctactcggacaatctcctctcccacatcctgtgagcaccggaggatttataccctgggccacctccacgactcataccccacagaccactatcacctcgatcagccgatgccaaggccctaccgccaggtgagcttcccggacgacgacgaggagatcgtgcgcatcaacccccgggagaagatctggatgacggggtacgaggattaccggcacgcacccgtcaggggcaagtacccggacccctcggaggacgcggactcctcctacgtgcgcttcgccaagggcgaggtccccaagcatgactacaactacccctacgtggactcctcagactttggcctaggcgaggaccccaaaggccgcgggggcagcgtgatcaagacgcagccctcccggggcaagtcgcggcggcggaaggaggacggagagcgctcgcggtgcgtgtactgcagggacatgttcaaccacgaggagaaccgccggggccactgccaggacgcgcccgactccgtgagaacttgcatccgccgggtgagctgcatgtggtgcgcggacagcatgctctatcactgtatgtcggaccccgagggagactatacagacccttgctcgtgcgatactagcgacgagaagttttgcctccggtggatggctcttattgccttgtctttcctggccccctgtatgtgctgttacctgccccttcgggcctgctaccactgcggagtgatgtgcaggtgctgtggcgggaagcacaaagcggccgcgtgactcagtttccctcccttctccctccatccgcagccacaggggaactcgtctcttacatactctcatcttctcccccgctcccttccactccaaggagcgaggagggcaagcggcctcccagctccctggtacctcgaggcaccattccagccagggacgctgccgggtagactctccactccccctgccgcccacactgcagcagccacatccatacacacacgctcgcacagtgttctgaggaaggaaccttcgccacagactcctgtactattaacaatctgtaaccaagctaactgtctcatccatgtgttgatttcctgtttcctcctcccccgcctcttccagttcaaaggagtctgcaattggaactgctgattttcggtgggttttgtagttgatttttccaagagcgtcgaagactctctttctcttggttcaccttgcctgtcgctagcaagcatctggttcagcggaaatgggatgtgagaatgatgaaacccgacagaagtatctcagcctgcagtcagttattatgtataggaggtgagctagttaacaaacttgtaccacaaaacaatatcgctttaacttttctaaagccaaatttcccatgtaagctgcagtttctatctttagcccatcatcattttctgcccccccaaaatctgttgaaatgattcactgatgcaaaacattcacccgtaatagactgagaattgagccttaacttcagattaacttgtgagaatcaggaaaattccttcaactgcattgcatcctcttggaccagggctagaatggggatttcaggtttctatgagcctccccattacccctaaagtagaacattttttaaattgtgttgccaccacttctaaaaatttagcaatattagaataggatactagtgaagtaagaaattttgcttgttgttttgcaaaccaaatagtttcctcaaacacaaatcagtgttcccacgaacaagttccaattgagaaacactaaggttatggtgaataaaaccatagggagcttcttccccccaacccctggctattttatattaatggggagaggggatttttaaatgtcataaatttgaagagtggtgggttgcattttcttcatgggtttatgttttcgtttcattttggactcaatttcacatcaccaaattcctcatttatacttggggaaaaaacaaggccatatgtaaaaacccttccaatgcctaagtgtctttctcctgcaactccaaacccagactcgccactttgggtgcacaggtggttaggtcagccaactggttctgcctgtcgccttgccacggaggaggcttctaattagctggaaaagagtatttttctaatacgttgcaaggattagccaaatcttcttattgaagaaagaagaaaagtgaagagtggttacctatacctagcatagtacaatcagaacctcgtggagaccaccggggacaggcttgcggacgccggctgttcttcccgccacgatctttctgtggtagcggccagcagaagacatggcctgctccccactccctttccccactccttctttattgcacacaggacaccagtcttcaaggaaagggacttttttccagtctgccaatcatattgggaaagtgctagctgtgctcaccttcatggggctgtttccagctcgtccacaagctcatcgattttctttagtagattaccggtgtaaatacccagtgtgcttatgagtcagttagtagacgtcttcattcattggagtaactggtttaggctttccagtttggaaaaggagcagagagctgtccatctggattgatggaagaaaagagaacctcatccatgcctggagaacatctagaaaaccttcagccagcctccagtgctgtcgagagaccaccttcccccgacccggaggcacttccttggggtctttctctagggtctcttctctacaaagcacaacactaatgttcgtttccttagacctcagttcaagtgcccctatttatttcaataagaacgcacatatcccagctgttttttgtttgtcacctctatttagttgttacctgtttctctcttcttttcaccccttgtcctttttccacccttttaagagttacgctagcagatcttactccacgtatactttttggtttgtgaaggcatcggttaagggcacaaagacagccatggggacatttatgtaaatacgtctctaattgccacactgcagctgaacagtgtgtagtattttcccagtcagctttgccatactgacgtcaatcatttgagagaaattattcagattttatttttgtatctgtggtaacaaaacattaaccaaaagattttctgtccagaagcctccccgaccccccaagctatttgctcacattaacaaattaaagtgcctgaagcataattcattctttacctgtatactaaaaaccctgttgtattgatttttttataataagcctttttacctctgtgtaaaaaatatatatacaagtgtatgatgtacattttagttcttaacttttttttatggtttctaatatgtatgaccaatgtagccattgctttaaaatgtaccgtgtaaatataaacacatcctatcagaaaaaaaaaaaaaaaaaaaa

SEQ ID NO: 6 - protein, 415 aa, Homo sapiens
MASPGSDSYIVRVKAVVMTRDDSSGGWFPQEGGGISRVGVCKVMHPEGNGRSGFLIHGDRQKDKLVVLECYVRKDLVYTKANPTFHHWKVDNRKFGLTFQSPADARAFDRGVRKAIEDLIEGSTTSSSTIHNEAELGDDDVFTTATDSSSNSSQKREQPTRTISSPTSCEHRRIYTLGHLHDSYPTDHYHLDQPMPRPYRQVSFPDDDEEIVRINPREKIWMTGYEDYRHAPVRGKYPDPSEDADSSYVRFAKGEVPKHDYNYPYVDSSDFGLGEDPKGRGGSVIKTQPSRGKSRRRKEDGERSRCVYCRDMFNHEENRRGHCQDAPDSVRTCIRRVSCMWCADSMLYHCMSDPEGDYTDPCSCDTSDEKFCLRWMALIALSFLAPCMCCYLPLRACYHCGVMCRCCGGKHKAAA

SEQ ID NO: 7 - protein, 20 aa, Homo sapiens
ETQEEISAEVAAKAERVHEL

SEQ ID NO: 8 - protein, 20 aa, Homo sapiens
VEQLGAEGNVEESQKVMDEV

SEQ ID NO: 9 - protein, 20 aa, Homo sapiens
EIREKLEELKRVVAEKQEKR

SEQ ID NO: 10 - protein, 50 aa, Homo sapiens
CEVCSAYLGLHDNDRRLADHFGGKLHLGFIEIREKLEELKRVVAEKQEKR

SEQ ID NO: 11 - protein, 50 aa, Homo sapiens
DYEIASKEQDFFFELDAMDHLQSFIADCDRRTEVAKKRLAETQEEISAEV

SEQ ID NO: 12 - protein, 20 aa, Homo sapiens
MEGKWLLCMLLVLGTAIVEA

SEQ ID NO: 13 - protein, 20 aa, Homo sapiens
SSPKVTYKAPVPTGEVYFAD

SEQ ID NO: 14 - protein, 20 aa, Homo sapiens
GGAYVKLLSKTPELNLDQFH

SEQ ID NO: 15 - protein, 20 aa, Homo sapiens
VEEMKESKLPGDKGLVLMSR

SEQ ID NO: 16 - protein, 20 aa, Homo sapiens
SGWILSKAKKDDTDDEIAKY

SEQ ID NO: 17 - protein, 50 aa, Homo sapiens
EGKWLLCMLLVLGTAIVEAHDGHDDDVIDIEDDLDDVIEEVEDSKPDTTA

SEQ ID NO: 18 - protein, 50 aa, Homo sapiens
DTTAPPSSPKVTYKAPVPTGEVYFADSFDRGTLSGWILSKAKKDDTDDEI

SEQ ID NO: 19 - protein, 20 aa, Homo sapiens
MAAAVPRAAFLSPLLPLLLG

SEQ ID NO: 20 - protein, 20 aa, Homo sapiens
DTQYPYGEKQDEFKRLAENS

SEQ ID NO: 21 - protein, 20 aa, Homo sapiens
ELSEKYKLDKESYPVFYLFR

SEQ ID NO: 22 - protein, 20 aa, Homo sapiens
ENPVPYTGAVKVGAIQRWLK

SEQ ID NO: 23 - protein, 20 aa, Homo sapiens
KQGQDNLSSVKETQKKWAEQ

SEQ ID NO: 24 - protein, 50 aa, Homo sapiens
YKLDKESYPVFYLFRDGDFENPVPYTGAVKVGAIQRWLKGQGVYLGMPGC

SEQ ID NO: 25 - protein, 50 aa, Homo sapiens
TFYKVIPKSKFVLVKFDTQYPYGEKQDEFKRLAENSASSDDLLVAEVGIS

SEQ ID NO: 26 - protein, 20 aa, Homo sapiens
MQAQQYQQQRRKFAAAFLAF

SEQ ID NO: 27 - protein, 20 aa, Homo sapiens
CGKLTKPKPQAESKKKKKEG

SEQ ID NO: 28 - protein, 50 aa, Homo sapiens
RTGAECKQTMKTQRCKIPCNWKKQFGAECKYQFQAWGECDLNTALKTRTG

SEQ ID NO: 29 - protein, 20 aa, Homo sapiens
AELKAKAAQLDHQQQCLEDA

SEQ ID NO: 30 - protein, 20 aa, Homo sapiens
LRGRYEKEIRCVVEQLTRTE

SEQ ID NO: 31 - protein, 20 aa, Homo sapiens
KDAYELEVLLRVKESEIQYL

SEQ ID NO: 32 - protein, 20 aa, Homo sapiens
ELNAHNQELNNRLAAEITRL

SEQ ID NO: 33 - protein, 20 aa, Homo sapiens
QRELEVLSEQYSQKCLENAH

SEQ ID NO: 34 - protein, 50 aa, Homo sapiens
GFAAMEETHQKKIEDLQRQHQRELEKLREEKDRLLAEETAATISAIEAMK

SEQ ID NO: 35 - protein, 50 aa, Homo sapiens
NAHREEMERELEKSQRSQISSVNSDVEALRRQYLEELQSVQRELEVLSEQ

SEQ ID NO: 36 - protein, 20 aa, Homo sapiens
ELEKEKDGFENSCKESEPSQ

SEQ ID NO: 37 - protein, 20 aa, Homo sapiens
PSNIFQAQDDSQIQNGYYVN

SEQ ID NO: 38 - protein, 20 aa, Homo sapiens
NNGLLKPDEEKKDLSNKAMP

SEQ ID NO: 39 - protein, 20 aa, Homo sapiens
FNSEKSEIDNELFQFYKEIE

SEQ ID NO: 40 - protein, 20 aa, Homo sapiens
QVIPTFCDTSFTSFRPEWQS

SEQ ID NO: 41 - protein, 50 aa, Homo sapiens
KAFIGPIYKPPEKKKRNEGRNEAHVLNGINDRGGQKEKQKFNSEKSEIDN

SEQ ID NO: 42 - protein, 50 aa, Homo sapiens
NSCKESEPSQEQFVPFYEGHNNGLLKPDEEKKDLSNKAMPSHCDYQQNLG

SEQ ID NO: 43 - protein, 20 aa, Homo sapiens
MALDGPEQMELEEGKAGSGL

SEQ ID NO: 44 - protein, 20 aa, Homo sapiens
SGLRQYYLSKIEELQLIVND

SEQ ID NO: 45 - protein, 50 aa, Homo sapiens
AGSGLRQYYLSKIEELQLIVNDKSQNLRRLQAQRNELNAKVRLLRRELQL

SEQ ID NO: 46 - protein, 20 aa, Homo sapiens
HDENLRKQYEKSSRFMKVGY

SEQ ID NO: 47 - protein, 20 aa, Homo sapiens
SFAAQEKQMEVCEVCGAFLI

SEQ ID NO: 48 - protein, 20 aa, Homo sapiens
SLLAEVERRIRRGHARLALS

SEQ ID NO: 49 - protein, 20 aa, Homo sapiens
VLTDKIDVLLQQIEELGSEG

SEQ ID NO: 50 - protein, 20 aa, Homo sapiens
AKIKATVEELKEKLRKRTEE

SEQ ID NO: 51 - protein, 50 aa, Homo sapiens
EKSSRFMKVGYERDFLRYLQSLLAEVERRIRRGHARLALSQNQQSSGAAG

SEQ ID NO: 52 - protein, 50 aa, Homo sapiens
SFAAQEKQMEVCEVCGAFLIVGDAQSRVDDHLMGKQHMGYAKIKATVEEL

SEQ ID NO: 53 - protein, 20 aa, Homo sapiens
FFSEHVPCNGFSDIENLEGP

SEQ ID NO: 54 - protein, 20 aa, Homo sapiens
ILNMEGRKALTWKYYAKKIL

SEQ ID NO: 55 - protein, 20 aa, Homo sapiens
MIMEIELQSQVLDAMNYVLY

SEQ ID NO: 56 - protein, 20 aa, Homo sapiens
RWCQGAEGATLDIFDYIYID

SEQ ID NO: 57 - protein, 20 aa, Homo sapiens
KECEYLIGQHVTAALYGVVN

SEQ ID NO: 58 - protein, 50 aa, Homo sapiens
SLAFKAGESSMIMEIELQSQVLDAMNYVLYDQLKFKGNRMDYYNALNLYM

SEQ ID NO: 59 - protein, 50 aa, Homo sapiens
FSDIENLEGPEIFFEDELVCILNMEGRKALTWKYYAKKILYYLRQQKILN

SEQ ID NO: 60 - protein, 20 aa, Homo sapiens
QKTVLINNMLRRIQEELKQE

SEQ ID NO: 61 - protein, 20 aa, Homo sapiens
SQPTTEPSDSYREAPPAFSH

SEQ ID NO: 62 - protein, 20 aa, Homo sapiens
SQPTTEPSDSYREAPPAFSH

SEQ ID NO: 63 - protein, 20 aa, Homo sapiens
PCTSSSGTASKMAPVSADDL

SEQ ID NO: 64 - protein, 20 aa, Homo sapiens
GPQESRADDSKLMDSLPGNF

SEQ ID NO: 65 - protein, 50 aa, Homo sapiens
CPTSTSTEAATAATDSVKGTSSEAGTQKLDGPQESRADDSKLMDSLPGNF

SEQ ID NO: 66 - protein, 50 aa, Homo sapiens
RRIQEELKQEGSLRPMFTPSSQPTTEPSDSYREAPPAFSHLASPSSHPCD

SEQ ID NO: 67 - protein, 20 aa, Homo sapiens
NHKVLHYGDLEESPQGEVPH

SEQ ID NO: 68 - protein, 20 aa, Homo sapiens
PVADIKAVVTGKDCPHMKVL

SEQ ID NO: 69 - protein, 50 aa, Homo sapiens
CRLSPNHKVLHYGDLEESPQGEVPHDSLQDKLPVADIKAVVTGKDCPHMK

SEQ ID NO: 70 - protein, 20 aa, Homo sapiens
SKTERNSQTNIIASPLVSTD

SEQ ID NO: 71 - protein, 20 aa, Homo sapiens
NRSMHMANQEANTMSRSQTN

SEQ ID NO: 72 - protein, 20 aa, Homo sapiens
MWAQPRFGAERLSKTERNSQ

SEQ ID NO: 73 - protein, 20 aa, Homo sapiens
AVGGARPKSKAKAIPVSRFK

SEQ ID NO: 74 - protein, 20 aa, Homo sapiens
PGSIDVIKKESCFWPEENAN

SEQ ID NO: 75 - protein, 50 aa, Homo sapiens
RKAHYQAGFQPSFRSKEETNMGSWCCPRPTSKQEASPNSDFKWVDKSVSS

SEQ ID NO: 76 - protein, 50 aa, Homo sapiens
KTNTWSGPREDPNSRSRFRSKKEVYVESSSGSEHEDHLESWFGAGKEGKF

SEQ ID NO: 77 - protein, 20 aa, Homo sapiens
LAEEEDFDSKEWVIIDKETE

SEQ ID NO: 78 - protein, 20 aa, Homo sapiens
FSHMLLTTPQVPLAPVQPQA

SEQ ID NO: 79 - protein, 50 aa, Homo sapiens
EWVIIDKETELKDFPPGAEPSTSGTTDEEPEELRPLPEEGEERRRLGAEP

SEQ ID NO: 80 - protein, 20 aa, Homo sapiens
STSEPRCSRFDPDGSGSPAT

SEQ ID NO: 81 - protein, 20 aa, Homo sapiens
IDEPILLPPSIKYGKPIPKI

SEQ ID NO: 82 - protein, 50 aa, Homo sapiens
QPHVNGRLAMTRSIGDLDLKTSGVIAEPETKRIKLHHADDSFLVLTTDGI

SEQ ID NO: 83 - protein, 20 aa, Homo sapiens
VTGTPPAEFVQRLQSDEAKN

SEQ ID NO: 84 - protein, 20 aa, Homo sapiens
KDFASILTNASPLAVNLLEK

SEQ ID NO: 85 - protein, 50 aa, Homo sapiens
TAGEALAHPYFESLHDTEDEPQVQKYDDSFDDVDRTLDEWKRVTYKEVLS

SEQ ID NO: 86 - protein, 20 aa, Homo sapiens
AMKITEGDLWIRTYGRLFQK

SEQ ID NO: 87 - protein, 20 aa, Homo sapiens
QSQISVNVEDCEDTREVKGP

SEQ ID NO: 88 - protein, 50 aa, Homo sapiens
CEDTREVKGPWGSRAGTGGSSQGRHTGGGDPAEHPLLRRKSLQWARRLSR

SEQ ID NO: 89 - protein, 20 aa, Homo sapiens
QKTVLINNMLRRIQEELKQE

SEQ ID NO: 90 - protein, 20 aa, Homo sapiens
SQPTTEPSDSYREAPPAFSH

SEQ ID NO: 91 - protein, 50 aa, Homo sapiens; identical to SEQ ID NO: 66

SEQ ID NO: 92 - protein, 20 aa, Homo sapiens
KNMTHKLCSDHCFNRYRMAN

SEQ ID NO: 93 - protein, 20 aa, Homo sapiens
SCVAKFQALSMQSSPNGQFV

SEQ ID NO: 94 - protein, 50 aa, Homo sapiens
QFCSKTCSDDYKKLHCIVTYCEYCQEEKTLHETVNFSGVKRPFCSEGCKL

SEQ ID NO: 95 - protein, 20 aa, Homo sapiens
AFSGLYYCDICHQDDASVIP

SEQ ID NO: 96 - protein, 20 aa, Homo sapiens
GDYLGLCRSGALKELSKRLN

SEQ ID NO: 97 - protein, 50 aa, Homo sapiens
RFSVADLQQIADGVYEGFLKALIEFASQHVYHCDLCTQRGFICQICQHHD

SEQ ID NO: 98 - protein, 20 aa, Homo sapiens
QYESLSLFHPSNVEMLSSMD

SEQ ID NO: 99 - protein, 20 aa, Homo sapiens
INTFGKFLADNIVGSVLVFS

SEQ ID NO: 100 - protein, 50 aa, Homo sapiens
NIVGSVLVFSLIFWIPFSILVHCVDKKLDKQYESLSLFHPSNVEMLSSMD

SEQ ID NO: 101 - protein, 20 aa, Homo sapiens
ELGAQPQQDHARWPRGSSLS

SEQ ID NO: 102 - protein, 20 aa, Homo sapiens
KANPNDRRFGILSNCNHSFC

SEQ ID NO: 103 - protein, 20 aa, Homo sapiens
LRDELPFSEDQWDLLHYELE

SEQ ID NO: 104 - protein, 50 aa, Homo sapiens
PGPGGGSFSAYWHQLVEPVRMGEGNMLYKSIKKELVVLRLASLLFKRFLS

SEQ ID NO: 105 - protein, 20 aa, Homo sapiens
YCRDMFNHEENRRGHCQDAP

SEQ ID NO: 106 - protein, 20 aa, Homo sapiens
SCMWCADSMLYHCMSDPEGD

SEQ ID NO: 107 - protein, 50 aa, Homo sapiens
YTDPCSCDTSDEKFCLRWMALIALSFLAPCMCCYLPLRACYHCGVMCRCC

SEQ ID NO: 108 - protein, 20 aa, Homo sapiens
WPTENYGPDSVPDGYQGSQT

SEQ ID NO: 109 - protein, 20 aa, Homo sapiens
VVTQPPPAAYSNSESLLGLL

SEQ ID NO: 110 - protein, 50 aa, Homo sapiens
TQAHTTQPTPFKFNSNFKSNDGDFTFSSPQVVTQPPPAAYSNSESLLGLL

SEQ ID NO: 111 - protein, 20 aa, Homo sapiens
MLLPLLLLLPMCWAVEVKRP

SEQ ID NO: 112 - protein, 20 aa, Homo sapiens
KEQHQKLWEEQLAAAKAQQE

SEQ ID NO: 113 - protein, 50 aa, Homo sapiens
PNGSFHCTNTGYKPLYIPSNRVNDGVCDCCDGTDEYNSGVICENTCKEKG

SEQ ID NO: 114 - protein, 20 aa, Homo sapiens
STSDTTGEENADPPIIHFGP

SEQ ID NO: 115 - protein, 20 aa, Homo sapiens
VDKNMKYIPTEDVSGLSLEE

SEQ ID NO: 116 - protein, 50 aa, Homo sapiens
VSALLNAEDEKREEEKEKQAEEMASDDLSLIRKNRMALFQQLTCVLPILD

SEQ ID NO: 117 - protein, 20 aa, Homo sapiens
MVSSVMKMPEDESTPEKRTE

SEQ ID NO: 118 - protein, 20 aa, Homo sapiens
KSDPSIVRLLQCDPSSAGQF

SEQ ID NO: 119 - protein, 50 aa, Homo sapiens
MVSSVMKMPEDESTPEKRTEKIFRQMDTNRDGKLSLEEFIRGAKSDPSIV

SEQ ID NO: 120 - protein, 20 aa, Homo sapiens
EPEPEEEIIAEDYDDDPVDY

SEQ ID NO: 121 - protein, 20 aa, Homo sapiens
HEKLDRGHDKSDRGHDKSDR

SEQ ID NO: 122 - protein, 50 aa, Homo sapiens
GLPYYWNADTDLVSWLSPHDPNSVVTKSAKKLRSSNADAEEKLDRSHDKS

SEQ ID NO: 123 - protein, 20 aa, Homo sapiens
PAEGDDRFSESILTYVAHTS

SEQ ID NO: 124 - protein, 20 aa, Homo sapiens
LKDLQRQPAEVRGLSGLELD

SEQ ID NO: 125 - protein, 50 aa, Homo sapiens
PESSLSSEEETAGVENVKSQTYSKDLLGQQPHSEPGAAAFGELQNQMPGP

SEQ ID NO: 126 - protein, 20 aa, Homo sapiens
GVGNGRAPDVAPEEVDESKK

SEQ ID NO: 127 - protein, 20 aa, Homo sapiens
STPATTSAPSSGFGTGLFGS

SEQ ID NO: 128 - protein, 20 aa, Homo sapiens
TTTTASTGLSLGGALAGLGG

SEQ ID NO: 129 - protein, 50 aa, Homo sapiens
LKVQEDIKALKQLLSLAANGIQRNTLNIDKLKIETAQELKNAEIALRTQK

SEQ ID NO: 130 - protein, 20 aa, Homo sapiens
DAQLKRMAQDLKDIIEHLNT

SEQ ID NO: 131 - protein, 20 aa, Homo sapiens
EVTKVCEGRRKEQERSFRIT

SEQ ID NO: 132 - protein, 50 aa, Homo sapiens
LKDIIEHLNTSGAPADTSDPLQQICKILNAHMDSLQWIDQNSALLQRKVE

SEQ ID NO: 133 - protein, 20 aa, Homo sapiens
MTLRCLEPSGNGGEGTRSQW

SEQ ID NO: 134 - protein, 50 aa, Homo sapiens
NGGEGTRSQWGTAGSAEEPSPQAARLAKALRELGQTGWYWGSMTVNEAKE

SEQ ID NO: 135 - protein, 20 aa, Homo sapiens
IYADSLKPNIPYKTILLSTT

SEQ ID NO: 136 - protein, 20 aa, Homo sapiens
LMVKGPRHKPGIVQETTFDL

SEQ ID NO: 137 - protein, 50 aa, Homo sapiens
KYAPDDIPNINSTCFKLNSLQLQALLQNYHCAPDEPFIPTDLIENVVTVA

SEQ ID NO: 138 - protein, 20 aa, Homo sapiens
DLDNSGSLSVEEFMSLPELQ

SEQ ID NO: 139 - protein, 20 aa, Homo sapiens
QFSVKGDKEQKLRFAFRIYD

SEQ ID NO: 140 - protein, 50 aa, Homo sapiens
GNNLKDTQLQQIVDKTIINADKDGDGRISFEEFCAVVGGLDIHKKMVVDV

SEQ ID NO: 141 - protein, 20 aa, Homo sapiens
ALGVISNFQSSPKYGSEEDC

SEQ ID NO: 142 - protein, 50 aa, Homo sapiens
AKVTGFSEGVVDSVKGGFSSFSQATHSAAGAVVSKPREIASLIRNKFGSA

SEQ ID NO: 143 - protein, 20 aa, Homo sapiens
TNHEDFIGFANAQQRSSQVH

SEQ ID NO: 144 - protein, 20 aa, Homo sapiens
NFSMDPQLERQVETIRNLVD

SEQ ID NO: 145 - protein, 50 aa, Homo sapiens
KKTTVGNQVIRKGWLTISNIGIMKGGSKGYWFVLTAESLSWYKDDEEKEK

SEQ ID NO: 146 - protein, 20 aa, Homo sapiens
LVLACRRFARAPSLSSGHGS

SEQ ID NO: 147 - protein, 20 aa, Homo sapiens
FHDKAAKDKGFAKWENEKPR

SEQ ID NO: 148 - protein, 50 aa, Homo sapiens
KISVRLQRATTSQATQTEPVWSEAGWGDSGSHTFPKMKSKFHDKAAKDKG

SEQ ID NO: 149 - protein, 20 aa, Homo sapiens
AGVNQRMDSYAHMNGWSNGS

SEQ ID NO: 150 - protein, 20 aa, Homo sapiens
GTPGMALGSMGSVVKSEASS

SEQ ID NO: 151 - protein, 50 aa, Homo sapiens
YSMMQDQLGYPQHPGLNAHGAAQMQPMHRYDVSALQYNSMTSSQTYMNGS

SEQ ID NO: 152 - protein, 20 aa, Homo sapiens
NCGEVNGVIAGVAPSVEAFD

SEQ ID NO: 153 - protein, 20 aa, Homo sapiens
NHLSAVSESIPALGWIAVSP

SEQ ID NO: 154 - protein, 50 aa, Homo sapiens
IWSELQAYIKEHHTTGLTWSKTGPVASTVSAFSVLSSGPGLPPPPPPLPP

SEQ ID NO: 155 - protein, 20 aa, Homo sapiens
KCGGAGHIASDCKFQRPGDP

SEQ ID NO: 156 - protein, 20 aa, Homo sapiens
QQQQQQPPPPPPPSSSMASS

SEQ ID NO: 157 - protein, 50 aa, Homo sapiens
TTQSRPPWMNSGPSESWPYHGMHGGGPGGPGGGPHSFPHPLPSLTGGHGG

SEQ ID NO: 158 - protein, 20 aa, Homo sapiens
LKRATVQRAQHVFQHAVPQE

SEQ ID NO: 159 - protein, 20 aa, Homo sapiens
RLRNLVHSGATKGEISATQD

SEQ ID NO: 160 - protein, 50 aa, Homo sapiens
GRCWIFSCLNVMRLPFMKKLNIEEFEFSQSYLFFWDKVERCYFFLSAFVD

SEQ ID NO: 161 - DNA, 20 bp, Homo sapiens
taactcatgaacacgttctg

SEQ ID NO: 162 - protein, 20 aa, Homo sapiens
CTTTTCTTGGCCACTTCTGT

SEQ ID NO: 163 - DNA, 20 bp, Homo sapiens
tcataatccgctcttaaagc

SEQ ID NO: 164 - DNA, 20 bp, Homo sapiens
cacttcccttccatgatctc

SEQ ID NO: 165 - DNA, 20 bp, Homo sapiens
cttcccttccatgatctcta

SEQ ID NO: 166 - DNA, 20 bp, Homo sapiens
tcccttccatgatctctagc

SEQ ID NO: 167 - DNA, 20 bp, Homo sapiens
acagcggcagccatatcgcc

SEQ ID NO: 168 - DNA, 20 bp, Homo sapiens
agcggcagccatatcgccgg

SEQ ID NO: 169 - DNA, 20 bp, Homo sapiens
cggcagccatatcgccggcc

SEQ ID NO: 170 - DNA, 20 bp, Homo sapiens
tctcggcccaggacttctcc

SEQ ID NO: 171 - DNA, 20 bp, Homo sapiens
ccctcactgactgtgtggca

SEQ ID NO: 172 - DNA, 20 bp, Homo sapiens
tatctggctgatcagcgagg

SEQ ID NO: 173 - DNA, 20 bp, Homo sapiens
tcaccataagacatcttact

SEQ ID NO: 174 - DNA, 20 bp, Homo sapiens
accataagacatcttactct

SEQ ID NO: 175 - DNA, 20 bp, Homo sapiens
cataagacatcttactctac

SEQ ID NO: 176 - DNA, 20 bp, Homo sapiens
ccgtcaagcgccatcttctc

SEQ ID NO: 177 - DNA, 20 bp, Homo sapiens
gtcaagcgccatcttctctc

SEQ ID NO: 178 - DNA, 20 bp, Homo sapiens
caagcgccatcttctctctt

SEQ ID NO: 179 - DNA, 20 bp, Homo sapiens
gcggccgaaatcatggtgct

SEQ ID NO: 180 - DNA, 20 bp, Homo sapiens
ggccgaaatcatggtgcttc

SEQ ID NO: 181 - DNA, 20 bp, Homo sapiens
ccgaaatcatggtgcttcct

SEQ ID NO: 182 - DNA, 20 bp, Homo sapiens
ataagaagtgacttgggaag

SEQ ID NO: 183 - DNA, 20 bp, Homo sapiens
ttgggcctggatgtctttga

SEQ ID NO: 184 - DNA, 20 bp, Homo sapiens
gaacctctttgagaacgagg

SEQ ID NO: 185 - DNA, 20 bp, Homo sapiens
cctttacccaacatatatca

SEQ ID NO: 186 - DNA, 20 bp, Homo sapiens
tttacccaacatatatcaca

SEQ ID NO: 187 - DNA, 20 bp, Homo sapiens
tacccaacatatatcacatg

SEQ ID NO: 188 - DNA, 20 bp, Homo sapiens
ttcaccacctgcattaccaa

SEQ ID NO: 189 - DNA, 20 bp, Homo sapiens
caccacctgcattaccaagc

SEQ ID NO: 190 - DNA, 20 bp, Homo sapiens
ccacctgcattaccaagctc

SEQ ID NO: 191 - DNA, 20 bp, Homo sapiens
cctgtgtccactgacagcat

SEQ ID NO: 192 - DNA, 20 bp, Homo sapiens
cctcagagggcctcccgggc

SEQ ID NO: 193 - DNA, 20 bp, Homo sapiens
caaaggcctcattctcggca

SEQ ID NO: 194 - DNA, 20 bp, Homo sapiens
gcagctgttgacataactca

SEQ ID NO: 195 - DNA, 20 bp, Homo sapiens
agctgttgacataactcagc

SEQ ID NO: 196 - DNA, 20 bp, Homo sapiens
ctgttgacataactcagctc

SEQ ID NO: 197 - DNA, 20 bp, Homo sapiens
ggcggagagctcatggcagg

SEQ ID NO: 198 - DNA, 20 bp, Homo sapiens
cggagagctcatggcaggcc

SEQ ID NO: 199 - DNA, 20 bp, Homo sapiens
gagagctcatggcaggcccg

SEQ ID NO: 200 - DNA, 20 bp, Homo sapiens
tcctccgactggtcgctcag

SEQ ID NO: 201 - DNA, 20 bp, Homo sapiens
cacccatgcggatgtggtac

SEQ ID NO: 202 - DNA, 20 bp, Homo sapiens
gagccctggaggtagatgac

SEQ ID NO: 203 - DNA, 20 bp, Homo sapiens
ggatagtagaacattgtccc

SEQ ID NO: 204 - DNA, 20 bp, Homo sapiens
atagtagaacattgtccctc

SEQ ID NO: 205 - DNA, 20 bp, Homo sapiens
agtagaacattgtccctctg

SEQ ID NO: 206 - DNA, 20 bp, Homo sapiens
gaacttgtgtccatgccaaa

SEQ ID NO: 207 - DNA, 20 bp, Homo sapiens
acttgtgtccatgccaaaga

SEQ ID NO: 208 - DNA, 20 bp, Homo sapiens
ttgtgtccatgccaaagaac

SEQ ID NO: 209 - DNA, 20 bp, Homo sapiens
accactgaaagcatctccac

SEQ ID NO: 210 - DNA, 20 bp, Homo sapiens
cactgaaagcatctccactc

SEQ ID NO: 211 - DNA, 20 bp, Homo sapiens
ctgaaagcatctccactcac

SEQ ID NO: 212 - DNA, 20 bp, Homo sapiens
agagactgcctcatgttccc

SEQ ID NO: 213 - DNA, 20 bp, Homo sapiens
agactgcctcatgttcccgg

SEQ ID NO: 214 - DNA, 20 bp, Homo sapiens
actgcctcatgttcccggcc

SEQ ID NO: 215 - DNA, 20 bp, Homo sapiens
tcccatttgttgtccagcac

SEQ ID NO: 216 - DNA, 20 bp, Homo sapiens
gaagagggagataagagaga

SEQ ID NO: 217 - DNA, 20 bp, Homo sapiens
gcgttctctatcgtctgcca

SEQ ID NO: 218 - DNA, 20 bp, Homo sapiens
gcaggctcttccatggctgc

SEQ ID NO: 219 - DNA, 20 bp, Homo sapiens
aggctcttccatggctgctt

SEQ ID NO: 220 - DNA, 20 bp, Homo sapiens
gctcttccatggctgctttg

SEQ ID NO: 221 - DNA, 20 bp, Homo sapiens
ccaggcgatgccatttcctc

SEQ ID NO: 222 - DNA, 20 bp, Homo sapiens
aggcgatgccatttcctcta

SEQ ID NO: 223 - DNA, 20 bp, Homo sapiens
gcgatgccatttcctctaca

SEQ ID NO: 224 - DNA, 20 bp, Homo sapiens
ttgctgcgcctcatcgcgcc

SEQ ID NO: 225 - DNA, 20 bp, Homo sapiens
gctgcgcctcatcgcgccgc

SEQ ID NO: 226 - DNA, 20 bp, Homo sapiens
tgcgcctcatcgcgccgcca

SEQ ID NO: 227 - DNA, 20 bp, Homo sapiens
agcggcaacagcatctcacc

SEQ ID NO: 228 - DNA, 20 bp, Homo sapiens
cggcaacagcatctcaccag

SEQ ID NO: 229 - DNA, 20 bp, Homo sapiens
gcaacagcatctcaccagac

SEQ ID NO: 230 - DNA, 20 bp, Homo sapiens
gcagttttgtgcatgagtag

SEQ ID NO: 231 - DNA, 20 bp, Homo sapiens
agttttgtgcatgagtaggt

SEQ ID NO: 232 - DNA, 20 bp, Homo sapiens
ttttgtgcatgagtaggtga

SEQ ID NO: 233 - DNA, 20 bp, Homo sapiens
ttctgtttccccatcctggc

SEQ ID NO: 234 - DNA, 20 bp, Homo sapiens
ctgtttccccatcctggcgg

SEQ ID NO: 235 - DNA, 20 bp, Homo sapiens
gtttccccatcctggcggca

SEQ ID NO: 236 - DNA, 20 bp, Homo sapiens
catctttgcggctcgtcgct

SEQ ID NO: 237 - DNA, 20 bp, Homo sapiens
catgactacgatctgacttc

SEQ ID NO: 238 - DNA, 20 bp, Homo sapiens
ggtggcagaccctctatccg

SEQ ID NO: 239 - DNA, 20 bp, Homo sapiens
tgctgggacgacattttttg

SEQ ID NO: 240 - DNA, 20 bp, Homo sapiens
ctgggacgacattttttggc

SEQ ID NO: 241 - DNA, 20 bp, Homo sapiens
gggacgacattttttggctt

SEQ ID NO: 242 - DNA, 20 bp, Homo sapiens
agcggtggccccatccccgc

SEQ ID NO: 243 - DNA, 20 bp, Homo sapiens
cggtggccccatccccgcag

SEQ ID NO: 244 - DNA, 20 bp, Homo sapiens
gtggccccatccccgcagcc

SEQ ID NO: 245 - DNA, 20 bp, Homo sapiens
cgggcgccgactcggcctcc

SEQ ID NO: 246 - DNA, 20 bp, Homo sapiens
cgctcctgcctcagcagccg

SEQ ID NO: 247 - DNA, 20 bp, Homo sapiens
cgtggctccgtcttcatcat

SEQ ID NO: 248 - DNA, 20 bp, Homo sapiens
aaccctgtggacatgtctgg

SEQ ID NO: 249 - DNA, 20 bp, Homo sapiens
ccctgtggacatgtctggcc

SEQ ID NO: 250 - DNA, 20 bp, Homo sapiens
ctgtggacatgtctggccag

SEQ ID NO: 251 - DNA, 20 bp, Homo sapiens
ttaaagccgctcatggctcc

SEQ ID NO: 252 - DNA, 20 bp, Homo sapiens
aaagccgctcatggctccgg

SEQ ID NO: 253 - DNA, 20 bp, Homo sapiens
agccgctcatggctccggac

SEQ ID NO: 254 - DNA, 20 bp, Homo sapiens
caccgcagggtcatgagaga

SEQ ID NO: 255 - DNA, 20 bp, Homo sapiens
ccgcagggtcatgagagaag

SEQ ID NO: 256 - DNA, 20 bp, Homo sapiens
gcagggtcatgagagaaggg

SEQ ID NO: 257 - DNA, 20 bp, Homo sapiens
ctggggtccacaaacttaaa

SEQ ID NO: 258 - DNA, 20 bp, Homo sapiens
tctactaaatagggcagctt

SEQ ID NO: 259 - DNA, 20 bp, Homo sapiens
agcataacccgggcgatgca

SEQ ID NO: 260 - DNA, 20 bp, Homo sapiens
gcctcatttcccattttgct

SEQ ID NO: 261 - DNA, 20 bp, Homo sapiens
ctcatttcccattttgctcg

SEQ ID NO: 262 - DNA, 20 bp, Homo sapiens
catttcccattttgctcggc

SEQ ID NO: 263 - DNA, 20 bp, Homo sapiens
cccgaaggctccatcagtag

SEQ ID NO: 264 - DNA, 20 bp, Homo sapiens
cgaaggctccatcagtagac

SEQ ID NO: 265 - DNA, 20 bp, Homo sapiens
aaggctccatcagtagactt

SEQ ID NO: 266 - DNA, 20 bp, Homo sapiens
gagagttccagggtatctac

SEQ ID NO: 267 - DNA, 20 bp, Homo sapiens
agggtatcccgaatgtggtt

SEQ ID NO: 268 - DNA, 20 bp, Homo sapiens
aggtccagtttggtgataac

SEQ ID NO: 269 - DNA, 20 bp, Homo sapiens
cctgctcatcctcctggctg

SEQ ID NO: 270 - DNA, 20 bp, Homo sapiens
ctgatggccctccctgtcaa

SEQ ID NO: 271 - DNA, 20 bp, Homo sapiens
gagaccctctggtaggagtg

SEQ ID NO: 272 - DNA, 20 bp, Homo sapiens
atcatgttgtacatgcgggc

SEQ ID NO: 273 - DNA, 20 bp, Homo sapiens
catgttgtacatgcgggcgc

SEQ ID NO: 274 - DNA, 20 bp, Homo sapiens
tgttgtacatgcgggcgctg

SEQ ID NO: 275 - DNA, 20 bp, Homo sapiens
tgcatgttggccattctgct

SEQ ID NO: 276 - DNA, 20 bp, Homo sapiens
catgttggccattctgcttc

SEQ ID NO: 277 - DNA, 20 bp, Homo sapiens
tgttggccattctgcttcgc

SEQ ID NO: 278 - DNA, 20 bp, Homo sapiens
gctccggtcgccatggcgcc

SEQ ID NO: 279 - DNA, 20 bp, Homo sapiens
tccggtcgccatggcgcccc

SEQ ID NO: 280 - DNA, 20 bp, Homo sapiens
cggtcgccatggcgcccccg

SEQ ID NO: 281 - DNA, 20 bp, Homo sapiens
cccgagctgctcatggcgcc

SEQ ID NO: 282 - DNA, 20 bp, Homo sapiens
cgagctgctcatggcgccca

SEQ ID NO: 283 - DNA, 20 bp, Homo sapiens
agctgctcatggcgcccacg